(19) United States
(12) Patent Application Publication
(10) Pub. No.: US 2004/0142335 A1
(43) Pub. Date: Jul. 22, 2004

(54) Method for determining skin stress or skin ageing in vitro

(76) Inventors: Dirk Petersohn, Koeln (DE); Marcus Conradt, Pretoria (ZA); Kay Hofmann, Koeln (DE)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/450,797

(22) PCT Filed: Dec. 20, 2001
(86) PCT No.: PCT/EP01/15178

(30) Foreign Application Priority Data
Jan. 3, 2001 (DE) 10100121.5

(51) Int. Cl. C12Q 1/68

(52) U.S. Cl. 435/6

(57) ABSTRACT

The invention relates to a method for determining skin stress and/or skin ageing in humans or animals in vitro, test kits and biochips for determining skin stress and/or skin ageing, and the use of proteins, mRNA molecules or fragments of proteins or mRNA molecules as skin stress and/or ageing markers. The invention also relates to a test method for demonstrating the effectiveness of cosmetic or pharmaceutical active ingredients against skin stress and/or skin ageing, a screening method for identifying cosmetic or pharmaceutical active ingredients against skin stress and/or skin ageing, and a method for producing a cosmetic and/or pharmaceutical preparation against skin stress and/or skin ageing. The invention further relates to a cosmetic or pharmaceutical preparation against skin stress and/or skin ageing.

This invention, relates to a process for the in vitro determination of skin stress and/or ageing of the skin in human beings or animals, to test kits and biochips for determining the skin stress and/or ageing of the skin and to the use of proteins, mRNA molecules or fragments of proteins or mRNA molecules as markers for skin stress and/or ageing of the skin; also to a test for demonstrating the effectiveness of cosmetic or pharmaceutical active substances against skin stress and/or ageing of the skin and to a screening process for identifying cosmetic or pharmaceutical active substances against skin stress and/or ageing of the skin and to a process for the production of a cosmetic or pharmaceutical preparation against skin stress and/or ageing of the skin.

Each living cell is capable of reacting to signals from its environment. The reactions of the cells are achieved through the ordered regulation of gene expression so that the metabolism of cells is not static but very dynamic. According to the most recent estimates, the human genome comprises ca. 140,000 genes. However, of this immense supply of information, each cell uses only a small part specific to it for the synthesis of proteins which is reflected in the gene expression pattern. Exogenous signals are received by cells and lead—partly through complex signal transduction cascades—to changes in the gene expression pattern. In this way, each cell reacts to signals from its environment by adaptation of its metabolism.

For example, the skin cells sense the high-energy radiation of the sun and react by reversing their RNA and protein synthesis rates. After a stress stimulus (for example sunlight), some molecules are synthesized to an increasing extent (for example MMP-1) while others are produced to a lesser extent (for example collagen α₁ (I)). In addition, in many of the synthesis processes, no significant change will occur (for example TIMP-1).

The skin is the largest organ of the human body. It is an organ of very complex structure which consists of a large number of different cell types and which forms the interface between the body and the environment. It follows from this that the cells of the skin are particularly exposed to exogenous physical and chemical environmental signals and, accordingly, continuously regulate their gene expression. The analysis of gene expression in the skin is therefore crucially important to understanding reactions of the skin to exogenous stimuli.

The macroscopic phenomena of ageing skin are based on the one hand on intrinsic or chronological ageing (skin ageing) and, on the other hand, on extrinsic ageing by environmental stress (skin stress). The ability of living cells to react to their environment changes with time—ageing processes leading to senescence and ultimately to cell death take place. The visible signs of old skin should be interpreted as the integral of intrinsic and extrinsic ageing (for example by sunlight), the events of extrinsic ageing accumulating in the skin over a prolonged period.

A key feature of the skin is that, with increasing age, the cells lose their ability to maintain the homeostasis of the organ. Which molecular mechanisms are behind this development has hitherto been largely unclear.

Effective anti-ageing products show their effect on a broad spectrum of molecular phenomena of skin ageing. However, only a few molecular events of skin ageing have hitherto been described which could be used as a target for cosmetic anti-ageing products. The identification of new ageing markers makes it possible to understand the complex processes of intrinsic and extrinsic ageing of the skin and their causal relationships. Only with this knowledge can new concepts be developed for cosmetic anti-ageing products which exert their effect on the broad spectrum of extrinsic and intrinsic ageing processes in the skin.

Each cell type of the skin expresses ca. 15,000 different genes and synthesizes a corresponding number of proteins therefrom. However, which of these genes play a role in the ageing of the skin has hitherto been largely unclear.

The skin consists of several different cell types (fibroblasts, keratinocytes in various states of differentiation, melanocytes, Merkel cells, Langerhans cells, hair follicle cells, sweat gland cells, etc.) so that the complexity of genes expressed in the skin is immense. It has not yet been possible to describe this immense complexity. Nor has it yet been possible to identify from this complexity those genes which are relevant to ageing of the skin and which could serve as molecular markers for ageing of the skin.

In addition, a complicating factor is that, in living cells, mRNA molecules occur in concentrations between just a few and several hundred copies. Hitherto, the weakly expressed genes have only been accessible to analyses with great difficulty, if at all. However, these molecules can play a crucial role in ageing processes and in the homeostasis of the skin.

The totality of all the mRNA molecules which are synthesized at a certain time by a cell or a tissue is known as a “transcriptome”. Hitherto, it has not been possible to describe the complete transcriptome, i.e. the totality of all transcribed genes, of the human skin. Although gene expression can be analyzed by quantification of specific mRNA molecules (for example northern blot, RNase protection experiments), only a relatively limited number of genes can be measured by these techniques.

The anti-ageing products available on the market exert their effects on one of the few known markers of extrinsic skin ageing such as, for example, collagen synthesis, collagenase activity or collagenase inhibitors.

Understanding the complex ageing processes in the skin and identifying suitable marker proteins enables a search to be specifically made for substances or combinations of substances with a broad anti-ageing action spectrum. However, product concepts of this type have not yet been developed because many of the skin ageing markers were not known.

Previous efforts to identify ageing markers were carried out using artificial systems. WO 99/52929 (Lifespan Bioscience Inc.) and the work of Danit, L. et al. (2000), Science 287, pp. 2486-2492 describe the identification of ageing markers from isolated and then in vitro cultivated fibroblasts of the skin. The cells are no longer in their natural environment in which they divide extremely slowly, but divide relatively quickly and transpose their gene expression. Another key point is the fact that fibroblasts in the skin are exposed to the influence of adjacent skin cells which is no longer the case with isolated and in vitro cultivated cells.

Accordingly, there is a need to identify as many as possible and preferably all of the genes that are important to ageing of the skin.

Accordingly, the problem addressed by the present invention was to identify as large a number of the genes relevant to ageing of the skin and/or skin stress as possible and to provide processes for determining skin stress and/or ageing of the skin by means of the identified genes.

According to the invention, the solution to this first problem is provided by a process (1) for the in vitro identification of the genes relevant to ageing of the skin and/or to skin stress in human beings or animals which is characterized in that

a) a first mixture of genetically coded factors expressed, i.e. transcribed and optionally translated, in human or animal skin, i.e. a mixture of proteins, mRNA molecules or fragments of proteins or mRNA molecules, is isolated from young human or animal skin,

b) a second mixture of genetically coded factors expressed, i.e. transcribed and optionally translated, in human or animal skin, i.e. a mixture of proteins, mRNA molecules or fragments of proteins or mRNA molecules, is isolated from old human or animal skin,

c) the mixtures isolated in a) and b) are subjected to a serial analysis of gene expression (SAGE) so that the genes expressed to different extents (differentially) in old and young skin are identified.

The process according to the invention, like the other subjects of the invention, is preferably applied to human skin although it may also be applied to animal skin and to skin models based on human or animal skin.

The technique of “serial analysis of gene expression” (SAGE™) was used to determine the transcriptome of the skin. This technique enables all the genes expressed in the skin to be simultaneously identified and quantified. By comparing the transcriptome of young skin with the transcriptome of old skin, it is possible to differentiate between genes that are relevant and non-relevant to ageing of the skin.

Human skin from healthy female donors was used for the SAGE™ analysis. The SAGE™ analysis was carried out as described in EP-A-0 761 822 and in Velculescu, V. E. et al., 1995, Science 270, 484-487. Two SAGE™ libraries of human skin of different age groups were analyzed. The first library comes from a 29-year-old female volunteer (30048 tags) and the other library from a 69-year-old female volunteer (32840 tags). For further analysis, the two SAGE™ libraries were standardized to the average number of tags (31594 tags). The two libraries were compared with one another to identify genes with age-dependent regulation. As expected for two libraries of the same tissue type, the tag repertoire of the two skin libraries is largely similar.

The first feature to be noticed is that the library of young skin has a clear over-expression of collagens. Some collagens have several alternative poly-adenylations; the resulting tags show consistent results. For example, collagen (I) α1 is over-expressed by a factor of 4-5 in the young skin, collagen (I) α2 by a factor of 4 and collagen (III) α1 by a factor of 8. Other gene classes over-expressed in the young skin correspond to marker proteins, for example of the cytoskeleton. Thus, transgelin, desmin, actin, myosin, calponin and tropomyosin are over-represented by a factor of 6to 37.

By contrast, in the library of old skin, some keratins and other keratinocyte-specific genes are over-represented. The epidermal keratins 5, 10 and 14 strongly expressed in both libraries are represented more strongly in the old skin, for example by a factor of about 2, whereas the equally strongly expressed keratin 1 does not show this tendency. Stratifin, desmocollin, MMP2 (gelatinase) and CLSP (keratinocyte-specific calmodulin) are over-represented by a factor of 5 to 8.

Tables 1 to 4 contain a detailed list of the genes differentially expressed in old and young skin as determined by the process according to the invention, indicating

consecutive order numbers in column 1,

the tag sequence used in column 2,

the relative expression frequency determined in young skin in column 3,

the relative expression frequency determined in old skin in column 4,

the quotient of the frequencies (from column 3 and column 4) in column 5,

the significance in column 6,

the UniGene Accession Number in column 7 and

a brief description of the gene or gene product in column 8.

The quotient in column 5 indicates the strength of the differential expression, i.e. the factor by which the particular gene is expressed more strongly in young skin than in old skin or vice versa.

The particular genes or gene products are disclosed under their UniGene Accession Number in the databank of the National Center for Biotechnology Information (NCBI). This databank is accessible on-line at the following address: http://www.ncbi.nlm.nih.qov/.

In addition, the genes or gene products are directly accessible at the following internet addresses:

http://www.ncbi.nlm.nih.gov/UniGene/Hs.Home.html or

http://www.ncbi.nlm.nih.gov/qenome/quide.

All genes which are expressed differentially by a factor of at least 2 and less than 5 are listed in Table 1.

All genes which are expressed differentially by a factor of at least 5 and less than 7 are listed in Table 2.

All genes which are expressed differentially by a factor of at least 7 and less than 10 are listed in Table 3.

All genes which are expressed differentially by a factor of at least 10 are listed in Table 4.

Genes which are expressed differentially by a factor of at least 2 but to which no data bank entry could be assigned, so that they can only be identified by the tag sequence in column 2 are listed in Table 5.

Genes which are expressed differentially by a factor of at least 2 and which are defined in column 2 by their UniGene Accession Number or in column 3 by their Swissprot or TREMBL number or in column 4 by their EMBL/Genbank number are listed in Table 6.

Genes which are expressed differentially by a factor of 2.89 to 11.10 are listed in Table 7. The assignment of the tags to the genes defined by their Unigene Accession Number in column 6 was done by manual annotation.

The following data banks were used for the annotation:

1. Unigene—30.10.01 version with the following data bank entries:

a. known genes from GenBank (12.10.01)

b. ESTs from dbEST (19.10.01)

2. mRNA—version released on 17.10.01

The data banks were downloaded from the NCBI, formatted for a local version of the BLAST program (also NCBI) and compared for identical hits with the tags detected in the SAGE analysis.

The genes/clones found were checked for redundancy and finished as indicated below:

1. tag sequences with several different hits: evaluation as non-annotatable

2. tag sequences with double or several identical hits: elimination of the hits situated furthest away from the poly-A-tail.

The results from the Unigene databank were evaluated first and then compared with the results from the mRNA databank. The latter do not appear in Table 7 because they can also be called off via the Unigene entries.

All the links shown in the Results Table were tested on the 30.10.2001 database documented in the following (Unigene databank release: UniGene Build #143):

Sequences Included in Unigene

Known genes are from GenBank (Oct. 12, 2001)

ESTs are from dbEST through Oct. 19, 2001

69367 mRNAs+gene CDSs

1147828 EST, 3′reads

1196006 EST, 5′reads

+598081 EST, other/unknown

3011282 total sequences in clusters

Final Number of Clusters (Sets)

96332 sets total

20516 sets contain at least one known gene

95171 sets contain at least one EST

19355 sets contain both genes and ESTs

Release Notes

The genes or gene products defined by their Swissprot or TREMBL numbers are disclosed at the following internet addresses:

http://www.ebi.ac.uk/swissprot/ or http://www.ncbi.nlm.nih.gov/.

The genes or gene products defined by their EMBL/Genbank numbers are disclosed at the following internet address:

http://www.ncbi.nlm.nih.gov/.

According to the invention, the solution to the second problem addressed by the present invention is provided by a process (2) for the in vitro determination of skin stress and/or ageing of the skin in human beings or animals, more particularly in females, which is characterized in that

a) a mixture of proteins, mRNA molecules or fragments of proteins or mRNA molecules is isolated from human or animal skin,

b) the mixture isolated is tested for the presence and optionally the quantity of at least one of the proteins, mRNA molecules or fragments of proteins or mRNA molecules identified as expressed differentially in old and young skin by serial analysis of gene expression (SAGE),

c) the test results from b) are compared with the expression patterns identified by serial analysis of gene expression (SAGE) and

d) the mixture tested in b) is assigned to old or stressed skin if it predominantly contains proteins, mRNA molecules or fragments of proteins or mRNA molecules which are expressed more strongly in old or stressed skin than in young or unstressed skin or the mixture tested in b) is assigned to young or unstressed skin if it predominantly contains proteins, mRNA molecules or fragments,of proteins or mRNA molecules which are expressed more strongly in young or unstressed skin than in old or stressed skin.

It may be sufficient in step b) of the process for determining skin stress and/or ageing of the skin to test the isolated mixture for the presence of at least one of the proteins, mRNA molecules or fragments of proteins or mRNA molecules identified as differentially expressed in old and young skin by serial analysis of gene expression (SAGE) if they are expressed solely in old skin or solely in young skin. In all other cases, the quantity of differentially expressed molecules must also be determined, i.e. the expression must be quantified, in step b).

In step d) of the process for determining skin stress and/or ageing of the skin, the mixture tested in b) is assigned to old or stressed skin if it predominantly contains proteins, mRNA molecules or fragments of proteins or mRNA molecules which are expressed more strongly in old or stressed skin than in young or unstressed skin, i.e. the mixture either contains more different compounds typically expressed in old skin than those which are typically expressed in young skin (qualitative differentiation) or more copies of compounds typically expressed in old skin than typically present in young skin (quantitative differentiation). A complementary procedure is adopted for assignment to young or unstressed skin.

A preferred embodiment of the process according to the invention for determining skin stress and/or ageing of the skin is characterized in that, in step b), the mixture isolated is tested for the presence and optionally the quantity of at least one of the proteins, mRNA molecules or fragments of proteins or mRNA molecules defined by their UniGene Accession Number in Tables 1 to 4, column 7; in step c), the test results from b) are compared with the relative expression frequencies shown in Tables 1 to 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least twice as strongly in old or stressed skin as in young or unstressed skin or the mixture tested in b) is assigned to young or unstressed skin if it predominantly contains proteins, mRNA molecules or fragments of proteins or mRNA molecules which are expressed at least twice as strongly in young or unstressed skin as in old or stressed skin.

Another preferred embodiment of the process according to the invention for determining skin stress and/or ageing of the skin is characterized in that, in step b), the mixture isolated is tested for the presence and optionally the quantity of at least one of the proteins, mRNA molecules or fragments of proteins or mRNA molecules defined by their UniGene Accession Number in Tables 2 to 4, column 7; in step c), the test results from b) are compared with the relative expression frequencies shown in Tables 2 to 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least five times as strongly in old or stressed skin as in young or unstressed skin or the mixture tested in b) is assigned to young or unstressed skin if it predominantly contains proteins, mRNA molecules or fragments of proteins or mRNA molecules which are expressed at least five times as strongly in young or unstressed skin as in old or stressed skin.

Another preferred embodiment of the process according to the invention for determining skin stress and/or ageing of the skin is characterized in that, in step b), the mixture isolated is tested for the presence and optionally the quantity of at least one of the proteins, mRNA molecules or fragments of proteins or mRNA molecules defined by their UniGene Accession Number in Tables 3 and 4, column 7; in step c), the test results from b) are compared with the relative expression frequencies shown in Tables 3 and 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least seven times as strongly in old or stressed skin as in young or unstressed skin or the mixture tested in b) is assigned to young or unstressed skin if it predominantly contains proteins, mRNA molecules or fragments of proteins or mRNA molecules which are expressed at least seven times as strongly in young or unstressed skin as in old or stressed skin.

Another preferred embodiment of the process according to the invention for determining skin stress and/or ageing of the skin is characterized in that, in step b), the mixture isolated is tested for the presence and optionally the quantity of at least one of the proteins, mRNA molecules or fragments of proteins or mRNA molecules defined by their UniGene Accession Number in Table 4, column 7; in step c), the test results from b) are compared with the relative expression frequencies shown in Table 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least ten times as strongly in old or stressed skin as in young or unstressed skin or the mixture tested in b) is assigned to young or unstressed skin if it predominantly contains proteins, mRNA molecules or fragments of proteins or mRNA molecules which are expressed at least ten times as strongly in young or unstressed skin as in old or stressed skin.

Another preferred embodiment of the process according to the invention for determining skin stress and/or ageing of the skin is characterized in that, in step b), the mixture isolated is tested for the presence and optionally the quantity of at least one of the proteins, mRNA molecules or fragments of proteins or mRNA molecules defined by their 11-base tag sequence in Table 5 or in Table 7, column 2; in step c), the test results from b) are compared with the relative expression frequencies shown in Table 5 or in Table 7, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least twice, more particularly five times, preferably seven times and more preferably ten times as strongly in old or stressed skin as in young or unstressed skin or the mixture tested in b) is assigned to young or unstressed skin if it predominantly contains proteins, mRNA molecules or fragments of proteins or mRNA molecules which are expressed at least twice, more particularly five times, preferably seven times and more preferably ten times as strongly in young or unstressed skin as in old or stressed skin.

The condition of the skin may also be described by quantifying several markers (expression products of the genes important to skin ageing and/or skin stress) which then have to be active in a characteristic ratio to one another in order to represent young skin or in a different characteristic ratio to represent old skin.

Accordingly, the present invention also relates to a process (3) for the in vitro determination of skin stress and/or ageing of the skin in human beings or animals, more particularly in females, which is characterized in that

a) a mixture of proteins, mRNA molecules or fragments of proteins or mRNA molecules is isolated from human or animal skin,

b) in the mixture isolated, at least two of the proteins, mRNA molecules or fragments of proteins or mRNA molecules identified as important to skin ageing and/or skin stress by process (1) are quantified,

c) the expression ratios of the at least two proteins, mRNA molecules or fragments of proteins or mRNA molecules to one another are determined,

d) the expression ratios from c) are compared with the expression ratios typically present in young skin or in old skin for the molecules quantified in b), more particularly with the expression ratios shown in Tables 1 to 5, columns 3 and 4 and

e) the mixture isolated in a) is assigned to old or stressed skin if the expression ratios of the skin under analysis correspond to the expression ratios in old skin or the mixture isolated in a) is assigned to young or unstressed skin if the expression ratios of the skin under analysis correspond to the expression ratios in young skin.

In step a) of the process according to the invention for determining skin stress and/or ageing of the skin, the mixture is preferably isolated from a skin sample, more particularly from a whole skin sample or from an epidermis sample. The whole skin sample offers more comprehensive possibilities for comparison with the SAGE libraries which are similarly obtained from whole skin. By contrast, the epidermis sample is easier to obtain, for example by applying an adhesive plaster to the skin and stripping it off, as described in WO 00/10579 to the whole of which reference is hereby made.

In another embodiment of the process according to the invention for determining skin stress and/or ageing of the skin, the mixture is isolated in step a) by microdialysis. The technique of microdialysis is described, for example, in “Microdialysis: A method for measurement of local tissue metabolism”, Nielsen, P. S., Winge, K., Petersen, L. M.; Ugeskr Laeger 1999, Mar. 22, 161:12 1735-8: and in “Cutaneous microdialysis for human in vivo dermal absorption studies”, Anderson, C. et al.; Drugs Pharm. Sci., 1998, 91, 231-244; and also on-line at http://www.microdialysis.se/techniqu.htm, to the whole of which reference is hereby made. In microdialysis, a probe is typically inserted into the skin and slowly rinsed with a suitable carrier solution. After the acute reactions have abated after insertion, microdialysis yields proteins, mRNA molecules or fragments of proteins or mRNA molecules which occur in the extracellular space and which can then be isolated in vitro, for example by fractionation of the carrier liquid, and analyzed. Microdialysis is less invasive than the removal of a whole skin sample but has the disadvantage that it is limited to the isolation of compounds occurring in the extracellular space.

Another preferred embodiment of the process according to the invention for determining skin stress and/or ageing of the skin is characterized in that, in step b) in process (2), testing for the presence and optionally the quantity of at least one of the proteins or protein fragments; or, in process (3), the quantification of at least two proteins or protein fragments is carried out by a method selected from

i. one- or two-dimensional gel electrophoresis

ii. affinity chromatography

iii. protein/protein complexing in solution

iv. mass spectrometry, more particularly Matrix Assisted Laser Desorption Ionization (MALDI) and, more particularly,

v. the use of protein chips,

or suitable combinations of these methods.

These methods suitable for use in accordance with the invention are described in the overview by Akhilesh Pandey and Matthias Mann: “Proteomics to study genes and genomes”, Nature, Volume 405, Number 6788, 837-846 (2000) and the references cited therein, to the whole of which reference is hereby made.

2D gel electrophoresis is described, for example, in L. D. Adams, Two-dimensional Gel Electrophoresis using the Isodalt System or in L. D. Adams and S. R. Gallagher, Two-dimensional Gel Electrophoresis using the O'Farrell System; both in Current Protocols in Molecular Biology (1997), Eds. F. M. Ausubel et al.), Unit 10.3.1- 10.4.13; or in 2-D Electrophoresis Manual; T. Berkelman, T. Senstedt; Amersham Pharmacia Biotech, 1998 (Order No. 80-6429-60).

The mass spectrometric characterization of the proteins or protein fragments is conducted in known manner, for example as described in the following literature references:

Methods in Molecular Biology, 1999; Vol. 112; 2-D Proteome Analysis Protocols; Editor: A. J. Link; Humana Press; Totowa; N.J.; cf. in particular Courchesne, P. L. and Patterson, S. D.; pp. 487-512.

Carr S. A. and Annan, R. S.; 1997; in: Current Protocols in Molecular Biology; Editor: Ausubel, F. M. et al.; John Wiley and Sons, Inc. 10.2.1-10.21.27.

Another preferred embodiment of the process according to the invention for determining skin stress and/or ageing of the skin is characterized in that, in step b) in process (2), testing for the presence and optionally the quantity of at least one of the mRNA molecules or mRNA molecule fragments; or, in process (3), the quantification of at least two mRNA molecules or mRNA molecule fragments is carried out by a method selected from

i. northern blots,

ii. reverse transcriptase polymerase chain reaction (RT-PCR),

iii. RNase protection experiments,

iv. dot blots,

v. cDNA sequencing,

vi. clone hybridization,

vii. differential display,

viii. subtractive hybridization,

ix. cDNA fragment fingerprinting,

x. total gene expression analysis (TOGA)

xi. serial analysis of gene expression (SAGE) and, more particularly,

xii. the use of nucleic acid chips

or suitable combinations of these methods.

These methods suitable for use in accordance with the invention are described in the overviews by Akhilesh Pandey and Matthias Mann: “Proteomics to study genes and genomes”, Nature, Volume 405, Number 6788, 837-846 (2000) and “Genomics, gene expression and DNA arrays”, Nature, Volume 405, Number 6788, 827-836 (2000) and the references cited therein, to the whole of which reference is hereby made.

The TOGA process is described in “J. Gregor Sutcliffe et al., TOGA:An automated parsing technology for analyzing expression of nearly all genes, Proceedings of the National Academy of Sciences of the United States of America (PNAS), Vol. 97, No. 5, pp. 1976-1981 (2000)”, to the whole of which reference is hereby made.

According to the invention, however, other methods known to the expert may also be used to test for the presence and optionally the quantity of at least one of the proteins, mRNA molecules or fragments of proteins or mRNA molecules.

Another preferred embodiment of the process according to the invention for determining skin stress and/or ageing of the skin is characterized in that step b) comprises testing for the presence and optionally the quantity of 1 to about 5,000, preferably 1 to about 1,000, more preferably about 10 to about 500, most preferably about 10 to about 250, more particularly about 10 to about 100 and most particularly about 10 to about 50 of the proteins, mRNA molecules or fragments of proteins or mRNA molecules which are defined

i. by their Unigene Accession Number in Tables 1 to 4, column 7, or

ii. by their UniGene Accession Number in Table 6 or 8, column 2, or by their Swissprot or TREMBL number in column 3 or by their EMBL/Genbank number in column 4 or by the name of the gene in Table 9 or

iii. by their 11-base tag sequence in Table 5 or in Table 7, column 2.

The present invention also relates to a test kit for the in vitro determination of skin stress and/or ageing of the skin in human beings or animals comprising means for carrying out the process according to the invention for determining skin stress and/or ageing of the skin.

The present invention also relates to a biochip for the in vitro determination of skin stress and/or ageing of the skin in human beings or animals comprising

i. a firm, i.e. rigid, or flexible support and,

ii. immobilized thereon, probes which are capable of binding specifically to at least one of the proteins, mRNA molecules or fragments of proteins or mRNA molecules which are defined by their UniGene Accession Number in Tables 1 to 4, column 7, or by their 11-base tag sequence in Table 5 or in Table 7, column 2.

A biochip is a miniaturized functional element with molecules, more particularly biomolecules, immobilized on a surface which are capable of acting as specific interaction partners. The structure of these functional elements often comprise rows and columns. They are then known as chip arrays. Since thousands of biological or biochemical functional elements can be arranged on one chip, they generally have to be made by microtechnical methods. Biological and biochemical functional elements include in particular DNA, RNA, PNA (in the case of nucleic acids and chemical derivatives thereof, single strands, triplex structures or combinations thereof, for example, may be present), saccharides, peptides, proteins (for example antibodies, antigens, receptors) and derivatives of combinatorial chemistry (for example organic molecules). Biochips generally have a two-dimensional base for coating with biologically or biochemically active materials. The bases may also be formed, for example, by walls of one or more capillaries or by channels. The prior art is represented, for example, by the following publications: Nature Genetics, Vol. 21, Supplement (entire), January 1999 (Biochips); Nature Biotechnology, Vol. 16, pp. 981-983, October 1998 (Biochips); Trends in Biotechnology, Vol. 16, pp. 301-306, July 1998 (Biochips) and the already cited overviews by Akhilesh Pandey and Matthias Mann: “Proteomics to study genes and genomes”, Nature, Volume 405, Number 6788, 837-846 (2000) and “Genomics, gene expression and DNA arrays”, Nature, Volume 405, Number 6788, 827-836 (2000) and the references cited therein, to the whole of which reference is hereby made.

A synoptic portrayal of the practical methods of using DNA chip technology can be found in the books “DNA Microarrays: A Practical Approach” (Editor: Mark Schena, 1999, Oxford University Press) and “Microarray Biochip Technology” (Editor: Mark Schena, 2000, Eaton Publishing), to the whole of which reference is hereby made.

The particularly preferred DNA chip technology in the context of the present invention is based on the ability of nucleic acids to enter into complementary base pairings. This technical principle known as hybridization has been used for years in southern blot and northern blot analysis. By comparison with these conventional methods where only a few genes are analyzed, DNA chip technology enables a few hundred to several thousand genes to be analyzed at the same time. A DNA chip consists essentially of a support material (for example glass or plastic) on which single-stranded gene-specific probes are immobilized in high density at a particular spot. The technique of probe application and the chemistry of probe immobilization are rated as problematical.

In the present state of the art, probe immobilization can be carried out in several ways:

E. M. Southern (E. M. Southern et al. (1992), Nucleic Acid Research 20, 1679-1684 and E. M. Southern et al. (1997), Nucleic Acid Research 25, 1155-1161) describes the production of oligonucleotide arrangements by direct synthesis on a glass surface derivatized with 3-glycidoxypropyl trimethoxysilane and then with a glycol. A similar process achieves the in situ synthesis of oligonucleotides by photosensitive combinatorial chemistry which may be compared with photolithographic techniques (Pease, A. C. et al. (1994), Proc. Natl. Acad. Sci. USA 91, 5022-5026).

Besides these techniques based on the in situ synthesis of oligonucleotides, already present DNA molecules can also be immobilized on surfaces of support material.

P. O. Brown (DeRisi et al. (1997), Science 278, 680-686) describes the immobilization of DNA on glass surfaces coated with polylysine.

The Article by L. M. Smith (Guo, Z. et al. (1994), Nucleic Acid Research 22, 5456-5465) discloses a similar process: oligonucleotides carrying a 5′-terminal amino group can be immobilized on a glass surface treated with 3-aminopropyl trimethoxysilane and then with 1,4-phenyl diisothiocyanate.

The DNA probes may be applied to a support using a so-called pin spotter. To this end, thin metal needles, for example 250 μm in diameter, dip into probe solutions and then transfer the adhering sample material in defined volumes to the support material of the DNA chip.

However, the probes are preferably applied by means of a piezo-controlled nanodispenser which—similarly to an ink jet printer—applies probe solutions with a volume of 100 picoliters to the surface of the support material without any contact.

The probes are immobilized as described, for example, in EP-A-0 965 647. DNA probes are generated by PCR using a sequence-specific primer pair, one primer being modified at the 5′-end and carrying a linker with a free amino group. This ensures that a defined strand of the PCR products can be immobilized on a glass surface treated with 3-aminopropyl trimethoxysilane and then with 1,4-phenyl diisothiocyanate. Ideally, the gene-specific PCR products should comprise a defined nucleic acid sequence with a length of 200-400 bp and non-redundant sequences. After immobilization of the PCR products via the derivatized primer, the counter-strand of the PCR product is removed by incubation for 10 mins. at 96° C.

In one application typical of DNA chips, mRNA is isolated from two cell populations to be compared. The isolated mRNAs are converted into cDNA by reverse transcription using, for example, fluorescence-marked nucleotides. The samples to be compared are marked, for example, with red- or green-fluorescing nucleotides. The cDNAs are then hybridized with the gene probes immobilized on the DNA chip and the fixed fluorescences are then quantified.

The biochip according to the invention preferably comprises 1 to about 5,000, preferably 1 to about 1,000, more preferably about 10 to about 500, most preferably about 10 to about 250, more particularly about 10 to about 100 and most particularly about 10 to about 50 different probes. The different probes may be present as multiple copies on the chip.

The biochip according to the invention preferably comprises nucleic acid probes, more particularly RNA or PNA probes and most particularly DNA probes. The nucleic acid probes preferably have a length of about 10 to about 1,000, more preferably a length of about 10 to about 800, most preferably a length of about 100 to about 600 and, in one most particularly preferred embodiment, a length of about 200 to about 400 nucleotides.

In another preferred embodiment, the biochip according to the invention comprises peptide or protein probes, more particularly antibodies.

In another preferred form, the biochip according to the invention comprises probes which are capable of binding specifically to at least one of the proteins, mRNA molecules or fragments of proteins or mRNA molecules which are identified by their UniGene Accession Number in Table 6 or 8, column 2, or by their Swissprot or TREMBL number in column 3 or by their EMBL/Genbank number in column 4 or by the name of the gene in Table 9.

The present invention also relates to the use of the proteins, mRNA molecules or fragments of proteins or mRNA molecules which are defined

i. by their Unigene Accession Number in Tables 1 to 4, column 7, or

ii. by their UniGene Accession Number in Table 6 or 8, column 2, or by their Swissprot or TREMBL number in column 3 or by their EMBL/Genbank number in column 4 or by the name of the gene in Table 9 or

iii. by their 11-base tag sequence in Table 5 or in Table 7, column 2,

as skin stress and/or skin ageing markers in human beings or animals.

The present invention also relates to a test for demonstrating the effectiveness of cosmetic or pharmaceutical active substances against skin stress and/or ageing of the skin in vitro, characterized in that

a) the status of the skin is determined by a process according to the invention for determining skin stress and/or ageing of the skin or with the aid of a test kit according to the invention for determining skin stress and/or ageing of the skin or with the aid of a biochip according to the invention,

b) an active substance against skin stress and/or ageing of the skin is applied one or more times to the skin,

c) the status of the skin is re-determined by a process according to the invention for determining skin stress and/or ageing of the skin or with the aid of a test kit according to the invention for determining skin stress and/or ageing of the skin or with the aid of a biochip according to the invention and

d) the effectiveness of the active substance is determined by comparing the results from a) and c).

In order to accelerate the test, various active substances or placebos may be simultaneously applied to different areas of the skin. For example, an active substance may be applied to the left forearm and a placebo to the right forearm or vice versa.

The present invention also relates to a test kit for demonstrating the effectiveness of cosmetic or pharmaceutical active substances against skin stress and/or ageing of the skin in vitro comprising means for carrying out the test according to the invention.

The present invention also relates to the use of the proteins, mRNA molecules or fragments of proteins or mRNA molecules which are defined

i. by their Unigene Accession Number in Tables 1 to 4, column 7, or

ii. by their UniGene Accession Number in Table 6 or 8, column 2, or by their Swissprot or TREMBL number in column 3 or by their EMBL/Genbank number in column 4 or by the name of the gene in Table 9 or

iii. by their 11-base tag sequence in Table 5 or in Table 7, column 2,

for demonstrating the effectiveness of cosmetic or pharmaceutical active substances against skin stress and/or ageing of the skin.

The present invention also relates to a screening process for identifying cosmetic or pharmaceutical active substances against skin stress and/or ageing of the skin in vitro, characterized in that

a) the status of the skin is determined by a process according to the invention for determining skin stress and/or ageing of the skin or with the aid of a test kit according to the invention for determining skin stress and/or ageing of the skin or with the aid of a biochip according to the invention,

b) a potential active substance against skin stress and/or ageing of the skin is applied one or more times to the skin,

c) the status of the skin is re-determined by a process according to the invention for determining skin stress and/or ageing of the skin or with the aid of a test kit according to the invention for determining skin stress and/or ageing of the skin or with the aid of a biochip according to the invention and

d) effective active substances are determined by comparing the results from a) and c).

The present invention also relates to the use of the proteins, mRNA molecules or fragments of proteins or mRNA molecules which are defined

i. by their Unigene Accession Number in Tables 1 to 4, column 7, or

ii. by their UniGene Accession Number in Table 6 or 8, column 2, or by their Swissprot or TREMBL number in column 3 or by their EMBL/Genbank number in column 4 or by the name of the gene in Table 9 or

iii. by their 11-base tag sequence in Table 5 or in Table 7, column 2,

for identifying cosmetic or pharmaceutical active substances against skin stress and/or ageing of the skin.

The present invention also relates to a process for the production of a cosmetic or pharmaceutical preparation against skin stress and/or ageing of the skin, characterized in that

a) active substances are determined by the screening process according to the invention or by the use for identifying cosmetic or pharmaceutical active substances skin stress and/or ageing of the skin and

b) active substances found to be effective are mixed with cosmetically and pharmacologically suitable and compatible carriers.

The present invention also relates to a cosmetic or pharmaceutical preparation against skin stress and/or ageing of the skin containing at least one nucleic acid construct which is capable of suppressing or reducing the activity of at least one of the proteins that are expressed more strongly in old or stressed skin than in young or unstressed skin or of inducing or strengthening the activity of at least one of the proteins that are expressed more strongly in young or unstressed skin than in old or stressed skin.

The proteins are preferably selected from those which are defined by their Unigene Accession Number in Tables 1 to 4, column 7, or by their UniGene Accession Number in Table 6 or 8, column 2, or by their Swissprot or TREMBL number in column 3 or by their EMBL/Genbank number in column 4 or by the name of the gene in Table 9 or by their 11-base tag sequence in Table 5 or in Table 7, column 2.

The nucleic acid construct is preferably selected from DNA, RNA or PNA. However, linear combinations of these nucleic acids or hybrid molecules, for example RNA/DNA molecules, are also possible. In addition, the nucleic acid construct is preferably selected from protein-coding sequences, ribozymes, antisense nucleic acids, triple helix formers and rRNA.

The preparation according to the invention may contain about 1,000, more particularly about 10 to about 500, preferably about 10 to about 250, more preferably about 10 to about 100 and most preferably about 10 to about 50 different nucleic acid constructs.

In one particular embodiment, the nucleic acid construct is present in the preparation according to the invention encapsulated in lipid vesicles, for example in liposomes, niosomes or transfersomes, preferably in liposomes.

Nucleic acid constructs in the context of the invention include DNA and RNA sequences which code for one of the ageing markers and constructs of these polynucleotides.

After their transport into the cells of the skin, these constructs are transcribed and/or translated and lead to the strengthened expression of the proteins coded by them.

The invention also encompasses partial sequences of age genes and genes whose sequence has been modified in relation to the age genes by directed or other forms of mutagenesis. Various techniques for modifying closed genes are known and are described, for example, in “Current Protocols in Molecular Biology”, Vol. 1, Chapter 8, Ausubel et al. (Ed.), John Wiley & Sons, Inc. (2001). These modifications may be made in such a way that, after transcription and/or translation, the resulting protein has the identical amino acid sequence to the protein which would result without this mutation (redundancy of the genetic code). However, the modified gene may also lead to the expression of a protein with a modified amino acid sequence so that its biological activity is modified, for example strengthened or weakened.

In addition, polynucleotides suitable for channeling into the skin include DNA and RNA sequences which comprise part of the sequence of one or more of the genes mentioned and which even have the desired biological activity (for example antisense RNA, rRNA or short double-stranded DNA molecules).

Each gene suitable for use in accordance with the invention, but preferably those found to be weakly expressed in old skin, can be channeled into the cells of the skin with the object of strengthened expression.

Each construct of one of the genes usable in accordance with the invention—functionally linked to a eukaryotic promoter—may be used.

The constructs with one of the age genes may be any eukaryotic expression constructs. For example, bacterial plasmids, viral constructs or other DNA constructs may be genetically modified to form a recombinant DNA (or RNA) molecule which contains a sequence that codes for one of the age genes and expressed the required gene product in cells of the skin.

Constructs capable of replicating both in eukaryotic and in prokaryotic host cells are preferred. Such constructs are known and commercially available.

The preparation according to the invention is preferably applied to the skin. The DNA present in it may be either linear or circular, circular DNA molecules being preferred. The polynucleotide or the polynucleotide-containing construct may be multiplied and purified by known methods and is used either as a pure molecule or in one of the formulations mentioned below.

Constructs carrying a promoter that enables the DNA in question to be expressed are preferred. Various promoters may be used according to the nature of the gene and the purpose of its use. Strong constitutive promoters may be used, including for example the immediate early gene of cytomegalovirus (CMV) or the promoter of the long terminal repeat of Rous sarcoma virus (RSV).

Alternatively, tissue-specific promoters or cell-type-specific promoters may be used. For example, the promoter may be selected so that it effects the specific expression into skin cells or certain skin cell types. Examples of tissue-specific or cell-type-specific promoters are inter alia the keratin promoters (for example human keratin 14 promoter (Wang et al. 1997 Proc. Natl. Acd. Sci. US 94:219-226)) or tyrosinase promoters (specific to melanocytes). Alternatively, inducible promoters may be used.

Besides the promoters, the constructs may contain other elements which strengthen the transcriptional or translational expression of the gene product. For example, the construct may contain an internal ribosomal entry site (IRES) to strengthen the translation of the downstream sequence (cf. Murakami et al. 1997, Gene 202:23-29). Other components which may be present on the construct include markers (for example antibiotic resistance genes, such as the ampicillin resistance gene) for selecting cells which contain the construct, a replication source which effects the stable replication of the construct in prokaryotic cells, a nucleus locating signal or other elements which support the production of the DNA construct and/or of the coded protein. The constructs may also contain a polyadenylation signal. The sequence of such a signal may be selected from various known polyadenylation signals. A preferred example is the SV40 early polyadenylation signal.

The constructs may also contain one or more introns which can strengthen the expression of the DNA.

The present invention also encompasses constructs which code for fusion proteins of one of the age markers with a second protein or for a fusion protein of two age markers.

Preferred nucleic acid constructs are those with several expression cassettes on which two or more age markers are coded or which code one of the age genes and a gene for another protein.

Co-transfection with one or more other vectors which are capable of supporting the expression of the gene in question is also possible.

Vectors which bear one or more of the features mentioned and which, in addition, may contain other functional units have often been described in the literature and are commercially available. Examples of such vectors include pCI and pSI (Promega GmbH) or PDEST (Gibco BRL).

The sequences of the age genes and partial gene sequences determinable in accordance with the invention and in particular those of the age genes and partial gene sequences listed in Tables 1 to 9 may be used for selective inhibition of the expression of individual genes.

Preferred targets for expression inhibition are those which were found to be expressed more strongly in old skin. Oligo- and polynucleotides suitable for this purpose include antisense nucleotides, ribozyme nucleotides and double-stranded RNAs.

Antisense nucleotides are well known for their ability to hybridize with sense strands of mRNA and thus to interfere with the expression of mRNA (cf. for example Wingers et al., Laboratory Investigation 79, 1415-1424 (1999). An overview of the application of antisense nucleotides in the skin is presented by Wraight et al., Pharmacol. & Ther. 90, 89-104 (2001).

Ribozyme nucleic acids are also known as single-stranded RNA molecules which are capable of selectively cleaving ssRNA and ssDNA and thus selectively inhibiting the expression of the target molecules.

Post-transcriptional gene repression can also be produced by double-stranded RNAs. These dsRNAs interfere with the target RNA after cleavage into shorter segments by ribonuclease III. Duplexes of short RNA sequences may also be used for gene repression (S. M. Elbashir et al., Nature 411, 494-498 (2001). These double-stranded RNA molecules may also be used in accordance with the invention for repressing the age genes found.

Unmodified DNA or RNA molecules undergo rapid degradation in tissue or cells. In order to increase the resistance of oligo- or polynucleotides to degradation by nucleases, modifications involving either the backbone or the pyridine or pyrimidine bases of an oligonucleotide were developed. Correspondingly modified DNA or RNA sequences may also be used in accordance with the invention. Suitable modifications are, for example, phosphorthioates, methyl phosphonates or peptide linkages (PNAs) for the sugar backbone and C5-propynyl-dU, dC for the nucleoside bases.

The DNA or RNA molecules of the invention may be applied, for example, topically. The molecules may be used either without other penetration-influencing substances or in admixture or association with molecules which influence penetration through the stratum corneum and the transfection of the skin cells.

A preferred embodiment of the topical application of the age genes is a formulation containing lipids, optionally in admixture with surfactants, preferably in the form of liposomes.

This formulation contains ca. 0.1 μg -5 mg DNA or RNA per mg liposome. The constituents of the liposomes may be neutral or charged and may be present, for example, in the form of multilamellar vesicles or unilamellar vesicles.

Suitable lipids for the production of liposomes are, for example, phosphatidyl choline which may be obtained, for example, from eggs, soybeans, olives, coconuts, spermaceti, saffrons, linseeds, evening primroses or primulas. Other suitable lipids are, for example, natural and synthetic phosphatidyl ethanolamine, synthetic phosphatidyl choline, phosphatidic acids or esters thereof, phosphatidyl serine and phosphatidyl (poly)alcohols such as, for example, phosphatidyl inositol or phosphatidyl glycerol. Examples of the lipids mentioned are DPPC (dipalmitoyl phosphatidyl choline), DOPE (dioleyl phosphatidyl ethanolamine), DOTMA (N[1-(2,3-dioleoyloxy)propyl]-N,N,N-trimethyl ammonium chloride), DOTAB (N-1-(2,3-dioleoyloxy)propyl-N,N,N-trimethylammonium chloride), DPPA (dipalmitoylphosphatidic acid), DPPG (dipalmitoyl phosphatidyl glycerol).

Individual surface-active compounds used, for example as detergents or emulsifiers may also be used for the formation of liposomes. Examples of such surface-active compounds are DODAC (dioctadecyl ammonium chloride) and CTAC (cetyl trimethyl ammonium chloride). Suitable constituents of liposomes and production processes are described in the literature (cf. for example “Liposome Drug Delivery Systems”, G. Betageri (Ed.), Lancaster Techonomic Publishing Company (1993) or “Liposome Technology”, Gregoriadis (Ed.), CRC Press)). Besides liposomes, other lipid vesicles such as, for example, niosomes and transfersomes (cf. for example WO 98/17255) may also be used.

The penetrative power of the DNA and RNA molecules may be improved by the previous or simultaneous application of penetration enhancers. Chemical penetration enhancers have often been described in the literature (cf. for example M. Foldvari, PSTT 3, 417-425 (2000) or N. Kanikkannan, Curr Med. Chem. 7, 593-608 (2000)). Suitable penetration enhancers are inter alia organic solvents (for example ethanol), pyrrolidones, sulfoxides (for example DMSO), fatty acids (saturated or unsaturated, branched or unbranched with a preferred chain length of 8 to 18), terpenes (for example L-menthol or 1,8-cineol), surfactants (for example polysorbates (Tween), polyethylene alkylphenols (Brij), alkyl ether sulfates and betaines and amphoteric glycinates).

Invasive or minimal-invasive methods may also be used in accordance with the invention for improving the penetration of oligo- or polynucleotides. Conventional minimal-invasive methods include eletroporation and iontophoresis. In both methods, a voltage is applied to the surface of the skin with the aid of electrodes. Electroporation uses a brief high-voltage pulse to permeate the skin. In iontophoresis, a small voltage with constant current is used for this purpose. Low-frequency ultrasound may also be used to increase the permeability of the skin to DNA or RNA.

The following Examples are intended to illustrate the invention without limiting it in any way.

### EXAMPLE 1

### Preparation of Liposome/DNA Complexes

18.6 μl (25 μmol) DOPE and 0.0175 g DOTAP (25 μmol) were dissolved together in 1 ml ethanol. The solution was dried overnight in an exsiccator and the lipid mixture was then rehydrated for 3-4 h in 3 ml PBS (58 mM NaHPO₄, 17 mM NaH₂PO₄, 69 mM NaCl, pH 7.4) with occasional mixing in the absence of light. The lipid dispersion thus formed was ultrasonicated twice for 5 mins. with a 2-hour pause in between. 6.1 μl of the liposome dispersion formed were diluted with 1.5 ml PBS and mixed with 20 μg plasmid dissolved in 1.5 ml HBS (150 mM NaCl, 20 mM hepes, pH 7.4). The fairly intense clouding occurring during addition of the plasmid solution to the liposome solution is an indication of the formation of the DNA/liposome complexes.

### EXAMPLE 2

### Preparation of Liposome/DNA Complexes

18.6 μl (25 μmol) DOPE and 0.0175 g DOTAP (25 μmol) were dissolved together in 1 ml ethanol. The solution was dried overnight in an exsiccator and the lipid mixture was then rehydrated for 3-4 h in 3 ml PBS (58 mM NaHPO₄, 17 mM NaH₂PO₄, 69 mM NaCl, pH 7.4) with occasional mixing in the absence of light. The lipid dispersion thus formed was ultrasonicated twice for 5 mins. with a 2-hour pause in between. 50 μl of the liposome dispersion formed were mixed with 50 μg plasmid dissolved in 50 μl HBS (150 mM NaCl, 20 mM hepes, pH 7.4). The liposomes were identified by TEM micrographs of the liposome/DNA complexes (FIG. 1).

### EXAMPLE 3

### Preparation of Liposomes with Cationic Detergent and Complexing with DNA

29.8 μl (40 μmol) DOPE and 4 mg CTAC (cetyl trimethyl ammonium chloride) (10 μmol) were dissolved together in 1 ml ethanol. The solvent ethanol was removed in a rotary evaporator and the mixture was then rehydrated for 3-4 h in 3 ml PBS (58 mM NaHPO₄, 17 mM NaH₂PO₄, 69 mM NaCl, pH 7.4) with occasional mixing in the absence of light. The lipid dispersion thus formed was ultrasonicated twice for 5 mins. with a 2-hour pause in between. 6.7 μl of the liposome dispersion formed were diluted with 1.5 ml PBS and mixed with 20 μg plasmid dissolved in 1.5 ml HBS (150 mM NaCl, 20 mM hepes, pH 7.4).

### LIST OF FIGURES

FIG. 1

TEM micrograph of the liposome/DNA complexes of Example 2 in cryomode. Magnification:×10,000

### FIG. 1

Tables:

TABLE 4

| No. | Tag sequence | Young | Old | Quotient | Significance | Annotation | Description |
|---|---|---|---|---|---|---|---|
| 1 | CCCCGGCCACC | 43.72 | 0.00 | 43.72 | 13.39 | Hs.279604 | Desmin, muscle |
| | | | | | | | intermediate filament |
| | | | | | | | protein |
| 2 | AAACATTAAAA | 20.82 | 0.96 | 21.69 | 5.35 | Hs.77443 | actin, gamma 2, |
| | | | | | | | smooth muscle, |
| | | | | | | | enteric |
| 3 | GGCTGTACCCA | 20.82 | 0.96 | 21.69 | 5.35 | Hs.108080 | cysteine and glycine- |
| | | | | | | | rich protein 1 |
| 4 | TTGAGGGGGTG | 0.00 | 16.35 | 16.35 | 4.85 | Hs.76549 | reverse tag of AHNAK |
| 5 | CCACGGGATTC | 14.57 | 0.00 | 14.57 | 4.48 | Hs.119571 | collagen, type III, |
| | | | | | | | alpha 1 (Ehlers- |
| | | | | | | | Danlos syndrome type |
| | | | | | | | IV) |
| 6 | AGCCACCGCAC | 12.49 | 0.96 | 13.01 | 3.01 | Hs.42612 | ESTs |
| 7 | TCCTCCCTACT | 1.04 | 12.51 | 12.03 | 2.88 | Hs.70266 | yeast Sec31p |
| | | | | | | | homolog |
| 8 | TCAAAAGACCT | 11.45 | 0.96 | 11.93 | 2.73 | Hs.25647 | v-fos FBJ murine |
| | | | | | | | osteosarcoma viral |
| | | | | | | | oncogene homolog |
| 9 | TTAGTGTCGTA | 11.45 | 0.00 | 11.45 | 3.52 | Hs.74649 | cytochrome c oxidase |
| | | | | | | | subunit VIc |
| 10 | TTTCTAGTTTG | 11.45 | 0.00 | 11.45 | 3.52 | Hs.111894 | membrane nucleoside |
| | | | | | | | transporter |
| 11 | GACCAGGCCCT | 20.82 | 1.92 | 10.84 | 4.59 | Hs.180266 | tropomyosin 2 (beta) |
| 12 | TTGAGCCAGCC | 10.41 | 0.96 | 10.84 | 2.45 | Hs.91142 | KH-type splicing |
| | | | | | | | regulatory protein |
| | | | | | | | (FUSE binding protein 2) |
| 13 | AGATTCAAACT | 10.41 | 0.96 | 10.84 | 2.45 | Hs.14368 | SH3 domain binding |
| | | | | | | | glutamic acid-rich |
| | | | | | | | protein like |
| 14 | ACAGGCTACGG | 111.39 | 10.58 | 10.53 | 22.24 | Hs.75777 | transgelin |
| 15 | AATTGAAAAGG | 10.41 | 0.00 | 10.41 | 3.20 | Hs.78344 | myosin, heavy |
| | | | | | | | polypeptide 11, |
| | | | | | | | smooth muscle |
| 16 | GTGGCGAATGA | 1.04 | 10.58 | 10.17 | 2.38 | Hs.69752 | desmocollin 1 |
| 17 | TGGCCCCACCC | 1.04 | 10.58 | 10.17 | 2.38 | Hs.198281 | pyruvate kinase, |
| | | | | | | | muscle |

TABLE 3

| | | | | | | | |
|---|---|---|---|---|---|---|---|
| No. | Tag sequence | Young | Old | Quotient | Significance | Annotation | Description |

| | | | | | | | |
|---|---|---|---|---|---|---|---|
| 18 | GAGACTCCTGC | 0.00 | 9.62 | 9.62 | 2.86 | Hs.169902 | solute carrier family 2, |
| | | | | | | | member 1 |
| 19 | TAGTCCCAGCT | 1.04 | 9.62 | 9.25 | 2.14 | Hs.274579 | ancient conserved |
| | | | | | | | domain protein 1 |
| 20 | CCCAGAGACCC | 17.70 | 1.92 | 9.22 | 3.77 | Hs.21223 | calponin 1, basic, |
| | | | | | | | smooth muscle |
| 21 | AGGCTCAGGTC | 8.33 | 0.96 | 8.68 | 1.90 | Hs.78344 | myosin, heavy |
| | | | | | | | polypeptide 11, |
| | | | | | | | smooth muscle |
| 22 | ATTTCTTCAAG | 8.33 | 0.96 | 8.68 | 1.90 | Hs.31386 | ESTs, similar to |
| | | | | | | | JE0174 frizzled |
| | | | | | | | protein-2-human |
| 23 | TGTGAGCCCCT | 8.33 | 0.96 | 8.68 | 1.90 | Hs.102948 | enigma (LIM domain |
| | | | | | | | protein) |
| 24 | CTGACTTGTGT | 0.00 | 8.66 | 8.66 | 2.58 | Hs.77961 | major |
| | | | | | | | histocompatibility |
| | | | | | | | complex, class I, B |
| 25 | ACGGAACAATA | 8.33 | 0.00 | 8.33 | 2.57 | Hs.8272 | prostaglandin D2 |
| | | | | | | | synthase (21kD, brain) |
| 26 | CTGTTTGTTCA | 8.33 | 0.00 | 8.33 | 2.57 | Hs.211582 | myosin, light |
| | | | | | | | polypeptide kinase |
| 27 | TGTGGCGTATA | 8.33 | 0.00 | 8.33 | 2.57 | Hs.211582 | myosin, light |
| | | | | | | | polypeptide kinase |
| 28 | C1TTTTGTGCC | 8.33 | 0.00 | 8.33 | 2.57 | Hs.182238 | GW128 protein |
| 29 | ATGGATACGGG | 1.04 | 8.66 | 8.33 | 1.89 | Hs.250722 | Reverse tag of MUG |
| | | | | | | | (Myeloid-upregulated |
| | | | | | | | protein) |
| 30 | TAGGATGGGGG | 0.00 | 7.70 | 7.70 | 2.29 | Hs.76941 | ATPase, Na+/K+ |
| | | | | | | | transporting beta 3 |
| | | | | | | | polypeptide |
| 31 | CCCCGCCAAGT | 0.00 | 7.70 | 7.70 | 2.29 | Hs.169718 | calponin 2 |
| 32 | TGCTGTGCATA | 0.00 | 7.70 | 7.70 | 2.29 | Hs.147916 | DEAD/H (Asp-Glu-Ala-Asp/His) |
| | | | | | | | box |
| | | | | | | | polypeptide 3 |
| 33 | CGTGGGACACT | 0.00 | 7.70 | 7.70 | 2.29 | Hs.110196 | NICE-1 protein |
| 34 | CCCTCCTGGGG | 7.29 | 0.96 | 7.59 | 1.63 | Hs.95867 | Homo sapiens |
| | | | | | | | EST00098 gene, last |
| | | | | | | | exon |
| 35 | GGGGTAAGAAA | 7.29 | 0.96 | 7.59 | 1.63 | Hs.80423 | prostatic binding |
| | | | | | | | protein |
| 36 | GACCTATCTCT | 7.29 | 0.96 | 7.59 | 1.63 | Hs.194431 | palladin |
| 37 | CAGGAGGAGTT | 7.29 | 0.96 | 7.59 | 1.63 | Hs.183760 | glucose regulated |
| | | | | | | | protein, 58kD |
| 38 | CTGAGACAAAG | 7.29 | 0.96 | 7.59 | 1.63 | Hs.101025 | basic transcription |
| | | | | | | | factor 3 |
| 39 | CTCCCCTGCCC | 1.04 | 7.70 | 7.40 | 1.66 | Hs.82422 | capping protein (actin |
| | | | | | | | filament(, gelsolin-like |
| 40 | TTCCAAGGCAG | 1.04 | 7.70 | 7.40 | 1.66 | Hs.317 | topoisomerase (DNA) |
| | | | | | | | I |
| 41 | GTGGCCAGAGG | 1.04 | 7.70 | 7.40 | 1.66 | Hs.1420 | fibroblast growth |
| | | | | | | | factor receptor 3 |
| 42 | GGTTTGGCTTA | 7.29 | 0.00 | 7.29 | 2.25 | Hs.73818 | ubiquinol-cytochrome |
| | | | | | | | c reductase hinge |
| | | | | | | | protein |
| 43 | CATCTAAACTG | 7.29 | 0.00 | 7.29 | 2.25 | Hs.180900 | Williams-Beuren |
| | | | | | | | syndrome |
| | | | | | | | chromosome region 1 |
| 44 | ACATAGACCGA | 7.29 | 0.00 | 7.29 | 2.25 | Hs.173594 | pigment epithelium-derived |
| | | | | | | | factor |
| 45 | CCACAGGGGAT | 13.53 | 1.92 | 7.05 | 2.71 | Hs.119571 | collagen, type III, |
| | | | | | | | alpha 1 |

TABLE 2

| No. | Tag sequence | Young | Old | Quotient | Significance | Annotation | Description |
|---|---|---|---|---|---|---|---|
| 46 | AGCCTGGACTG | 0.00 | 6.73 | 6.73 | 2.01 | Hs.90107 | cell membrane |
| | | | | | | | glycoprotein |
| | | | | | | | 110000M(r) (surface |
| | | | | | | | antigen) |
| 47 | TCTGTTTATCA | 0.00 | 6.73 | 6.73 | 2.01 | Hs.180394 | signal recognition |
| | | | | | | | particle 14kD (Alu |
| | | | | | | | RNA-binding protein) |
| 48 | GAGCAGCGCCC | 0.00 | 6.73 | 6.73 | 2.01 | Hs.112408 | S100 calcium-binding |
| | | | | | | | protein A7 |
| | | | | | | | (psoriasin 1) |
| 49 | CCGGGGGAGCC | 38.52 | 5.77 | 6.68 | 6.72 | Hs.172928 | collagen, type I, |
| | | | | | | | alpha 1 |
| 50 | TCTGCTTACAG | 6.25 | 0.96 | 6.51 | 1.37 | Hs.74267 | ribosomal protein |
| | | | | | | | L15 |
| 51 | TTGGTGTGCTG | 6.25 | 0.96 | 6.51 | 1.37 | Hs.240399 | EST |
| 52 | TTGCCCAGCAC | 6.25 | 0.96 | 6.51 | 1.37 | Hs.23954 | cerebral cell |
| | | | | | | | adhesion molecule |
| 53 | CACCCCTGATG | 6.25 | 0.96 | 6.51 | 1.37 | Hs.173724 | creatine kinase, |
| | | | | | | | brain |
| 54 | CCCTTGTCCGA | 6.25 | 0.96 | 6.51 | 1.37 | Hs.127824 | ESTs, Weakly |
| | | | | | | | similar to weak |
| | | | | | | | similarity to |
| | | | | | | | collagens |
| | | | | | | | [C.elegans] |
| 55 | AAACAATAAAA | 1.04 | 6.73 | 6.47 | 1.42 | Hs.229971 | EST |
| 56 | AAATAAAAGCT | 1.04 | 6.73 | 6.47 | 1.42 | Hs.155191 | villin 2 (ezrin) |
| 57 | ACAAAACCCCA | 1.04 | 6.73 | 6.47 | 1.42 | Hs.140208 | ESTs |
| 58 | GTACGTATTCT | 6.25 | 0.00 | 6.25 | 1.93 | Hs.76325 | immunoglobulin J |
| | | | | | | | polypeptide |
| 59 | CCTATAATTCC | 6.25 | 0.00 | 6.25 | 1.93 | Hs.135491 | ESTs |
| 60 | TTTCCTCTCAA | 5.21 | 31.75 | 6.09 | 5.29 | Hs.184510 | stratifin |
| 61 | CGGCTGGTGAA | 0.00 | 5.77 | 5.77 | 1.72 | Hs.75748 | proteasome |
| | | | | | | | (prosome, |
| | | | | | | | macropain) subunit, |
| | | | | | | | beta type, 1 |
| 62 | TGCAGATGGTT | 0.00 | 5.77 | 5.77 | 1.72 | Hs.3628 | mitogen-activated |
| | | | | | | | protein kinase |
| | | | | | | | kinase kinase kinase |
| | | | | | | | 4 |
| 63 | ACAACTTTTAT | 0.00 | 5.77 | 5.77 | 1.72 | Hs.283213 | EST |
| 64 | GTGCGCTGAGC | 0.00 | 5.77 | 5.77 | 1.72 | Hs.277477 | major |
| | | | | | | | histocompatibility |
| | | | | | | | complex, class I, C |
| 65 | CCACTGTATTC | 0.00 | 5.77 | 5.77 | 1.72 | Hs.235041 | EST |
| 66 | AGGGAGGGGCC | 0.00 | 5.77 | 5.77 | 1.72 | Hs.172153 | glutathione |
| | | | | | | | peroxidase 3 |
| | | | | | | | (plasma) |
| 67 | TTGTAAATGCG | 0.00 | 5.77 | 5.77 | 1.72 | Hs.149436 | kinesin family |
| | | | | | | | member 5B |
| 68 | CTTCTACTAAT | 0.00 | 5.77 | 5.77 | 1.72 | Hs.109857 | Homo sapiens |
| | | | | | | | mRNA; cDNA |
| | | | | | | | DKFZp434H0820 |
| 69 | AGCCTGCAGAA | 0.00 | 5.77 | 5.77 | 1.72 | Hs.10927 | hypothetical protein |
| | | | | | | | R33729_1 |
| 70 | GCAAAACCCTA | 0.00 | 5.77 | 5.77 | 1.72 | Hs.108740 | DKFZP586A0522 |
| | | | | | | | protein |
| 71 | GCCCAAGGACC | 27.07 | 4.81 | 5.63 | 4.49 | Hs.195464 | filamin A, alpha |
| | | | | | | | (actin-binding |
| | | | | | | | protein-280) |
| 72 | CAAGAGGCAAA | 1.04 | 5.77 | 5.55 | 1.20 | Hs.5734 | K1AA0679 protein |
| 73 | TGGGACGTGAG | 1.04 | 5.77 | 5.55 | 1.20 | Hs.3796 | EphB6 |
| 74 | CCTGTTATCCC | 1.04 | 5.77 | 5.55 | 1.20 | Hs.228142 | EST |
| 75 | GGGGGACGGCT | 1.04 | 5.77 | 5.55 | 1.20 | Hs.21346 | hypothetical protein |
| | | | | | | | LOC58481 |
| 76 | CCAGGCACGCT | 1.04 | 5.77 | 5.55 | 1.20 | Hs.198427 | hexokinase 2 |
| 77 | TTTTTAATGTT | 1.04 | 5.77 | 5.55 | 1.20 | Hs.181307 | H3 histone, family |
| | | | | | | | 3A |
| 78 | TACAGTATGTT | 1.04 | 5.77 | 5.55 | 1.20 | Hs.170171 | glutamate-ammonia |
| | | | | | | | ligase (glutamine |
| | | | | | | | synthase) |
| 79 | GTATTCCCCTT | 1.04 | 5.77 | 5.55 | 1.20 | Hs.117176 | poly(A)-binding |
| | | | | | | | protein, nuclear 1 |
| 80 | GCCTGGGCTGG | 1.04 | 5.77 | 5.55 | 1.20 | Hs.112184 | DKFZP586J0619 |
| | | | | | | | protein |
| 81 | GTGGGGGGGAG | 1.04 | 5.77 | 5.55 | 1.20 | Hs.10700 | hypothetical protein |
| 82 | TCACCAAAAAA | 5.21 | 0.96 | 5.43 | 1.12 | Hs.84753 | KIAA0246 protein |
| 83 | GACTTGTATAT | 5.21 | 0.96 | 5.43 | 1.12 | Hs.81328 | NF of kappa light |
| | | | | | | | polypep.gene |
| | | | | | | | enhancer in B-cells |
| | | | | | | | inhibitor, alpha |
| 84 | ATCACACAGCT | 5.21 | 0.96 | 5.43 | 1.12 | Hs.79386 | leiomodin 1 (smooth |
| | | | | | | | muscle) |
| 85 | CTGCTGAGTGA | 5.21 | 0.96 | 5.43 | 1.12 | Hs.79259 | hypothetical protein |
| 86 | GAAACAAGATG | 5.21 | 0.96 | 5.43 | 1.12 | Hs.78771 | phosphoglycerate |
| | | | | | | | kinase 1 |
| 87 | TCTGTAGTCCC | 5.21 | 0.96 | 5.43 | 1.12 | Hs.7358 | Homo sapiens |
| | | | | | | | mRNA; cDNA |
| | | | | | | | DKFZp566D1146 |
| 88 | AACCCGGGGGG | 5.21 | 0.96 | 5.43 | 1.12 | Hs.6214 | KIAA0731 protein |
| 89 | AATAAAGCCTT | 5.21 | 0.96 | 5.43 | 1.12 | Hs.3314 | selenoprotein P, |
| | | | | | | | plasma, 1 |
| 90 | AGCTGGTTTCC | 5.21 | 0.96 | 5.43 | 1.12 | Hs.286027 | etoposide-induced |
| | | | | | | | mRNA |
| 91 | GTTGTCTTTGG | 5.21 | 0.96 | 5.43 | 1.12 | Hs.284394 | complement |
| | | | | | | | component 3 |
| 92 | GAAGCAATAAA | 5.21 | 0.96 | 5.43 | 1.12 | Hs.198253 | major |
| | | | | | | | histocompatibility |
| | | | | | | | complex, class II, |
| | | | | | | | DQ alpha 1 |
| 93 | GTGATGGTGTA | 5.21 | 0.96 | 5.43 | 1.12 | Hs.197345 | thyroid autoantigen |
| | | | | | | | 70kD (Ku antigen) |
| 94 | CTATTGCACTC | 5.21 | 0.96 | 5.43 | 1.12 | Hs.160483 | erythrocyte |
| | | | | | | | membrane protein |
| | | | | | | | band 7.2 (stomatin) |
| 95 | TTACTAAATGG | 5.21 | 0.96 | 5.43 | 1.12 | Hs.155560 | calnexin |
| 96 | TATTTCACCGT | 5.21 | 0.96 | 5.43 | 1.12 | Hs.138860 | Rho GTPase |
| | | | | | | | activating protein 1 |
| 97 | CAGGATCCAGA | 5.21 | 0.96 | 5.43 | 1.12 | Hs.119222 | suppression of |
| | | | | | | | tumorigenicity 13 |
| | | | | | | | (Hsp 70-interacting |
| | | | | | | | protein) |
| 98 | GCAGTCGCTTG | 5.21 | 0.96 | 5.43 | 1.12 | Hs.100002 | HSPC162 protein |
| 99 | GTTCCACAGAA | 10.41 | 1.92 | 5.42 | 1.95 | Hs.179573 | collagen, type I, |
| | | | | | | | alpha 2 |
| 100 | ATGTCTTTTCT | 15.62 | 2.89 | 5.40 | 2.74 | Hs.1516 | insulin-like growth |
| | | | | | | | factor-binding |
| | | | | | | | protein 4 |
| 101 | CCAGGGCAACA | 6.25 | 32.71 | 5.23 | 5.01 | Hs.120980 | ORF-less transcript |
| | | | | | | | in MEN1 region, long |
| | | | | | | | 19 kB version |
| 102 | TCCCTGTACAT | 5.21 | 0.00 | 5.21 | 1.61 | Hs.89563 | nuclear cap binding |
| | | | | | | | protein subunit 1, |
| | | | | | | | 80kD |
| 103 | TTATGGATCTC | 5.21 | 0.00 | 5.21 | 1.61 | Hs.5662 | G protein, beta |
| | | | | | | | polypeptide 2-like 1 |
| 104 | GACCCTAGCTC | 5.21 | 0.00 | 5.21 | 1.61 | Hs.30570 | polyglutamine |
| | | | | | | | binding protein 1 |
| 105 | TACCCCATAAA | 5.21 | 0.00 | 5.21 | 1.61 | Hs.281083 | ESTs |
| 106 | CTTAAAAAAAA | 5.21 | 0.00 | 5.21 | 1.61 | Hs.176626 | hypothetical protein |
| | | | | | | | EDAG-1 |

TABLE 1

| | | | | | | | |
|---|---|---|---|---|---|---|---|
| No. | Tag sequence | Young | Old | Quotient | Significance | Annotation | Description |

| | | | | | | | | |
|---|---|---|---|---|---|---|---|---|
| 107 | TTCTTGAACAA | 9.37 | 1.92 | 4.88 | 1.71 | Hs.76228 | amplified in | |
| | | | | | | | osteosarcoma | |
| 108 | TCGGAGCTGTT | 9.37 | 1.92 | 4.88 | 1.71 | Hs.4055 | chromosome 21 open | |
| | | | | | | | reading fram 50 | |
| 109 | TTTGGTTTTCC | 97.86 | 20.20 | 4.84 | 13.20 | Hs.179573 | collagen, type I, alpha | |
| | | | | | | | 2 | |
| 110 | TGTTGCTCCCA | 0.00 | 4.81 | 4.81 | 1.44 | Hs.82210 | zinc finger protein | |
| | | | | | | | 220 | |
| 111 | GGTTATTTAGT | 0.00 | 4.81 | 4.81 | 1.44 | Hs.8110 | adducin 3 (gamma) | |
| 112 | GAGGTCCCTGG | 0.00 | 4.81 | 4.81 | 1.44 | Hs.74077 | proteasome subunit, | |
| | | | | | | | alpha type, 6 | |
| 113 | CAGCAGCAAAA | 0.00 | 4.81 | 4.81 | 1.44 | Hs.285090 | ESTs | |
| 114 | TTTCTTCCCTT | 0.00 | 4.81 | 4.81 | 1.44 | Hs.283009 | tuftelin 1 | |
| 115 | AGTCTGCTGGG | 0.00 | 4.81 | 4.81 | 1.44 | Hs.259508 | ESTs | |
| 116 | GTGGCGGGCAT | 0.00 | 4.81 | 4.81 | 1.44 | Hs.230564 | EST | |
| 117 | TGTGCGGCTTC | 0.00 | 4.81 | 4.81 | 1.44 | Hs.162196 | hypothetical protein | |
| | | | | | | | FLJ20321 | |
| 118 | GAAGAGGACAA | 0.00 | 4.81 | 4.81 | 1.44 | Hs.120451 | ESTs | |
| 119 | GATCCCAACTG | 0.00 | 4.81 | 4.81 | 1.44 | Hs.118786 | metallothionein 2A | |
| 120 | ATGCTAAAAAA | 0.00 | 4.81 | 4.81 | 1.44 | Hs.116455 | EST | |
| 121 | TGATAATTCAA | 0.00 | 4.81 | 4.81 | 1.44 | Hs.100688 | H, sapiens cDNA | |
| | | | | | | | FLJ11279 fis, clone | |
| | | | | | | | PLACE1009444 | |
| 122 | GATAGCACAGT | 22.90 | 4.81 | 4.76 | 3.54 | Hs.103391 | Human IGFBP5 | |
| | | | | | | | mRNA | |
| 123 | TGAAATAAAAG | 2.08 | 9.62 | 4.63 | 1.68 | Hs.48516 | ESTs | |
| 124 | CCACTGCGCTC | 2.08 | 9.62 | 4.63 | 1.68 | Hs.260287 | ESTs | |
| 125 | TCCTTGCTTCT | 1.04 | 4.81 | 4.63 | 0.98 | Hs.94491 | hypothetical protein | |
| | | | | | | | FLJ20297 | |
| 126 | CCCTCAATCCC | 1.04 | 4.81 | 4.63 | 0.98 | Hs.83077 | interleukin 18 | |
| | | | | | | | (interferon-gamma-inducing | |
| | | | | | | | factor) | |
| 127 | TCCATCAAGAA | 1.04 | 4.81 | 4.63 | 0.98 | Hs.79387 | proteasome 26S | |
| | | | | | | | subunit, ATPase, 5 | |
| 128 | GAAATGAGCAG | 1.04 | 4.81 | 4.63 | 0.98 | Hs.77293 | KIAA0127 gene | |
| | | | | | | | product | |
| 129 | AAGAAGACTTC | 1.04 | 4.81 | 4.63 | 0.98 | Hs.7719 | GABA(A) receptor-associated | |
| | | | | | | | protein | |
| 130 | TGCAATATGCC | 1.04 | 4.81 | 4.63 | 0.98 | Hs.750 | fibrillin 1 (Marfan | |
| | | | | | | | syndrome) | |
| 131 | GTGGCGGGAGC | 1.04 | 4.81 | 4.63 | 0.98 | Hs.68257 | general TF IIF | |
| | | | | | | | polypeptide 1 (74kD | |
| | | | | | | | subunit) | |
| 132 | TGTTCATCATC | 1.04 | 4.81 | 4.63 | 0.98 | Hs.65450 | reticulon 4 | |
| 133 | CTGGATGGGCA | 1.04 | 4.81 | 4.63 | 0.98 | Hs.44017 | SIR2-like | |
| 134 | GGGAAACCCCG | 1.04 | 4.81 | 4.63 | 0.98 | Hs.254283 | EST | |
| 135 | TAAGTAGCAAA | 1.04 | 4.81 | 4.63 | 0.98 | Hs.239625 | integral membrane | |
| | | | | | | | protein 2B | |
| 136 | CCTGTGGTCTC | 1.04 | 4.81 | 4.63 | 0.98 | Hs.236504 | EST | |
| 137 | CGGGCACCTTC | 1.04 | 4.81 | 4.63 | 0.98 | Hs.198249 | gap junction protein, | |
| | | | | | | | beta 5 (connexin | |
| | | | | | | | 31,1) | |
| 138 | TTCAGTGCCTG | 1.04 | 4.81 | 4.63 | 0.98 | Hs.180933 | CpG binding protein | |
| 139 | ACAAACTTAGG | 1.04 | 4.81 | 4.63 | 0.98 | Hs.177656 | calmodulin 1 | |
| | | | | | | | (phosphorylase | |
| | | | | | | | kinase, delta) | |
| 140 | GATTTGTGTTC | 1.04 | 4.81 | 4.63 | 0.98 | Hs.173125 | peptidyiprolyl | |
| | | | | | | | isomerase F | |
| | | | | | | | (cyclophilin F) | |
| 141 | TTGAATTCCCC | 1.04 | 4.81 | 4.63 | 0.98 | Hs.171921 | semaphorin 3C | |
| 142 | TAGTTGAAGTC | 1.04 | 4.81 | 4.63 | 0.98 | Hs.131255 | ubiquinol-cytochrome | |
| | | | | | | | c reductase binding | |
| | | | | | | | protein | |
| 143 | TTTGCACCTTT | 17.70 | 3.85 | 4.60 | 2.79 | Hs.75511 | connective tissue | |
| | | | | | | | growth factor | |
| 144 | CCTGTAATCCA | 8.33 | 1.92 | 4.34 | 1.48 | Hs.253369 | EST | |
| 145 | TTAAATAGCAC | 8.33 | 1.92 | 4.34 | 1.48 | Hs.172928 | collagen, type I, alpha | |
| | | | | | | | 1 | |
| 146 | AAGATCAAGAT | 8.33 | 1.92 | 4.34 | 1.48 | Hs.1288 | actin, alpha 1, | |
| | | | | | | | skeletal muscle | |
| 147 | ACCTTGTGCCC | 4.16 | 0.96 | 4.33 | 0.88 | Hs.878 | sorbitol | |
| | | | | | | | dehydrogenase | |
| 148 | GCTCCGAGCGT | 4.16 | 0.96 | 4.33 | 0.88 | Hs.80617 | ribosomal protein S16 | |
| 149 | CCTTTGTAAGT | 4.16 | 0.96 | 4.33 | 0.88 | Hs.78465 | v-jun avian sarcoma | |
| | | | | | | | virus 17 oncogene | |
| | | | | | | | homolog | |
| 150 | TTCACTGCCGA | 4.16 | 0.96 | 4.33 | 0.88 | Hs.78089 | ATPase, vacuolar, 14 | |
| | | | | | | | kD | |
| 151 | TTCTGTGAATC | 4.16 | 0.96 | 4.33 | 0.88 | Hs.77870 | ESTs | |
| 152 | TTGCCGGTTAA | 4.16 | 0.96 | 4.33 | 0.88 | Hs.75925 | proteasome inhibitor | |
| | | | | | | | subunit 1 (P131) | |
| 153 | TGCATCTGGTG | 4.16 | 0.96 | 4.33 | 0.88 | Hs.75410 | heat shock 70kD | |
| | | | | | | | protein 5 (GRP78, | |
| | | | | | | | BiP) | |
| 154 | TTTACAAAGAG | 4.16 | 0.96 | 4.33 | 0.88 | Hs.75360 | carboxypeptidase E | |
| 155 | CAGACTTTTGG | 4.16 | 0.96 | 4.33 | 0.88 | Hs.63348 | DKFZP586M121 | |
| | | | | | | | protein | |
| 156 | CTGTCCTTGTG | 4.16 | 0.96 | 4.33 | 0.88 | Hs.6101 | Human DNA | |
| | | | | | | | sequence from clone | |
| | | | | | | | 511E16 | |
| 157 | TCCGTGTATAA | 4.16 | 0.96 | 4.33 | 0.88 | Hs.3321 | EST | |
| 158 | ACCCTGCCAAA | 4.16 | 0.96 | 4.33 | 0.88 | Hs.284546 | EST | |
| 159 | CTCCCCCAAGC | 4.16 | 0.96 | 4.33 | 0.88 | Hs.283305 | immunoglobulin | |
| | | | | | | | heavy contant alpha | |
| | | | | | | | 1 | |
| 160 | CTTGCAGTCCT | 4.16 | 0.96 | 4.33 | 0.88 | Hs.27018 | Ris | |
| 161 | AACAGGGGCCA | 4.16 | 0.96 | 4.33 | 0.88 | Hs.262958 | EST | |
| 162 | CCTGGCCAGAA | 4.16 | 0.96 | 4.33 | 0.88 | Hs.261734 | EST | |
| 163 | AGGGAAAAAAA | 4.16 | 0.96 | 4.33 | 0.88 | Hs.215595 | G protein, beta | |
| | | | | | | | polypeptide 1 | |
| 164 | AAGAAAGGAGT | 4.16 | 0.96 | 4.33 | 0.88 | Hs.202097 | procollagen C-endopeptidase | |
| | | | | | | | enhancer | |
| 165 | CCCGACGTGCC | 4.16 | 0.96 | 4.33 | 0.88 | Hs.198269 | NADH | |
| | | | | | | | dehydrogenase 1 | |
| | | | | | | | alpha subcomplex, 3 | |
| 166 | AAGGGAGGGTC | 4.16 | 0.96 | 4.33 | 0.88 | Hs.182248 | sequestosome 1 | |
| 167 | CCCCAGGAGAA | 4.16 | 0.96 | 4.33 | 0.88 | Hs.169902 | solute carrier family | |
| | | | | | | | 2, member 1 | |
| 168 | CTGGGCGTGTC | 4.16 | 0.96 | 4.33 | 0.88 | Hs.161554 | hypothetical protein | |
| | | | | | | | FLJ20159 | |
| 169 | TACTTGGGAGG | 4.16 | 0.96 | 4.33 | 0.88 | Hs.154103 | LIM protein (similar to | |
| | | | | | | | PKC-binding enigma) | |
| 170 | AAAGAAAGTGG | 4.16 | 0.96 | 4.33 | 0.88 | Hs.151513 | beta-1,2-N-acetylglucosaminyl- | |
| | | | | | | | transferase | |
| 171 | TATCTGTCTAC | 4.16 | 0.96 | 4.33 | 0.88 | Hs.145279 | SET translocation | |
| | | | | | | | (myeloid leukemia-associated) | |
| 172 | GAATCACTGCC | 4.16 | 0.96 | 4.33 | 0.88 | Hs.14454 | chromosome 2 open | |
| | | | | | | | reading frame 1 | |
| 173 | ATTAACAAAGC | 4.16 | 0.96 | 4.33 | 0.88 | Hs.113368 | neuroendocrine | |
| | | | | | | | secretory protein 55 | |
| 174 | TTCTGCTCTTG | 4.16 | 0.96 | 4.33 | 0.88 | Hs.110802 | von Willebrand factor | |
| 175 | GACATAAATCC | 4.16 | 0.96 | 4.33 | 0.88 | Hs.109281 | Nef-associated factor | |
| | | | | | | | 1 | |
| 176 | ACGGTGATGTC | 4.16 | 0.96 | 4.33 | 0.88 | Hs.10453 | ESTs | |
| 177 | GATCAGGCCAG | 24.99 | 5.77 | 4.33 | 3.59 | Hs.119571 | collagen, type III, | |
| | | | | | | | alpha 1 | |
| 178 | CTTTATTCCAG | 41.64 | 9.62 | 4.33 | 5.59 | Hs.172928 | collagen, type I, alpha | |
| | | | | | | | 1 | |
| 179 | GTGTTAACCAG | 12.49 | 2.89 | 4.32 | 2.03 | Hs.74267 | ribosomal protein L15 | |
| 180 | CCATTGTACTC | 12.49 | 2.89 | 4.32 | 2.03 | Hs.108740 | DKFZP586A0522 | |
| | | | | | | | protein | |
| 181 | GGAAATGTCAA | 3.12 | 13.47 | 4.32 | 2.12 | Hs.111301 | matrix | |
| | | | | | | | metalloproteinase 2 | |
| 182 | ACCAAAAACCA | 100.98 | 24.05 | 4.20 | 12.20 | Hs.172928 | collagen, type I, alpha | |
| | | | | | | | 1 | |
| 183 | AGAAAGATGTC | 2.08 | 8.66 | 4.16 | 1.47 | Hs.78225 | annexin A1 | |
| 184 | GAAGATGTGGG | 2.08 | 8.66 | 4.16 | 1.47 | Hs.250911 | Homo sapiens clone | |
| | | | | | | | 23967 unknown | |
| | | | | | | | mRNA | |
| 185 | TCACCTTAGGT | 2.08 | 8.66 | 4.16 | 1.47 | Hs.239625 | integral membrane | |
| | | | | | | | protein 2B | |
| 186 | TGGTTGGTGGT | 2.08 | 8.66 | 4.16 | 1.47 | Hs.12701 | plasmolipin | |
| 187 | TGCACACACAC | 4.16 | 0.00 | 4.16 | 1.29 | Hs.99816 | beta-catenin-interacting | |
| | | | | | | | protein | |
| 188 | TTAAAGATTTA | 4.16 | 0.00 | 4.16 | 1.29 | Hs.77899 | tropomyosin 1 (alpha) | |
| 189 | GTGCTATTCTG | 4.16 | 0.00 4.16 | 1.29 | Hs.77873 | mRNA full length | | |
| | | | | | | | insert cDNA | |
| | | | | | | | (EUROIMAGE | |
| | | | | | | | 2176457) | |
| 190 | TTTTCAAGAAG | 4.16 | 0.00 | 4.16 | 1.29 | Hs.75447 | ralA binding protein 1 | |
| 191 | TACATTGCTTT | 4.16 | 0.00 | 4.16 | 1.29 | Hs.75104 | RNA-binding protein | |
| | | | | | | | S1, serine-rich | |
| | | | | | | | domain | |
| 192 | AGGCTGGATGC | 4.16 | 0.00 | 4.16 | 1.29 | Hs.5898 | K1AA0668 protein | |
| 193 | TGTCCACACAT | 4.16 | 0.00 | 4.16 | 1.29 | Hs.5897 | Homo sapiens | |
| | | | | | | | mRNA; cDNA | |
| | | | | | | | DKFZp586P1622 | |
| 194 | TGATCTGCCTG | 4.16 | 0.00 | 4.16 | 1.29 | Hs.5723 | EST | |
| 195 | ATAGGTCAGAA | 4.16 | 0.00 | 4.16 | 1.29 | Hs.29665 | KIAA0911 protein | |
| 196 | GGTGAAACCCC | 4.16 | 0.00 | 4.16 | 1.29 | Hs.284878 | EST | |
| 197 | TCCATCTGTTG | 4.16 | 0.00 | 4.16 | 1.29 | Hs.252189 | syndecan 4 | |
| | | | | | | | (amphiglycan, | |
| | | | | | | | ryudocan) | |
| 198 | ACTGGGCAGTG | 4.16 | 0.00 | 4.16 | 1.29 | Hs.241257 | latent transforming | |
| | | | | | | | growth factor beta | |
| | | | | | | | binding protein 1 | |
| 199 | TAAAAACTTTC | 4.16 | 0.00 | 4.16 | 1.29 | Hs.204096 | lipophilin B | |
| | | | | | | | (uteroglobin family | |
| | | | | | | | member), prostatein-like | |
| 200 | TCCGGCCGCGA | 4.16 | 0.00 | 4.16 | 1.29 | Hs.171774 | hypothetical protein | |
| 201 | CATCTGTAATC | 4.16 | 0.00 | 4.16 | 1.29 | Hs.153290 | EST | |
| 202 | AGGTCAAAAAA | 4.16 | 0.00 | 4.16 | 1.29 | Hs.149570 | actin related protein | |
| | | | | | | | 2/3 complex, subunit | |
| | | | | | | | 4 (20 kD) | |
| 203 | CTCCCTGAACG | 4.16 | 0.00 | 4.16 | 1.29 | Hs.11006 | ESTs | |
| 204 | TGGAAATGACC | 87.45 | 21.17 | 4.13 | 10.54 | Hs.172928 | collagen, type I, alpha | |
| | | | | | | | 1 | |
| 205 | GCCCCCAATAA | 54.13 | 13.47 | 4.02 | 6.67 | Hs.227751 | lectin, galactoside-binding, | |
| | | | | | | | soluble, 1 | |
| | | | | | | | (galectin 1) | |
| 206 | AGAACCTTAAA | 11.45 | 2.89 | 3.96 | 1.81 | Hs.181244 | major | |
| | | | | | | | histocompatibility | |
| | | | | | | | complex, class I, A | |
| 207 | ATGTGAAGAGT | 48.93 | 12.51 | 3.91 | 5.97 | Hs.111779 | secreted protein, | |
| | | | | | | | acidic, cystein-rich | |
| | | | | | | | (osteonectin) | |
| 208 | GATGAGGAGAC | 37.48 | 9.62 | 3.90 | 4.72 | Hs.179573 | collagen, type I, alpha | |
| | | | | | | | 2 | |
| 209 | ATAGCCAGGGA | 0.00 | 3.85 | 3.85 | 1.15 | Hs.95582 | SRY (sex determining | |
| | | | | | | | region Y)-box 20 | |
| 210 | AAAAGCAGAAA | 0.00 | 3.85 | 3.85 | 1.15 | Hs.84728 | Kruppel-like factor 5 | |
| | | | | | | | (intestinal) | |
| 211 | TAATTTGCGTT | 0.00 | 3.85 | 3.85 | 1.15 | Hs.79368 | epithelial membrane | |
| | | | | | | | protein 1 | |
| 212 | TGAGGCCAGGC | 0.00 | 3.85 | 3.85 | 1.15 | Hs.79162 | structure specific | |
| | | | | | | | recognition protein 1 | |
| 213 | GTTTTTGCTTC | 0.00 | 3.85 | 3.85 | 1.15 | Hs.79110 | nucleolin | |
| 214 | TAGGCCCAAGT | 0.00 | 3.85 | 3.85 | 1.15 | Hs.78880 | iIvB (bacterial | |
| | | | | | | | acetolactate | |
| | | | | | | | synthase)-like | |
| 215 | CAGTTACAAAG | 0.00 | 3.85 | 3.85 | 1.15 | Hs.77508 | glutamate | |
| | | | | | | | dehydrogenase 1 | |
| 216 | GACCACGAATA | 0.00 | 3.85 | 3.85 | 1.15 | Hs.76476 | cathepsin H | |
| 217 | CTGGGCCAGCC | 0.00 | 3.85 | 3.85 | 1.15 | Hs.74669 | vesicle-associated | |
| | | | | | | | membrane protein 5 | |
| | | | | | | | (myobrevin) | |
| 218 | AATTACAGCCA | 0.00 | 3.85 | 3.85 | 1.15 | Hs.74471 | gap junction protein, | |
| | | | | | | | alpha 1, 43kD | |
| | | | | | | | (connexin 43) | |
| 219 | AGGATGACCAG | 0.00 | 3.85 | 3.85 | 1.15 | Hs.69554 | hypothetical protein | |
| | | | | | | | FLJ20552 | |
| 220 | GAAACCGAGGG | 0.00 | 3.85 | 3.85 | 1.15 | Hs.279813 | hypothetical protein | |
| 221 | AGCCGAGATCG | 0.00 | 3.85 | 3.85 | 1.15 | Hs.278053 | EST | |
| 222 | CCGGCCCTACC | 0.00 | 3.85 | 3.85 | 1.15 | Hs.271473 | epithelial protein up-regulated | |
| | | | | | | | in carcinoma | |
| 223 | CTGTCTGTGGC | 0.00 | 3.85 | 3.85 | 1.15 | Hs.260150 | hypothetical protein | |
| | | | | | | | FLJ20552 | |
| 224 | AACGCTGCGAA | 0.00 | 3.85 | 3.85 | 1.15 | Hs.24174 | K1AA0876 protein | |
| 225 | CAAGCGCTCTA | 0.00 | 3.85 | 3.85 | 1.15 | Hs.23598 | CREB binding protein | |
| | | | | | | | (Rubinstein-Taybi | |
| | | | | | | | syndrome) | |
| 226 | CCTGTAGTTCT | 0.00 | 3.85 | 3.85 | 1.15 | Hs.231918 | EST | |
| 227 | GCAAAACACTG | 0.00 | 3.85 | 3.85 | 1.15 | Hs.198552 | Homo sapiens | |
| | | | | | | | mRNA; cDNA | |
| | | | | | | | DKFZp566B193 | |
| 228 | CCTGCTCCCTG | 0.00 | 3.85 | 3.85 | 1.15 | Hs.184601 | solute carrier family 7 | |
| | | | | | | | member 5 | |
| 229 | GAAGCTTTGCA | 0.00 | 3.85 | 3.85 | 1.15 | Hs.180532 | heat shock 90kD | |
| | | | | | | | protein 1, alpha | |
| 230 | CCACTAATGGA | 0.00 | 3.85 | 3.85 | 1.15 | Hs.180139 | SMT3 (suppressor of | |
| | | | | | | | mif two 3, yeast) | |
| | | | | | | | homolog 2 | |
| 231 | GGCCCCATTGC | 0.00 | 3.85 | 3.85 | 1.15 | Hs.173421 | clone CE29 8,1 | |
| | | | | | | | (CAC)n/(GTG)n | |
| | | | | | | | repeat-containing | |
| 232 | GGATGCGCAGG | 0.00 | 3.85 | 3.85 | 1.15 | Hs.168541 | H, sapiens mRNA full | |
| | | | | | | | length (EUROIMAGE | |
| | | | | | | | 50374) | |
| 233 | GCCGACGCCAG | 0.00 | 3.85 | 3.85 | 1.15 | Hs.165565 | ESTs | |
| 234 | CCTTGAGTACA | 0.00 | 3.85 | 3.85 | 1.15 | Hs.155247 | aldolase C, fructose-bisphosphate | |
| 235 | TGGAATTCCCT | 0.00 | 3.85 | 3.85 | 1.15 | Hs.154846 | phosphatidylinositol | |
| | | | | | | | 4-kinase, catalytic, | |
| | | | | | | | beta | |
| 236 | AAGCGGGACCT | 0.00 | 3.85 | 3.85 | 1.15 | Hs.153436 | N-acetyltransferase, | |
| | | | | | | | homolog of S, | |
| | | | | | | | cerevisiae ARD1 | |
| 237 | CCAGGCTGCGT | 0.00 | 3.85 | 3.85 | 1.15 | Hs.149846 | integrin, beta 5 | |
| 238 | CCTCTAGTCCC | 0.00 | 3.85 | 3.85 | 1.15 | Hs.145501 | ESTs | |
| 239 | ACGGAAGTTTT | 0.00 | 3.85 | 3.85 | 1.15 | Hs.144974 | ESTs | |
| 240 | ATGGCACATTC | 0.00 | 3.85 | 3.85 | 1.15 | Hs.14328 | Homo sapiens | |
| | | | | | | | mRNA; cDNA | |
| | | | | | | | DKFZp762O124 | |
| 241 | GAGCAAACGGA | 0.00 | 3.85 | 3.85 | 1.15 | Hs.108847 | Homo sapiens | |
| | | | | | | | chromosome 19, | |
| | | | | | | | cosmid R26445 | |
| 242 | CAGCTGGCCAT | 7.29 | 1.92 | 3.80 | 1.26 | Hs.79732 | fibulin 1 | |
| 243 | ATCAAATGCAA | 7.29 | 1.92 | 3.80 | 1.26 | Hs.79070 | v-myc avian | |
| | | | | | | | myelocytomatosis | |
| | | | | | | | viral oncogene | |
| | | | | | | | homolog | |
| 244 | CACCAGCATTG | 7.29 | 1.92 | 3.80 | 1.26 | Hs.75847 | chromosome 15 open | |
| | | | | | | | reading frame 3 | |
| 245 | CAGTTACTTAG | 7.29 | 1.92 | 3.80 | 1.26 | Hs.279920 | tyrosine 3-monoxygenase/tryptophan | |
| | | | | | | | 5-monooxygenase | |
| | | | | | | | activation protein, | |
| | | | | | | | beta polypeptide | |
| 246 | ATGGCGGGTGC | 7.29 | 1.92 | 3.80 | 1.26 | Hs.172382 | hypothetical protein | |
| | | | | | | | FLJ20001 | |
| 247 | TTGCATATCAG | 2.08 | 7.70 | 3.70 | 1.28 | Hs.82237 | ataxia-telangiectasia | |
| | | | | | | | group D-associated | |
| | | | | | | | protein | |
| 248 | TCTGCTAAAGA | 1.04 | 3.85 | 3.70 | 0.77 | Hs.95958 | solute carrier family | |
| | | | | | | | 2, member 4 | |
| 249 | TTGGACCTGGG | 1.04 | 3.85 | 3.70 | 0.77 | Hs.89761 | ATP synthase, H+ | |
| | | | | | | | transporting, delta | |
| | | | | | | | subunit | |
| 250 | GCTTCCTCCTC | 1.04 | 3.85 | 3.70 | 0.77 | Hs.85289 | CD34 antigen | |
| 251 | TGTTTGTGTGT | 1.04 | 3.85 | 3.70 | 0.77 | Hs.85015 | ESTs | |
| 252 | CATTATAACTT | 1.04 | 3.85 | 3.70 | 0.77 | Hs.84359 | hypothetical protein | |
| 253 | AGATACATAGC | 1.04 | 3.85 | 3.70 | 0.77 | Hs.84045 | Homo sapiens cDNA | |
| | | | | | | | FLJ20288 fis, clone | |
| | | | | | | | HEP04414 | |
| 254 | TGTGGTGGTGT | 1.04 | 3.85 | 3.70 | 0.77 | Hs.83422 | MLN51 protein | |
| 255 | GGAGATGAGGA | 1.04 | 3.85 | 3.70 | 0.77 | Hs.83419 | KIAA0252 protein | |
| 256 | GGCTGAGCTCA | 1.04 | 3.85 | 3.70 | 0.77 | Hs.83004 | interleukin 14 | |
| 257 | GCCCGCCTTGT | 1.04 | 3.85 | 3.70 | 0.77 | Hs.80475 | polymerase (RNA) II | |
| | | | | | | | polypeptide J | |
| | | | | | | | (13,3kD) | |
| 258 | GGGTTTGTTTC | 1.04 | 3.85 | 3.70 | 0.77 | Hs.75969 | proline-rich protein | |
| | | | | | | | with nuclear targeting | |
| | | | | | | | signal | |
| 259 | CTATGGCTTCA | 1.04 | 3.85 | 3.70 | 0.77 | Hs.75618 | RAB11A, member | |
| | | | | | | | RAS oncogene family | |
| 260 | CTGGCCGCAAG | 1.04 | 3.85 | 3.70 | 0.77 | Hs.74649 | cytochrome c oxidase | |
| | | | | | | | subunit Vic | |
| 261 | GTGTATCTTTT | 1.04 | 3.85 | 3.70 | 0.77 | Hs.73965 | splicing factor, | |
| | | | | | | | arginine/serine-rich 2 | |
| 262 | CTCCATCGGCT | 1.04 | 3.85 | 3.70 | 0.77 | Hs.65238 | 95 kDa RB protein | |
| | | | | | | | binding protein; | |
| | | | | | | | (KIAA0661) | |
| 263 | CACCACCACGC | 1.04 | 3.85 | 3.70 | 0.77 | Hs.5862 | hypothetical protein | |
| 264 | GCGACCAACAT | 1.04 | 3.85 | 3.70 | 0.77 | Hs.4055 | chromosome 21 open | |
| | | | | | | | reading frame 50 | |
| 265 | GCTAAGGAGAT | 1.04 | 3.85 | 3.70 | 0.77 | Hs.286250 | ras-related C3 BTx | |
| | | | | | | | substrate 1(G-Protein | |
| | | | | | | | Rac1) | |
| 266 | AGGCCTCGGCA | 1.04 | 3.85 | 3.70 | 0.77 | Hs.286202 | H, sapiens cDNA | |
| | | | | | | | FLJ11346 fis, clone | |
| | | | | | | | PLACE1010900 | |
| 267 | TGCTGCTGCTT | 1.04 | 3.85 | 3.70 | 0.77 | Hs.283685 | hypothetical protein | |
| | | | | | | | FLJ20396 | |
| 268 | GCCCCGCCCTC | 1.04 | 3.85 | 3.70 | 0.77 | Hs.280666 | Homo sapiens | |
| | | | | | | | chromosome 19, | |
| | | | | | | | cosmid R32184 | |
| 269 | AGCCGAGATCA | 1.04 | 3.85 | 3.70 | 0.77 | Hs.277663 | EST | |
| 270 | GTGGTATGTGC | 1.04 | 3.85 | 3.70 | 0.77 | Hs.277102 | EST | |
| 271 | CCCTGGCAATG | 1.04 | 3.85 | 3.70 | 0.77 | Hs.273369 | hematopoietic | |
| | | | | | | | stem/progenitor cells | |
| | | | | | | | protein MDS027 | |
| 272 | GTGAAGCCTCA | 1.04 | 3.85 | 3.70 | 0.77 | Hs.271823 | ESTs | |
| 273 | CGAGGGCACTC | 1.04 | 3.85 | 3.70 | 0.77 | Hs.26915 | spectrin, beta, non-erythrocytic 2 | |
| 274 | CTGAAATCTAT | 1.04 | 3.85 | 3.70 | 0.77 | Hs.253467 | ESTs | |
| 275 | CCTGTGATCCT | 1.04 | 3.85 | 3.70 | 0.77 | Hs.240395 | potassium channel, | |
| | | | | | | | subfamily K, member | |
| | | | | | | | 6 (TWIK-2) | |
| 276 | TCACTGCATTC | 1.04 | 3.85 | 3.70 | 0.77 | Hs.235587 | EST | |
| 277 | ACAACACCCCA | 1.04 | 3.85 | 3.70 | 0.77 | Hs.21453 | mRNA for inositol | |
| | | | | | | | 1,4,5-trisphosphate 3-kinase | |
| 278 | ATTGCATCACT | 1.04 | 3.85 | 3.70 | 0.77 | Hs.209111 | EST | |
| 279 | TAGCTCCCTTG | 1.04 | 3.85 | 3.70 | 0.77 | Hs.199160 | myeloid/lymphoid or | |
| | | | | | | | mixed-lineage | |
| | | | | | | | leukemia (trithorax | |
| | | | | | | | (Drosophila) | |
| | | | | | | | homolog) | |
| 280 | AAGCTCTGTGT | 1.04 | 3.85 | 3.70 | 0.77 | Hs.19813 | ESTs | |
| 281 | GTGATGGATGG | 1.04 | 3.85 | 3.70 | 0.77 | Hs.181046 | Homo sapiens | |
| | | | | | | | mRNA; cDNA | |
| | | | | | | | DKFZp586O1919 | |
| | | | | | | | (from clone | |
| | | | | | | | DKFZp586O1919) | |
| 282 | GACCAGAAAAA | 1.04 | 3.85 | 3.70 | 0.77 | Hs.180714 | cytochrome c oxidase | |
| | | | | | | | subunit Via | |
| | | | | | | | polypeptide 1 | |
| 283 | GCCTGGTGACC | 1.04 | 3.85 | 3.70 | 0.77 | Hs.180224 | death-associated | |
| | | | | | | | protein 6 | |
| 284 | GTGCTCTGTAC | 1.04 | 3.85 | 3.70 | 0.77 | Hs.177556 | melanoma antigen, | |
| | | | | | | | family D, 1 | |
| 285 | CAGGAGGAAAG | 1.04 | 3.85 | 3.70 | 0.77 | Hs.177425 | KIAA0964 protein | |
| 286 | GACAATGCCAG | 1.04 | 3.85 | 3.70 | 0.77 | Hs.155433 | ATP synthase, H+ | |
| | | | | | | | transporting | |
| | | | | | | | mitochondrial F1 | |
| | | | | | | | complex, gamma | |
| | | | | | | | polypeptide 1 | |
| 287 | AGCTTCCAGCC | 1.04 | 3.85 | 3.70 | 0.77 | Hs.144974 | ESTs, Highly similar | |
| | | | | | | | to unnamed protein | |
| | | | | | | | produce [H,sapiens] | |
| 288 | TGGGCCCGTGT | 1.04 | 3.85 | 3.70 | 0.77 | Hs.11607 | ESTs | |
| 289 | GATCTCTTGGG | 1.04 | 3.85 | 3.70 | 0.77 | Hs.115947 | keratin 16 (focal non-epidermolytic | |
| | | | | | | | palmoplantar | |
| | | | | | | | keratoderma) | |
| 290 | TGTGACCTCTC | 1.04 | 3.85 | 3.70 | 0.77 | Hs.108973 | dolichyl-phosphate | |
| | | | | | | | mannosyltransferase | |
| | | | | | | | polypeptide 2, | |
| | | | | | | | regulatory subunit | |
| 291 | TGCTTCATCTG | 1.04 | 3.85 | 3.70 | 0.77 | Hs.10842 | RAN, member RAS | |
| | | | | | | | oncogene family | |
| 292 | GTGCTCAAACC | 1.04 | 3.85 | 3.70 | 0.77 | Hs.103915 | KIAA0346 protein | |
| 293 | AGCAGGGCTCC | 1.04 | 3.85 | 3.70 | 0.77 | Hs.100623 | phospholipase C, | |
| | | | | | | | beta 3, neighbor | |
| | | | | | | | pseudogene | |
| 294 | TGCACTTCAAG | 10.41 | 2.89 | 3.60 | 1.60 | Hs.75445 | SPARC-like 1 (mast9, | |
| | | | | | | | hevin) | |
| 295 | ACGCAGGGAGA | 11.45 | 40.41 | 3.53 | 4.58 | Hs.180532 | heat shock 90kD | |
| | | | | | | | protein 1, alpha | |
| 296 | CAAGACGGGGG | 4.16 | 14.43 | 3.47 | 1.97 | Hs.106185 | ral guanine | |
| | | | | | | | nucleotide | |
| | | | | | | | dissociation | |
| | | | | | | | stimulator | |
| 297 | TACTCTTGGCA | 3.12 | 10.58 | 3.39 | 1.54 | Hs.2730 | heterogeneous | |
| | | | | | | | nuclear | |
| | | | | | | | ribonucleoprotein L | |
| 298 | CTCTAAGAAGC | 6.25 | 1.92 | 3.26 | 1.05 | Hs.9641 | complement | |
| | | | | | | | component 1, q | |
| | | | | | | | subcomponent, alpha | |
| | | | | | | | polypeptide | |
| 299 | GTGCGCTAGGG | 6.25 | 1.92 | 3.26 | 1.05 | Hs.9408 | IKK-related kinase | |
| | | | | | | | epsilon; inducible | |
| | | | | | | | ikappaB kinase | |
| 300 | TGATCTCCAAA | 6.25 | 1.92 | 3.26 | 1.05 | Hs.83190 | fatty acid synthase | |
| 301 | AGCACATTTGA | 6.25 | 1.92 | 3.26 | 1.05 | Hs.80562 | gelsolin (amyloidosis, | |
| | | | | | | | Finnish type) | |
| 302 | TCTTGTGCATA | 6.25 | 1.92 | 3.26 | 1.05 | Hs.2795 | lactate | |
| | | | | | | | dehydrogenase A | |
| 303 | GCCTATGGTCC | 6.25 | 1.92 | 3.26 | 1.05 | Hs.16561 | HSPC141 protein | |
| 304 | CCTGTAATCTT | 6.25 | 1.92 | 3.26 | 1.05 | Hs.120882 | ESTs | |
| 305 | ATCTCGAAAGG | 6.25 | 1.92 | 3.26 | 1.05 | Hs.10784 | hypothetical protein | |
| | | | | | | | FLJ20037 | |
| 306 | TGTCTTTGCTC | 3.12 | 0.96 | 3.25 | 0.66 | Hs.9589 | ubiquilin 1 | |
| 307 | TTTTATTTCCA | 3.12 | 0.96 | 3.25 | 0.66 | Hs.93780 | ESTs | |
| 308 | CAGTACTGTAT | 3.12 | 0.96 | 3.25 | 0.66 | Hs.9295 | elastin (supravalvular | |
| | | | | | | | aortic stenosis | |
| | | | | | | | Williams-Beuren | |
| | | | | | | | syndrome) | |
| 309 | GCCAAGATGCC | 3.12 | 0.96 | 3.25 | 0.66 | Hs.83135 | p53-responsive gene | |
| | | | | | | | 6 | |
| 310 | CCAACAAGAAT | 3.12 | 0.96 | 3.25 | 0.66 | Hs.82749 | transmembrane 4 | |
| | | | | | | | superfamily member | |
| | | | | | | | 2 | |
| 311 | TCCACGCACCA | 3.12 | 0.96 | 3.25 | 0.66 | Hs.82023 | hypothetical protein | |
| | | | | | | | similar to mouse | |
| | | | | | | | Fbw5 | |
| 312 | GGGGGTCACCG | 3.12 | 0.96 | 3.25 | 0.66 | Hs.80986 | ATP synthase, H+ | |
| | | | | | | | transporting, | |
| | | | | | | | mitochondrial F0 | |
| | | | | | | | complex, subunit c | |
| | | | | | | | (subunit 9), isoform 1 | |
| 313 | TAATAAAGAAT | 3.12 | 0.96 | 3.25 | 0.66 | Hs.80342 | keratin 15 | |
| 314 | GC1TVTCAGAC | 3.12 | 0.96 | 3.25 | 0.66 | Hs.78781 | vascular endothelial | |
| | | | | | | | growth factor B | |
| 315 | TTGTTATTGCC | 3.12 | 0.96 | 3.25 | 0.66 | Hs.78637 | annexin A7 | |
| 316 | TAATCCTCAAG | 3.12 | 0.96 | 3.25 | 0.66 | Hs.78409 | collagen, type XVIII, | |
| | | | | | | | alpha 1 | |
| 317 | GAGCGGCCTCT | 3.12 | 0.96 | 3.25 | 0.66 | Hs.77868 | ORF | |
| 318 | AAACCAAAAAA | 3.12 | 0.96 | 3.25 | 0.66 | Hs.76753 | endoglin (Osler-Rendu-Weber | |
| | | | | | | | syndrome 1) | |
| 319 | TTTCAGAGAGA | 3.12 | 0.96 | 3.25 | 0.66 | Hs.75975 | signal recognition | |
| | | | | | | | particle 9kD | |
| 320 | TAGCCGCTGAG | 3.12 | 0.96 | 3.25 | 0.66 | Hs.75932 | N-ethylmaleimide-sensitive | |
| | | | | | | | factor | |
| | | | | | | | attachment protein, | |
| | | | | | | | alpha | |
| 321 | ATCACTAAAGA | 3.12 | 0.96 | 3.25 | 0.66 | Hs.75888 | phosphogluconate | |
| | | | | | | | dehydrogenase | |
| 322 | ATATGTATATT | 3.12 | 0.96 | 3.25 | 0.66 | Hs.75839 | zinc finger protein 6 | |
| | | | | | | | (CMPX1) | |
| 323 | TTTTCTCTGAA | 3.12 | 0.96 | 3.25 | 0.66 | Hs.75516 | tyrosine kinase 2 | |
| 324 | TGGCCTCTCTG | 3.12 | 0.96 | 3.25 | 0.66 | Hs.75437 | peroxisomal long-chain | |
| | | | | | | | acyl-coA | |
| | | | | | | | thioesterase; | |
| | | | | | | | putative protein | |
| 325 | CTGTTAGTGTG | 3.12 | 0.96 | 3.25 | 0.66 | Hs.75375 | malate | |
| | | | | | | | dehydrogenase 1, | |
| | | | | | | | NAD (soluble) | |
| 326 | GAGGAGGGTGA | 3.12 | 0.96 | 3.25 | 0.66 | Hs.75318 | tubulin, alpha 1 (testis | |
| | | | | | | | specific) | |
| 327 | TCTACTTTTGT | 3.12 | 0.96 | 3.25 | 0.66 | Hs.74598 | polymerase (DNA | |
| | | | | | | | directed), delta 2, | |
| | | | | | | | regulatory subunit | |
| | | | | | | | (50kD) | |
| 328 | CTGGGCCTGGC | 3.12 | 0.96 | 3.25 | 0.66 | Hs.74573 | similar to vaccinia | |
| | | | | | | | virus HINDIII K4L ORF | |
| 329 | AAAATAAACCT | 3.12 | 0.96 | 3.25 | 0.66 | Hs.74304 | periplakin | |
| 330 | GATTTCGTTTT | 3.12 | 0.96 | 3.25 | 0.66 | Hs.738 | early growth | |
| | | | | | | | response 1 | |
| 331 | GCCACTACCCC | 3.12 | 0.96 | 3.25 | 0.66 | Hs.71475 | hypothetical protein | |
| 332 | ACCGCCGTGGT | 3.12 | 0.96 | 3.25 | 0.66 | Hs.68877 | cytochrome b-245, | |
| | | | | | | | alpha polypeptide | |
| 333 | AGCTACCGGGC | 3.12 | 0.96 | 3.25 | 0.66 | Hs.6059 | EGE-containing | |
| | | | | | | | fibulin-like | |
| | | | | | | | extracellular matrix | |
| | | | | | | | protein 2 | |
| 334 | TGGGACTCCAG | 3.12 | 0.96 | 3.25 | 0.66 | Hs.59384 | Homo sapiens | |
| | | | | | | | mRNA; cDNA | |
| | | | | | | | DKFZp586E2023 | |
| | | | | | | | (from clone | |
| | | | | | | | DKFZp586E2023) | |
| 335 | CTGTTCTCTTG | 3.12 | 0.96 | 3.25 | 0.66 | Hs.46824 | ESTs | |
| 336 | TGGAGAGCAAC | 3.12 | 0.96 | 3.25 | 0.66 | Hs.4113 | S-adenosylhomocystein | |
| | | | | | | | 3 hydrolase-like 1 | |
| 337 | GCAAAGAAAAA | 3.12 | 0.96 | 3.25 | 0.66 | Hs.3844 | LIM domain only 4 | |
| 338 | TGAGTGGACAG | 3.12 | 0.96 | 3.25 | 0.66 | Hs.3743 | ESTs, Weakly similar | |
| | | | | | | | to A28996 proline-rich | |
| | | | | | | | protein M14 | |
| | | | | | | | precursor-mouse_[M,musculus] | |
| 339 | TGGATCAACCA | 3.12 | 0.96 | 3.25 | 0.66 | Hs.286030 | caveolin 1 caveolae | |
| | | | | | | | protein, 22kD | |
| 340 | AGCCGGATGCT | 3.12 | 0.96 | 3.25 | 0.66 | Hs.284232 | KIAA0720 protein | |
| 341 | GTGAAACCACA | 3.12 | 0.96 | 3.25 | 0.66 | Hs.283788 | hypothetical protein | |
| | | | | | | | DKFZp547A023 | |
| 342 | GTCCCTGCCTT | 3.12 | 0.96 | 3.25 | 0.66 | Hs.279837 | glutathione S-transferase | |
| | | | | | | | M2 | |
| | | | | | | | (muscle) | |
| 343 | CTCCACAAATT | 3.12 | 0.96 | 3.25 | 0.66 | Hs.278426 | PDGE associated | |
| | | | | | | | protein | |
| 344 | TGGCCCCAGGT | 3.12 | 0.96 | 3.25 | 0.66 | Hs.268571 | apolipoprotein C-I | |
| 345 | ATGGTGGGCGC | 3.12 | 0.96 | 3.25 | 0.66 | Hs.266417 | EST | |
| 346 | GGGAAGTCACC | 3.12 | 0.96 | 3.25 | 0.66 | Hs.264428 | tissue specific | |
| | | | | | | | transplantation | |
| | | | | | | | antigen P35B | |
| 347 | GTGGCGCGCAC | 3.12 | 0.96 | 3.25 | 0.66 | Hs.261403 | ESTs | |
| 348 | GTGGTAGGTGC | 3.12 | 0.96 | 3.25 | 0.66 | Hs.254237 | EST | |
| 349 | TGCCATCTGTA | 3.12 | 0.96 | 3.25 | 0.66 | Hs.23960 | cyclin B1 | |
| 350 | GTGAAATTCCA | 3.12 | 0.96 | 3.25 | 0.66 | Hs.228168 | ESTs | |
| 351 | CCTGTGATTCC | 3.12 | 0.96 | 3.25 | 0.66 | Hs.227961 | EST | |
| 352 | GCGGGGTACCC | 3.12 | 0.96 | 3.25 | 0.66 | Hs.227823 | pM5 protein | |
| 353 | GGGATTAAAGC | 3.12 | 0.96 | 3.25 | 0.66 | Hs.211579 | melanoma adhesion | |
| | | | | | | | molecule | |
| 354 | AATTCAATTAA | 3.12 | 0.96 | 3.25 | 0.66 | Hs.211568 | eukaryotic translation | |
| | | | | | | | initiation factor 4 | |
| | | | | | | | gamma, 1 | |
| 355 | AGGAACACAAA | 3.12 | 0.96 | 3.25 | 0.66 | Hs.211539 | eukaryotic translation | |
| | | | | | | | initiation factor 2, | |
| | | | | | | | subunit 3 (gamma, | |
| | | | | | | | 52kD) | |
| 356 | TTGGCCAGGGT | 3.12 | 0.96 | 3.25 | 0.66 | Hs.209396 | ESTs, Weakly similar | |
| | | | | | | | to plakophilin 2b | |
| | | | | | | | [H,sapiens] | |
| 357 | TTGCTGGAGAA | 3.12 | 0.96 | 3.25 | 0.66 | Hs.197114 | RNA binding protein; | |
| | | | | | | | AT-rich element | |
| | | | | | | | binding factor | |
| 358 | AGCAAACTGAA | 3.12 | 0.96 | 3.25 | 0.66 | Hs.182579 | leucine | |
| | | | | | | | aminopeptidase | |
| 359 | GTGGCGGACGC | 3.12 | 0.96 | 3.25 | 0.66 | Hs.182577 | inositol | |
| | | | | | | | polyphosphate-5-phosphatase, | |
| | | | | | | | 75kD | |
| 360 | TTTTTGATAAA | 3.12 | 0.96 | 3.25 | 0.66 | Hs.181165 | eukaryotic translation | |
| | | | | | | | elongation factor 1 | |
| | | | | | | | alpha 1 | |
| 361 | TAATGGTAACT | 3.12 | 0.96 | 3.25 | 0.66 | Hs.181028 | cytochrome c oxidase | |
| | | | | | | | subunit Va | |
| 362 | TTTTTGTATTA | 3.12 | 0.96 | 3.25 | 0.66 | Hs.179526 | upregulated by 1,25-dihydroxyvitamin | |
| | | | | | | | D-3 | |
| 363 | GGATACAACCT | 3.12 | 0.96 | 3.25 | 0.66 | Hs.173993 | RNA binding motif | |
| | | | | | | | protein 6 | |
| 364 | CAAGGGTGACA | 3.12 | 0.96 | 3.25 | 0.66 | Hs.170222 | solute carrier family 9 | |
| | | | | | | | (sodium/hydrogen | |
| | | | | | | | exchanger), isoform 1 | |
| | | | | | | | (antiporter, Na+/H+, | |
| | | | | | | | amiloride sensitive) | |
| 365 | CGGAGTCCATT | 3.12 | 0.96 | 3.25 | 0.66 | Hs.155595 | neural precursor cell | |
| | | | | | | | expressed, | |
| | | | | | | | developmentally | |
| | | | | | | | down-regulated 5 | |
| 366 | TGCGCGCCCTG | 3.12 | 0.96 | 3.25 | 0.66 | Hs.15093 | hypothetical protein | |
| 367 | GGAAGCACGGA | 3.12 | 0.96 | 3.25 | 0.66 | Hs.148495 | proteasome | |
| | | | | | | | (prosome, | |
| | | | | | | | macropain) 26S | |
| | | | | | | | subunit, non-ATPase, | |
| | | | | | | | 4 | |
| 368 | GAGCCCCCGTG | 3.12 | 0.96 | 3.25 | 0.66 | Hs.12908 | CDC42-binding | |
| | | | | | | | protein kinase beta | |
| | | | | | | | (DMPK-like) | |
| 369 | CAGATGGAGGC | 3.12 | 0.96 | 3.25 | 0.66 | Hs.127273 | hypothetical protein | |
| | | | | | | | FLJ0044 | |
| 370 | GCTGGTGCCTG | 3.12 | 0.96 | 3.25 | 0.66 | Hs.125359 | Thy-1 cell surface | |
| | | | | | | | antigen | |
| 371 | GCCTTTCCCTC | 3.12 | 0.96 | 3.25 | 0.66 | Hs.12064 | ubiquitin specific | |
| | | | | | | | protease 22 | |
| 372 | ACGAAACCCCA | 3.12 | 0.96 | 3.25 | 0.66 | Hs.117582 | CGI-43 protein | |
| 373 | ATTTAAAAAAA | 3.12 | 0.96 | 3.25 | 0.66 | Hs.1139 | cold shock domain | |
| | | | | | | | protein A | |
| 374 | CATTTGGGAAG | 3.12 | 0.96 | 3.25 | 0.66 | Hs.111334 | ferritin, light | |
| | | | | | | | polypeptide | |
| 375 | CCTTCCAAATT | 3.12 | 0.96 | 3.25 | 0.66 | Hs.111076 | malate | |
| | | | | | | | dehydrogenase 2, | |
| | | | | | | | NAD (mitochondrial) | |
| 376 | TTTCTGCTCCT | 3.12 | 0.96 | 3.25 | 0.66 | Hs.108124 | ribosomal protein L41 | |
| 377 | AATATTGAGAA | 3.12 | 0.96 | 3.25 | 0.66 | Hs.106673 | eukaryotic translation | |
| | | | | | | | initiation factor 3, | |
| | | | | | | | subunit 6 (48kD) | |
| 378 | CTGAAACAGCT | 3.12 | 0.96 | 3.25 | 0.66 | Hs.106469 | suppressor of var1 | |
| | | | | | | | (s,cerevisiae) 3-like 1 | |
| 379 | CACTGCCTTTG | 3.12 | 0.96 | 3.25 | 0.66 | Hs.106019 | protein phosphatase | |
| | | | | | | | 1, regulatory subunit | |
| | | | | | | | 10 | |
| 380 | TTGCAACCAAA | 3.12 | 0.96 | 3.25 | 0.66 | Hs.10101 | ESTs, Weakly similar | |
| | | | | | | | to coded for by C, | |
| | | | | | | | elegans cDNA | |
| | | | | | | | yk27g3,5 [C,elegans] | |
| 381 | ATCTCAGCTCA | 9.37 | 2.89 | 3.24 | 1.39 | Hs.246192 | ESTs, Weakly similar | |
| | | | | | | | to RMS1_HUMAN | |
| | | | | | | | REGULATOR OF | |
| | | | | | | | MITOTIC SPINDLE | |
| | | | | | | | ASSEMBLY 1 | |
| | | | | | | | [H,sapiens] | |
| 382 | GCTTTGATGAT | 2.08 | 6.73 | 3.24 | 1.09 | Hs.89649 | epoxide hydrolase 1, | |
| | | | | | | | microsomal | |
| | | | | | | | (xenobiotic) | |
| 383 | AAGCTAATAAA | 2.08 | 6.73 | 3.24 | 1.09 | Hs.88474 | prostaglandin-endoperoxide | |
| | | | | | | | synthase 1 | |
| | | | | | | | (prostaglandin G/H | |
| | | | | | | | synthase and | |
| | | | | | | | cyclooxygenase) | |
| 384 | ATGATGATGAT | 2.08 | 6.73 | 3.24 | 1.09 | Hs.79172 | solute carrier family | |
| | | | | | | | 25 (mitochondrial | |
| | | | | | | | carrier; adenine | |
| | | | | | | | nucleotide | |
| | | | | | | | translocator), | |
| | | | | | | | member 5 | |
| 385 | TAGAAAGGCAG | 2.08 | 6.73 | 3.24 | 1.09 | Hs.78909 | butyrate response | |
| | | | | | | | factor 2 (EGF-response | |
| | | | | | | | factor 2) | |
| 386 | TACCCCACCCT | 2.08 | 6.73 | 3.24 | 1.09 | Hs.7647 | MYC-associated zinc | |
| | | | | | | | finger protein (purine-binding | |
| | | | | | | | transcription | |
| | | | | | | | factor) | |
| 387 | GGAATGTACGT | 2.08 | 6.73 | 3.24 | 1.09 | Hs.429 | ATP synthase, H+ | |
| | | | | | | | transporting, | |
| | | | | | | | mitochondrial F0 | |
| | | | | | | | complex, subunit c | |
| | | | | | | | (subunit 9) isoform 3 | |
| 388 | GTGAAAACCTG | 2.08 | 6.73 | 3.24 | 1.09 | Hs.283606 | ESTs | |
| 389 | TGCCACCACAC | 2.08 | 6.73 | 3.24 | 1.09 | Hs.239993 | ESTs | |
| 390 | ATGTAGGTGCC | 2.08 | 6.73 | 3.24 | 1.09 | Hs.173717 | phosphatidic acid | |
| | | | | | | | phosphatase type 2B | |
| 391 | AATCTAGTTCT | 5.21 | 16.35 | 3.14 | 2.02 | Hs.251440 | Human profilaggrin | |
| | | | | | | | gene exons 1-3, 5′ | |
| | | | | | | | end | |
| 392 | TTGA1TGAGTG | 3.12 | 0.00 | 3.12 | 0.97 | Hs.9879 | ESTs | |
| 393 | AAGCTGCTGGA | 3.12 | 0.00 | 3.12 | 0.97 | Hs.9822 | HCNP protein | |
| 394 | AGTGTCTGTGA | 3.12 | 0.00 | 3.12 | 0.97 | Hs.8867 | cysteine-rich, | |
| | | | | | | | angiogenic inducer, | |
| | | | | | | | 61 | |
| 395 | ATTTTGTGCAA | 3.12 | 0.00 | 3.12 | 0.97 | Hs.8750 | uncharacterized bone | |
| | | | | | | | marrow protein | |
| | | | | | | | BM045 | |
| 396 | CCTGCCCCCCT | 3.12 | 0.00 | 3.12 | 0.97 | Hs.861 | mitogen-activated | |
| | | | | | | | protein kinase 3 | |
| 397 | AAAAATAAAGC | 3.12 | 0.00 | 3.12 | 0.97 | Hs.85100 | WD repeat domain 1 | |
| 398 | TGCTGGTGTGG | 3.12 | 0.00 | 3.12 | 0.97 | Hs.84883 | KIAA0864 protein | |
| 399 | TGAAGAGAATT | 3.12 | 0.00 | 3.12 | 0.97 | Hs.82306 | destrin (actin | |
| | | | | | | | depolymerizing | |
| | | | | | | | factor) | |
| 400 | ATGGCTAAGCT | 3.12 | 0.00 | 3.12 | 0.97 | Hs.82280 | regulator of G-protein | |
| | | | | | | | signalling 10 | |
| 401 | CATCACGGATC | 3.12 | 0.00 | 3.12 | 0.97 | Hs.82112 | interleukin 1 receptor, | |
| | | | | | | | type I | |
| 402 | ACAAGAATTGT | 3.12 | 0.00 | 3.12 | 0.97 | Hs.80919 | synaptophysin-like | |
| | | | | | | | protein | |
| 403 | TGTGAACACAT | 3.12 | 0.00 | 3.12 | 0.97 | Hs.80645 | interferon regulatory | |
| | | | | | | | factor 1 | |
| 404 | GGACCTTGGAG | 3.12 | 0.00 | 3.12 | 0.97 | Hs.78877 | inositol 1,4,5-trisphosphate | |
| | | | | | | | 3-kinase B | |
| 405 | CTTCTATGTAG | 3.12 | 0.00 | 3.12 | 0.97 | Hs.77225 | ADP-ribosyltransferase | |
| | | | | | | | (NAD+; poly (ADP-ribose) | |
| | | | | | | | polymerase)-like 1 | |
| 406 | TTCTTATTTTA | 3.12 | 0.00 | 3.12 | 0.97 | Hs.75916 | splicing factor 3b, | |
| | | | | | | | subunit 2, 145kD | |
| 407 | TTCTCCCAAAT | 3.12 | 0.00 | 3.12 | 0.97 | Hs.75617 | collagen, type IV, | |
| | | | | | | | alpha 2 | |
| 408 | TATTGACAACA | 3.12 | 0.00 | 3.12 | 0.97 | Hs.75608 | tight junction protein | |
| | | | | | | | 2 (zona occludens 2) | |
| 409 | AAAAAGCAGAT | 3.12 | 0.00 | 3.12 | 0.97 | Hs.75428 | superoxide dismutase | |
| | | | | | | | 1, soluble | |
| | | | | | | | (amyotrophic lateral | |
| | | | | | | | sclerosis 1 (adult) | |
| 410 | AGCTATTCCTC | 3.12 | 0.00 | 3.12 | 0.97 | Hs.75334 | exostoses (multiple) 2 | |
| 411 | GTTCAAAGACT | 3.12 | 0.00 | 3.12 | 0.97 | Hs.75260 | mitogen inducible 2 | |
| 412 | AGTTGTCACTT | 3.12 | 0.00 | 3.12 | 0.97 | Hs.74649 | cytochrome c oxidase | |
| | | | | | | | subunit Vlc | |
| 413 | TGGTAGTTACC | 3.12 | 0.00 | 3.12 | 0.97 | Hs.66881 | Homo sapiens | |
| | | | | | | | mRNA; cDNA | |
| | | | | | | | DKFZp564A026 | |
| | | | | | | | (from clone | |
| | | | | | | | DKFZp434A1518); | |
| | | | | | | | partial cds | |
| 414 | TGTAACGTGGG | 3.12 | 0.00 | 3.12 | 0.97 | Hs.66762 | Homo sapiens | |
| | | | | | | | mRNA; cDNA | |
| | | | | | | | DKFZp564A026 | |
| | | | | | | | (from clone | |
| | | | | | | | DKFZp564A026) | |
| 415 | CCCTTCTGCCA | 3.12 | 0.00 | 3.12 | 0.97 | Hs.6214 | KIAA0731 protein | |
| 416 | GACAGTCACTC | 3.12 | 0.00 | 3.12 | 0.97 | Hs.6066 | Rho guanine | |
| | | | | | | | nucleotide exchange | |
| | | | | | | | factor (GEF) 4 | |
| 417 | CAGCTCAGCTG | 3.12 | 0.00 | 3.12 | 0.97 | Hs.58414 | filamin C, gamma | |
| | | | | | | | (actin-binding protein-280) | |
| 418 | GCGAAACCCCT | 3.12 | 0.00 | 3.12 | 0.97 | Hs.46468 | chemokine (C-C | |
| | | | | | | | motif) receptor 6 | |
| 419 | CCATAATGTTG | 3.12 | 0.00 | 3.12 | 0.97 | Hs.39957 | pleckstrin 2 (mouse) | |
| | | | | | | | homolog | |
| 420 | AAAGCATTTCT | 3.12 | 0.00 | 3.12 | 0.97 | Hs.36688 | ESTs, Moderately | |
| | | | | | | | similar to WAP four-disulfide | |
| | | | | | | | core domain | |
| | | | | | | | protein [R,norvegicus] | |
| 421 | ATGACCCGCAG | 3.12 | 0.00 | 3.12 | 0.97 | Hs.286254 | ESTs, Weakly similar | |
| | | | | | | | to AF170723_1 | |
| | | | | | | | protein kinase STK10 | |
| | | | | | | | [H,sapiens] | |
| 422 | GAGCTTACATT | 3.12 | 0.00 | 3.12 | 0.97 | Hs.285706 | ESTs | |
| 423 | TTGGTTTGCTG | 3.12 | 0.00 | 3.12 | 0.97 | Hs.284326 | Human clone 23960 | |
| | | | | | | | mRNA sequence | |
| 424 | GTTACCAGTTT | 3.12 | 0.00 | 3.12 | 0.97 | Hs.28264 | Homo sapiens | |
| | | | | | | | mRNA; cDNA | |
| | | | | | | | DKFZp564L0822 | |
| | | | | | | | (from clone | |
| | | | | | | | DKFZp564L0822) | |
| 425 | TGGAACTGTGA | 3.12 | 0.00 | 3.12 | 0.97 | Hs.279751 | sialic acid binding Ig-like | |
| | | | | | | | lectin 8 | |
| 426 | AAACCCCGTCT | 3.12 | 0.00 | 3.12 | 0.97 | Hs.273464 | ESTs | |
| 427 | GAGGGTCTTGT | 3.12 | 0.00 | 3.12 | 0.97 | Hs.256310 | SH3 domain-containing | |
| | | | | | | | protein | |
| | | | | | | | 6511 | |
| 428 | GTGGTGCGCGC | 3.12 | 0.00 | 3.12 | 0.97 | Hs.252075 | Homo sapiens | |
| | | | | | | | mRNA; cDNA | |
| | | | | | | | DKFZp434D179 | |
| | | | | | | | (from clone | |
| | | | | | | | DKFZp434D179) | |
| 429 | ATCCACCCGCC | 3.12 | 0.00 | 3.12 | 0.97 | Hs.251337 | ESTs | |
| 430 | TGGAGGCCAGG | 3.12 | 0.00 | 3.12 | 0.97 | Hs.250581 | SWI/SNE related, | |
| | | | | | | | matrix associated, | |
| | | | | | | | actin dependent | |
| | | | | | | | regulator of | |
| | | | | | | | chromatin, subfamily | |
| | | | | | | | d, member 2 | |
| 431 | GGGTGCAAAAA | 3.12 | 0.00 | 3.12 | 0.97 | Hs.249495 | heterogeneous | |
| | | | | | | | nuclear | |
| | | | | | | | ribonucleoprotein A1 | |
| 432 | TACTGCAAAAA | 3.12 | 0.00 | 3.12 | 0.97 | Hs.24557 | DKFZP434H018 | |
| | | | | | | | protein | |
| 433 | TTATTTATGAA | 3.12 | 0.00 | 3.12 | 0.97 | Hs.245188 | tissue inhibitor of | |
| | | | | | | | metalloproteinase 3 | |
| | | | | | | | (Sorsby fundus | |
| | | | | | | | dystrophy, | |
| | | | | | | | pseudoinflammatory) | |
| 434 | GTATAAACGTC | 3.12 | 0.00 | 3.12 | 0.97 | Hs.237356 | stromal cell-derived | |
| | | | | | | | factor 1 | |
| 435 | CGTGTTAATGG | 3.12 | 0.00 | 3.12 | 0.97 | Hs.2110 | zinc finger protein 9 | |
| | | | | | | | (a cellular retroviral | |
| | | | | | | | nucleic acid binding | |
| | | | | | | | protein) | |
| 436 | CCTGTAGGCCC | 3.12 | 0.00 | 3.12 | 0.97 | Hs.207938 | EST | |
| 437 | AACTGTCCTTC | 3.12 | 0.00 | 3.12 | 0.97 | Hs.194673 | phosphoprotein | |
| | | | | | | | enriched in astrocytes | |
| | | | | | | | 15 | |
| 438 | CCATTGCATTC | 3.12 | 0.00 | 3.12 | 0.97 | Hs.185156 | ESTs | |
| 439 | TTACTTCCCCA | 3.12 | 0.00 | 3.12 | 0.97 | Hs.184641 | delta-6 fatty acid | |
| | | | | | | | desaturase | |
| 440 | TCTGGCCCAGC | 3.12 | 0.00 | 3.12 | 0.97 | Hs.183 | Duffy blood group | |
| 441 | CCTCTCCCATT | 3.12 | 0.00 | 3.12 | 0.97 | Hs.177533 | Homo sapiens | |
| | | | | | | | mRNA, chromosome | |
| | | | | | | | 1 specific transcript | |
| | | | | | | | KIAA0503 | |
| 442 | TTGTGAGAATA | 3.12 | 0.00 | 3.12 | 0.97 | Hs.177425 | KIAA0964 protein | |
| 443 | AGCTAGGGAAG | 3.12 | 0.00 | 3.12 | 0.97 | Hs.172180 | KIAA044O protein | |
| 444 | GGGAGGTAGCA | 3.12 | 0.00 | 3.12 | 0.97 | Hs.171825 | basic helix-loop-helix | |
| | | | | | | | domain containing, | |
| | | | | | | | classs B, 2 | |
| 445 | GAATGAGGACA | 3.12 | 0.00 | 3.12 | 0.97 | Hs.167791 | reticulocalbin 1, EF-hand | |
| | | | | | | | calcium binding | |
| | | | | | | | domain | |
| 446 | CAACTTAAGTG | 3.12 | 0.00 | 3.12 | 0.97 | Hs.16492 | DKFZP564G2022 | |
| | | | | | | | protein | |
| 447 | CCTAAACTCAA | 3.12 | 0.00 | 3.12 | 0.97 | Hs.16187 | uncharacterized | |
| | | | | | | | hematopoietic | |
| | | | | | | | stem/progenitor cells | |
| | | | | | | | protein MDS032 | |
| 448 | ATCGCACTACT | 3.12 | 0.00 | 3.12 | 0.97 | Hs.161721 | ESTs | |
| 449 | CCTGTAATCTG | 3.12 | 0.00 | 3.12 | 0.97 | Hs.159975 | ESTs | |
| 450 | CTTGTAGTTCC | 3.12 | 0.00 | 3.12 | 0.97 | Hs.155983 | KIAA0677 gene | |
| | | | | | | | product | |
| 451 | ATACAATAAAA | 3.12 | 0.00 | 3.12 | 0.97 | Hs.151734 | nuclear transport | |
| | | | | | | | factor 2 (placental | |
| | | | | | | | protein 15) | |
| 452 | TGATTCTGTTT | 3.12 | 0.00 | 3.12 | 0.97 | Hs.146428 | collagen, type V, | |
| | | | | | | | alpha 1 | |
| 453 | ATTTGTCCCAG | 3.12 | 0.00 | 3.12 | 0.97 | Hs.139800 | high-mobility group | |
| | | | | | | | (nonhistone | |
| | | | | | | | chromosomal) protein | |
| | | | | | | | isoforms I and Y | |
| 454 | AGCTGGGTTGG | 3.12 | 0.00 | 3.12 | 0.97 | Hs.131731 | hypothetical protein | |
| | | | | | | | FLJ11099 | |
| 455 | GGGCTACGTCC | 3.12 | 0.00 | 3.12 | 0.97 | Hs.123107 | kallikrein 1, | |
| | | | | | | | renal/pancreas/salivary | |
| 456 | TGCTGCCTGTT | 3.12 | 0.00 | 3.12 | 0.97 | Hs.118110 | bone marrow stromal | |
| | | | | | | | cell antigen 2 | |
| 457 | CCCCCAATGCT | 3.12 | 0.00 | 3.12 | 0.97 | Hs.115232 | splicing factor 3a, | |
| | | | | | | | subunit 2, 66kD | |
| 458 | AAAGCAGCACA | 3.12 | 0.00 | 3.12 | 0.97 | Hs.108802 | N-ethylmaleimide-sensitive | |
| | | | | | | | factor | |
| 459 | TGGTAACTGGC | 3.12 | 0.00 | 3.12 | 0.97 | Hs.108741 | ESTs | |
| 460 | GCGAGTCTCCG | 3.12 | 0.00 | 3.12 | 0.97 | Hs.10632 | hypothetical protein | |
| | | | | | | | DKFZp762M136 | |
| 461 | TGCCCCTTGCC | 3.12 | 0.00 | 3.12 | 0.97 | Hs.105700 | secreted frizzled-related | |
| | | | | | | | protein 4 | |
| 462 | GCAGGGCCTCA | 3.12 | 9.62 | 3.08 | 1.36 | Hs.92323 | FXYD domain-containing | |
| | | | | | | | ion | |
| | | | | | | | transport regulator 3 | |
| 463 | GCGAAACCCAG | 3.12 | 9.62 | 3.08 | 1.36 | Hs.142442 | HP1-BP74 | |
| 464 | ACAGCGGCAAT | 16.66 | 50.99 | 3.06 | 4.90 | Hs.74316 | desmoplakin (DPI, | |
| | | | | | | | DPII) | |
| 465 | CCCTACCCTGT | 22.90 | 7.70 | 2.97 | 2.53 | Hs.75736 | apolipoprotein D | |
| 466 | CACACGGGCGA | 19.78 | 6.73 | 2.94 | 2.24 | Hs.194679 | WNT1 inducible | |
| | | | | | | | signaling pathway | |
| | | | | | | | protein 2 | |
| 467 | CCCCAGGCTGC | 0.00 | 2.89 | 2.89 | 0.87 | Hs.9645 | ESTs | |
| 468 | ACAAACTGTGG | 0.00 | 2.89 | 2.89 | 0.87 | Hs.90370 | actin related protein | |
| | | | | | | | 2/3 complex, subunit | |
| | | | | | | | 1A (41 kD) | |
| 469 | GACCACCTTTA | 0.00 | 2.89 | 2.89 | 0.87 | Hs.83551 | microfibrillar-associated | |
| | | | | | | | protein 2 | |
| 470 | GCAGCTAATTT | 0.00 | 2.89 | 2.89 | 0.87 | Hs.8207 | GK001 protein | |
| 471 | GAATCGGTTAT | 0.00 | 2.89 | 2.89 | 0.87 | Hs.80595 | NADH | |
| | | | | | | | dehydrogenase | |
| | | | | | | | (ubiquinone) Fe-S | |
| | | | | | | | protein 5 (15kD) | |
| | | | | | | | (NADH-coenzyme aspartyl | |
| | | | | | | | reductase) | |
| 472 | GCAGCTCAGGC | 0.00 | 2.89 | 2.89 | 0.87 | Hs.79572 | cathepsin D | |
| | | | | | | | (lysosomal aspartyl | |
| | | | | | | | protease) | |
| 473 | GCAGGTCAGCC | 0.00 | 2.89 | 2.89 | 0.87 | Hs.78950 | branched chain keto | |
| | | | | | | | acid dehydrogenase | |
| | | | | | | | E1, alpha polypeptide | |
| | | | | | | | (maple syrup urine | |
| | | | | | | | disease) | |
| 474 | TAAACTATTGG | 0.00 | 2.89 | 2.89 | 0.87 | Hs.78851 | KIAA0217 protein | |
| 475 | AGAGCAAGTAC | 0.00 | 2.89 | 2.89 | 0.87 | Hs.78050 | small acidic protein | |
| 476 | GTGAGCAAGAC | 0.00 | 2.89 | 2.89 | 0.87 | Hs.78040 | KDEL (Lys-Asp-Glu-Leu) | |
| | | | | | | | endoplasmic | |
| | | | | | | | reticulum protein | |
| | | | | | | | retention receptor 1 | |
| 477 | CGGCTGAATTC | 0.00 | 2.89 | 2.89 | 0.87 | Hs.75888 | phosphogluconate | |
| | | | | | | | dehydrogenase | |
| 478 | GCTCCACTGGA | 0.00 | 2.89 | 2.89 | 0.87 | Hs.75709 | mannose-6-phosphate | |
| | | | | | | | receptor | |
| | | | | | | | (cation dependent) | |
| 479 | CAAAATCTTGA | 0.00 | 2.89 | 2.89 | 0.87 | Hs.75431 | fibrinogen, gamma | |
| | | | | | | | polypeptide | |
| 480 | GGACAGATGTA | 0.00 | 2.89 | 2.89 | 0.87 | Hs.75356 | transcription factor 4 | |
| 481 | GGGGGTGGATG | 0.00 | 2.89 | 2.89 | 0.87 | Hs.75087 | Fas-activated | |
| | | | | | | | serine/threonine | |
| | | | | | | | kinase | |
| 482 | AGTATGACCTA | 0.00 | 2.89 | 2.89 | 0.87 | Hs.74649 | cytochrome c oxidase | |
| | | | | | | | subunit Vic | |
| 483 | ACAAAGGGCCC | 0.00 | 2.89 | 2.89 | 0.87 | Hs.7416 | KIAA0397 gene | |
| | | | | | | | product | |
| 484 | GGCCAGTAACA | 0.00 | 2.89 | 2.89 | 0.87 | Hs.69559 | KIAA1096 protein | |
| 485 | TACATCAGTAA | 0.00 | 2.89 | 2.89 | 0.87 | Hs.65029 | growth arrest-specific | |
| | | | | | | | 1 | |
| 486 | CGGCTGCCCAC | 0.00 | 2.89 | 2.89 | 0.87 | Hs.63236 | synuclein, gamma | |
| | | | | | | | (breast cancer-specific | |
| | | | | | | | protein 1) | |
| 487 | TGGCAGTCTGC | 0.00 | 2.89 | 2.89 | 0.87 | Hs.6179 | DEAD/H (Asp-Glu-Ala-Asp/His) | |
| | | | | | | | box | |
| | | | | | | | polypeptide 17 | |
| | | | | | | | (72kD) | |
| 488 | GAGCTGGTGAA | 0.00 | 2.89 | 2.89 | 0.87 | Hs.6163 | ESTs, Weakly similar | |
| | | | | | | | to myotonic dystrophy | |
| | | | | | | | kinase [H,sapiens] | |
| 489 | TGGCAGCTTTT | 0.00 | 2.89 | 2.89 | 0.87 | Hs.6153 | CGI-48 protein | |
| 490 | AAGCTGGAGGA | 0.00 | 2.89 | 2.89 | 0.87 | Hs.55682 | eukaryotic translation | |
| | | | | | | | initiation factore 3, | |
| | | | | | | | subunit 7 (zeta, | |
| | | | | | | | 66/67kD) | |
| 491 | GCTTCCATCTT | 0.00 | 2.89 | 2.89 | 0.87 | Hs.55296 | HLA-B associated | |
| | | | | | | | transcript-1 | |
| 492 | GAGTGCAACCC | 0.00 | 2.89 | 2.89 | 0.87 | Hs.54680 | ESTs | |
| 493 | TGGATCCTAGA | 0.00 | 2.89 | 2.89 | 0.87 | Hs.5273 | NADH | |
| | | | | | | | dehydrogenase | |
| | | | | | | | (ubiquinone) Fe-S | |
| | | | | | | | protein 3 (30kD) | |
| | | | | | | | (NADH-coenzyme Q | |
| | | | | | | | reductase) | |
| 494 | TATGTGATTTG | 0.00 | 2.89 | 2.89 | 0.87 | Hs.5216 | HSPC028 protein | |
| 495 | GGGACGAGTGA | 0.00 | 2.89 | 2.89 | 0.87 | Hs.3337 | transmembrane 4 | |
| | | | | | | | superfamily member | |
| | | | | | | | 1 | |
| 496 | ATTGTTTCAAG | 0.00 | 2.89 | 2.89 | 0.87 | Hs.32366 | ESTs, Moderately | |
| | | | | | | | similar to | |
| | | | | | | | TWST_HUMAN | |
| | | | | | | | TWIST RELATED | |
| | | | | | | | PROTEIN [H,sapiens] | |
| 497 | TTTCAGTGGGT | 0.00 | 2.89 | 2.89 | 0.87 | Hs.31218 | secretory carrier | |
| | | | | | | | membrane protein 1 | |
| 498 | GTGAAACTCTT | 0.00 | 2.89 | 2.89 | 0.87 | Hs.29759 | RNA POLYMERASE | |
| | | | | | | | I AND TRANSCRIPT | |
| | | | | | | | RELEASE FACTOR | |
| 499 | CTGGTGGCCAC | 0.00 | 2.89 | 2.89 | 0.87 | Hs.286028 | Human alpha-2 | |
| | | | | | | | collagen type VI | |
| | | | | | | | mRNA, 3′end | |
| 500 | TAAACGTGGCA | 0.00 | 2.89 | 2.89 | 0.87 | Hs.284146 | hypothetical protein | |
| | | | | | | | DKFZp762N0610 | |
| 501 | GTGGCTCATAC | 0.00 | 2.89 | 2.89 | 0.87 | Hs.281094 | ESTs | |
| 502 | CPAACTCAAAA | 0.00 | 2.89 | 2.89 | 0.87 | Hs.279809 | hypothetical protein | |
| | | | | | | | PRO1741 | |
| 503 | GTGAGACCCCT | 0.00 | 2.89 | 2.89 | 0.87 | Hs.269952 | ESTs | |
| 504 | CCTGTAGTCAC | 0.00 | 2.89 | 2.89 | 0.87 | Hs.268900 | ESTs | |
| 505 | CACTTGTAATC | 0.00 | 2.89 | 2.89 | 0.87 | Hs.268488 | KIAA1185 protein | |
| 506 | TTCAGTTGCTT | 0.00 | 2.89 | 2.89 | 0.87 | Hs.26700 | Homo sapiens cDNA | |
| | | | | | | | FLJ10309 fis, clone | |
| | | | | | | | NT2RM2000287 | |
| 507 | TTGACACTTTC | 0.00 | 2.89 | 2.89 | 0.87 | Hs.26136 | ESTs | |
| 508 | GAGTAGCTGAG | 0.00 | 2.89 | 2.89 | 0.87 | Hs.260039 | sarcospan (Kras | |
| | | | | | | | oncogene-associated | |
| | | | | | | | gene) | |
| 509 | TCCTGACCACC | 0.00 | 2.89 | 2.89 | 0.87 | Hs.26002 | LIM domain binding 1 | |
| 510 | CCACTGGACTC | 0.00 | 2.89 | 2.89 | 0.87 | Hs.253913 | ESTs | |
| 511 | GTGCGGTACCT | 0.00 | 2.89 | 2.89 | 0.87 | Hs.25313 | microspherule protein | |
| | | | | | | | 1 | |
| 512 | GTGAGAACTCG | 0.00 | 2.89 | 2.89 | 0.87 | Hs.250639 | ESTs | |
| 513 | AGACCCTGTCT | 0.00 | 2.89 | 2.89 | 0.87 | Hs.239283 | ESTs | |
| 514 | GCGGCTGACAG | 0.00 | 2.89 | 2.89 | 0.87 | Hs.236511 | ESTs, Moderately | |
| | | | | | | | similar to RNA | |
| | | | | | | | splicing-related | |
| | | | | | | | protein [R,norvegicus] | |
| 515 | AGCGAGAGAGG | 0.00 | 2.89 | 2.89 | 0.87 | Hs.232146 | ESTs, Weakly similar | |
| | | | | | | | to bromodomain | |
| | | | | | | | containing protein | |
| | | | | | | | [H,sapiens] | |
| 516 | AGCCACCACCC | 0.00 | 2.89 | 2.89 | 0.87 | Hs.232045 | ESTs | |
| 517 | TTAAACTCTAA | 0.00 | 2.89 | 2.89 | 0.87 | Hs.226213 | cytochrome P450, 51 | |
| | | | | | | | (lanosterol 14-alpha-demethylase) | |
| 518 | AACACAGGAGG | 0.00 | 2.89 | 2.89 | 0.87 | Hs.222874 | ESTs, Moderately | |
| | | | | | | | similar to zinc | |
| | | | | | | | transporter 4 | |
| | | | | | | | [H,sapiens] | |
| 519 | AGATCAGTTGA | 0.00 | 2.89 | 2.89 | 0.87 | Hs.191805 | ESTs | |
| 520 | AATCATTGAGG | 0.00 | 2.89 | 2.89 | 0.87 | Hs.19150 | Homo sapiens | |
| | | | | | | | mRNA; cDNA | |
| | | | | | | | DKFZp564A2164 | |
| | | | | | | | (from clone | |
| | | | | | | | DKFZp564A2164) | |
| 521 | TAACTTAAGCA | 0.00 | 2.89 | 2.89 | 0.87 | Hs.184542 | CGI-127 protein | |
| 522 | CGACTGCACTC | 0.00 | 2.89 | 2.89 | 0.87 | Hs.182061 | Novel human gene | |
| | | | | | | | mapping to | |
| | | | | | | | chomosome 22 | |
| 523 | AGGAGTCGACA | 0.00 | 2.89 | 2.89 | 0.87 | Hs.181369 | ubiquitin fusion | |
| | | | | | | | degradation 1-like | |
| 524 | AAATATGAGCT | 0.00 | 2.89 | 2.89 | 0.87 | Hs.181368 | US snRNP-specific | |
| | | | | | | | protein (220 kD), | |
| | | | | | | | ortholog of S,cerevisiae | |
| | | | | | | | Prp8p | |
| 525 | AAGTGATTCTG | 0.00 | 2.89 | 2.89 | 0.87 | Hs.180677 | zinc finger protein | |
| | | | | | | | 162 | |
| 526 | GAGCTTTTGAA | 0.00 | 2.89 | 2.89 | 0.87 | Hs.180638 | Homo sapiens cDNA | |
| | | | | | | | FLJ11066 fis,clone | |
| | | | | | | | PLACE1004885 | |
| 527 | GACTGTTGCTG | 0.00 | 2.89 | 2.89 | 0.87 | Hs.179902 | Homo sapiens CTL1 | |
| | | | | | | | gene | |
| 528 | ACCATTCTGCT | 0.00 | 2.89 | 2.89 | 0.87 | Hs.174195 | interferon induced | |
| | | | | | | | transmembrane | |
| | | | | | | | protein 2 (1-8D) | |
| 529 | TGGACCCCCCG | 0.00 | 2.89 | 2.89 | 0.87 | Hs.173501 | ESTs, Moderately | |
| | | | | | | | similar to | |
| | | | | | | | AF151825_1 CGI-67 | |
| 530 | GTGCCAAACAC | 0.00 | 2.89 | 2.89 | 0.87 | Hs.172216 | chromogranin A | |
| | | | | | | | (parathyroid secretory | |
| | | | | | | | protein 1) | |
| 531 | CACTTTACCAG | 0.00 | 2.89 | 2.89 | 0.87 | Hs.170019 | runt-related | |
| | | | | | | | transcription factor 3 | |
| 532 | TGTTCTGATTT | 0.00 | 2.89 | 2.89 | 0.87 | Hs.167835 | acyl-Coenzyme A | |
| | | | | | | | oxidase 1, palmitoyl | |
| 533 | TCTAAAAAGGC | 0.00 | 2.89 | 2.89 | 0.87 | Hs.16622 | zinc finger protein | |
| | | | | | | | 185 (LIM domain) | |
| 534 | CCTCTGTCTCC | 0.00 | 2.89 | 2.89 | 0.87 | Hs.161031 | Homo sapiens | |
| | | | | | | | mRNA; cDNA | |
| | | | | | | | DKFZp434K0322 | |
| | | | | | | | (from clone | |
| | | | | | | | DKFZp434K0322); | |
| | | | | | | | partial cds | |
| 535 | GCTCAGATCGG | 0.00 | 2.89 | 2.89 | 0.87 | Hs.158286 | KIAA0446 gene | |
| | | | | | | | product | |
| 536 | GCCAACAGCAT | 0.00 | 2.89 | 2.89 | 0.87 | Hs.155606 | paired mesoderm | |
| | | | | | | | homeo box 1 | |
| 537 | CGGAACACCGT | 0.00 | 2.89 | 2.89 | 0.87 | Hs.155191 | villin 2 (ezrin) | |
| 538 | GAAACAAAATG | 0.00 | 2.89 | 2.89 | 0.87 | Hs.14896 | DHHC1 protein | |
| 539 | CACTCGTGTGA | 0.00 | 2.89 | 2.89 | 0.87 | Hs.146409 | wingless-type MMTV | |
| | | | | | | | integration site family, | |
| | | | | | | | member 4 | |
| 540 | CCCGGCCCAAA | 0.00 | 2.89 | 2.89 | 0.87 | Hs.133207 | PTPRF interacting | |
| | | | | | | | protein, binding | |
| | | | | | | | protein 1 (liprin beta | |
| | | | | | | | 1) | |
| 541 | GTGCCTAGGGA | 0.00 | 2.89 | 2.89 | 0.87 | Hs.12854 | ATRAP protein | |
| 542 | CTGCTGCTGGT | 0.00 | 2.89 | 2.89 | 0.87 | Hs.12289 | Cdc42 effector | |
| | | | | | | | protein 2 | |
| 543 | GCTCGTGGTCA | 0.00 | 2.89 | 2.89 | 0.87 | Hs.119475 | cold inducible RNA-binding | |
| | | | | | | | protein | |
| 544 | GTGGCTCATTC | 0.00 | 2.89 | 2.89 | 0.87 | Hs.116577 | prostate | |
| | | | | | | | differentiation factor | |
| 545 | CCTGTGTGCAT | 0.00 | 2.89 | 2.89 | 0.87 | Hs.11611 | KIAA1424 protein | |
| 546 | TGTAAAAAAAA | 0.00 | 2.89 | 2.89 | 0.87 | Hs.112743 | synaptonemal | |
| | | | | | | | complex protein 1 | |
| 547 | GAAAATAAAGT | 0.00 | 2.89 | 2.89 | 0.87 | Hs.111334 | ferritin, light | |
| | | | | | | | polypeptide | |
| 548 | CAGAGTTGTAT | 0.00 | 2.89 | 2.89 | 0.87 | Hs.109144 | ESTs | |
| 549 | GGTAGCCTGGG | 0.00 | 2.89 | 2.89 | 0.87 | Hs.108327 | damage-specific DNA | |
| | | | | | | | binding protein 1 | |
| | | | | | | | (127kD) | |
| 550 | GCGGAACCTCA | 0.00 | 2.89 | 2.89 | 0.87 | Hs.10700 | hypothetical protein | |
| 551 | GGAGGTGGGAG | 0.00 | 2.89 | 2.89 | 0.87 | Hs.105097 | thymidine kinase 1, | |
| | | | | | | | soluble | |
| 552 | GGCCCTAGGCA | 8.33 | 24.05 | 2.89 | 2.53 | Hs.78909 | butyrate response | |
| | | | | | | | factor 2 (EGF-response | |
| | | | | | | | factor 2) | |
| 553 | GTGACCTCCTT | 8.33 | 2.89 | 2.88 | 1.20 | Hs.81097 | cytochrome c oxidase | |
| | | | | | | | subunit VIII | |
| 554 | CAACTAATTCA | 8.33 | 2.89 | 2.88 | 1.20 | Hs.75106 | clusterin | |
| | | | | | | | (complement lysis | |
| | | | | | | | inhibitor, SP-40, 40, | |
| | | | | | | | sulfated glycoprotein | |
| | | | | | | | 2, testosterone-repressed | |
| | | | | | | | prostate | |
| | | | | | | | message 2, | |
| | | | | | | | apolipoprotein J) | |
| 555 | CCCTTAGCTTT | 8.33 | 2.89 | 2.88 | 1.20 | Hs.233936 | myosin, light | |
| | | | | | | | polypeptide | |
| | | | | | | | regulatory, non-sacrcomeric | |
| | | | | | | | (20kD) | |
| 556 | AGGGAGCAGAG | 8.33 | 2.89 | 2.88 | 1.20 | Hs.118223 | microfibrillar-associated | |
| | | | | | | | protein 4 | |
| 557 | GGAGTGTGCTC | 21.86 | 7.70 | 2.84 | 2.35 | Hs.9615 | myosin regulatory | |
| | | | | | | | light chain 2, smooth | |
| | | | | | | | muscle isoform | |
| 558 | CGGCAGAGCTG | 1.04 | 2.89 | 2.78 | 0.59 | Hs.9610 | purinergic receptor | |
| | | | | | | | P2X, ligand-gated ion | |
| | | | | | | | channel, 4 | |
| 559 | GTACAAAAGTA | 1.04 | 2.89 | 2.78 | 0.59 | Hs.9552 | binder of Arl Two | |
| 560 | CGTGGGGTGGC | 1.04 | 2.89 | 2.78 | 0.59 | Hs.92679 | ESTs, Weakly similar | |
| | | | | | | | to microtubule-based | |
| | | | | | | | motor [H,sapiens] | |
| 561 | TTTACAAGTTA | 1.04 | 2.89 | 2.78 | 0.59 | Hs.91246 | hypothetical protein | |
| | | | | | | | DKFZp547O146 | |
| 562 | AGGAGCTGCTG | 1.04 | 2.89 | 2.78 | 0.59 | Hs.90443 | NADH | |
| | | | | | | | dehydrogenase | |
| | | | | | | | (ubiquinone) Fe-S | |
| | | | | | | | protein 8 (23kD) | |
| | | | | | | | (NADH-coenzyme Q | |
| | | | | | | | reductase) | |
| 563 | GGTGACCACCA | 1.04 | 2.89 | 2.78 | 0.59 | Hs.83623 | nuclear receptor | |
| | | | | | | | subfamily 1, group I, | |
| | | | | | | | member 3 | |
| 564 | CCACTCCTCCA | 1.04 | 2.89 | 2.78 | 0.59 | Hs.82890 | defender against cell | |
| | | | | | | | death 1 | |
| 565 | TAAAATACTCC | 1.04 | 2.89 | 2.78 | 0.59 | Hs.8125 | Homo sapiens | |
| | | | | | | | mRNA; cDNA | |
| | | | | | | | DKFZp586E1521 | |
| | | | | | | | (from clone | |
| | | | | | | | DKFZp586E1521); | |
| | | | | | | | partial cds | |
| 566 | TTTTGAAGCAG | 1.04 | 2.89 | 2.78 | 0.59 | Hs.80464 | hepatitis B virus x-interacting | |
| | | | | | | | protein | |
| | | | | | | | (9,6kD) | |
| 567 | CTGCCTCCTTA | 1.04 | 2.89 | 2.78 | 0.59 | Hs.7918 | uncharacterized | |
| | | | | | | | hypothalamus protein | |
| | | | | | | | HSMNP1 | |
| 568 | GTGTCCTCCTC | 1.04 | 2.89 | 2.78 | 0.59 | Hs.78979 | Golgi apparatus | |
| | | | | | | | protein 1 | |
| 569 | CGCAAGCTGGT | 1.04 | 2.89 | 2.78 | 0.59 | Hs.77886 | lamin A/C | |
| 570 | AGGGGCCGGGG | 1.04 | 2.89 | 2.78 | 0.59 | Hs.77448 | aldehyde | |
| | | | | | | | dehydrogenase 4 | |
| 571 | CTCACTTTTTT | 1.04 | 2.89 | 2.78 | 0.59 | Hs.76722 | CCAAT/enhancer | |
| | | | | | | | binding protein | |
| | | | | | | | (C/EBP), delta | |
| 572 | TGGCTCCTCCC | 1.04 | 2.89 | 2.78 | 0.59 | Hs.76506 | lymphocyte cytosolic | |
| | | | | | | | protein 1 (L-plastin) | |
| 573 | TTTTCTGAAAA | 1.04 | 2.89 | 2.78 | 0.59 | Hs.76136 | thioredoxin | |
| 574 | GTGGCAGAGAC | 1.04 | 2.89 | 2.78 | 0.59 | Hs.75813 | polycystic kidney | |
| | | | | | | | disease 1 (autosomal | |
| | | | | | | | dominant | |
| 575 | TGGTTTTGGCA | 1.04 | 2.89 | 2.78 | 0.59 | Hs.75721 | profilin 1 | |
| 576 | TACCCCACCTT | 1.04 | 2.89 | 2.78 | 0.59 | Hs.75258 | H2A histone family, | |
| | | | | | | | member Y | |
| 577 | TCTGTCCTCAG | 1.04 | 2.89 | 2.78 | 0.59 | Hs.75216 | protein tyrosine | |
| | | | | | | | phosphatase, | |
| | | | | | | | receptor type, F | |
| 578 | CACAGAGTCCT | 1.04 | 2.89 | 2.78 | 0.59 | Hs.75140 | low density | |
| | | | | | | | lipoprotein-related | |
| | | | | | | | protein-associated | |
| | | | | | | | protein 1 | |
| 579 | GGCCCTGAGCG | 1.04 | 2.89 | 2.78 | 0.59 | Hs.71618 | polymerase (RNA) II | |
| | | | | | | | (DNA directed) | |
| | | | | | | | polypeptide L (7,6kD) | |
| 580 | GAGGCCATCCC | 1.04 | 2.89 | 2.78 | 0.59 | Hs.70830 | U6 snRNA-associated | |
| | | | | | | | Sm-like | |
| | | | | | | | protein LSm7 | |
| 581 | TTGTGATGTAA | 1.04 | 2.89 | 2.78 | 0.59 | Hs.6975 | PRO1073 protein | |
| 582 | GAGTCCCTGGT | 1.04 | 2.89 | 2.78 | 0.59 | Hs.68398 | period (Drosophila) | |
| | | | | | | | homolog 1 | |
| 583 | CACACACACAC | 1.04 | 2.89 | 2.78 | 0.59 | Hs.63984 | cadherin 13, H-cadherin | |
| | | | | | | | (heart) | |
| 584 | GCTCACACCTG | 1.04 | 2.89 | 2.78 | 0.59 | Hs.60617 | sialyltransferase 4A | |
| | | | | | | | (beta-galactosidase | |
| | | | | | | | alpha-2,3-sialytransferase) | |
| 585 | TGATTGATTTG | 1.04 | 2.89 | 2.78 | 0.59 | Hs.5912 | F-box only protein 7 | |
| 586 | AAATGCGAACA | 1.04 | 2.89 | 2.78 | 0.59 | Hs.5672 | ESTs, Weakly similar | |
| | | | | | | | to Similarity to Yeast | |
| | | | | | | | D-lactate | |
| | | | | | | | dehydrogenase | |
| | | | | | | | [C,elegans] | |
| 587 | TAGTTGTAGGG | 1.04 | 2.89 | 2.78 | 0.59 | Hs.5324 | hypothetical protein | |
| 588 | GGCCCCGGACC | 1.04 | 2.89 | 2.78 | 0.59 | Hs.4742 | anchor attachment | |
| | | | | | | | protein 1 (Gaa1p, | |
| | | | | | | | yeast) homolog | |
| 589 | GGGCCCAGGGG | 1.04 | 2.89 | 2.78 | 0.59 | Hs.3803 | reticulon 2 | |
| 590 | TAAACTGAAAA | 1.04 | 2.89 | 2.78 | 0.59 | Hs.3491 | ribosomal protein S14 | |
| 591 | GCTTTTATTCA | 1.04 | 2.89 | 2.78 | 0.59 | Hs.31819 | HT014 | |
| 592 | TACTGGTTTAT | 1.04 | 2.89 | 2.78 | 0.59 | Hs.30299 | IGF-II mRNA-binding | |
| | | | | | | | protein 2 | |
| 593 | GCAGTTGGATC | 1.04 | 2.89 | 2.78 | 0.59 | Hs.284932 | Homo sapiens clone | |
| | | | | | | | 24650 ubiquitin | |
| | | | | | | | hydrolase mRNA, | |
| | | | | | | | partial cds | |
| 594 | ACCTTCAAAAA | 1.04 | 2.89 | 2.78 | 0.59 | Hs.28444 | hypothetical protein | |
| | | | | | | | FLJ10567 | |
| 595 | GACAGTGTGGG | 1.04 | 2.89 | 2.78 | 0.59 | Hs.279863 | nuclear mitotic | |
| | | | | | | | apparatus protein 1 | |
| 596 | AAACCAGGGCC | 1.04 | 2.89 | 2.78 | 0.59 | Hs.279836 | HSPC166 protein | |
| 597 | CTGAGGGTGGT | 1.04 | 2.89 | 2.78 | 0.59 | Hs.279761 | HSPC134 protein | |
| 598 | CTCGGAGGCCT | 1.04 | 2.89 | 2.78 | 0.59 | Hs.279623 | selenoprotein X | |
| 599 | TGAATGATACG | 1.04 | 2.89 | 2.78 | 0.59 | Hs.278614 | protease, serine, 15 | |
| 600 | GCTGCCCTTGA | 1.04 | 2.89 | 2.78 | 0.59 | Hs.278242 | tubulin, alpha, | |
| | | | | | | | ubiquitous | |
| 601 | TGATGTTCCAC | 1.04 | 2.89 | 2.78 | 0.59 | Hs.277401 | bromodomain | |
| | | | | | | | adjacent to zinc finger | |
| | | | | | | | domain, 2A | |
| 602 | GTGTCGGCTGT | 1.04 | 2.89 | 2.78 | 0.59 | Hs.275959 | eukaryotic translation | |
| | | | | | | | elongation factor 1 | |
| | | | | | | | beta 2 | |
| 603 | AAGTCATTCAG | 1.04 | 2.89 | 2.78 | 0.59 | Hs.274416 | NADH | |
| | | | | | | | dehydrogenase | |
| | | | | | | | (ubiquinone) 1 alpha | |
| | | | | | | | subcomplex, 6 (14kD, | |
| | | | | | | | B14) | |
| 604 | GCGAAAACCCC | 1.04 | 2.89 | 2.78 | 0.59 | Hs.272837 | ESTs | |
| 605 | GTGGCACTTGC | 1.04 | 2.89 | 2.78 | 0.59 | Hs.272322 | Homo sapiens | |
| | | | | | | | mRNA; cDNA | |
| | | | | | | | DKFZp434L092 (from | |
| | | | | | | | clone | |
| | | | | | | | DKFZp434L092) | |
| 606 | CCTGTATCCCA | 1.04 | 2.89 | 2.78 | 0.59 | Hs.270072 | ESTs | |
| 607 | CCTCCCCCGTC | 1.04 | 2.89 | 2.78 | 0.59 | Hs.268763 | Breakpoint cluster | |
| | | | | | | | region protein, uterine | |
| | | | | | | | leiomyoma, 1; barrier | |
| | | | | | | | to autointegration | |
| | | | | | | | factor | |
| 608 | GCCCCTGCGCA | 1.04 | 2.89 | 2.78 | 0.59 | Hs.267200 | ESTs, Moderately | |
| | | | | | | | similar to T20D3,3 | |
| | | | | | | | [C,elegans] | |
| 609 | GTGGCTCAGGC | 1.04 | 2.89 | 2.78 | 0.59 | Hs.259047 | ESTs | |
| 610 | GGGCCCTGGCC | 1.04 | 2.89 | 2.78 | 0.59 | Hs.25895 | ESTs, Weakly similar | |
| | | | | | | | to PI-3 kinase | |
| | | | | | | | [H,sapiens] | |
| 611 | GAGTTGGCAGT | 1.04 | 2.89 | 2.78 | 0.59 | Hs.258730 | heme-regulated | |
| | | | | | | | initiation factor 2-alpha | |
| | | | | | | | kinase | |
| 612 | TGGCTGTGTGG | 1.04 | 2.89 | 2.78 | 0.59 | Hs.25709 | ESTs, Weakly similar | |
| | | | | | | | to PSF_HUMAN | |
| | | | | | | | PTB-ASSOCIATED | |
| | | | | | | | SPLICING FACTOR | |
| | | | | | | | [H,sapiens] | |
| 613 | TTCTTTTTCTT | 1.04 | 2.89 | 2.78 | 0.59 | Hs.250722 | (Manual assignment) | |
| | | | | | | | MUG, Myeloid-upregulated | |
| | | | | | | | protein | |
| 614 | GTTCCAGCAGC | 1.04 | 2.89 | 2.78 | 0.59 | Hs.23918 | Homo sapiens clone | |
| | | | | | | | 25116 mRNA | |
| | | | | | | | sequence | |
| 615 | TCTCCAGGAAC | 1.04 | 2.89 | 2.78 | 0.59 | Hs.237924 | CGI-69 protein | |
| 616 | TAATCCCAGCA | 1.04 | 2.89 | 2.78 | 0.59 | Hs.236710 | EST | |
| 617 | CCTCTAATCCC | 1.04 | 2.89 | 2.78 | 0.59 | Hs.236150 | ESTs, Weakly similar | |
| | | | | | | | to AF090942_1 | |
| | | | | | | | PRO657 [H,sapiens] | |
| 618 | AGTTCGAGACC | 1.04 | 2.89 | 2.78 | 0.59 | Hs.232540 | ESTs | |
| 619 | AAGTGAGGAGA | 1.04 | 2.89 | 2.78 | 0.59 | Hs.231840 | WW domain binding | |
| | | | | | | | protein 2 | |
| 620 | GCTGGGAGGGG | 1.04 | 2.89 | 2.78 | 0.59 | Hs.20733 | ESTs | |
| 621 | GTGGCTGACAC | 1.04 | 2.89 | 2.78 | 0.59 | Hs.202234 | ESTs | |
| 622 | TTACAGTCTTA | 1.04 | 2.89 | 2.78 | 0.59 | Hs.194110 | Homo sapiens | |
| | | | | | | | mRNA; cDNA | |
| | | | | | | | DKFZp434C0814 | |
| | | | | | | | (from clone | |
| | | | | | | | DKFZp434C0814) | |
| 623 | TAGCTCTATGG | 1.04 | 2.89 | 2.78 | 0.59 | Hs.190703 | ATPase, Na+/K+ | |
| | | | | | | | transporting, alpha 1 | |
| | | | | | | | polypeptide | |
| 624 | GGGGCCCCCTC | 1.04 | 2.89 | 2.78 | 0.59 | Hs.18528 | Sjogren's syndrome | |
| | | | | | | | nuclear autoantigen 1 | |
| 625 | CCGCTGATCCA | 1.04 | 2.89 | 2.78 | 0.59 | Hs.184161 | exostoses (multiple) 1 | |
| 626 | GGGAAACAGGT | 1.04 | 2.89 | 2.78 | 0.59 | Hs.18368 | DKFZP564B0769 | |
| | | | | | | | protein | |
| 627 | AAATACAGCAG | 1.04 | 2.89 | 2.78 | 0.59 | Hs.182429 | protein disulfide | |
| | | | | | | | isomerase-related | |
| | | | | | | | protein | |
| 628 | AAAAAAAAAAG | 1.04 | 2.89 | 2.78 | 0.59 | Hs.180842 | ribosomal protein L13 | |
| 629 | CTGGGTCTCCA | 1.04 | 2.89 | 2.78 | 0.59 | Hs.180842 | ribosomal protein L13 | |
| 630 | AGGAAGGAACA | 1.04 | 2.89 | 2.78 | 0.59 | Hs.173664 | v-erb-b2 avian | |
| | | | | | | | erythroblastic | |
| | | | | | | | leukemia viral | |
| | | | | | | | oncogene homolog 2 | |
| | | | | | | | (neuro/glioblastoma | |
| | | | | | | | derived oncogene | |
| | | | | | | | homolog) | |
| 631 | GCACGCGTAAC | 1.04 | 2.89 | 2.78 | 0.59 | Hs.169552 | ESTs, Weakly similar | |
| | | | | | | | to BRDT [H,sapiens] | |
| 632 | ATTTCAAGATG | 1.04 | 2.89 | 2.78 | 0.59 | Hs.155097 | carbonic anhydrase II | |
| 633 | GCTGGCAGGCC | 1.04 | 2.89 | 2.78 | 0.59 | Hs.154886 | choline kinase-like | |
| 634 | TCAATAAAACC | 1.04 | 2.89 | 2.78 | 0.59 | Hs.151411 | KIAA0916 protein | |
| 635 | TCTTCCCCAGT | 1.04 | 2.89 | 2.78 | 0.59 | Hs.14231 | selenoprotein W, 1 | |
| 636 | ATGGTGGGCAC | 1.04 | 2.89 | 2.78 | 0.59 | Hs.132390 | zinc finger protein 36 | |
| | | | | | | | (KOX 18) | |
| 637 | GTTTCTATCAA | 1.04 | 2.89 | 2.78 | 0.59 | Hs.12540 | lysophospholipase I | |
| 638 | GTGGCACCTGC | 1.04 | 2.89 | 2.78 | 0.59 | Hs.1244 | CD9 antigen (p24) | |
| 639 | ACTGCAGAGCG | 1.04 | 2.89 | 2.78 | 0.59 | Hs.12186 | Homo sapiens cDNA | |
| | | | | | | | FLJ20792 fis, clone | |
| | | | | | | | COL01292 | |
| 640 | CTCTGCCCTCC | 1.04 | 2.89 | 2.78 | 0.59 | Hs.115412 | ESTs,Weakly similar | |
| | | | | | | | to dJ68O2.2 | |
| | | | | | | | [H,sapiens] | |
| 641 | GACCGCGGCTT | 1.04 | 2.89 | 2.78 | 0.59 | Hs.110903 | claudin 5 | |
| | | | | | | | (transmembrane | |
| | | | | | | | protein deleted in | |
| | | | | | | | velocardiofacial | |
| | | | | | | | syndrome) | |
| 642 | TTTGGTGTTTG | 1.04 | 2.89 | 2.78 | 0.59 | Hs.11050 | F-box only protein 9 | |
| 643 | GGAAGGACAGA | 1.04 | 2.89 | 2.78 | 0.59 | Hs.106876 | Vacuolar proton-ATPase, | |
| | | | | | | | subunit D; | |
| | | | | | | | V-ATPase, subunit D | |
| 644 | AAGATAATGCC | 1.04 | 2.89 | 2.78 | 0.59 | Hs.102696 | MCT-1 protein | |
| 645 | GATGCTGCCAA | 3.12 | 8.66 | 2.78 | 1.19 | Hs.99914 | ribosomal protein L22 | |
| 646 | ACTGCCCGCTG | 3.12 | 8.66 | 2.78 | 1.19 | Hs.81071 | extracellular matrix | |
| | | | | | | | protein 1 | |
| 647 | GTGAAACCCGG | 3.12 | 8.66 | 2.78 | 1.19 | Hs.229170 | ESTs | |
| 648 | ATGTACTCTGG | 2.08 | 5.77 | 2.77 | 0.91 | Hs.75432 | IMP (inosine | |
| | | | | | | | monophosphate) | |
| | | | | | | | dehydrogenase 2 | |
| 649 | TTCTTGTTTTG | 2.08 | 5.77 | 2.77 | 0.91 | Hs.74621 | prion protein (p27-30) | |
| | | | | | | | (Creutzfeld-Jakob | |
| | | | | | | | disease, Gerstmann-Strausler- | |
| | | | | | | | Scheinker syndrome, fatal | |
| | | | | | | | familial insomnia) | |
| 650 | GCGAGACCCTG | 2.08 | 5.77 | 2.77 | 0.91 | Hs.278531 | Homo sapiens | |
| | | | | | | | mRNA; cDNA | |
| | | | | | | | DKFZp434A1014 | |
| | | | | | | | (from clone | |
| | | | | | | | DKFZp434A1014); | |
| | | | | | | | partial cds | |
| 651 | GGGTCAAAAGG | 2.08 | 5.77 | 2.77 | 0.91 | Hs.181307 | H3 histone, family 3A | |
| 652 | TGGAGAAGAGC | 2.08 | 5.77 | 2.77 | 0.91 | Hs.179526 | upregulated by 1,25-dihydroxyvitamin | |
| | | | | | | | D-3 | |
| 653 | AAATCAATACA | 5.21 | 1.92 | 2.71 | 0.86 | Hs.94953 | ESTs, Highly similar | |
| | | | | | | | to C1QC_HUMAN | |
| | | | | | | | COMPLEMENT C1Q | |
| | | | | | | | SUBCOMPONENT, | |
| | | | | | | | C CHAIN | |
| | | | | | | | PRECURSOR₋[H,sapiens] | |
| 654 | TGCTTTGGGAT | 5.21 | 1.92 | 2.71 | 0.86 | Hs.84344 | CGI-135 protein | |
| 655 | TCCGTGGTTGG | 5.21 | 1.92 | 2.71 | 0.86 | Hs.79516 | brain acid-soluble | |
| | | | | | | | protein 1 | |
| 656 | CAAGGGTAAGA | 5.21 | 1.92 | 2.71 | 0.86 | Hs.76224 | EGF-containing | |
| | | | | | | | fibulin-like | |
| | | | | | | | extracellular matrix | |
| | | | | | | | protein 1 | |
| 657 | TTTGCACTTGT | 5.21 | 1.92 | 2.71 | 0.86 | Hs.75188 | wee1+ (S, pombe) | |
| | | | | | | | homolog | |
| 658 | CAGCCCAACCG | 5.21 | 1.92 | 2.71 | 0.86 | Hs.28081 | eukaryotic translation | |
| | | | | | | | initiation factor 3, | |
| | | | | | | | subunit 4 (delta, | |
| | | | | | | | 44kD) | |
| 659 | ATGAACCGCAG | 5.21 | 1.92 | 2.71 | 0.86 | Hs.252259 | ribosomal protein S3 | |
| 660 | GCCCAGCGGCC | 5.21 | 1.92 | 2.71 | 0.86 | Hs.194385 | hypothetical protein | |
| | | | | | | | FLJ20234 | |
| 661 | ATGGCACGTGC | 5.21 | 1.92 | 2.71 | 0.86 | Hs.179999 | stromal cell protein | |
| 662 | TCTCTTTTTCT | 5.21 | 1.92 | 2.71 | 0.86 | Hs.119529 | epididymal secretory | |
| | | | | | | | protein (19,5kD) | |
| 663 | CCTGTCCTGCA | 5.21 | 1.92 | 2.71 | 0.86 | Hs.11417 | Rab acceptor 1 | |
| | | | | | | | (prenylated) | |
| 664 | CTGAGAGCTGG | 10.41 | 3.85 | 2.70 | 1.34 | Hs.78501 | growth arrest-specific | |
| | | | | | | | 6 | |
| 665 | AGTCTGATGTT | 10.41 | 3.85 | 2.70 | 1.34 | Hs.173255 | small nuclear | |
| | | | | | | | ribonucleoprotein | |
| | | | | | | | polypeptide A | |
| 666 | CTAAAAAAAAA | 22.90 | 8.66 | 2.64 | 2.28 | Hs.23740 | KIAA1598 protein | |
| 667 | TGTGCTAAATG | 7.29 | 19.24 | 2.64 | 1.98 | Hs.250895 | ribosomal protein L34 | |
| 668 | TACCATCAATA | 30.19 | 11.54 | 2.62 | 2.77 | Hs.169476 | glyceraldehyde-3-phosphate | |
| | | | | | | | dehydrogenase | |
| 669 | CCCGTAATCCC | 12.49 | 4.81 | 2.60 | 1.47 | Hs.274168 | Homo sapiens | |
| | | | | | | | mRNA; cDNA | |
| | | | | | | | DKFZp761P0212 | |
| | | | | | | | (from clone | |
| | | | | | | | DKFZp761P0212); | |
| | | | | | | | partial cds | |
| 670 | AGAACCTTCCA | 12.49 | 4.81 | 2.60 | 1.47 | Hs.181244 | major | |
| | | | | | | | histocompatibility | |
| | | | | | | | complex, class I, A | |
| 671 | GTGGCGCACAC | 4.16 | 10.58 | 2.54 | 1.28 | Hs.246717 | ESTs | |
| 672 | GCCTACCCGAG | 4.16 | 10.58 | 2.54 | 1.28 | Hs.23582 | tumor-associated | |
| | | | | | | | calcium signal | |
| | | | | | | | transducer 2 | |
| 673 | GTGGCGTGTGC | 8.33 | 21.17 | 2.54 | 2.05 | Hs.278627 | prenylcysteine lyase | |
| 674 | CTAACCAGACA | 7.29 | 2.89 | 2.52 | 1.02 | Hs.76368 | capping protein (actin | |
| | | | | | | | filament) muscle Z-line, | |
| | | | | | | | beta | |
| 675 | ACTGGGTCTAT | 7.29 | 2.89 | 2.52 | 1.02 | Hs.275163 | non-metastatic cells | |
| | | | | | | | 2, protein (NM23B) | |
| | | | | | | | expressed in | |
| 676 | GCCAGCCAGTG | 7.29 | 2.89 | 2.52 | 1.02 | Hs.149098 | smoothelin | |
| 677 | GCCCCTGCTGA | 41.64 | 104.86 | 2.52 | 7.23 | Hs.195850 | keratin 5 | |
| | | | | | | | (epidermolysis | |
| | | | | | | | bullosa simplex, | |
| | | | | | | | Dowling-Meara/Kobner/Weber-Cockayne | |
| | | | | | | | types) | |
| 678 | GGCGACAGAGC | 3.12 | 7.70 | 2.47 | 1.03 | Hs.92254 | hypothetical protein | |
| | | | | | | | FLJ20163 | |
| 679 | TAACAGCCAGG | 3.12 | 7.70 | 2.47 | 1.03 | Hs.81328 | nuclear factor of | |
| | | | | | | | kappa light | |
| | | | | | | | polypeptide gene | |
| | | | | | | | enhancer in B-cells | |
| | | | | | | | inhibitor, alpha | |
| 680 | GGCCCCTCACC | 3.12 | 7.70 | 2.47 | 1.03 | Hs.274313 | insulin-like growth | |
| | | | | | | | factor binding protein | |
| | | | | | | | 6 | |
| 681 | GTTCTGGTTTA | 3.12 | 7.70 | 2.47 | 1.03 | Hs.241336 | Homo sapiens | |
| | | | | | | | mRNA; cDNA | |
| | | | | | | | DKFZp564G0422 | |
| | | | | | | | (from clone | |
| | | | | | | | DKFZp564G0422) | |
| 682 | TCAAAAAAAAA | 3.12 | 7.70 | 2.47 | 1.03 | Hs.200188 | deleted in lung and | |
| | | | | | | | esophageal cancer 1 | |
| 683 | CACTTGCCCTA | 3.12 | 7.70 | 2.47 | 1.03 | Hs.15977 | NADH | |
| | | | | | | | dehydrogenase | |
| | | | | | | | (ubiquinone) 1 beta | |
| | | | | | | | subcomplex, 9 (22kD, | |
| | | | | | | | B22) | |
| 684 | CAATAAACTGA | 3.12 | 7.70 | 2.47 | 1.03 | Hs.150580 | putative translation | |
| | | | | | | | initiation factor | |
| 685 | CCATTGCACTG | 3.12 | 7.70 | 2.47 | 1.03 | Hs.142457 | ESTs, Moderately | |
| | | | | | | | similar to alternatively | |
| | | | | | | | spliced product using | |
| | | | | | | | exon 13A [H,sapiens] | |
| 686 | TAAACCTGCTG | 31.23 | 76.96 | 2.46 | 5.39 | Hs.99923 | lectin, galactoside | |
| | | | | | | | binding, soluble, 7 | |
| | | | | | | | (galectin 7) | |
| 687 | CCACCGCACTC | 6.25 | 15.39 | 2.46 | 1.60 | Hs.222669 | EST | |
| 688 | AGCCTTTGTTG | 18.74 | 7.70 | 2.43 | 1.83 | Hs.9930 | collagen-binding | |
| | | | | | | | protein 2 (colligen 2) | |
| 689 | CGCAGTGTCCT | 9.37 | 3.85 | 2.43 | 1.17 | Hs.76159 | ATPase, H+ | |
| | | | | | | | transporting, | |
| | | | | | | | lysosomal (vacuolar | |
| | | | | | | | proton pump) 16kD | |
| 690 | GTGGAGGGCAC | 5.21 | 12.51 | 2.40 | 1.37 | Hs.83393 | cystatin E/M | |
| 691 | TTTGCTCTCCC | 11.45 | 4.81 | 2.38 | 1.30 | Hs.75350 | vinculin | |
| 692 | CAGGCCCCACC | 11.45 | 4.81 | 2.38 | 1.30 | Hs.256290 | S100 calcium-binding | |
| | | | | | | | protein A11 | |
| | | | | | | | (calgizzarin) | |
| 693 | TGGCCAGCTCC | 13.53 | 5.77 | 2.34 | 1.43 | Hs.170121 | protein tyrosine | |
| | | | | | | | phosphatase | |
| | | | | | | | receptor type, C | |
| 694 | CCGTGACTCTG | 13.53 | 5.77 | 2.34 | 1.43 | Hs.155712 | Homo sapiens | |
| | | | | | | | mRNA; cDNA | |
| | | | | | | | DKFZp586O2223 | |
| | | | | | | | (from clone | |
| | | | | | | | DKFZp586O2223) | |
| 695 | AATAAATTCCT | 4.16 | 9.62 | 2.31 | 1.13 | Hs.76307 | neuroblastoma, | |
| | | | | | | | suppression of | |
| | | | | | | | tumorigenicity 1 | |
| 696 | CATCTGTACTC | 4.16 | 9.62 | 2.31 | 1.13 | Hs.180255 | major | |
| | | | | | | | histocompatibility | |
| | | | | | | | complex, class II, DR | |
| | | | | | | | beta 1 | |
| 697 | ATCGCTTTCTA | 4.16 | 9.62 | 2.31 | 1.13 | Hs.177486 | amyloid beta (A4) | |
| | | | | | | | precursor protein | |
| | | | | | | | (protease nexin-II, | |
| | | | | | | | Alzheimer disease) | |
| 698 | CAAAAAAAAAA | 4.16 | 9.62 | 2.31 | 1.13 | Hs.112272 | histone deacetylase 8 | |
| 699 | TCTGCAATGAA | 2.08 | 4.81 | 2.31 | 0.75 | Hs.8170 | hypothetical protein | |
| 700 | GCTTAACCTGG | 2.08 | 4.81 | 2.31 | 0.75 | Hs.77508 | glutamate | |
| | | | | | | | dehydrogenase 1 | |
| 701 | ATGAGCTGACC | 2.08 | 4.81 | 2.31 | 0.75 | Hs.695 | cystatin B (stefin B) | |
| 702 | ACTACCATAAC | 2.08 | 4.81 | 2.31 | 0.75 | Hs.57929 | slit (Drosophila) | |
| | | | | | | | homolog 3 | |
| 703 | CATTGTAAATA | 2.08 | 4.81 | 2.31 | 0.75 | Hs.57929 | protease inhibitor 5 | |
| | | | | | | | (maspin) | |
| 704 | CGGATAACCAG | 2.08 | 4.81 | 2.31 | 0.75 | Hs.5181 | proliferation-associated | |
| | | | | | | | 2G4, | |
| | | | | | | | 38kD | |
| 705 | GCTCCCAGACT | 2.08 | 4.81 | 2.31 | 0.75 | Hs.5097 | synaptogyrin 2 | |
| 706 | GCCTGCAGTCT | 2.08 | 4.81 | 2.31 | 0.75 | Hs.31439 | serine protease | |
| | | | | | | | inhibitor, Kunitz type, | |
| | | | | | | | 2 | |
| 707 | CCTGTAGCCCC | 2.08 | 4.81 | 2.31 | 0.75 | Hs.277320 | EST | |
| 708 | CCGGTAATCCC | 2.08 | 4.81 | 2.31 | 0.75 | Hs.272813 | dual oxidase 1 | |
| 709 | CTTCCTGTGAT | 2.08 | 4.81 | 2.31 | 0.75 | Hs.2533 | aldehyde | |
| | | | | | | | dehydrogenase 9 | |
| | | | | | | | (gamma-aminobutyraldehyde | |
| | | | | | | | dehydrogenase, E3 | |
| | | | | | | | isozyme) | |
| 710 | CCAGTAATCCC | 2.08 | 4.81 | 2.31 | 0.75 | Hs.237078 | ESTs | |
| 711 | ACACTGCACTC | 2.08 | 4.81 | 2.31 | 0.75 | Hs.200454 | ESTs | |
| 712 | GTGGGTTGGCT | 2.08 | 4.81 | 2.31 | 0.75 | Hs.195432 | aldehyde | |
| | | | | | | | dehydrogenase 2, | |
| | | | | | | | mitochondrial | |
| 713 | AACGCGAACAC | 2.08 | 4.81 | 2.31 | 0.75 | Hs.18946 | squamous cell | |
| | | | | | | | carcinoma antigen | |
| | | | | | | | recognised by T cells | |
| 714 | GCCAGGAGCTA | 2.08 | 4.81 | 2.31 | 0.75 | Hs.18141 | ladinin 1 | |
| 715 | GGAAAAAAAAA | 2.08 | 4.81 | 2.31 | 0.75 | Hs.177530 | ATP synthase, H+ | |
| | | | | | | | transporting, | |
| | | | | | | | mitochondrial F1 | |
| | | | | | | | complex, epsilon | |
| | | | | | | | subunit | |
| 716 | TATGACTTAAT | 2.08 | 4.81 | 2.31 | 0.75 | Hs.173737 | Homo sapiens | |
| | | | | | | | mRNA, | |
| | | | | | | | clone:PO2ST9 | |
| 717 | GCCAAGGGGCC | 2.08 | 4.81 | 2.31 | 0.75 | Hs.168669 | oxoglutarate | |
| | | | | | | | dehydrogenase | |
| | | | | | | | (lipoamide) | |
| 718 | AAAAATAAAGG | 2.08 | 4.81 | 2.31 | 0.75 | Hs.155101 | ATP synthase, H+ | |
| | | | | | | | transporting, | |
| | | | | | | | mitochondrial F1 | |
| | | | | | | | complex, alpha | |
| | | | | | | | subunit, isoform 1, | |
| | | | | | | | cardiac muscle | |
| 719 | ATTATTTTTCT | 2.08 | 4.81 | 2.31 | 0.75 | Hs.153 | ribosomal protein L7 | |
| 720 | GCGAAACTCCA | 2.08 | 4.81 | 2.31 | 0.75 | Hs.112860 | KIAA1353 protein | |
| 721 | GCGCTGGAGTG | 2.08 | 4.81 | 2.31 | 0.75 | Hs.110695 | ESTs, Weakly similar | |
| | | | | | | | to B0495,6 | |
| | | | | | | | [C,elegans] | |
| 722 | CTGACCTGTGT | 19.78 | 8.66 | 2.28 | 1.79 | Hs.77961 | major | |
| | | | | | | | histocompatibility | |
| | | | | | | | complex, class I, B | |
| 723 | ATCCGCGAGGC | 13.53 | 30.79 | 2.28 | 2.39 | Hs.180142 | (Manual assignment) | |
| | | | | | | | CLSP Calmodulin-like | |
| | | | | | | | skin protein | |
| 724 | ACCTCCACTGG | 42.68 | 95.24 | 2.23 | 5.58 | Hs.1 12457 | ESTs | |
| 725 | TACCCTAGAAC | 8.33 | 18.28 | 2.19 | 1.62 | Hs.252588 | Homo sapiens | |
| | | | | | | | mRNA; cDNA | |
| | | | | | | | DKFZp564F172 (from | |
| | | | | | | | clone | |
| | | | | | | | DKFZp564F172) | |
| 726 | ATCATAGCTCA | 4.16 | 1.92 | 2.17 | 0.68 | Hs.97876 | ESTs | |
| 727 | TCTATAATCCC | 4.16 | 1.92 | 2.17 | 0.68 | Hs.96866 | ESTs | |
| 728 | GAATAAATGTT | 4.16 | 1.92 | 2.17 | 0.68 | Hs.8762 | FK506-binding | |
| | | | | | | | protein 9 (63 kD) | |
| 729 | CTTGAGCAATA | 4.16 | 1.92 | 2.17 | 0.68 | Hs.848 | FK506-binding | |
| | | | | | | | protein 4 (59kD) | |
| 730 | AGCCCTCCCTG | 4.16 | 1.92 | 2.17 | 0.68 | Hs.74111 | RNA-binding protein | |
| | | | | | | | (autoantigenic) | |
| 731 | CTACCAGGCCT | 4.16 | 1.92 | 2.17 | 0.68 | Hs.54457 | CD81 antigen (target | |
| | | | | | | | of antiproliferative | |
| | | | | | | | antibody 1) | |
| 732 | GCGAAACCTCA | 4.16 | 1.92 | 2.17 | 0.68 | Hs.42644 | thioredoxin-like | |
| 733 | GACTCTGAAAA | 4.16 | 1.92 | 2.17 | 0.68 | Hs.2953 | ribosomal protein | |
| | | | | | | | S15a | |
| 734 | GTTTGGCAGTG | 4.16 | 1.92 | 2.17 | 0.68 | Hs.283690 | hypothetical protein | |
| 735 | AGACCTCCTTC | 4.16 | 1.92 | 2.17 | 0.68 | Hs.281706 | sortilin 1 | |
| 736 | GGAAGGGAGGC | 4.16 | 1.92 | 2.17 | 0.68 | Hs.279581 | hypothetical protein | |
| | | | | | | | FLJ20568 | |
| 737 | TTTGTGACTGT | 4.16 | 1.92 | 2.17 | 0.68 | Hs.239737 | C-terminal binding | |
| | | | | | | | protein 1 | |
| 738 | AGTGGTGGCTA | 4.16 | 1.92 | 2.17 | 0.68 | Hs.230 | fibromodulin | |
| 739 | AGAACAAAACC | 4.16 | 1.92 | 2.17 | 0.68 | Hs.180909 | peroxiredoxin 1 | |
| 740 | GCTGGATGCGG | 4.16 | 1.92 | 2.17 | 0.68 | Hs.18075 | chromosome 9 open | |
| | | | | | | | reading frame 3 | |
| 741 | CCAGGAGGAAT | 4.16 | 1.92 | 2.17 | 0.68 | Hs.180414 | heat shock 70kd | |
| | | | | | | | protein 10 (HSC71) | |
| 742 | CACCACAACAA | 4.16 | 1.92 | 2.17 | 0.68 | Hs.174139 | chloride channel 3 | |
| 743 | GTCTGACCCCA | 4.16 | 1.92 | 2.17 | 0.68 | Hs.173902 | protein phosphatase | |
| | | | | | | | 2 (formerly 2A), | |
| | | | | | | | regulatory subunit A | |
| | | | | | | | (PR 65), alpha | |
| | | | | | | | isoform | |
| 744 | AAGCGCTCTCG | 4.16 | 1.92 | 2.17 | 0.68 | Hs.168913 | serine/threonine | |
| | | | | | | | kinase 24 (Ste20, | |
| | | | | | | | yeast homolog) | |
| 745 | ATCCGCCTGCC | 4.16 | 1.92 | 2.17 | 0.68 | Hs.167956 | ESTs, Weakly similar | |
| | | | | | | | to KIAA0309 | |
| | | | | | | | [H,sapiens] | |
| 746 | CTTGTGTGTAG | 4.16 | 1.92 | 2.17 | 0.68 | Hs.158203 | actin binding LIM | |
| | | | | | | | protein 1 | |
| 747 | TGCTAAAAAAA | 4.16 | 1.92 | 2.17 | 0.68 | Hs.146550 | myosin, heavy | |
| | | | | | | | polypeptide 9, non-muscle | |
| 748 | ATCCGTGCCCT | 4.16 | 1.92 | 2.17 | 0.68 | Hs.141011 | calmodulin 3 | |
| | | | | | | | (phosphorylase | |
| | | | | | | | kinase, delta) | |
| 749 | GTGGCGTGCGC | 4.16 | 1.92 | 2.17 | 0.68 | Hs.117582 | CGI-43 protein | |
| 750 | CCTTTGTCTTT | 2.08 | 0.96 | 2.17 | 0.46 | Hs.99654 | protein-O-mannosyltransferase | |
| | | | | | | | 1 | |
| 751 | GCAACAGCAAT | 2.08 | 0.96 | 2.17 | 0.46 | Hs.9950 | Sec61 gamma | |
| 752 | GCCTGGGACTC | 2.08 | 0.96 | 2.17 | 0.46 | Hs.98057 | ESTs, Weakly similar | |
| | | | | | | | to I68667 | |
| | | | | | | | transcription factor | |
| | | | | | | | ZFM, splice from | |
| | | | | | | | ABCDF-human_[H,sapiens] | |
| 753 | CTCTAGAGAAA | 2.08 | 0.96 | 2.17 | 0.46 | Hs.97925 | hypothetical protein | |
| 754 | CCCTCCTGCTC | 2.08 | 0.96 | 2.17 | 0.46 | Hs.96731 | huntingtin interacting | |
| | | | | | | | protein-1-related | |
| 755 | AAGGTGGAGTG | 2.08 | 0.96 | 2.17 | 0.46 | Hs.9573 | ATP-binding | |
| | | | | | | | cassette, sub-family F | |
| | | | | | | | (GCN20), member 1 | |
| 756 | AACAAGGTGAG | 2.08 | 0.96 | 2.17 | 0.46 | Hs.94952 | ESTs, Highly similar | |
| | | | | | | | to transcription | |
| | | | | | | | elongation factor | |
| | | | | | | | TFIIS,h [H,sapiens] | |
| 757 | ACTGAAGGCGC | 2.08 | 0.96 | 2.17 | 0.46 | Hs.92208 | a disintegrin and | |
| | | | | | | | metalloproteinase | |
| | | | | | | | domain 15 | |
| | | | | | | | (metargidin) | |
| 758 | CTAATTTAACT | 2.08 | 0.96 | 2.17 | 0.46 | Hs.9194 | putative glialblastoma | |
| | | | | | | | cell differentiation-related | |
| 759 | GCCTTGATCTC | 2.08 | 0.96 | 2.17 | 0.46 | Hs.91146 | DKFZP586E0820 | |
| | | | | | | | protein | |
| 760 | CCTCCCTGCTC | 2.08 | 0.96 | 2.17 | 0.46 | Hs.90790 | ESTs | |
| 761 | GAGCCTGGATA | 2.08 | 0.96 | 2.17 | 0.46 | Hs.9004 | chondroitin sulfate | |
| | | | | | | | proteoglycan 4 | |
| | | | | | | | (melanoma-associated) | |
| 762 | TTTACAGCTGG | 2.08 | 0.96 | 2.17 | 0.46 | Hs.89981 | diacylglycerol kinase, | |
| | | | | | | | zeta (104kD) | |
| 763 | GCTTTACTTTG | 2.08 | 0.96 | 2.17 | 0.46 | Hs.8966 | integral membrane | |
| | | | | | | | protein 1 | |
| 764 | ACCTAGCCACT | 2.08 | 0.96 | 2.17 | 0.46 | Hs.89463 | potassium large | |
| | | | | | | | conductance calcium-activated | |
| | | | | | | | channel, | |
| | | | | | | | subfamily M, alpha | |
| | | | | | | | member 1 | |
| 765 | GACCTCCTGCC | 2.08 | 0.96 | 2.17 | 0.46 | Hs.89449 | mitogen-activated | |
| | | | | | | | protein kinase kinase | |
| | | | | | | | kinase 11 | |
| 766 | CTCATATGTTA | 2.08 | 0.96 | 2.17 | 0.46 | Hs.8939 | yes-associated | |
| | | | | | | | protein 65 kDa | |
| 767 | TTATACAAAAA | 2.08 | 0.96 | 2.17 | 0.46 | Hs.88558 | ESTs | |
| 768 | GCCCCCCCGTG | 2.08 | 0.96 | 2.17 | 0.46 | Hs.85573 | Homo sapiens | |
| | | | | | | | mRNA; cDNA | |
| | | | | | | | DKFZp566N034 | |
| | | | | | | | (from clone | |
| | | | | | | | DKFZp566N034); | |
| | | | | | | | partial cds | |
| 769 | CTTTGATGTTC | 2.08 | 0.96 | 2.17 | 0.46 | Hs.85100 | WO repeat domain 1 | |
| 770 | CGGACTCACTG | 2.08 | 0.96 | 2.17 | 0.46 | Hs.84700 | similar to | |
| | | | | | | | phosphatidylcholine | |
| | | | | | | | transfer protein 2 | |
| 771 | CATTTGTAAAA | 2.08 | 0.96 | 2.17 | 0.46 | Hs.84429 | KIAA0971 protei | |
| 772 | CTTCTCACCGT | 2.08 | 0.96 | 2.17 | 0.46 | Hs.84285 | ubiquitin-conjugating | |
| | | | | | | | enzyme E2I | |
| | | | | | | | (homologous to yeast | |
| | | | | | | | UBC9) | |
| 773 | ACCAGCTGTCC | 2.08 | 0.96 | 2.17 | 0.46 | Hs.84153 | dynamitin (dynactin | |
| | | | | | | | complex 50 kD | |
| | | | | | | | subunit) | |
| 774 | ACAAAATAAAA | 2.08 | 0.96 | 2.17 | 0.46 | Hs.83469 | nuclear factor | |
| | | | | | | | (erythroid-derived 2)-like 1 | |
| 775 | AAACATTAGCC | 2.08 | 0.96 | 2.17 | 0.46 | Hs.82911 | protein tyrosine | |
| | | | | | | | phosphatase type | |
| | | | | | | | IVA, member 2 | |
| 776 | TAAATGAAAAA | 2.08 | 0.96 | 2.17 | 0.46 | Hs.82120 | nuclear receptor | |
| | | | | | | | subfamily 4, group A, | |
| | | | | | | | member 2 | |
| 777 | GAGACCCTGGA | 2.08 | 0.96 | 2.17 | 0.46 | Hs.8088 | similar to S, | |
| | | | | | | | cerevisiae Sec6p and | |
| | | | | | | | R, norvegicus rsec6 | |
| 778 | TTCCCTCGTGA | 2.08 | 0.96 | 2.17 | 0.46 | Hs.80758 | aspartyl-tRNA | |
| | | | | | | | synthetase | |
| 779 | AGGATAAAAAA | 2.08 | 0.96 | 2.17 | 0.46 | Hs.79404 | neuron-specific | |
| | | | | | | | protein | |
| 780 | GCAAATCCTGT | 2.08 | 0.96 | 2.17 | 0.46 | Hs.79059 | transforming growth | |
| | | | | | | | factor, beta receptor | |
| | | | | | | | III (betaglycan, | |
| | | | | | | | 300kD) | |
| 781 | GTCTCAGTCAT | 2.08 | 0.96 | 2.17 | 0.46 | Hs.78943 | bleomycin hydrolase | |
| 782 | ACCAGACAGAC | 2.08 | 0.96 | 2.17 | 0.46 | Hs.7882 | ESTs | |
| 783 | GTTGTAAAATA | 2.08 | 0.96 | 2.17 | 0.46 | Hs.7869 | lysophosphatidic acid | |
| | | | | | | | acyltransferase-delta | |
| 784 | CTCACTAGTGG | 2.08 | 0.96 | 2.17 | 0.46 | Hs.78683 | ubiquitin specific | |
| | | | | | | | protease 7 (herpes | |
| | | | | | | | virus-associated) | |
| 785 | AAACGAAGTTG | 2.08 | 0.96 | 2.17 | 0.46 | Hs.78353 | SFRS protein kinase | |
| | | | | | | | 2 | |
| 786 | AGCTCTTGGAG | 2.08 | 0.96 | 2.17 | 0.46 | Hs.7833 | selenium binding | |
| | | | | | | | protein 1 | |
| 787 | AGTCGCCTTCA | 2.08 | 0.96 | 2.17 | 0.46 | Hs.7811 | eukaryotic translation | |
| | | | | | | | initiation factor 3, | |
| | | | | | | | subunit 5 (epsilon, | |
| | | | | | | | 47kD) | |
| 788 | CGATGGTCCCC | 2.08 | 0.96 | 2.17 | 0.46 | Hs.7771 | B-cell associated | |
| | | | | | | | protein | |
| 789 | ACTGCTTGCCC | 2.08 | 0.96 | 2.17 | 0.46 | Hs.77502 | methionine | |
| | | | | | | | adenosyltransferase | |
| | | | | | | | II, alpha | |
| 790 | TAAAAGACAAA | 2.08 | 0.96 | 2.17 | 0.46 | Hs.77196 | spectrin, alpha, non-erythrocytic | |
| | | | | | | | 1 (alpha-fodrin) | |
| 791 | GACTCGCCCAC | 2.08 | 0.96 | 2.17 | 0.46 | Hs.77171 | minichromosome | |
| | | | | | | | maintenance deficient | |
| | | | | | | | (S, cerevisiae) 5 (cell | |
| | | | | | | | division cycle 46) | |
| 792 | TATATTGATTG | 2.08 | 0.96 | 2.17 | 0.46 | Hs.77054 | B-cell translocation | |
| | | | | | | | gene 1, anti-proliferative | |
| 793 | CTGGGACTGAC | 2.08 | 0.96 | 2.17 | 0.46 | Hs.76719 | U6 snRNA-associated | |
| | | | | | | | Sm-like | |
| | | | | | | | protein | |
| 794 | CTCTTCGAGAA | 2.08 | 0.96 | 2.17 | 0.46 | Hs.76686 | glutathione | |
| | | | | | | | peroxidase 1 | |
| 795 | TCTGTCAAGAC | 2.08 | 0.96 | 2.17 | 0.46 | Hs.76572 | ATP synthase, H+ | |
| | | | | | | | transporting, | |
| | | | | | | | mitochondrial F1 | |
| | | | | | | | complex, O subunit | |
| | | | | | | | (oligomycin sensitivity | |
| | | | | | | | conferring protein) | |
| 796 | ATGAAAAGAAA | 2.08 | 0.96 | 2.17 | 0.46 | Hs.76550 | Homo sapiens | |
| | | | | | | | mRNA; cDNA | |
| | | | | | | | DKFZp564B1264 | |
| | | | | | | | (from clone | |
| | | | | | | | DKFZp564B1264) | |
| 797 | AACACATCAGC | 2.08 | 0.96 | 2.17 | 0.46 | Hs.76253 | spinocerebellar ataxia 2 | |
| | | | | | | | (olivopontocerebellar | |
| | | | | | | | ataxia 2, autosomal | |
| | | | | | | | dominant, ataxin 2) | |
| 798 | AAAACCTGTAA | 2.08 | 0.96 | 2.17 | 0.46 | Hs.75914 | coated vesicle | |
| | | | | | | | membrane protein | |
| 799 | CCTTGG1TTTG | 2.08 | 0.96 | 2.17 | 0.46 | Hs.75875 | ubiquitin-conjugating | |
| | | | | | | | enzyme E2 variant 1 | |
| 800 | AGTTTCCCAAT | 2.08 | 0.96 | 2.17 | 0.46 | Hs.75854 | sulfotransferase | |
| | | | | | | | family, cytosolic, 1C, | |
| | | | | | | | member 2 | |
| 801 | GATTTTTAAAA | 2.08 | 0.96 | 2.17 | 0.46 | Hs.75447 | raIA binding protein 1 | |
| 802 | AGGGGATTCCC | 2.08 | 0.96 | 2.17 | 0.46 | Hs.75412 | Arginine-rich protein | |
| 803 | ACAAATCCTTG | 2.08 | 0.96 | 2.17 | 0.46 | Hs.752 | FK506-binding | |
| | | | | | | | protein 1A (12kD) | |
| 804 | GTGACAGACAT | 2.08 | 0.96 | 2.17 | 0.46 | Hs.75117 | interleukin enhancer | |
| | | | | | | | binding factor 2, 45kD | |
| 805 | CAGACTTTTTT | 2.08 | 0.96 | 2.17 | 0.46 | Hs.74649 | cytochrome c oxidase | |
| | | | | | | | subunit Vic | |
| 806 | TGCGGCTGGTT | 2.08 | 0.96 | 2.17 | 0.46 | Hs.74617 | dynactin 1 (p150, | |
| | | | | | | | Glued (Drosophila) | |
| | | | | | | | homolog) | |
| 807 | GGCTGCCCTGG | 2.08 | 0.96 | 2.17 | 0.46 | Hs.74566 | dihydropyrimidinase-like | |
| | | | | | | | 3 | |
| 808 | CCTGTAACACC | 2.08 | 0.96 | 2.17 | 0.46 | Hs.74304 | periplakin | |
| 809 | GTTTCAGTTAC | 2.08 | 0.96 | 2.17 | 0.46 | Hs.7016 | RAB7, member RAS | |
| | | | | | | | oncogene family | |
| 810 | CTGGAAATAAA | 2.08 | 0.96 | 2.17 | 0.46 | Hs.69745 | ferredoxin reductase | |
| 811 | GTGATGTACGG | 2.08 | 0.96 | 2.17 | 0.46 | Hs.6639 | Homo sapiens cDNA | |
| | | | | | | | FLJ20818 fis, clone | |
| | | | | | | | ADSE00627 | |
| 812 | CAGCCTTGGAC | 2.08 | 0.96 | 2.17 | 0.46 | Hs.65648 | RNA binding motif | |
| | | | | | | | protein 8 | |
| 813 | GTGGATGGACT | 2.08 | 0.96 | 2.17 | 0.46 | Hs.6418 | seven | |
| | | | | | | | transmembrane | |
| | | | | | | | domain orphan | |
| | | | | | | | receptor | |
| 814 | TGCCAGAAATG | 2.08 | 0.96 | 2.17 | 0.46 | Hs.63510 | KIAA0141 gene | |
| | | | | | | | product | |
| 815 | AATAATCCTGG | 2.08 | 0.96 | 2.17 | 0.46 | Hs.62908 | ESTs | |
| 816 | GGAGGGATCAG | 2.08 | 0.96 | 2.17 | 0.46 | Hs.6196 | integrin-linked kinase | |
| 817 | GGATTCCAGTT | 2.08 | 0.96 | 2.17 | 0.46 | Hs.5321 | ARP3 (actin-related | |
| | | | | | | | protein 3, yeast) | |
| | | | | | | | homolog | |
| 818 | TAATTTCTCAA | 2.08 | 0.96 | 2.17 | 0.46 | Hs.5306 | Homo sapiens | |
| | | | | | | | mRNA; cDNA | |
| | | | | | | | DKFZp586F1122 | |
| | | | | | | | (from clone | |
| | | | | | | | DKFZp586F1122) | |
| 819 | CCTGTAGACCC | 2.08 | 0.96 | 2.17 | 0.46 | Hs.5123 | inorganic | |
| | | | | | | | pyrophosphatase | |
| 820 | CTGCAACCTAA | 2.08 | 0.96 | 2.17 | 0.46 | Hs.50785 | SEC22, vesicle | |
| | | | | | | | trafficking protein (S, | |
| | | | | | | | cerevisiae)-like 1 | |
| 821 | GACGGCTGCAA | 2.08 | 0.96 | 2.17 | 0.46 | Hs.4909 | dickkopf (Xenopus | |
| | | | | | | | laevis) homolog 3 | |
| 822 | CATTGCAGGAT | 2.08 | 0.96 | 2.17 | 0.46 | Hs.4288 | hypothetical protein | |
| | | | | | | | DKFZp434K046 | |
| 823 | GCAGAGATGGG | 2.08 | 0.96 | 2.17 | 0.46 | Hs.39850 | hypothetical protein | |
| | | | | | | | FLJ20517 | |
| 824 | TCAGTTTGGAG | 2.08 | 0.96 | 2.17 | 0.46 | Hs.3873 | palmitoyl-protein | |
| | | | | | | | thioesterase 1 | |
| | | | | | | | (ceroid-lipofuscinosis, | |
| | | | | | | | neuronal 1, infantile) | |
| 825 | ATAGCTGGGGC | 2.08 | 0.96 | 2.17 | 0.46 | Hs.3446 | mitogen-activated | |
| | | | | | | | protein kinase kinase | |
| | | | | | | | 1 | |
| 826 | CGTACAGCCCC | 2.08 | 0.96 | 2.17 | 0.46 | Hs.32580 | KIAA1448 protein | |
| 827 | GTGAAACCGTC | 2.08 | 0.96 | 2.17 | 0.46 | Hs.30596 | Homo sapiens mRNA | |
| | | | | | | | full length insert | |
| | | | | | | | cDNA clone | |
| | | | | | | | EUROIMAGE 43465 | |
| 828 | ATTACAAACCT | 2.08 | 0.96 | 2.17 | 0.46 | Hs.30376 | hypothetical protein | |
| 829 | AGGATCACTTG | 2.08 | 0.96 | 2.17 | 0.46 | Hs.287355 | ESTs | |
| 830 | CAGATTAGTTA | 2.08 | 0.96 | 2.17 | 0.46 | Hs.286195 | Homo sapiens clone | |
| | | | | | | | 25244 DEAD-box | |
| | | | | | | | protein p72 mRNA | |
| | | | | | | | sequence, complete | |
| | | | | | | | cds | |
| 831 | AGGGGGGAGGG | 2.08 | 0.96 | 2.17 | 0.46 | Hs.284181 | hypothetical protein | |
| | | | | | | | DKFZp434P0531 | |
| 832 | ATTTCCATTAA | 2.08 | 0.96 | 2.17 | 0.46 | Hs.284126 | hairless (mouse) | |
| | | | | | | | homolog | |
| 833 | TCATTGTAATG | 2.08 | 0.96 | 2.17 | 0.46 | Hs.283722 | GTT1 protein | |
| 834 | GTAACAAGCTC | 2.08 | 0.96 | 2.17 | 0.46 | Hs.279849 | KIAA0438 gene | |
| | | | | | | | product | |
| 835 | CTAATAAACTT | 2.08 | 0.96 | 2.17 | 0.46 | Hs.279583 | CGI-81 protein | |
| 836 | ACATCCTCACC | 2.08 | 0.96 | 2.17 | 0.46 | Hs.279554 | proteasome | |
| | | | | | | | (prosome | |
| | | | | | | | macropain) 26S | |
| | | | | | | | subunit, non-ATPase, | |
| | | | | | | | 13 | |
| 837 | CTCCAATAAAA | 2.08 | 0.96 | 2.17 | 0.46 | Hs.278559 | talin | |
| 838 | CCACTGCATTG | 2.08 | 0.96 | 2.17 | 0.46 | Hs.278551 | ESTs | |
| 839 | ATTTTTTTCAG | 2.08 | 0.96 | 2.17 | 0.46 | Hs.278004 | EST | |
| 840 | TTCTCTCAACT | 2.08 | 0.96 | 2.17 | 0.46 | Hs.27445 | unknown | |
| 841 | GTGGCGAGCAC | 2.08 | 0.96 | 2.17 | 0.46 | Hs.261831 | EST | |
| 842 | CACCTTCTGCC | 2.08 | 0.96 | 2.17 | 0.46 | Hs.25511 | transforming growth | |
| | | | | | | | factor beta 1 induced | |
| | | | | | | | transcript 1 | |
| 843 | TCTCTGCAAAA | 2.08 | 0.96 | 2.17 | 0.46 | Hs.25489 | hypothetical protein | |
| | | | | | | | FLJ20640 | |
| 844 | AAAGGGGGCAG | 2.08 | 0.96 | 2.17 | 0.46 | Hs.249247 | heterogeneous | |
| | | | | | | | nuclear protein | |
| | | | | | | | similar to rat helix | |
| | | | | | | | destabilizing protein | |
| 845 | CGGAGGTGGGA | 2.08 | 0.96 | 2.17 | 0.46 | Hs.2491 | DiGeorge syndrome | |
| | | | | | | | critical region gene 2 | |
| 846 | TAACTCCAAAG | 2.08 | 0.96 | 2.17 | 0.46 | Hs.24743 | hypothetical protein | |
| | | | | | | | FLJ20171 | |
| 847 | ATGTCCAATTT | 2.08 | 0.96 | 2.17 | 0.46 | Hs.247309 | succinate-CoA ligase, | |
| | | | | | | | GDP-forming, beta | |
| | | | | | | | subunit | |
| 848 | CATCCAAAACA | 2.08 | 0.96 | 2.17 | 0.46 | Hs.245710 | heterogeneous | |
| | | | | | | | nuclear | |
| | | | | | | | ribonucleoprotein H1 | |
| | | | | | | | (H) | |
| 849 | GGAGTCTAACT | 2.08 | 0.96 | 2.17 | 0.46 | Hs.240170 | ESTs, Moderately | |
| | | | | | | | similar to alternatively | |
| | | | | | | | spliced product using | |
| | | | | | | | exon 13A [H,sapiens] | |
| 850 | TGCTAGATTGG | 2.08 | 0.96 | 2.17 | 0.46 | Hs.239663 | myeloid/lymphoid or | |
| | | | | | | | mixed-lineage | |
| | | | | | | | leukemia | |
| 851 | GCCCCAGCGAG | 2.08 | 0.96 | 2.17 | 0.46 | Hs.238296 | ADP-ribosylation | |
| | | | | | | | factor binding protein | |
| | | | | | | | GGA1 | |
| 852 | CTGTGAAATGC | 2.08 | 0.96 | 2.17 | 0.46 | Hs.23618 | hypothetical protein | |
| | | | | | | | FLJ10704 | |
| 853 | GATCACAGTTT | 2.08 | 0.96 | 2.17 | 0.46 | Hs.234489 | lactate | |
| | | | | | | | dehydrogenase B | |
| 854 | GTGAAACACCA | 2.08 | 0.96 | 2.17 | 0.46 | Hs.231777 | EST | |
| 855 | ATCCACCTGCC | 2.08 | 0.96 | 2.17 | 0.46 | Hs.231656 | EST | |
| 856 | GAGGCCAGTGA | 2.08 | 0.96 | 2.17 | 0.46 | Hs.2280 | ribophorin I | |
| 857 | AAGTACGAGGA | 2.08 | 0.96 | 2.17 | 0.46 | Hs.22660 | ESTs | |
| 858 | CCTACTGCACT | 2.08 | 0.96 | 2.17 | 0.46 | Hs.225641 | ESTs, Moderately | |
| | | | | | | | similar to KIAA0680 | |
| | | | | | | | protein [H,sapiens] | |
| 859 | TTCTCTGCTCA | 2.08 | 0.96 | 2.17 | 0.46 | Hs.21907 | histone | |
| | | | | | | | acetyltransferase | |
| 860 | TACGTTGCAGC | 2.08 | 0.96 | 2.17 | 0.46 | Hs.21756 | translation factor sui1 | |
| | | | | | | | homolog | |
| 861 | TACCAAGGATT | 2.08 | 0.96 | 2.17 | 0.46 | Hs.21729 | splicing factor 3a, | |
| | | | | | | | subunit 1, 120kD | |
| 862 | CTGTAGAAATG | 2.08 | 0.96 | 2.17 | 0.46 | Hs.215595 | guanine nucleotide | |
| | | | | | | | binding protein (G | |
| | | | | | | | protein), beta | |
| | | | | | | | polypeptide 1 | |
| 863 | GTGAAACCCTT | 2.08 | 0.96 | 2.17 | 0.46 | Hs.206955 | ESTs | |
| 864 | ACTGCTGAACC | 2.08 | 0.96 | 2.17 | 0.46 | Hs.200600 | secretory carrier | |
| | | | | | | | membrane protein 3 | |
| 865 | TTGCGGAGCCC | 2.08 | 0.96 | 2.17 | 0.46 | Hs.199695 | hypothetical protein | |
| 866 | TGCCGTAAATG | 2.08 | 0.96 | 2.17 | 0.46 | Hs.199067 | v-erb-b2 avian | |
| | | | | | | | erythroblastic | |
| | | | | | | | leukemia viral | |
| | | | | | | | oncogene homolog 3 | |
| 867 | ATCAGTGTGCA | 2.08 | 0.96 | 2.17 | 0.46 | Hs.194662 | calponin 3, acidic | |
| 868 | ACCAGCCAAAG | 2.08 | 0.96 | 2.17 | 0.46 | Hs.193090 | ESTs, Highly similar | |
| | | | | | | | to AF161437_1 | |
| | | | | | | | HSPC319 [H,sapiens] | |
| 869 | AACAGATATTG | 2.08 | 0.96 | 2.17 | 0.46 | Hs.190161 | LR8 protein | |
| 870 | TTGGCAAGGCT | 2.08 | 0.96 | 2.17 | 0.46 | Hs.184720 | ESTs | |
| 871 | TCTGGGGAACA | 2.08 | 0.96 | 2.17 | 0.46 | Hs.184390 | similar to aspartate | |
| | | | | | | | beta hydroxylase | |
| | | | | | | | (ASPH) | |
| 872 | GCTCTCGGCGG | 2.08 | 0.96 | 2.17 | 0.46 | Hs.183994 | protein phosphatase | |
| | | | | | | | 1, catalytic subunit, | |
| | | | | | | | alpha isoform | |
| 873 | GGACTGAGTCA | 2.08 | 0.96 | 2.17 | 0.46 | Hs.18387 | transcription factor | |
| | | | | | | | AP-2 alpha | |
| | | | | | | | (activating enhancer-binding | |
| | | | | | | | protein 2 | |
| | | | | | | | alpha) | |
| 874 | GAGCACTTGGG | 2.08 | 0.96 | 2.17 | 0.46 | Hs.182937 | peptidylprolyl | |
| | | | | | | | isomerase A | |
| | | | | | | | (cyclophilin A) | |
| 875 | TTTTGTGTGAA | 2.08 | 0.96 | 2.17 | 0.46 | Hs.182698 | hypothetical protein | |
| | | | | | | | FLJ10024 | |
| 876 | CCTGTAATTGC | 2.08 | 0.96 | 2.17 | 0.46 | Hs.181464 | ESTs | |
| 877 | ACCCCCTTCCT | 2.08 | 0.96 | 2.17 | 0.46 | Hs.181392 | major | |
| | | | | | | | histocompatibility | |
| | | | | | | | complex, class I, E | |
| 878 | GCCTGGGACCT | 2.08 | 0.96 | 2.17 | 0.46 | Hs.180871 | protein kinase C, | |
| | | | | | | | alpha binding protein | |
| 879 | AGGAAAAAAAA | 2.08 | 0.96 | 2.17 | 0.46 | Hs.180639 | EST | |
| 880 | GTTTGGAGCTG | 2.08 | 0.96 | 2.17 | 0.46 | Hs.180533 | mitogen-activated | |
| | | | | | | | protein kinase kinase | |
| | | | | | | | 3 | |
| 881 | GGCAACAAAAG | 2.08 | 0.96 | 2.17 | 0.46 | Hs.180446 | karyopherin (importin) | |
| | | | | | | | beta 1 | |
| 882 | TTCCATACCCC | 2.08 | 0.96 | 2.17 | 0.46 | Hs.180398 | LIM domain-containing | |
| | | | | | | | preferred | |
| | | | | | | | translocation partner | |
| | | | | | | | in lipoma | |
| 883 | CAACTTAGTTT | 2.08 | 0.96 | 2.17 | 0.46 | Hs.180224 | death-associated | |
| | | | | | | | protein 6 | |
| 884 | GCATATTAAAA | 2.08 | 0.96 | 2.17 | 0.46 | Hs.178658 | RAD23 (S, | |
| | | | | | | | cerevisiae) homolog | |
| | | | | | | | B | |
| 885 | GACTCTCTCAG | 2.08 | 0.96 | 2.17 | 0.46 | Hs.178576 | similar to Bos taurus | |
| | | | | | | | P14 protein | |
| 886 | TATCCCAGAAT | 2.08 | 0.96 | 2.17 | 0.46 | Hs.175819 | EST | |
| 887 | TGAACTTTCCT | 2.08 | 0.96 | 2.17 | 0.46 | Hs.17567 | ESTs | |
| 888 | GAAATGGGGAA | 2.08 | 0.96 | 2.17 | 0.46 | Hs.173933 | Homo sapiens mRNA | |
| | | | | | | | for KIAA1439 protein, | |
| | | | | | | | partial cds | |
| 889 | TACTAAAAAAG | 2.08 | 0.96 | 2.17 | 0.46 | Hs.173611 | NADH | |
| | | | | | | | dehydrogenase | |
| | | | | | | | (ubiquinone) Fe-S | |
| | | | | | | | protein 2 (49kD) | |
| 890 | AACTGGCTGCT | 2.08 | 0.96 | 2.17 | 0.46 | Hs.173381 | dihydropyrimidinase-like | |
| | | | | | | | 2 | |
| 891 | TGGAAATGAAA | 2.08 | 0.96 | 2.17 | 0.46 | Hs.172928 | collagen, type I, alpha | |
| | | | | | | | 1 | |
| 892 | TGAGGGATGGA | 2.08 | 0.96 | 2.17 | 0.46 | Hs.172740 | microtubule-associated | |
| | | | | | | | protein, | |
| | | | | | | | RP/EB family, | |
| | | | | | | | member 3 | |
| 893 | CAGTGGGGTTA | 2.08 | 0.96 | 2.17 | 0.46 | Hs.17138 | hypothetical protein | |
| | | | | | | | FLJ20303 | |
| 894 | GAGGGTTCCAG | 2.08 | 0.96 | 2.17 | 0.46 | Hs.167835 | acyl-Coenzyme A | |
| | | | | | | | oxidate 1, palmitoyl | |
| 895 | ACCCATCGCCT | 2.08 | 0.96 | 2.17 | 0.46 | Hs.165428 | ESTs | |
| 896 | CACTGTGTGTA | 2.08 | 0.96 | 2.17 | 0.46 | Hs.164207 | ESTs | |
| 897 | AGGCAGAGGTT | 2.08 | 0.96 | 2.17 | 0.46 | Hs.164129 | ESTs | |
| 898 | TTCTGGACCCA | 2.08 | 0.96 | 2.17 | 0.46 | Hs.155543 | proteasome | |
| | | | | | | | (prosome, | |
| | | | | | | | macropain) 26S | |
| | | | | | | | subunit, non-ATPase, | |
| | | | | | | | 7 (Mov34 homolog) | |
| 899 | ATGGCCATAGA | 2.08 | 0.96 | 2.17 | 0.46 | Hs.155206 | serine/threonine | |
| | | | | | | | kinase 25 (Ste20, | |
| | | | | | | | yeast homolog) | |
| 900 | GCGGGAGGGCT | 2.08 | 0.96 | 2.17 | 0.46 | Hs.154162 | ADP-ribosylation | |
| | | | | | | | factor-like 2 | |
| 901 | AGGCATTGAAA | 2.08 | 0.96 | 2.17 | 0.46 | Hs.151734 | nuclear transport | |
| | | | | | | | factor 2 (placental | |
| | | | | | | | protein 15) | |
| 902 | GTCTTTCTTGG | 2.08 | 0.96 | 2.17 | 0.46 | Hs.151536 | RAB13, member RAS | |
| | | | | | | | oncogene family | |
| 903 | GCAAAACCAGC | 2.08 | 0.96 | 2.17 | 0.46 | Hs.15071 | chaperonin | |
| | | | | | | | containing TCP1, | |
| | | | | | | | subunit 8 (theta) | |
| 904 | TGAAGTAACAA | 2.08 | 0.96 | 2.17 | 0.46 | Hs.150580 | putative translation | |
| | | | | | | | initiation factor | |
| 905 | CAATTAAAAGG | 2.08 | 0.96 | 2.17 | 0.46 | Hs.149923 | X-box binding protein | |
| | | | | | | | 1 | |
| 906 | TTTGAGGATTG | 2.08 | 0.96 | 2.17 | 0.46 | Hs.147916 | DEAD/H (Asp-Glu-Ala-Asp/His) | |
| | | | | | | | box | |
| | | | | | | | polypeptide 3 | |
| 907 | GAGTAGAGAAA | 2.08 | 0.96 | 2.17 | 0.46 | Hs.145279 | SET translocation | |
| | | | | | | | (myeloid leukemia-associated) | |
| 908 | AAGCCAGCCCC | 2.08 | 0.96 | 2.17 | 0.46 | Hs.1432 | protein kinase C | |
| | | | | | | | substrate 80K-H | |
| 909 | TATCTGGTCTT | 2.08 | 0.96 | 2.17 | 0.46 | Hs.142258 | signal transducer and | |
| | | | | | | | activator of | |
| | | | | | | | transcription 3 (acute-phase | |
| | | | | | | | response | |
| | | | | | | | factor) | |
| 910 | GTTCTCCCACT | 2.08 | 0.96 | 2.17 | 0.46 | Hs.14038 | sec61 homolog | |
| 911 | CCGAGTTTTTG | 2.08 | 0.96 | 2.17 | 0.46 | Hs.139709 | ESTs | |
| 912 | TGGAAGGGCAC | 2.08 | 0.96 | 2.17 | 0.46 | Hs.132785 | EAP30 subunit of | |
| | | | | | | | ELL complex | |
| 913 | AAGGCGTTTCC | 2.08 | 0.96 | 2.17 | 0.46 | Hs.13255 | KIAA0930 protein | |
| 914 | TGCCTTAGTAA | 2.08 | 0.96 | 2.17 | 0.46 | Hs.13015 | ESTs, Highly similar | |
| | | | | | | | to MTJ1_MOUSE | |
| | | | | | | | DNAJPROTEIN | |
| | | | | | | | HOMOLOG MTJ1_[M,musculus] | |
| 915 | TTTCTGGAGGT | 2.08 | 0.96 | 2.17 | 0.46 | Hs.129943 | KIAA0545 protein | |
| 916 | CCTGGCCAAAA | 2.08 | 0.96 | 2.17 | 0.46 | Hs.126824 | EST | |
| 917 | CAGAATAATGT | 2.08 | 0.96 | 2.17 | 0.46 | Hs.125031 | choline/ethanolamine | |
| | | | | | | | phosphotransferase | |
| 918 | CGGGGACGAGG | 2.08 | 0.96 | 2.17 | 0.46 | Hs.124942 | protein phosphatase | |
| | | | | | | | 2A 48 kDa regulatory | |
| | | | | | | | subunit | |
| 919 | ACAGCCGTGGG | 2.08 | 0.96 | 2.17 | 0.46 | Hs.123090 | SWI/SNE related, | |
| | | | | | | | matrix associated, | |
| | | | | | | | actin dependent | |
| | | | | | | | regulator of | |
| | | | | | | | chromatin, subfamily | |
| | | | | | | | f, member 1 | |
| 920 | AGTCTCCCCTA | 2.08 | 0.96 | 2.17 | 0.46 | Hs.12303 | suppressor of Ty | |
| | | | | | | | (S,cerevisiae) 6 | |
| | | | | | | | homolog | |
| 921 | TGATGTGATCA | 2.08 | 0.96 | 2.17 | 0.46 | Hs.12272 | beclin 1 (coiled-coil, | |
| | | | | | | | myosin-like BCL2-interacting | |
| | | | | | | | protein) | |
| 922 | ACCAGGCCACC | 2.08 | 0.96 | 2.17 | 0.46 | Hs.12068 | carnitine | |
| | | | | | | | acetyltransferase | |
| 923 | TCCTTCTCCAC | 2.08 | 0.96 | 2.17 | 0.46 | Hs.119000 | actinin, alpha | |
| 924 | AATGAATAAAA | 2.08 | 0.96 | 2.17 | 0.46 | Hs.118797 | ubiquitin-conjugating | |
| | | | | | | | enzyme E2D 3 | |
| | | | | | | | (homologous to yeast | |
| | | | | | | | UBC4/5) | |
| 925 | GATGGGGACAA | 2.08 | 0.96 | 2.17 | 0.46 | Hs.118724 | DR1-associated | |
| | | | | | | | protein 1 (negative | |
| | | | | | | | cofactor 2 alpha) | |
| 926 | TTGGGAGGCTG | 2.08 | 0.96 | 2.17 | 0.46 | Hs.118269 | ESTs, Weakly similar | |
| | | | | | | | to A46010 X-linked | |
| | | | | | | | retinopathy protein | |
| | | | | | | | [H,sapiens] | |
| 927 | CCTTATATTTG | 2.08 | 0.96 | 2.17 | 0.46 | Hs.118174 | tetratricopeptide | |
| | | | | | | | repeat domain 3 | |
| 928 | CAGCAGAACTG | 2.08 | 0.96 | 2.17 | 0.46 | Hs.117582 | CGI-43 protein | |
| 929 | CCACCACACCC | 2.08 | 0.96 | 2.17 | 0.46 | Hs.117582 | CGI-43 protein | |
| 930 | ACTCGCTCTGT | 2.08 | 0.96 | 2.17 | 0.46 | Hs.11669 | laminin, alpha 5 | |
| 931 | AGTATCTGGGA | 2.08 | 0.96 | 2.17 | 0.46 | Hs.11538 | actin related protein | |
| | | | | | | | 2/3 complex, subunit | |
| | | | | | | | 1A (41 kD) | |
| 932 | AATGAAAAAAA | 2.08 | 0.96 | 2.17 | 0.46 | Hs.11393 | RAD51 (S, | |
| | | | | | | | cerevisiae) homolog | |
| | | | | | | | C | |
| 933 | TAGTTGGAACT | 2.08 | 0.96 | 2.17 | 0.46 | Hs.1119 | nuclear receptor | |
| | | | | | | | subfamily 4, group A, | |
| | | | | | | | member 1 | |
| 934 | AACCCAAACTC | 2.08 | 0.96 | 2.17 | 0.46 | Hs.11184 | hypothetical protein | |
| | | | | | | | FLJ20419 | |
| 935 | GAGGCCTCAGC | 2.08 | 0.96 | 2.17 | 0.46 | Hs.11184 | hypothetical protein | |
| | | | | | | | FLJ20419 | |
| 936 | TTTGTTAAAAC | 2.08 | 0.96 | 2.17 | 0.46 | Hs.111244 | hypothetical protein | |
| 937 | TTCAGCGTTCT | 2.08 | 0.96 | 2.17 | 0.46 | Hs.109929 | hypothetical protein | |
| | | | | | | | MPMGp800B12492Q | |
| | | | | | | | 3 | |
| 938 | GCCAGACCCCT | 2.08 | 0.96 | 2.17 | 0.46 | Hs.108945 | KIAA0515 protein | |
| 939 | GCTGGCTGGCT | 2.08 | 0.96 | 2.17 | 0.46 | Hs.108809 | chaperonin | |
| | | | | | | | containing TCP1, | |
| | | | | | | | subunit 7 (eta) | |
| 940 | GTTGGGAGTCC | 2.08 | | 0.96 | 2.17 | 0.46 | Hs.108504 | hypothetical protein |
| | | | | | | | FLJ20113 | |
| 941 | TCTTCTAAAAA | 2.08 | 0.96 | 2.17 | 0.46 | Hs.108112 | histone fold protein | |
| | | | | | | | CHRAC17; DNA | |
| | | | | | | | polymerase epsilon | |
| | | | | | | | p17 subunit | |
| 942 | AGAAAGAATCT | 2.08 | 0.96 | 2.17 | 0.46 | Hs.107979 | small membrane | |
| | | | | | | | protein 1 | |
| 943 | CCCATCTAGCT | 2.08 | 0.96 | 2.17 | 0.46 | Hs.106070 | cyclin-dependent | |
| | | | | | | | kinase inhibitor 1C | |
| | | | | | | | (p57, Kip2) | |
| 944 | TCTGCAAGCAG | 2.08 | 0.96 | 2.17 | 0.46 | Hs.105598 | ESTs, Weakly similar | |
| | | | | | | | to neural variant | |
| | | | | | | | mena++ protein | |
| | | | | | | | [M,musculus] | |
| 945 | AAAGAACATAG | 2.08 | 0.96 | 2.17 | 0.46 | Hs.104558 | ESTs | |
| 946 | GGCAAACTTTA | 2.08 | 0.96 | 2.17 | 0.46 | Hs.102497 | paxillin | |
| 947 | AGGGACATAAA | 2.08 | 0.96 | 2.17 | 0.46 | Hs.101516 | BAl1-associated | |
| | | | | | | | protein 3 | |
| 948 | TAAAGATCCTC | 2.08 | 0.96 | 2.17 | 0.46 | Hs.100407 | Homo sapiens | |
| | | | | | | | mRNA; cDNA | |
| | | | | | | | DKFZp564H2416 | |
| | | | | | | | (from clone | |
| | | | | | | | DKFZp564H2416) | |
| 949 | TCAAGCCATCA | 24.99 | 11.54 | 2.17 | 1.97 | Hs.738 | early growth | |
| | | | | | | | response 1 | |
| 950 | CCTGGCTAATT | 20.82 | 9.62 | 2.16 | 1.75 | Hs.25661 | ESTs | |
| 951 | GCTGTAATCCC | 10.41 | 4.81 | 2.16 | 1.15 | Hs.184019 | Homo sapiens clone | |
| | | | | | | | 23551 mRNA | |
| | | | | | | | sequence | |
| 952 | AAGCACAAAAA | 8.33 | 3.85 | 2.16 | 1.01 | Hs.9963 | TYRO protein | |
| | | | | | | | tyrosine kinase | |
| 953 | CCCACTTGTAA | 8.33 | 3.85 | 2.16 | 1.01 | Hs.75922 | brain protein 13 | |
| 954 | GCTTGGATCTC | 8.33 | 3.85 | 2.16 | 1.01 | Hs.250723 | FK506 binding | |
| | | | | | | | protein 12-rapamycin | |
| | | | | | | | associated protein 1 | |
| 955 | CCACTGCTCTC | 8.33 | 3.85 | 2.16 | 1.01 | Hs.23510 | Kruppel-like factor 12 | |
| 956 | TTGGCCAGACT | 6.25 | 2.89 | 2.16 | 0.86 | Hs.91728 | polymyositis/sclerode | |
| | | | | | | | rma autoantigen 1 | |
| | | | | | | | (75kD) | |
| 957 | AGGTCCTAGCC | 6.25 | 2.89 | 2.16 | 0.86 | Hs.226795 | glutathione S-transferase | |
| | | | | | | | pi | |
| 958 | GTGGTGTACGC | 6.25 | 2.89 | 2.16 | 0.86 | Hs.182225 | RNA binding motif | |
| | | | | | | | protein 3 | |
| 959 | AGCCCAGGAGG | 6.25 | 2.89 | 2.16 | 0.86 | Hs.136340 | ESTs, Weakly similar | |
| | | | | | | | to unnamed protein | |
| | | | | | | | product [H,sapiens] | |
| 960 | CAGATCTTTGT | 6.25 | 2.89 | 2.16 | 0.86 | Hs.119502 | ubiquitin A-52 residue | |
| | | | | | | | ribosomal protein | |
| | | | | | | | fusion product 1 | |
| 961 | CCGTGGTCGTG | 3.12 | 6.73 | 2.16 | 0.88 | Hs.99853 | fibrillarin | |
| 962 | AGACCAAAGTG | 3.12 | 6.73 | 2.16 | 0.88 | Hs.82646 | heat shock 40kD | |
| | | | | | | | protein 1 | |
| 963 | TGAGTCTGGCT | 3.12 | 6.73 | 2.16 | 0.88 | Hs.4055 | chromosome 21 open | |
| | | | | | | | reading frame 50 | |
| 964 | GCAAAACTCTG | 3.12 | 6.73 | 2.16 | 0.88 | Hs.278746 | ESTs | |
| 965 | CCAGCTGCCAA | 3.12 | 6.73 | 2.16 | 0.88 | Hs.2055 | ubiquitin-activating | |
| | | | | | | | enzyme E1 | |
| 966 | GCGAAATCCCG | 3.12 | 6.73 | 2.16 | 0.88 | Hs.194251 | ESTs | |
| 967 | AAGGATGCCAA | 3.12 | 6.73 | 2.16 | 0.88 | Hs.169946 | GATA-binding protein | |
| | | | | | | | 3 | |
| 968 | CAGCTATTTCA | 3.12 | 6.73 | 2.16 | 0.88 | Hs.153179 | fatty acid binding | |
| | | | | | | | protein 5 (psoriasis-associated) | |
| 969 | GAATTATACTT | 3.12 | 6.73 | 2.16 | 0.88 | Hs.104800 | hypothetical protein | |
| | | | | | | | FLJ10134 | |
| 970 | GGAGGGGGCTT | 9.37 | 20.20 | 2.16 | 1.70 | Hs.77886 | lamin A/C | |
| 971 | AAGGAGATGGG | 7.29 | 15.39 | 2.11 | 1.41 | Hs.184014 | ribosomal protein L31 | |
| 972 | GGAGGTGGGGC | 7.29 | 15.39 | 2.11 | 1.41 | Hs.180577 | granulin | |
| 973 | GAAAACAAAGT | 153.04 | 319.40 | 2.09 | 14.34 | Hs.99936 | keratin 10 | |
| | | | | | | | (epidermolytic | |
| | | | | | | | hyperkeratosis; | |
| | | | | | | | keratosis palmaris et | |
| | | | | | | | plantaris) | |
| 974 | TTGGCTTTTCT | 4.16 | 8.66 | 2.08 | 1.00 | Hs.218329 | hypothetical protein | |
| 975 | GTGAAGCCCCA | 4.16 | 8.66 | 2.08 | 1.00 | Hs.171501 | ubiquitin specific | |
| | | | | | | | protease 11 | |
| 976 | ATGGCAACAGA | 4.16 | 8.66 | 2.08 | 1.00 | Hs.149609 | integrin, alpha 5 | |
| | | | | | | | (fibronectin receptor, | |
| | | | | | | | alpha polypeptide) | |
| 977 | CGGGAGCGCTA | 4.16 | 8.66 | 2.08 | 1.00 | Hs.148590 | ESTs, Weakly similar | |
| | | | | | | | to AF208846_1 BM-004 | |
| | | | | | | | [H,sapiens] | |
| 978 | TGTGATCAGAC | 4.16 | 8.66 | 2.08 | 1.00 | Hs.107476 | ATP synthase, H+ | |
| | | | | | | | transporting, | |
| | | | | | | | mitochondrial F1F0, | |
| | | | | | | | subunit g | |
| 979 | CTTCTCCAAAA | 2.08 | 0.00 | 2.08 | 0.65 | Hs.99949 | prolactin-induced | |
| | | | | | | | protein | |
| 980 | CAGTTTTTTTC | 2.08 | 0.00 | 2.08 | 0.65 | Hs.99597 | ESTs | |
| 981 | TCAGAGAATAA | 2.08 | 0.00 | 2.08 | 0.65 | Hs.99486 | ESTs | |
| 982 | TGGGTCATTTG | 2.08 | 0.00 | 2.08 | 0.65 | Hs.98073 | ESTs | |
| 983 | AGGTTTCCTCC | 2.08 | 0.00 | 2.08 | 0.65 | Hs.9736 | proteasome | |
| | | | | | | | (prosome, | |
| | | | | | | | macropain) 26S | |
| | | | | | | | subunit, non-ATPase, | |
| | | | | | | | 3 | |
| 984 | AACGGGGCCCT | 2.08 | 0.00 | 2.08 | 0.65 | Hs.97203 | small inducible | |
| | | | | | | | cytokine subfamily A | |
| | | | | | | | (Cys-Cys), member | |
| | | | | | | | 22 | |
| 985 | CTTAGCCCCAG | 2.08 | 0.00 | 2.08 | 0.65 | Hs.96908 | ESTs | |
| 986 | CCTGGTCAAGA | 2.08 | 0.00 | 2.08 | 0.65 | Hs.95972 | silver (mouse | |
| | | | | | | | homolog) like | |
| 987 | TACCCCTTGAA | 2.08 | 0.00 | 2.08 | 0.65 | Hs.95834 | ESTs | |
| 988 | TTTTGTTTTGT | 2.08 | 0.00 | 2.08 | 0.65 | Hs.95583 | transmembrane 4 | |
| | | | | | | | superfamily member | |
| | | | | | | | (tetraspan NET-7) | |
| 989 | TTTGCCTGGAT | 2.08 | 0.00 | 2.08 | 0.65 | Hs.95260 | Autosomal Highly | |
| | | | | | | | Conserved Protein | |
| 990 | TTATTCCACAA | 2.08 | 0.00 | 2.08 | 0.65 | Hs.93765 | lipoma HMGIC fusion | |
| | | | | | | | partner | |
| 991 | GTCTCATTTGA | 2.08 | 0.00 | 2.08 | 0.65 | Hs.92381 | nudix (nucleoside | |
| | | | | | | | diphosphate linked | |
| | | | | | | | moiety X)-type motif 4 | |
| 992 | GTGGTCAAGTT | 2.08 | 0.00 | 2.08 | 0.65 | Hs.92127 | ESTs | |
| 993 | CACACCCCTGA | 2.08 | 0.00 | 2.08 | 0.65 | Hs.90061 | progesterone binding | |
| | | | | | | | protein | |
| 994 | AATGAATGAAA | 2.08 | 0.00 | 2.08 | 0.65 | Hs.8986 | complement | |
| | | | | | | | component 1, q | |
| | | | | | | | subcomponent, beta | |
| | | | | | | | polypeptide | |
| 995 | ATGCGAAAGGC | 2.08 | 0.00 | 2.08 | 0.65 | Hs.89466 | dodecenoyl-Coenzyme | |
| | | | | | | | A delta | |
| | | | | | | | isomerase (3,2 transenoyl-Coenzyme A | |
| | | | | | | | isomerase) | |
| 996 | AGCACGACCCG | 2.08 | 0.00 | 2.08 | 0.65 | Hs.89434 | drebrin 1 | |
| 997 | GCAATAAATGG | 2.08 | 0.00 | 2.08 | 0.65 | Hs.89434 | drebrin 1 | |
| 998 | GGGGCTTAGGA | 2.08 | 0.00 | 2.08 | 0.65 | Hs.89135 | KIAA1528 protein | |
| 999 | GTTAAATCCTG | 2.08 | 0.00 | 2.08 | 0.65 | Hs.8881 | ESTs, Weakly similar | |
| | | | | | | | to RMS1_HUMAN | |
| | | | | | | | REGULATOR OF | |
| | | | | | | | MITOTIC SPINDLE | |
| | | | | | | | ASSEMBLY 1 | |
| | | | | | | | [H,sapiens] | |
| 1000 | GACTTCTGTCC | 2.08 | 0.00 | 2.08 | 0.65 | Hs.87539 | aldehyde | |
| | | | | | | | dehydrogenase 8 | |
| 1001 | GCACAATGGGA | 2.08 | 0.00 | 2.08 | 0.65 | Hs.85838 | solute carrier family | |
| | | | | | | | 16 (monocarboxylic | |
| | | | | | | | acid transporters), | |
| | | | | | | | member 3 | |
| 1002 | GAGCGCAGCGA | 2.08 | 0.00 | 2.08 | 0.65 | Hs.83727 | cleavage and | |
| | | | | | | | polyadenylation | |
| | | | | | | | specific factor 1, | |
| | | | | | | | 160kD subunit | |
| 1003 | CAGGCTTTTTG | 2.08 | 0.00 | 2.08 | 0.65 | Hs.83484 | SRY (sex determining | |
| | | | | | | | region Y)-box 4 | |
| 1004 | ACACTTCTTTC | 2.08 | 0.00 | 2.08 | 0.65 | Hs.83381 | guanine nucleotide | |
| | | | | | | | binding protein 11 | |
| 1005 | TACAGTATTTT | 2.08 | 0.00 | 2.08 | 0.65 | Hs.82921 | solute carrier family | |
| | | | | | | | 35 (CMP-sialic acid | |
| | | | | | | | transporter), member | |
| | | | | | | | 1 | |
| 1006 | CAGCCCCTCTT | 2.08 | 0.00 | 2.08 | 0.65 | Hs.82503 | H,sapiens mRNA for | |
| | | | | | | | 3′UTR of unknown | |
| | | | | | | | protein | |
| 1007 | AAAAGGCACTT | 2.08 | 0.00 | 2.08 | 0.65 | Hs.82425 | actin related protein | |
| | | | | | | | 2/3 complex, subunit | |
| | | | | | | | 5 (16 kD) | |
| 1008 | ACCATAATGTG | 2.08 | 0.00 | 2.08 | 0.65 | Hs.821 | zinc finger protein | |
| | | | | | | | homologous to Zfp92 | |
| | | | | | | | in mouse | |
| 1009 | GGGCCCCCTGG | 2.08 | 0.00 | 2.08 | 0.65 | Hs.81994 | glycophorin C | |
| | | | | | | | (Gerbich blood group) | |
| 1010 | TCAGAAAAAAA | 2.08 | 0.00 | 2.08 | 0.65 | Hs.8118 | KIAA0650 protein | |
| 1011 | AAAACATTATG | 2.08 | 0.00 | 2.08 | 0.65 | Hs.80917 | adaptor-related | |
| | | | | | | | protein complex 3, | |
| | | | | | | | sigma 1 subunit | |
| 1012 | GCTCCGTAAGG | 2.08 | 0.00 | 2.08 | 0.65 | Hs.80712 | KIAA0202 protein | |
| 1013 | AGTAAACCATC | 2.08 | 0.00 | 2.08 | 0.65 | Hs.80285 | Homo sapiens | |
| | | | | | | | mRNA; cDNA | |
| | | | | | | | DKFZp586C1723 | |
| | | | | | | | (from clone | |
| | | | | | | | DKFZp586C1723) | |
| 1014 | GAAATTTGAAA | 2.08 | 0.00 | 2.08 | 0.65 | Hs.79457 | hypothetical protein | |
| | | | | | | | FLJ20519 | |
| 1015 | CACTCAATAAA | 2.08 | 0.00 | 2.08 | 0.65 | Hs.79361 | Kalikrein 6 (neurosin, | |
| | | | | | | | zyme) | |
| 1016 | ATACTTTAATC | 2.08 | 0.00 | 2.08 | 0.65 | Hs.79274 | annexin A5 | |
| 1017 | CAGGTTGAAGT | 2.08 | 0.00 | 2.08 | 0.65 | Hs.79219 | RalGDS-like gene; | |
| | | | | | | | KIAA0959 protein | |
| 1018 | CTGCTTCCTGA | 2.08 | 0.00 | 2.08 | 0.65 | Hs.78921 | A kinase (PRKA) | |
| | | | | | | | anchor protein 1 | |
| 1019 | CTTTGCACTCT | 2.08 | 0.00 | 2.08 | 0.65 | Hs.78869 | transcription | |
| | | | | | | | elongation factor A | |
| | | | | | | | (SII), 1 | |
| 1020 | TGGGCGCCTTT | 2.08 | 0.00 | 2.08 | 0.65 | Hs.78601 | uroporphyrinogen | |
| | | | | | | | decarboxylase | |
| 1021 | TTTTCCTTTTG | 2.08 | 0.00 | 2.08 | 0.65 | Hs.78546 | ATPase, Ca++ | |
| | | | | | | | transporting, plasma | |
| | | | | | | | membrane 1 | |
| 1022 | CTGGGGGGAAG | 2.08 | 0.00 | 2.08 | 0.65 | Hs.77864 | KIAA0638 protein | |
| 1023 | GATTGTGCAAG | 2.08 | 0.00 | 2.08 | 0.65 | Hs.76666 | C9orf10 protein | |
| 1024 | GCCCTGTAGTT | 2.08 | 0.00 | 2.08 | 0.65 | Hs.76578 | protein inhibitor of | |
| | | | | | | | activated STAT3 | |
| 1025 | TGACAATTTTG | 2.08 | 0.00 | 2.08 | 0.65 | Hs.75912 | KIAA0257 protein | |
| 1026 | GATGTATTCTA | 2.08 | 0.00 | 2.08 | 0.65 | Hs.75844 | ESTs, Highly similar | |
| | | | | | | | to AF151903_1 CGI-145 | |
| | | | | | | | protein | |
| | | | | | | | [H,sapiens] | |
| 1027 | TATTTTTCTAG | 2.08 | 0.00 | 2.08 | 0.65 | Hs.7579 | hypothetical protein | |
| | | | | | | | FLJ10402 | |
| 1028 | CTGGATCTGGG | 2.08 | 0.00 | 2.08 | 0.65 | Hs.75658 | phosphorylase, | |
| | | | | | | | glycogen; brain | |
| 1029 | TATTTTGTGAG | 2.08 | 0.00 | 2.08 | 0.65 | Hs.75607 | myristoylated alanine-rich | |
| | | | | | | | rich protein kinase C | |
| | | | | | | | substrate (MARCKS, | |
| | | | | | | | 80K-L) | |
| 1030 | TTGTTTAATTT | 2.08 | 0.00 | 2.08 | 0.65 | Hs.75546 | capping protein (actin | |
| | | | | | | | filament) muscle Z-line, | |
| | | | | | | | alpha 2 | |
| 1031 | CGTTCCTGCGG | 2.08 | 0.00 | 2.08 | 0.65 | Hs.75424 | inhibitor of DNA | |
| | | | | | | | binding 1, dominant | |
| | | | | | | | negtive helix-loop-helix | |
| | | | | | | | protein | |
| 1032 | CACTCAGTGTG | 2.08 | 0.00 | 2.08 | 0.65 | Hs.75379 | solute carrier family 1 | |
| | | | | | | | (glial high affinity | |
| | | | | | | | glutamate | |
| | | | | | | | transporter), member | |
| | | | | | | | 3 | |
| 1033 | GCTAGGTCTGG | 2.08 | 0.00 | 2.08 | 0.65 | Hs.75354 | GCN1 (general | |
| | | | | | | | control of amino-acid | |
| | | | | | | | synthesis 1, yeast)-like | |
| | | | | | | | 1 | |
| 1034 | GGAAGAGCACT | 2.08 | 0.00 | 2.08 | 0.65 | Hs.75268 | sialyltransferase 4C | |
| | | | | | | | (beta-galactosidase | |
| | | | | | | | alpha-2,3-sialytransferase) | |
| 1035 | ATGCAGCCATA | 2.08 | 0.00 | 2.08 | 0.65 | Hs.75212 | ornithine | |
| | | | | | | | decarboxylase 1 | |
| 1036 | CATCTGTGAGC | 2.08 | 0.00 | 2.08 | 0.65 | Hs.75189 | death-associated | |
| | | | | | | | protein | |
| 1037 | CACTTTTGGGC | 2.08 | 0.00 | 2.08 | 0.65 | Hs.75080 | LIM and SH3 protein | |
| | | | | | | | 1 | |
| 1038 | ATTTTGTGTCA | 2.08 | 0.00 | 2.08 | 0.65 | Hs.75056 | adaptor-related | |
| | | | | | | | protein complex 3, | |
| | | | | | | | delta 1 subunit | |
| 1039 | GCCGGGTGGGC | 2.08 | 0.00 | 2.08 | 0.65 | Hs.74631 | basigin | |
| 1040 | CATCCTGCTGC | 2.08 | 0.00 | 2.08 | 0.65 | Hs.74619 | proteasome | |
| | | | | | | | (prosome, | |
| | | | | | | | macropain) 26S | |
| | | | | | | | subunit, non-ATPase, | |
| | | | | | | | 2 | |
| 1041 | GCTGTATAATT | 2.08 | 0.00 | 2.08 | 0.65 | Hs.74170 | metallothionein 1E | |
| | | | | | | | (functional) | |
| 1042 | GGAACCAGGTC | 2.08 | 0.00 | 2.08 | 0.65 | Hs.7404 | ESTs | |
| 1043 | AAAAAGAAACT | 2.08 | 0.00 | 2.08 | 0.65 | Hs.73287 | KIAA1235 protein | |
| 1044 | TCTGTATCCCC | 2.08 | 0.00 | 2.08 | 0.65 | Hs.724 | thyroid hormone | |
| | | | | | | | receptor, alpha (avian | |
| | | | | | | | erythroblastic | |
| | | | | | | | leukemia viral (v-erb-a) | |
| | | | | | | | oncogene | |
| | | | | | | | homolog) | |
| 1045 | CACTGGACGAG | 2.08 | 0.00 | 2.08 | 0.65 | Hs.71574 | ESTs | |
| 1046 | CAAATAAAATG | 2.08 | 0.00 | 2.08 | 0.65 | Hs.71465 | squalene epoxidase | |
| 1047 | TGTCAAAAAAA | 2.08 | 0.00 | 2.08 | 0.65 | Hs.7120 | cytokine receptor-like | |
| | | | | | | | molecule 9 | |
| 1048 | ATGTCGTGGTC | 2.08 | 0.00 | 2.08 | 0.65 | Hs.6900 | ring finger protein 13 | |
| 1049 | TCTTTACTTGA | 2.08 | 0.00 | 2.08 | 0.65 | Hs.6895 | actin related protein | |
| | | | | | | | 2/3 complex, subunit | |
| | | | | | | | 3 (21 kD) | |
| 1050 | TGCCTGGAACT | 2.08 | 0.00 | 2.08 | 0.65 | Hs.6820 | ESTs, Weakly similar | |
| | | | | | | | to putative | |
| | | | | | | | [C,elegans] | |
| 1051 | GGGCTCTGAGC | 2.08 | 0.00 | 2.08 | 0.65 | Hs.6770 | LCAT-like | |
| | | | | | | | lysophospholipase | |
| 1052 | TTGGACTGAGC | 2.08 | 0.00 | 2.08 | 0.65 | Hs.6518 | ganglioside | |
| | | | | | | | expression factor 2 | |
| 1053 | ATGCAGTTCAA | 2.08 | 0.00 | 2.08 | 0.65 | Hs.65135 | Homo sapiens | |
| | | | | | | | mRNA; cDNA | |
| | | | | | | | DKFZp434E0121 | |
| | | | | | | | (from clone | |
| | | | | | | | DKFZp434E0121) | |
| 1054 | ATTGAGCCACA | 2.08 | 0.00 | 2.08 | 0.65 | Hs.63290 | 2-hydroxyphytanoyl-CoA | |
| | | | | | | | lyase | |
| 1055 | TGTTTCAGGAT | 2.08 | 0.00 | 2.08 | 0.65 | Hs.6216 | tumorous imaginal | |
| | | | | | | | discs (Drosophila) | |
| | | | | | | | homolog | |
| 1056 | CCTGCCTCGTA | 2.08 | 0.00 | 2.08 | 0.65 | Hs.61490 | schwannomin | |
| | | | | | | | interacting protein 1 | |
| 1057 | AATAGGGGAAA | 2.08 | 0.00 | 2.08 | 0.65 | Hs.6147 | KIAA1075 protein | |
| 1058 | GCCGAGACCAA | 2.08 | 0.00 | 2.08 | 0.65 | Hs.61258 | argininosuccinate | |
| | | | | | | | lyase | |
| 1059 | TTCAGGAGGGG | 2.08 | 0.00 | 2.08 | 0.65 | Hs.5890 | ESTs, Weakly similar | |
| | | | | | | | to A49134 Ig kappa | |
| | | | | | | | chain V-I region | |
| | | | | | | | [H,sapiens] | |
| 1060 | TTGTTATATTG | 2.08 | 0.00 | 2.08 | 0.65 | Hs.5862 | hypothetical protein | |
| 1061 | TGAGTTTTACA | 2.08 | 0.00 | 2.08 | 0.65 | Hs.58373 | ESTs | |
| 1062 | TGCTTATTGAA | 2.08 | 0.00 | 2.08 | 0.65 | Hs.5822 | lectin, mannose-binding, | |
| | | | | | | | 1 | |
| 1063 | AGTCAAGCCCC | 2.08 | 0.00 | 2.08 | 0.65 | Hs.57687 | four and a half LIM | |
| | | | | | | | domains 3 | |
| 1064 | GTATTCCTAAA | 2.08 | 0.00 | 2.08 | 0.65 | Hs.5724 | ESTs, Weakly similar | |
| | | | | | | | to multi PDZ domain | |
| | | | | | | | protein MUPP1 | |
| | | | | | | | [H,sapiens] | |
| 1065 | AGCCGGGCTTT | 2.08 | 0.00 | 2.08 | 0.65 | Hs.57079 | ESTs | |
| 1066 | CCTCTCTGGTC | 2.08 | 0.00 | 2.08 | 0.65 | Hs.56874 | heat shock 27kD | |
| | | | | | | | protein family, | |
| | | | | | | | member 7 | |
| | | | | | | | (cardiovascular) | |
| 1067 | AGTGTGTTGCA | 2.08 | 0.00 | 2.08 | 0.65 | Hs.56105 | ESTs, Weakly similar | |
| | | | | | | | to WDNM_RAT | |
| | | | | | | | WDNM1 PROTEIN | |
| | | | | | | | PRECURSOR_[R,norvegicus] | |
| 1068 | TCAGGCATTTT | 2.08 | 0.00 | 2.08 | 0.65 | Hs.5566 | gap junction protein, | |
| | | | | | | | beta 2, 26kD | |
| | | | | | | | (connexin 26) | |
| 1069 | TGTGTGTGACA | 2.08 | 0.00 | 2.08 | 0.65 | Hs.55148 | ESTs | |
| 1070 | TGCAGACCCAT | 2.08 | 0.00 | 2.08 | 0.65 | Hs.5437 | Taxi (human T-cell | |
| | | | | | | | leukemia virus type I) | |
| | | | | | | | binding protein 1 | |
| 1071 | TAATTTTTACT | 2.08 | 0.00 | 2.08 | 0.65 | Hs.52256 | hypothetical protein | |
| | | | | | | | FLJ20624 | |
| 1072 | ATTGTTTCTTG | 2.08 | 0.00 | 2.08 | 0.65 | Hs.52081 | KIAA0867 protein | |
| 1073 | CTTTTGTTTGG | 2.08 | 0.00 | 2.08 | 0.65 | Hs.5094 | ring finger protein 10 | |
| 1074 | GTCTTAACTCA | 2.08 | 0.00 | 2.08 | 0.65 | Hs.5074 | similar to S. pombe | |
| | | | | | | | dim1+ | |
| 1075 | TAATAAAGCAT | 2.08 | 0.00 | 2.08 | 0.65 | Hs.4888 | seryl-tRNA | |
| | | | | | | | synthetase | |
| 1076 | CCACGCACTGT | 2.08 | 0.00 | 2.08 | 0.65 | Hs.48778 | Homo sapiens | |
| | | | | | | | mRNA; cDNA | |
| | | | | | | | DKFZp586O0221 | |
| | | | | | | | (from clone | |
| | | | | | | | DKFZp586O0221) | |
| 1077 | ACTCACGATTG | 2.08 | 0.00 | 2.08 | 0.65 | Hs.4814 | mannosidase, alpha, | |
| | | | | | | | class 1B, member 1 | |
| 1078 | TAACCAATCAG | 2.08 | 0.00 | 2.08 | 0.65 | Hs.479 | RAB5C, member | |
| | | | | | | | RAS oncogene family | |
| 1079 | GGCCTCTGATG | 2.08 | 0.00 | 2.08 | 0.65 | Hs.46670 | PRO1575 protein | |
| 1080 | CCCAATTTTCA | 2.08 | 0.00 | 2.08 | 0.65 | Hs.46405 | polymerase (RNA) II | |
| | | | | | | | (DNA directed) | |
| | | | | | | | polypeptide F | |
| 1081 | TTTGTTGAATG | 2.08 | 0.00 | 2.08 | 0.65 | Hs.44856 | ESTs | |
| 1082 | TCTTTGCTCTT | 2.08 | 0.00 | 2.08 | 0.65 | Hs.44077 | hypothetical protein | |
| | | | | | | | FLJ10793 | |
| 1083 | AGGACTTCTGA | 2.08 | 0.00 | 2.08 | 0.65 | Hs.43847 | ESTs, Weakly similar | |
| | | | | | | | to SFR7_HUMAN | |
| | | | | | | | SPLICING FACTOR, | |
| | | | | | | | ARGININE/SERINE-RICH | |
| | | | | | | | 7 [H,sapiens] | |
| 1084 | ATAATAAAGCT | 2.08 | 0.00 | 2.08 | 0.65 | Hs.37682 | retinoic acid receptor | |
| | | | | | | | responder | |
| | | | | | | | (tazarotene induced) | |
| | | | | | | | 2 | |
| 1085 | CTTTTCATCAT | 2.08 | 0.00 | 2.08 | 0.65 | Hs.3726 | x 003 protein | |
| 1086 | TGGTCCCTCTC | 2.08 | 0.00 | 2.08 | 0.65 | Hs.36587 | protein phosphatase | |
| | | | | | | | 1, regulatory subunit | |
| | | | | | | | 7 | |
| 1087 | TCTAGTCACTG | 2.08 | 0.00 | 2.08 | 0.65 | Hs.36565 | ESTs | |
| 1088 | GACTGCTCTGG | 2.08 | 0.00 | 2.08 | 0.65 | Hs.36475 | ESTs | |
| 1089 | GGGAAAGAGGG | 2.08 | 0.00 | 2.08 | 0.65 | Hs.35096 | KIAA1538 protein | |
| 1090 | GAGCTCCACAG | 2.08 | 0.00 | 2.08 | 0.65 | Hs.3407 | protein kinase | |
| | | | | | | | (cAMP-dependent, | |
| | | | | | | | catalytic) inhibitor | |
| | | | | | | | gamma | |
| 1091 | TATGAAAACAT | 2.08 | 0.00 | 2.08 | 0.65 | Hs.3337 | transmembrane 4 | |
| | | | | | | | superfamily member | |
| | | | | | | | 1 | |
| 1092 | GCCACGTTGTC | 2.08 | 0.00 | 2.08 | 0.65 | Hs.32352 | hypothetical protein | |
| | | | | | | | DKFZp434K1210 | |
| 1093 | TCCTCTACCTG | 2.08 | 0.00 | 2.08 | 0.65 | Hs.32018 | SNARE associated | |
| | | | | | | | protein snapin | |
| 1094 | GGAGCAGACGC | 2.08 | 0.00 | 2.08 | 0.65 | Hs.31718 | Homo sapiens cDNA | |
| | | | | | | | FLJ11034 fis, clone | |
| | | | | | | | PLACE1004258 | |
| 1095 | AATATTTTTAT | 2.08 | 0.00 | 2.08 | 0.65 | Hs.31386 | ESTs, Highly similar | |
| | | | | | | | to JE0174 frizzled | |
| | | | | | | | protein-2-human | |
| | | | | | | | [H,sapiens] | |
| 1096 | GAGAGCCTGCC | 2.08 | 0.00 | 2.08 | 0.65 | Hs.31305 | transducin-like | |
| | | | | | | | enhancer of split 3, | |
| | | | | | | | homolog of | |
| | | | | | | | Drosophila E(sp1) | |
| 1097 | GGCATTGTTCA | 2.08 | 0.00 | 2.08 | 0.65 | Hs.3128 | polymerase (RNA) II | |
| | | | | | | | (DNA directed) | |
| | | | | | | | polypeptide H | |
| 1098 | GAAATGGCAGT | 2.08 | 0.00 | 2.08 | 0.65 | Hs.30853 | ESTs | |
| 1099 | TTACCAAAGCA | 2.08 | 0.00 | 2.08 | 0.65 | Hs.30246 | solute carrier family | |
| | | | | | | | 19 (thiamine | |
| | | | | | | | transporter), member | |
| | | | | | | | 2 | |
| 1100 | GTATTGGCCTT | 2.08 | 0.00 | 2.08 | 0.65 | Hs.28757 | transmembrane 9 | |
| | | | | | | | superfamily member | |
| | | | | | | | 2 | |
| 1101 | GTGTAAATGGA | 2.08 | 0.00 | 2.08 | 0.65 | Hs.286131 | CGI-101 protein | |
| 1102 | TTCACATTGTC | 2.08 | 0.00 | 2.08 | 0.65 | Hs.285804 | ESTs | |
| 1103 | CAGCTCATCTA | 2.08 | 0.00 | 2.08 | 0.65 | Hs.285634 | Homo sapiens | |
| | | | | | | | HSPC222 mRNA, | |
| | | | | | | | complete cds | |
| 1104 | AAACCCCAATA | 2.08 | 0.00 | 2.08 | 0.65 | Hs.285501 | Human rearranged | |
| | | | | | | | immunoglobulin | |
| | | | | | | | lambda light chain | |
| | | | | | | | mRNA | |
| 1105 | TCATTTGGTGT | 2.08 | 0.00 | 2.08 | 0.65 | Hs.285439 | ESTs | |
| 1106 | ACTGTGGACTG | 2.08 | 0.00 | 2.08 | 0.65 | Hs.285122 | ESTs, Weakly similar | |
| | | | | | | | to S53869 laminin | |
| | | | | | | | beta-2 chain | |
| | | | | | | | precursor [H,sapiens] | |
| 1107 | ACTGAGGTGCC | 2.08 | 0.00 | 2.08 | 0.65 | Hs.284159 | FIBP-1 protein | |
| 1108 | AGAGAAGAATG | 2.08 | 0.00 | 2.08 | 0.65 | Hs.2841 | neuromedin U | |
| 1109 | CTGGAGGCACA | 2.08 | 0.00 | 2.08 | 0.65 | Hs.283976 | Homo sapiens clone | |
| | | | | | | | TCBA00888 mRNA | |
| | | | | | | | sequence | |
| 1110 | GAGCCAACAAT | 2.08 | 0.00 | 2.08 | 0.65 | Hs.283680 | hypothetical protein | |
| 1111 | AGGATTGTTTG | 2.08 | 0.00 | 2.08 | 0.65 | Hs.283545 | ESTs | |
| 1112 | ATGTATGGGGA | 2.08 | 0.00 | 2.08 | 0.65 | Hs.283429 | SMC (mouse) | |
| | | | | | | | homolog, X | |
| | | | | | | | chromosome | |
| 1113 | TTGTAATAAAA | 2.08 | 0.00 | 2.08 | 0.65 | Hs.283429 | SMC (mouse) | |
| | | | | | | | homolog, X | |
| | | | | | | | chromosome | |
| 1114 | ATGAAACCCTA | 2.08 | 0.00 | 2.08 | 0.65 | Hs.282671 | EST | |
| 1115 | ACGTGGTGATG | 2.08 | 0.00 | 2.08 | 0.65 | Hs.279945 | HSPC023 protein | |
| 1116 | GAAATCCGCAC | 2.08 | 0.00 | 2.08 | 0.65 | Hs.279854 | mannosidase, alpha, | |
| | | | | | | | class 2B, member 1 | |
| 1117 | CTTTACTGTGT | 2.08 | 0.00 | 2.08 | 0.65 | Hs.279853 | HSPC018 protein | |
| 1118 | TGTGTTGTGTC | 2.08 | 0.00 | 2.08 | 0.65 | Hs.279806 | Homo sapiens | |
| | | | | | | | mRNA; cDNA | |
| | | | | | | | DKFZp434E109 | |
| | | | | | | | (from clone | |
| | | | | | | | DKFZp434E109) | |
| 1119 | TGAGATTTCTT | 2.08 | 0.00 | 2.08 | 0.65 | Hs.279061 | CGI-150 protein | |
| 1120 | GTAAAGATTTG | 2.08 | 0.00 | 2.08 | 0.65 | Hs.278629 | ESTs | |
| 1121 | GTCGGACACTG | 2.08 | 0.00 | 2.08 | 0.65 | Hs.278559 | talin | |
| 1122 | GCTGGGCGCGG | 2.08 | 0.00 | 2.08 | 0.65 | Hs.278070 | EST | |
| 1123 | CTTGTAATCTC | 2.08 | 0.00 | 2.08 | 0.65 | Hs.278002 | EST | |
| 1124 | GTGGGTGTCCT | 2.08 | 0.00 | 2.08 | 0.65 | Hs.27633 | DKFZP586B0519 | |
| | | | | | | | protein | |
| 1125 | GGCAATGCAGT | 2.08 | 0.00 | 2.08 | 0.65 | Hs.275505 | ESTs | |
| 1126 | GTATAAAAAAA | 2.08 | 0.00 | 2.08 | 0.65 | Hs.27337 | hypothetical protein | |
| | | | | | | | FLJ20623 | |
| 1127 | ACACCTCTAAA | 2.08 | 0.00 | 2.08 | 0.65 | Hs.273230 | hypothetical protein | |
| | | | | | | | FLJ10830 | |
| 1128 | CACCTGTAGTT | 2.08 | 0.00 | 2.08 | 0.65 | Hs.271053 | ESTs, Weakly similar | |
| | | | | | | | to A46010 X-linked | |
| | | | | | | | retinopathy protein | |
| | | | | | | | [H,sapiens] | |
| 1129 | TCACTCCAGCC | 2.08 | 0.00 | 2.08 | 0.65 | Hs.270497 | ESTs | |
| 1130 | TGTACATATGT | 2.08 | 0.00 | 2.08 | 0.65 | Hs.268384 | homolog of yeast | |
| | | | | | | | CDH1/HCT1 | |
| 1131 | GCTCTGTAAGC | 2.08 | 0.00 | 2.08 | 0.65 | Hs.268149 | putative | |
| | | | | | | | methyltransferase | |
| 1132 | AATGTCCAGTA | 2.08 | 0.00 | 2.08 | 0.65 | Hs.26373 | ESTs | |
| 1133 | TTGTTAAGCCT | 2.08 | 0.00 | 2.08 | 0.65 | Hs.26243 | Homo sapiens cDNA | |
| | | | | | | | FLJ11177 fis, clone | |
| | | | | | | | PLACE1007402 | |
| 1134 | TCTGGCTAATT | 2.08 | 0.00 | 2.08 | 0.65 | Hs.262198 | ESTs | |
| 1135 | TGTTAATGTTA | 2.08 | 0.00 | 2.08 | 0.65 | Hs.261828 | Homo sapiens | |
| | | | | | | | mRNA; cDNA | |
| | | | | | | | DKFZp434N0211 | |
| | | | | | | | (from clone | |
| | | | | | | | DKFZp434N0211) | |
| 1136 | TCTGTAACACC | 2.08 | 0.00 | 2.08 | 0.65 | Hs.260622 | butyrate-induced | |
| | | | | | | | transcript 1 | |
| 1137 | GGGGTTTGTTT | 2.08 | 0.00 | 2.08 | 0.65 | Hs.258455 | EST | |
| 1138 | ACATAGTCTGA | 2.08 | 0.00 | 2.08 | 0.65 | Hs.25766 | ESTs | |
| 1139 | CGGATAAGGCC | 2.08 | 0.00 | 2.08 | 0.65 | Hs.256526 | nuclear prelamin A | |
| | | | | | | | recognition factor | |
| 1140 | ATCTGAAGCAA | 2.08 | 0.00 | 2.08 | 0.65 | Hs.256311 | granin-like | |
| | | | | | | | neuroendocrine | |
| | | | | | | | peptide precursor | |
| 1141 | AAAGGCATCAG | 2.08 | 0.00 | 2.08 | 0.65 | Hs.256297 | integrin, alpha 11 | |
| 1142 | CCCGCCAGTGC | 2.08 | 0.00 | 2.08 | 0.65 | Hs.256297 | integrin, alpha 11 | |
| 1143 | TATGCTGAAAT | 2.08 | 0.00 | 2.08 | 0.65 | Hs.255277 | ESTs | |
| 1144 | AGGAGCGGGGT | 2.08 | 0.00 | 2.08 | 0.65 | Hs.252189 | syndecan 4 | |
| | | | | | | | (amphiglycan, | |
| | | | | | | | ryudocan) | |
| 1145 | TTCACTTCAAC | 2.08 | 0.00 | 2.08 | 0.65 | Hs.250911 | Homo sapiens clone | |
| | | | | | | | 23967 unknown | |
| | | | | | | | mRNA, partial cds | |
| 1146 | TTGTATCAGAA | 2.08 | 0.00 | 2.08 | 0.65 | Hs.250723 | FK506 binding | |
| | | | | | | | protein 12-rapamycin | |
| | | | | | | | associated protein 1 | |
| 1147 | AGGTATATATC | 2.08 | 0.00 | 2.08 | 0.65 | Hs.24715 | Homo sapiens | |
| | | | | | | | mRNA; cDNA | |
| | | | | | | | DKFZp434D0215 | |
| | | | | | | | (from clone | |
| | | | | | | | DKFZp434D0215); | |
| | | | | | | | partial cds | |
| 1148 | ATGGAAAGGAA | 2.08 | 0.00 | 2.08 | 0.65 | Hs.243901 | Homo sapiens cDNA | |
| | | | | | | | FLJ20738 fis, clone | |
| | | | | | | | HEP08257 | |
| 1149 | CCTCCAGCTAC | 2.08 | 0.00 | 2.08 | 0.65 | Hs.242463 | keratin 8 | |
| 1150 | TATCTAGCTGC | 2.08 | 0.00 | 2.08 | 0.65 | Hs.241545 | hypothetical protein | |
| 1151 | GCTGTAATCCT | 2.08 | 0.00 | 2.08 | 0.65 | Hs.241382 | tumor necrosis factor | |
| | | | | | | | (ligand) superfamily, | |
| | | | | | | | member 15 | |
| 1152 | TGAAGAGACTT | 2.08 | 0.00 | 2.08 | 0.65 | Hs.240767 | Human DNA | |
| | | | | | | | sequence from clone | |
| | | | | | | | RP1-12G14 on | |
| | | | | | | | chromosome 6q24, 1-25,2. | |
| 1153 | GGACCACCCAA | 2.08 | 0.00 | 2.08 | 0.65 | Hs.239298 | microtubule | |
| | | | | | | | associated protein 4 | |
| 1154 | ATCCTACTGTT | 2.08 | 0.00 | 2.08 | 0.65 | Hs.239218 | uncharacterized | |
| | | | | | | | hypothalamus protein | |
| | | | | | | | HCDASE | |
| 1155 | CTTAGGAGTCA | 2.08 | 0.00 | 2.08 | 0.65 | Hs.23853 | ESTs | |
| 1156 | GGTGACAGAAC | 2.08 | 0.00 | 2.08 | 0.65 | Hs.234890 | EST | |
| 1157 | TGCCACCACGC | 2.08 | 0.00 | 2.08 | 0.65 | Hs.233480 | EST | |
| 1158 | GGCTTGTCTAT | 2.08 | 0.00 | 2.08 | 0.65 | Hs.23294 | ESTs, Weakly similar | |
| | | | | | | | to weak similarity to | |
| | | | | | | | HSP90 [C,elegans] | |
| 1159 | AAACTGGGAGG | 2.08 | 0.00 | 2.08 | 0.65 | Hs.231722 | ESTs | |
| 1160 | AACCCGGGGAG | 2.08 | 0.00 | 2.08 | 0.65 | Hs.228009 | EST | |
| 1161 | TTCTCCTCTTT | 2.08 | 0.00 | 2.08 | 0.65 | Hs.22451 | hypothetical protein | |
| | | | | | | | FLJ10357 | |
| 1162 | CCAATGTTGTT | 2.08 | 0.00 | 2.08 | 0.65 | Hs.22209 | EST | |
| 1163 | CAGCTCTTAGG | 2.08 | 0.00 | 2.08 | 0.65 | Hs.22208 | Homo sapiens mRNA | |
| | | | | | | | full length insert | |
| | | | | | | | cDNA clone | |
| | | | | | | | EUROIMAGE 30872 | |
| 1164 | GAAGTGCTGCT | 2.08 | 0.00 | 2.08 | 0.65 | Hs.21812 | ESTs | |
| 1165 | GAATGTTTTTT | 2.08 | 0.00 | 2.08 | 0.65 | Hs.21432 | SEX gen | |
| 1166 | TACATCCGAAT | 2.08 | 0.00 | 2.08 | 0.65 | Hs.21321 | Homo sapiens | |
| | | | | | | | mRNA; cDNA | |
| | | | | | | | DKFZp564E1363 | |
| | | | | | | | (from clone | |
| | | | | | | | DKFZp564E1363) | |
| 1167 | GCGAACCCCCC | 2.08 | 0.00 | 2.08 | 0.65 | Hs.211862 | EST | |
| 1168 | CCCTCACTCCT | 2.08 | 0.00 | 2.08 | 0.65 | Hs.21143 | (Manual assignment) | |
| | | | | | | | MEMOREC PSL4 | |
| | | | | | | | presenilin-like protein, | |
| | | | | | | | primary tag | |
| 1169 | TGCAGGTGTGT | 2.08 | 0.00 | 2.08 | 0.65 | Hs.20993 | high-glucose-regulated | |
| | | | | | | | protein 8 | |
| 1170 | GCTAACTTAAA | 2.08 | 0.00 | 2.08 | 0.65 | Hs.20787 | ESTs | |
| 1171 | AAGTTTATAGA | 2.08 | 0.00 | 2.08 | 0.65 | Hs.206097 | oncogene TC21 | |
| 1172 | AGACGCTTCTG | 2.08 | 0.00 | 2.08 | 0.65 | Hs.203772 | FSHD region gene 1 | |
| 1173 | CCACTGCACGC | 2.08 | 0.00 | 2.08 | 0.65 | Hs.202669 | thiopurine S-methyltransferase | |
| 1174 | TAACCAAATAC | 2.08 | 0.00 | 2.08 | 0.65 | Hs.201623 | ESTs | |
| 1175 | TATTCCCCACC | 2.08 | 0.00 | 2.08 | 0.65 | Hs.199316 | ESTs | |
| 1176 | TGACTGTATTA | 2.08 | 0.00 | 2.08 | 0.65 | Hs.198241 | amine oxidase, | |
| | | | | | | | copper containing 3 | |
| | | | | | | | (vascular adhesion | |
| | | | | | | | protein 1) | |
| 1177 | TTTTTCTTAAA | 2.08 | 0.00 | 2.08 | 0.65 | Hs.197955 | KIAA0704 protein | |
| 1178 | CACTGCATATG | 2.08 | 0.00 | 2.08 | 0.65 | Hs.196177 | phosphorylase | |
| | | | | | | | kinase, gamma 2 | |
| | | | | | | | (testis) | |
| 1179 | CCCTGAATGAA | 2.08 | 0.00 | 2.08 | 0.65 | Hs.19545 | frizzled (Drosophila) | |
| | | | | | | | homolog 4 | |
| 1180 | TTGGCCAAGAT | 2.08 | 0.00 | 2.08 | 0.65 | Hs.19522 | hypothetical protein | |
| | | | | | | | PRO2849 | |
| 1181 | TACAAAAGTGG | 2.08 | 0.00 | 2.08 | 0.65 | Hs.194662 | calponin 3, acidic | |
| 1182 | TGCTCAGTGGT | 2.08 | 0.00 | 2.08 | 0.65 | Hs.194625 | dynein, cytoplasmic, | |
| | | | | | | | light intermediate | |
| | | | | | | | polypeptide 2 | |
| 1183 | CCCCCAATTCT | 2.08 | 0.00 | 2.08 | 0.65 | Hs.194534 | vesicle-associated | |
| | | | | | | | membrane protein 2 | |
| | | | | | | | (synaptobrevin 2) | |
| 1184 | TGAGCACATAA | 2.08 | 0.00 | 2.08 | 0.65 | Hs.194208 | suc1-associated | |
| | | | | | | | neurotrophic factor | |
| | | | | | | | target 2 (FGFR | |
| | | | | | | | signalling adaptor) | |
| 1185 | TTCCAGCTGCT | 2.08 | 0.00 | 2.08 | 0.65 | Hs.19121 | adaptor-related | |
| | | | | | | | protein complex 2, | |
| | | | | | | | alpha 2 subunit | |
| 1186 | CCACTCCACTC | 2.08 | 0.00 | 2.08 | 0.65 | Hs.190452 | KIAA0365 gene | |
| | | | | | | | product | |
| 1187 | CAATTGTAAAT | 2.08 | 0.00 | 2.08 | 0.65 | Hs.18792 | thioredoxin-like, 32kD | |
| 1188 | AAGAACTAAAA | 2.08 | 0.00 | 2.08 | 0.65 | Hs.18778 | hypothetical protein | |
| 1189 | GGGGTACCCCT | 2.08 | 0.00 | 2.08 | 0.65 | Hs.187520 | ESTs, Weakly similar | |
| | | | | | | | to dJ353E16,2 | |
| | | | | | | | [H,sapiens] | |
| 1190 | CCCTGAATCCC | 2.08 | 0.00 | 2.08 | 0.65 | Hs.184592 | Human clone | |
| | | | | | | | A9A2BRB5 | |
| | | | | | | | (CAC)n/(GTG)n | |
| | | | | | | | repeat-containing | |
| | | | | | | | mRNA | |
| 1191 | AAAACAGTGGC | 2.08 | 0.00 | 2.08 | 0.65 | Hs.184109 | ribosomal protein | |
| | | | | | | | L37a | |
| 1192 | CAGCATCTAAT | 2.08 | 0.00 | 2.08 | 0.65 | Hs.184062 | putative Rab5-interacting | |
| | | | | | | | protein | |
| 1193 | CCACTGTACTT | 2.08 | 0.00 | 2.08 | 0.65 | Hs.183475 | Homo sapiens clone | |
| | | | | | | | 25061 mRNA | |
| | | | | | | | sequence | |
| 1194 | CCTTGAAATCA | 2.08 | 0.00 | 2.08 | 0.65 | Hs.183161 | ESTs | |
| 1195 | GCAGTCATACA | 2.08 | 0.00 | 2.08 | 0.65 | Hs.182626 | chromosome 22 open | |
| | | | | | | | reading frame 5 | |
| 1196 | AAAGGTTGGTT | 2.08 | 0.00 | 2.08 | 0.65 | Hs.182423 | ES1 (zebrafish) | |
| | | | | | | | protein, human | |
| | | | | | | | homolog of | |
| 1197 | GCTCTGTTCAT | 2.08 | 0.00 | 2.08 | 0.65 | Hs.18192 | Ser/Arg-related | |
| | | | | | | | nuclear matrix protein | |
| | | | | | | | (plenty of prolines | |
| | | | | | | | 101-like | |
| 1198 | GCTCAGGTCTG | 2.08 | 0.00 | 2.08 | 0.65 | Hs.181406 | endothelin converting | |
| | | | | | | | enzyme 1 | |
| 1199 | GTGAAAAAAAA | 2.08 | 0.00 | 2.08 | 0.65 | Hs.181373 | accessory proteins | |
| | | | | | | | BAP31/BAP29 | |
| 1200 | TGATGTGATAG | 2.08 | 0.00 | 2.08 | 0.65 | Hs.181159 | Homo sapiens | |
| | | | | | | | mRNA; cDNA | |
| | | | | | | | DKFZp434F0217 | |
| | | | | | | | (from clone | |
| | | | | | | | DKFZp434F0217) | |
| 1201 | TTTCTGTATGT | 2.08 | 0.00 | 2.08 | 0.65 | Hs.180877 | H3 histone, family 3B | |
| | | | | | | | (H3,3B) | |
| 1202 | AAATCAGGAAC | 2.08 | 0.00 | 2.08 | 0.65 | Hs.180549 | ESTs, Highly similar | |
| | | | | | | | in R26660_1, partial | |
| | | | | | | | CDS [H,sapiens] | |
| 1203 | AGTTGAAATTC | 2.08 | 0.00 | 2.08 | 0.65 | Hs.180428 | KIAA1181 protein | |
| 1204 | AGTGCCTTGGG | 2.08 | 0.00 | 2.08 | 0.65 | Hs.178604 | ESTs | |
| 1205 | GCTTGGCTCCC | 2.08 | 0.00 | 2.08 | 0.65 | Hs.175260 | EST | |
| 1206 | CACATCCTTAC | 2.08 | 0.00 | 2.08 | 0.65 | Hs.173717 | phosphatidic acid | |
| | | | | | | | phosphatase type 2B | |
| 1207 | ATAGAGGCAAT | 2.08 | 0.00 | 2.08 | 0.65 | Hs.173714 | MORF-related gene | |
| | | | | | | | X | |
| 1208 | CCGTTCTGGAT | 2.08 | 0.00 | 2.08 | 0.65 | Hs.173638 | Homo sapiens partial | |
| | | | | | | | TCF-4 gene for T-cell | |
| | | | | | | | transcription factor-4, | |
| | | | | | | | exon 1 and joined | |
| | | | | | | | CDS features | |
| 1209 | TGCAGGGACCT | 2.08 | 0.00 | 2.08 | 0.65 | Hs.173043 | metastasis-associated | |
| | | | | | | | 1-like 1 | |
| 1210 | GAAGGCTTATC | 2.08 | 0.00 | 2.08 | 0.65 | Hs.172674 | nuclear factor of | |
| | | | | | | | activated T-cells, | |
| | | | | | | | cytoplasmic 3 | |
| 1211 | AATGAGCAACT | 2.08 | 0.00 | 2.08 | 0.65 | Hs.171862 | guanylate binding | |
| | | | | | | | protein 2, interferon-inducible | |
| 1212 | TTTTGCTACAG | 2.08 | 0.00 | 2.08 | 0.65 | Hs.171545 | HIV-1 Rev binding | |
| | | | | | | | protein | |
| 1213 | TAATTCTTCTC | 2.08 | 0.00 | 2.08 | 0.65 | Hs.1708 | chaperonin | |
| | | | | | | | containing TCP1, | |
| | | | | | | | subunit 3 (gamma) | |
| 1214 | TTCTAATTTTT | 2.08 | 0.00 | 2.08 | 0.65 | Hs.170414 | paired basic amino | |
| | | | | | | | acid cleaving system | |
| | | | | | | | 4 | |
| 1215 | ATGATAATTAA | 2.08 | 0.00 | 2.08 | 0.65 | Hs.170142 | ESTs | |
| 1216 | TTCTTGCTTAA | 2.08 | 0.00 | 2.08 | 0.65 | Hs.169895 | ubiquitin-conjugating | |
| | | | | | | | enzyme E2L 6 | |
| 1217 | CGAGGGGGGCG | 2.08 | 0.00 | 2.08 | 0.65 | Hs.169875 | thrombospondin 3 | |
| 1218 | CTCCTGTGGTC | 2.08 | 0.00 | 2.08 | 0.65 | Hs.169851 | ESTs | |
| 1219 | GATCTGTTTCT | 2.08 | 0.00 | 2.08 | 0.65 | Hs.169743 | Homo sapiens clone | |
| | | | | | | | 25121 neuronal | |
| | | | | | | | offactomedin related | |
| | | | | | | | ER localized protein | |
| | | | | | | | mRNA sequence, | |
| | | | | | | | complete cds | |
| 1220 | AAGATTGGGGT | 2.08 | 0.00 | 2.08 | 0.65 | Hs.169610 | CD44 antigen | |
| | | | | | | | (homing function and | |
| | | | | | | | indian blood group | |
| | | | | | | | system) | |
| 1221 | TAACCAAAAAC | 2.08 | 0.00 | 2.08 | 0.65 | Hs.169241 | ELK4, ETS-domain | |
| | | | | | | | protein (SRF | |
| | | | | | | | accessory protein 1) | |
| 1222 | GATTCAACCAA | 2.08 | 0.00 | 2.08 | 0.65 | Hs.168213 | ESTs | |
| 1223 | TTCTGTGCATA | 2.08 | 0.00 | 2.08 | 0.65 | Hs.16803 | hypothetical protein | |
| | | | | | | | FLJ10231 | |
| 1224 | CATAACCTTCC | 2.08 | 0.00 | 2.08 | 0.65 | Hs.167460 | splicing factor, | |
| | | | | | | | arginine/serine-rich 3 | |
| 1225 | ATAAATAAATT | 2.08 | 0.00 | 2.08 | 0.65 | Hs.16677 | hypothetical protein | |
| | | | | | | | FLJ10506 | |
| 1226 | GCTAGGTA1TT | 2.08 | 0.00 | 2.08 | 0.65 | Hs.165986 | testin | |
| 1227 | CTTTGA11TAT | 2.08 | 0.00 | 2.08 | 0.65 | Hs.165590 | ribosomal protein S13 | |
| 1228 | ACAGCCCTGAT | 2.08 | 0.00 | 2.08 | 0.65 | Hs.163593 | ribosomal protein | |
| | | | | | | | L18a | |
| 1229 | GCTCACTGCAA | 2.08 | 0.00 | 2.08 | 0.65 | Hs.163385 | EST | |
| 1230 | TTGAATATTAA | 2.08 | 0.00 | 2.08 | 0.65 | Hs.161554 | hypothetical protein | |
| | | | | | | | FLJ20159 | |
| 1231 | GGGATGGCAGC | 2.08 | 0.00 | 2.08 | 0.65 | Hs.159637 | valyl-tRNA | |
| | | | | | | | synthetase 2 | |
| 1232 | AGCTGGGATGG | 2.08 | 0.00 | 2.08 | 0.65 | Hs.15898 | peroxisomal 2,4-dienoyl-CoA | |
| | | | | | | | reductase | |
| 1233 | ATCGCATCACT | 2.08 | 0.00 | 2.08 | 0.65 | Hs.158126 | ESTs | |
| 1234 | ACGCACATTAT | 2.08 | 0.00 | 2.08 | 0.65 | Hs.156007 | Down syndrome | |
| | | | | | | | critical region gene 1-like 1 | |
| 1235 | CCTCACTTTCT | 2.08 | 0.00 | 2.08 | 0.65 | Hs.155560 | calnexin | |
| 1236 | AAGAAGGCAAG | 2.08 | 0.00 | 2.08 | 0.65 | Hs.1554020 | site of albumin | |
| | | | | | | | promoter (albumin D-box) | |
| | | | | | | | binding protein | |
| 1237 | GAATCATTTAT | 2.08 | 0.00 | 2.08 | 0.65 | Hs.154668 | KIAA0391 gene | |
| | | | | | | | product | |
| 1238 | GACTCTGGAGA | 2.08 | 0.00 | 2.08 | 0.65 | Hs.154567 | supervillin | |
| 1239 | GGCCGCTGCTC | 2.08 | 0.00 | 2.08 | 0.65 | Hs.151531 | protein phosphatase | |
| | | | | | | | 3 (formerly 2B), | |
| | | | | | | | catalytic subunit, beta | |
| | | | | | | | isoform (calcineurin A | |
| | | | | | | | beta) | |
| 1240 | AACTCTGATAT | 2.08 | 0.00 | 2.08 | 0.65 | Hs.151046 | hypothetical protein | |
| | | | | | | | FLJ11193 | |
| 1241 | CTTCTCTTGAG | 2.08 | 0.00 | 2.08 | 0.65 | Hs.150557 | basic transcription | |
| | | | | | | | element binding | |
| | | | | | | | protein 1 | |
| 1242 | CCCCTCCCCAG | 2.08 | 0.00 | 2.08 | 0.65 | Hs.150540 | Homo sapiens | |
| | | | | | | | chromosome 22q13 | |
| | | | | | | | BAC clone | |
| | | | | | | | CIT987SK-384D8 | |
| | | | | | | | complete sequence | |
| 1243 | TGAGGACACAG | 2.08 | 0.00 | 2.08 | 0.65 | Hs.14541 | cullin 1 | |
| 1244 | ATGTCTTCGTT | 2.08 | 0.00 | 2.08 | 0.65 | Hs.144926 | ESTs | |
| 1245 | GTGCCTCGGAG | 2.08 | 0.00 | 2.08 | 0.65 | Hs.143046 | Homo sapiens cDNA | |
| | | | | | | | FLJ20418 fis, clone | |
| | | | | | | | KAT02427 | |
| 1246 | CCTGCAGTCCC | 2.08 | 0.00 | 2.08 | 0.65 | Hs.141746 | ESTs | |
| 1247 | TTGATAAATAA | 2.08 | 0.00 | 2.08 | 0.65 | Hs.139226 | replication factor C | |
| | | | | | | | (activator 1) 2 (40kD) | |
| 1248 | CGCCTGTGGTC | 2.08 | 0.00 | 2.08 | 0.65 | Hs.138263 | Homo sapiens clone | |
| | | | | | | | 24528 mRNA | |
| | | | | | | | sequence | |
| 1249 | ACCTCACCTGG | 2.08 | 0.00 | 2.08 | 0.65 | Hs.137585 | UDP | |
| | | | | | | | glycosyltransferase 2 | |
| | | | | | | | family, polypeptide | |
| | | | | | | | B11 | |
| 1250 | GTGTCTGTCTC | 2.08 | 0.00 | 2.08 | 0.65 | Hs.137432 | ESTs | |
| 1251 | TTCAGTAATAA | 2.08 | 0.00 | 2.08 | 0.65 | Hs.13479 | hypothetical protein | |
| | | | | | | | FLJ20847 | |
| 1252 | CTTTAAGAAAG | 2.08 | 0.00 | 2.08 | 0.65 | Hs.13456 | Homo sapiens clone | |
| | | | | | | | 24747 mRNA | |
| | | | | | | | sequence | |
| 1253 | TAAAGTGTCTG | 2.08 | 0.00 | 2.08 | 0.65 | Hs.132875 | Homo sapiens | |
| | | | | | | | HSPC309 mRNA, | |
| | | | | | | | partial cds | |
| 1254 | GGCCTCTCCGA | 2.08 | 0.00 | 2.08 | 0.65 | Hs.132834 | hematopoietic protein | |
| | | | | | | | 1 | |
| 1255 | GCACCTTCTGG | 2.08 | 0.00 | 2.08 | 0.65 | Hs.132744 | hypothetical protein | |
| 1256 | CATTGAGCTCC | 2.08 | 0.00 | 2.08 | 0.65 | Hs.12820 | SnRNP assembly | |
| | | | | | | | defective 1 homolog | |
| 1257 | TCAATCAGTGA | 2.08 | 0.00 | 2.08 | 0.65 | Hs.127270 | ESTs | |
| 1258 | ACCTGCCCCTC | 2.08 | 0.00 | 2.08 | 0.65 | Hs.125262 | DKFZP586G1624 | |
| | | | | | | | protein | |
| 1259 | GCACCTTATTG | 2.08 | 0.00 | 2.08 | 0.65 | Hs.125078 | ornithine | |
| | | | | | | | decarboxylase | |
| | | | | | | | antizyme 1 | |
| 1260 | ACAACATAGAA | 2.08 | 0.00 | 2.08 | 0.65 | Hs.12436 | ESTs | |
| 1261 | TGCTGCTTGAA | 2.08 | 0.00 | 2.08 | 0.65 | Hs.12152 | APMCF1 protein | |
| 1262 | TTATTGTTCCC | 2.08 | 0.00 | 2.08 | 0.65 | Hs.12126 | hepatocellular | |
| | | | | | | | carcinoma-associated | |
| | | | | | | | antigen 112 | |
| 1263 | GGTGATGAGGA | 2.08 | 0.00 | 2.08 | 0.65 | Hs.12107 | putative breast | |
| | | | | | | | adenocarcinoma | |
| | | | | | | | marker (32kD) | |
| 1264 | CTGAACTGTGA | 2.08 | 0.00 | 2.08 | 0.65 | Hs.121031 | ESTs | |
| 1265 | AACATAGGAAA | 2.08 | 0.00 | 2.08 | 0.65 | Hs.119663 | CDS9 antigen p18-20 | |
| | | | | | | | (antigen identified by | |
| | | | | | | | monoclonal | |
| | | | | | | | antibodies 16,3A5, | |
| | | | | | | | EJ16, EJ30, EL32 | |
| | | | | | | | and G344) | |
| 1266 | CTGGTGAGTGC | 2.08 | 0.00 | 2.08 | 0.65 | Hs.11902 | MYLE protein | |
| 1267 | GCCTGGGAGAC | 2.08 | 0.00 | 2.08 | 0.65 | Hs.118346 | ESTs | |
| 1268 | AGCTGAGCTAA | 2.08 | 0.00 | 2.08 | 0.65 | Hs.118243 | deoxyribonuclease II, | |
| | | | | | | | lysosomal | |
| 1269 | CCGGACCTGTG | 2.08 | 0.00 | 2.08 | 0.65 | Hs.117582 | CGI-43 protein | |
| 1270 | CGGAGCCGGCT | 2.08 | 0.00 | 2.08 | 0.65 | Hs.117582 | CGI-43 protein | |
| 1271 | GAGAGGTGATT | 2.08 | 0.00 | 2.08 | 0.65 | Hs.114062 | protein tyrosine | |
| | | | | | | | phosphatase-like | |
| | | | | | | | (proline instead of | |
| | | | | | | | catalytic arginine), | |
| | | | | | | | member a | |
| 1272 | AAGATCCTTGT | 2.08 | 0.00 | 2.08 | 0.65 | Hs.113503 | karyopherin (importin) | |
| | | | | | | | beta 3 | |
| 1273 | AATGAACAATA | 2.08 | 0.00 | 2.08 | 0.65 | Hs.11342 | ninjurin 1 | |
| 1274 | TTGGTCAGGTT | 2.08 | 0.00 | 2.08 | 0.65 | Hs.113111 | Homo sapiens | |
| | | | | | | | familial | |
| | | | | | | | Mediterranean fever | |
| | | | | | | | locus region, mRNA | |
| | | | | | | | sequence | |
| 1275 | GACTCTGGGAT | 2.08 | 0.00 | 2.08 | 0.65 | Hs.11282 | ESTs, Weakly similar | |
| | | | | | | | to cleft lip and palate | |
| | | | | | | | transmembrane | |
| | | | | | | | protein 1 [H,sapiens] | |
| 1276 | TACACGTGAGG | 2.08 | 0.00 | 2.08 | 0.65 | Hs.1 1156 | hypothetical protein | |
| 1277 | AGCACTGCAGC | 2.08 | 0.00 | 2.08 | 0.65 | Hs.111039 | N-myristoyltransferase | |
| | | | | | | | 1 | |
| 1278 | AACTCCCAGTT | 2.08 | 0.00 | 2.08 | 0.65 | Hs.110571 | growth arrest and | |
| | | | | | | | DNA-damage-inducible, | |
| | | | | | | | beta | |
| 1279 | GCATAATGTTT | 2.08 | 0.00 | 2.08 | 0.65 | Hs.11050 | F-box only protein 9 | |
| 1280 | CCCAGGACACC | 2.08 | 0.00 | 2.08 | 0.65 | Hs.110443 | Homo sapiens | |
| | | | | | | | mRNA; cDNA | |
| | | | | | | | DKFZp761O051 | |
| | | | | | | | (from clone | |
| | | | | | | | DKFZp761O051) | |
| 1281 | AAAGGAAAGTC | 2.08 | 0.00 | 2.08 | 0.65 | Hs.109706 | HN1 protein | |
| 1282 | AATAAATGGAT | 2.08 | 0.00 | 2.08 | 0.65 | Hs.109052 | chromosome 14 open | |
| | | | | | | | reading frame 2 | |
| 1283 | ACCAACACGGG | 2.08 | 0.00 | 2.08 | 0.65 | Hs.109005 | ESTs | |
| 1284 | CTTCCGGGTAA | 2.08 | 0.00 | 2.08 | 0.65 | Hs.108924 | DKFZP586P1422 | |
| | | | | | | | protein | |
| 1285 | GGAGTCCTAGC | 2.08 | 0.00 | 2.08 | 0.65 | Hs.108894 | hypothetical protein | |
| | | | | | | | FLJ20411 | |
| 1286 | CTTCTGTCTCC | 2.08 | 0.00 | 2.08 | 0.65 | Hs.108824 | ESTs, Weakly similar | |
| | | | | | | | to cDNA EST | |
| | | | | | | | yk415c12,5 comes | |
| | | | | | | | from this gene | |
| | | | | | | | [C,elegans] | |
| 1287 | TTTAGGGGGAA | 2.08 | 0.00 | 2.08 | 0.65 | Hs.108319 | thyroid hormone | |
| | | | | | | | receptor-associated | |
| | | | | | | | protein, 150 kDa | |
| | | | | | | | subunit | |
| 1288 | TGTAGCTGCAA | 2.08 | 0.00 | 2.08 | 0.65 | Hs.107882 | hypothetical protein | |
| | | | | | | | FLJ10659 | |
| 1289 | TGAAACTTTTC | 2.08 | 0.00 | 2.08 | 0.65 | Hs.107528 | androgen induced | |
| | | | | | | | protein | |
| 1290 | TTACAGAGCTT | 2.08 | 0.00 | 2.08 | 0.65 | Hs.10590 | zinc finger protein | |
| | | | | | | | 313 | |
| 1291 | TCCTTTAAAAT | 2.08 | 0.00 | 2.08 | 0.65 | Hs.10587 | KIAA0353 protein | |
| 1292 | GGAACTTGGCT | 2.08 | 0.00 | 2.08 | 0.65 | Hs.105613 | ESTs | |
| 1293 | GAGAACCGTAG | 2.08 | 0.00 | 2.08 | 0.65 | Hs.105547 | neural proliferation, | |
| | | | | | | | differentiation and | |
| | | | | | | | control, 1 | |
| 1294 | TCATCTGCAAA | 2.08 | 0.00 | 2.08 | 0.65 | Hs.105189 | ESTs, Weakly similar | |
| | | | | | | | to AF148856_2 | |
| | | | | | | | unknown [H,sapiens] | |
| 1295 | CATAATTTCTC | 2.08 | 0.00 | 2.08 | 0.65 | Hs.104660 | eIF-5A2 protein | |
| 1296 | CAATCTTGTGA | 2.08 | 0.00 | 2.08 | 0.65 | Hs.104353 | ESTs | |
| 1297 | TTTCCTTCCTT | 2.08 | 0.00 | 2.08 | 0.65 | Hs.104143 | clathrin, light | |
| | | | | | | | polypeptide (Lca) | |
| 1298 | AGACAGAGTGG | 2.08 | 0.00 | 2.08 | 0.65 | Hs.103833 | ESTs, Weakly similar | |
| | | | | | | | to AF151869_1 CGI-111 | |
| | | | | | | | protein | |
| | | | | | | | [H,sapiens] | |
| 1299 | TGGGGAGAGGA | 5.21 | 10.58 | 2.03 | 1.10 | Hs.75799 | protease, serine, 8 | |
| | | | | | | | (prostasin) | |
| 1300 | CCACCACGCTT | 5.21 | 10.58 | 2.03 | 1.10 | Hs.285275 | ESTs | |
| 1301 | TGCCTGTGGTC | 5.21 | 10.58 | 2.03 | 1.10 | Hs.277100 | ESTs | |
| 1302 | ATGGCAGGTGC | 5.21 | 10.58 | 2.03 | 1.10 | Hs.236479 | EST | |
| 1303 | AAGTTGCTATT | 15.62 | 7.70 | 2.03 | 1.38 | Hs.78575 | prosaposin (variant | |
| | | | | | | | Gaucher disease and | |
| | | | | | | | variant | |
| | | | | | | | metachromatic | |
| | | | | | | | leukodystrophy) | |
| 1304 | AGCCACTGCAC | 15.62 | 7.70 | 2.03 | 1.38 | Hs.122126 | ESTs | |
| 1305 | GCAAAAAAAAA | 6.25 | 12.51 | 2.00 | 1.19 | Hs.76293 | thymosin, beta 10 | |
| 1306 | GTGGCACGCGC | 6.25 | 12.51 | 2.00 | 1.19 | Hs.187346 | ESTs | |

TABLE 5

| | | | | | | | |
|---|---|---|---|---|---|---|---|
| No. | Tag sequence | Young | Old | Quotient | Significance | Annotation | Description |

| | | | | | | | |
|---|---|---|---|---|---|---|---|
| 1307 | TCTCCATACCC | 175.94 | 0.00 | 175.94 | 53.84 | manual | Mitochondrial |
| | | | | | | | polymorphic tag, |
| | | | | | | | pos:4216 |
| 1308 | ATGAAACTTCG | 63.50 | 0.96 | 66.15 | 17.94 | manual | Mitochondrial minor |
| | | | | | | | tag, pos:14832 |
| 1309 | CACTACTCACC | 2.08 | 79.85 | 38.39 | 20.69 | manual | Mitochondrial major |
| | | | | | | | tag, pos:14902 |
| 1310 | ACCCTTGGCCA | 10.41 | 109.67 | 10.54 | 21.39 | manual | Mitochondrial major |
| | | | | | | | tag, pos:3839 |
| 1311 | AAACATCCTAT | 1.04 | 4.81 | 4.63 | 0.98 | manual | Mitochondrial minor |
| | | | | | | | tag, pos:7249 |
| 1312 | GTAGGGGTAAA | 4.16 | 0.00 | 4.16 | 1.29 | manual | Mitochondrial |
| | | | | | | | antisense tag, pos:- |
| | | | | | | | 6282 |
| 1313 | TTGGAACAATG | 0.00 | 3.85 | 3.85 | 1.15 | manual | rRNA major tag |
| 1314 | ACCCGCCGGGC | 1.04 | 3.85 | 3.70 | 0.77 | manual | rRNA major tag |
| 1315 | TGGCGTACGGA | 1.04 | 3.85 | 3.70 | 0.77 | manual | rRNA major tag |
| 1316 | CCGACGGGCGC | 4.16 | 15.39 | 3.70 | 2.15 | manual | rRNA intermediate |
| | | | | | | | tag |
| 1317 | GGTCAGTCGGT | 6.25 | 1.92 | 3.26 | 1.05 | manual | rRNA major tag |
| 1318 | TGCCTAGACCA | 3.12 | 0.96 | 3.25 | 0.66 | manual | Mitochondrial minor |
| | | | | | | | tag, pos:12498 |
| 1319 | AGCTGTCCCCA | 0.00 | 2.89 | 2.89 | 0.87 | manual | Mitochondrial minor |
| | | | | | | | tag, pos:8070 |
| 1320 | ATGGCAGGAGT | 0.00 | 2.89 | 2.89 | 0.87 | manual | Mitochondrial |
| | | | | | | | antisense tag, pos:- |
| | | | | | | | 12728 |
| 1321 | TGAGAAGAAGC | 0.00 | 2.89 | 2.89 | 0.87 | manual | Mitochondrial |
| | | | | | | | antisense tag, pos:- |
| | | | | | | | 11703 |
| 1322 | CATTTGGTATT | 8.33 | 2.89 | 2.88 | 1.20 | manual | Mitochondrial minor |
| | | | | | | | tag, pos:44 |
| 1323 | TACTGCTCGGA | 1.04 | 2.89 | 2.78 | 0.59 | manual | Mitochondrial |
| | | | | | | | antisense tag, pos:- |
| | | | | | | | 13715 |
| 1324 | GCTAGGTTTAT | 2.08 | 5.77 | 2.77 | 0.91 | manual | Mitochondrial |
| | | | | | | | antisense tag, pos:- |
| | | | | | | | 7732 |
| 1325 | TGGTGTATGCA | 5.21 | 1.92 | 2.71 | 0.86 | manual | Mitochondrial |
| | | | | | | | antisense tag, pos:- |
| | | | | | | | 9318 |
| 1326 | AATGGATGAAC | 5.21 | 12.51 | 2.40 | 1.37 | manual | rRNA intermediate |
| | | | | | | | tag |
| 1327 | GTAATCCTGCT | 27.07 | 61.57 | 2.27 | 4.03 | manual | rRNA major tag |
| 1328 | TTGCTCAGGCT | 2.08 | 0.96 | 2.17 | 0.46 | manual | rRNA intermediate |
| | | | | | | | tag, Alu |
| 1329 | GAAGTCGGAAT | 3.12 | 6.73 | 2.16 | 0.88 | manual | rRNA major tag |
| 1330 | AGAATCGCTTG | 22.90 | 48.10 | 2.10 | 2.98 | manual | Alu-repeat |
| 1331 | CATTTGTAATA | 4.16 | 8.66 | 2.08 | 1.00 | manual | Mitochondrial |
| | | | | | | | intermediate tag, |
| | | | | | | | pos:6084 |
| 1332 | CTTACAAGCAA | 2.08 | 0.00 | 2.08 | 0.65 | manual | Mitochondrial minor |
| | | | | | | | tag, pos:16193 |
| 1333 | TTACTTATACT | 2.08 | 0.00 | 2.08 | 0.65 | manual | Mitochondrial |
| | | | | | | | antisense tag, pos:- |
| | | | | | | | 14253 |
| 1334 | GGGGTCAGGGG | 5.21 | 10.58 | 2.03 | 1.10 | manual | Mitochondrial |
| | | | | | | | antisense tag, pos:- |
| | | | | | | | 2138 |
| 1335 | AAAACATTCTC | 18.74 | 37.52 | 2.00 | 2.34 | manual | Mitochondrial major |
| | | | | | | | tag, pos:2314 |

TABLE 7

| | | | | | | |
|---|---|---|---|---|---|---|
| | | | | | Unigene | |
| | | | | | No. | |
| No. | Tag sequence | Young | Old | Quotient | (NCBI) | Description |

| | | | | | | |
|---|---|---|---|---|---|---|
| 1336 | TGTGCCAGTGT | 1.04 | 11.54 | 11.10 | 331555 | Homo sapiens serine protease |
| | | | | | | inhibitor, Kazal type, 5 |
| | | | | | | (SPINK5), mRNA |
| 1337 | CATCTGCTGAT | 8.33 | 0 | 8.33 | 28338 | Homo sapiens mRNA for |
| | | | | | | KIAA1546 protein, partial cds |
| 1338 | TTCCCCCTTCC | 7.29 | 0.96 | 7.59 | 332967 | nx11f05.s1 Homo sapiens |
| | | | | | | cDNA, 3′ end |
| 1339 | CGGCTTTTCTG | 7.29 | 0.96 | 7.59 | 324648 | Homo sapiens cDNA FLJ13700 |
| | | | | | | fis, clone PLACE2000216, |
| | | | | | | highly similar to SPECTRIN |
| | | | | | | BETA CHAIN, BRAIN |
| 1340 | CGCCGGGAGCT | 1.04 | 7.7 | 7.40 | 61460 | Homo sapiens cDNA FLJ14847 |
| | | | | | | fis, clone PLACE1000401, |
| | | | | | | weakly similar to POLIOVIRUS |
| | | | | | | RECEPTOR PRECURSOR |
| 1341 | CTGTGGGAAAC | 0 | 6.73 | 6.73 | 225997 | “Homo sapiens mRNA; cDNA |
| | | | | | | DKFZp564C0962 (from clone |
| | | | | | | DKFZp564C0962)” |
| 1342 | GCGTCGGTGCA | 6.25 | 0.96 | 6.51 | 155597 | Homo sapiens D component of |
| | | | | | | complement (adipsin) (DF), |
| | | | | | | mRNA |
| 1343 | GACCAGCTGGC | 6.25 | 0.96 | 6.51 | 74120 | Homo sapiens adipose specific |
| | | | | | | 2 (APM2), mRNA |
| 1344 | GTGTGGTGGAG | 0 | 5.77 | 5.77 | 177486 | Homo sapiens amyloid beta |
| | | | | | | (A4) precursor protein |
| | | | | | | (protease nexin-II, Alzheimer |
| | | | | | | disease) (APP), mRNA |
| 1345 | AAAGTCATTGA | 5.21 | 0.96 | 5.43 | 77899 | Homo sapiens tropomyosin 1 |
| | | | | | | (alpha) (TPM1), mRNA |
| 1346 | ACCTGGAGGGG | 3.12 | 16.35 | 5.24 | 135188 | 602625439F1 Homo sapiens |
| | | | | | | cDNA, 5′ end |
| 1347 | GGTAATCCGTT | 5.21 | 0 | 5.21 | 30942 | Homo sapiens ephrin-B2 |
| | | | | | | (EFNB2), mRNA |
| 1348 | GTGGCGGGCTC | 0 | 4.81 | 4.81 | 142634 | Homo sapiens zinc finger |
| | | | | | | protein (AF020591), mRNA |
| 1349 | CTGATCTCGAA | 0 | 4.81 | 4.81 | 177932 | ie10c08.y1 Homo sapiens |
| | | | | | | cDNA, 5′ end |
| 1350 | CTCGGTACATT | 0 | 4.81 | 4.81 | 74316 | Homo sapiens desmoplakin |
| | | | | | | (DPI, DPII) (DSP), mRNA |
| 1351 | CAGCGGCGGGA | 0 | 4.81 | 4.81 | 2420 | Homo sapiens superoxide |
| | | | | | | dismutase 3, extracellular |
| | | | | | | (SOD3), mRNA |
| 1352 | TAGCTGCTGGT | 1.04 | 4.81 | 4.63 | 11482 | Homo sapiens splicing factor, |
| | | | | | | arginine/serine-rich 11 |
| | | | | | | (SFRS11), mRNA |
| 1353 | GTGGCACATTC | 1.04 | 4.81 | 4.63 | 319567 | Homo sapiens cDNA FLJ12130 |
| | | | | | | fis, clone MAMMA1000251 |
| 1354 | GTCAGTTCCTG | 1.04 | 4.81 | 4.63 | 3796 | Homo sapiens EphB6 (EPHB6) |
| | | | | | | mRNA |
| 1355 | GGTGACAGAGA | 4.16 | 0.96 | 4.33 | 324611 | 602370629F1 Homo sapiens |
| | | | | | | cDNA, 5′ end |
| 1356 | AAACTTTGCCT | 4.16 | 0.96 | 4.33 | 194431 | Homo sapiens palladin |
| | | | | | | (KIAA0992), mRNA |
| 1357 | CAGCTCACTGA | 4.16 | 17.32 | 4.16 | 738 | Homo sapiens ribosomal |
| | | | | | | protein L14 (RPL14), mRNA |
| 1358 | ATGACAGATGG | 2.08 | 8.66 | 4.16 | 13775 | Homo sapiens hypothetical |
| | | | | | | protein SMAP31 (SMAP31), |
| | | | | | | mRNA |
| 1359 | GCACTCTAGCC | 4.16 | 0 | 4.16 | 306735 | Homo sapiens cDNA: |
| | | | | | | FLJ21297 fis, clone COL02035 |
| 1360 | AAATGCTTGGA | 4.16 | 0 | 4.16 | 252998 | xl80g03.x1 Homo sapiens |
| | | | | | | cDNA, 3′ end |
| 1361 | CCTCTTTGCAT | 5.21 | 21.17 | 4.06 | 707 | Homo sapiens keratin 2A |
| | | | | | | (epidermal ichthyosis bullosa of |
| | | | | | | Siemens) (KRT2A), mRNA |
| 1362 | TGGTCCCAGCT | 0 | 3.85 | 3.85 | 275481 | ni38b03.s1 Homo sapiens |
| | | | | | | cDNA, 3′ end |
| 1363 | TGAAGGTGGTG | 0 | 3.85 | 3.85 | 250528 | Homo sapiens, clone |
| | | | | | | IMAGE:4098694, mRNA |
| 1364 | TCAACTTGAAA | 0 | 3.85 | 3.85 | 38891 | 602385216F1 Homo sapiens |
| | | | | | | cDNA, 5′ end |
| 1365 | GTTGTCATCAC | 0 | 3.85 | 3.85 | 2633 | Homo sapiens desmoglein 1 |
| | | | | | | (DSG1), mRNA |
| 1366 | GTGGCGCACGT | 0 | 3.85 | 3.85 | 249720 | Homo sapiens cDNA, 3′ end |
| 1367 | GCACCGTAAGA | 0 | 3.85 | 3.85 | 168232 | Homo sapiens hypothetical |
| | | | | | | protein FLJ13855 (FLJ13855), |
| | | | | | | mRNA |
| 1368 | GACCCGGGAGG | 0 | 3.85 | 3.85 | 41974 | Homo sapiens, clone |
| | | | | | | IMAGE:4100953, mRNA |
| 1369 | CTGCGGAAGAT | 0 | 3.85 | 3.85 | 99816 | Homo sapiens beta-catenin- |
| | | | | | | interacting protein ICAT |
| | | | | | | (LOC56998), mRNA |
| 1370 | AACAGGCAAGA | 0 | 3.85 | 3.85 | 73995 | Human DNA sequence from |
| | | | | | | clone RP1-14N1 on |
| | | | | | | chromosome 1q21.1-21.3 |
| | | | | | | Contains ESTs, GSSs and |
| | | | | | | STSs. Contains the FLG gene |
| | | | | | | for profilaggrin and part of a |
| | | | | | | gene for a novel S-100/ICaBP |
| | | | | | | type calcium binding domain |
| | | | | | | protein similar to trichohyalin.n |
| 1371 | GGCCGCGAGGT | 7.29 | 1.92 | 3.80 | 78344 | Homo sapiens myosin, heavy |
| | | | | | | polypeptide 11, smooth muscle |
| | | | | | | (MYH11), transcript variant |
| | | | | | | SM2, mRNA |
| 1372 | CTAACGCAGCA | 7.29 | 1.92 | 3.80 | 78465 | Homo sapiens v-jun avian |
| | | | | | | sarcoma virus 17 oncogene |
| | | | | | | homolog (JUN), mRNA |
| 1373 | TGGCCTCCCCG | 1.04 | 3.85 | 3.70 | 159161 | Homo sapiens Rho GDP |
| | | | | | | dissociation inhibitor (GDI) |
| | | | | | | alpha (ARHGDIA), mRNA |
| 1374 | GGAGAGAAAAG | 1.04 | 3.85 | 3.70 | 158291 | Human DNA sequence from |
| | | | | | | clone RP1-233K16 on |
| | | | | | | chromosome 1p36.21-36.33 |
| | | | | | | Contains the gene KIAA0444, a |
| | | | | | | putative chromodomain |
| | | | | | | helicase DNA binding protein 3 |
| | | | | | | (CHD3), the gene for |
| | | | | | | potassium channel beta 2 |
| | | | | | | subunit (KCNK2), two CpG |
| | | | | | | island, ESTs, STSs and GSSs |
| 1375 | GGACTCTGCCC | 1.04 | 3.85 | 3.70 | 225948 | Homo sapiens small inducible |
| | | | | | | cytokine subfamily A (Cys- |
| | | | | | | Cys), member 27 (SCYA27), |
| | | | | | | mRNA |
| 1376 | GACGGCGCAGG | 1.04 | 3.85 | 3.70 | 73946 | Homo sapiens endothelial cell |
| | | | | | | growth factor 1 (platelet- |
| | | | | | | derived) (ECGF1), mRNA |
| 1377 | ATGAGATCCTG | 1.04 | 3.85 | 3.70 | 275741 | nx39e06.s1 Homo sapiens |
| | | | | | | cDNA, 3′ end |
| 1378 | ACCTGGAGGGT | 1.04 | 3.85 | 3.70 | 127723 | “Homo sapiens mRNA; cDNA |
| | | | | | | DKFZp586J211 (from clone |
| | | | | | | DKFZp586J211)” |
| 1379 | GTGGCACGTGA | 3.12 | 0.96 | 3.25 | 147996 | Homo sapiens protein kinase, |
| | | | | | | X-linked (PRKX), mRNA |
| 1380 | GTAGCGGGCGC | 3.12 | 0.96 | 3.25 | 323114 | Homo sapiens cDNA FLJ13784 |
| | | | | | fis, clone PLACE4000593 | |
| 1381 | GGGCTGTTTGG | 3.12 | 0.96 | 3.25 | 189658 | Homo sapiens CGI-149 protein |
| | | | | | | (LOC51652), mRNA |
| 1382 | GGCCTCTGAGC | 3.12 | 0.96 | 3.25 | 69559 | Novel human mRNA from |
| | | | | | | chromosome 1, which has |
| | | | | | | similarities to BAT2 genes |
| 1383 | GGCAGTGCCCA | 3.12 | 0.96 | 3.25 | 110915 | Homo sapiens interleukin 22 |
| | | | | | | receptor (IL22R), mRNA |
| 1384 | GCAGTGCGTGC | 3.12 | 0.96 | 3.25 | 202508 | 601649719F1 Homo sapiens |
| | | | | | | cDNA, 5′ end |
| 1385 | GCAAAGCCCTG | 3.12 | 0.96 | 3.25 | 210509 | EST371827 Homo sapiens |
| | | | | | | cDNA |
| 1386 | GATCCCAACAT | 3.12 | 0.96 | 3.25 | 25 | Homo sapiens ATP synthase, |
| | | | | | | H+ transporting, mitochondrial |
| | | | | | | F1 complex, beta polypeptide |
| | | | | | | (ATP5B) |
| 1387 | GAGAAACACCG | 3.12 | 0.96 | 3.25 | 2479 | 602153463F1 Homo sapiens |
| | | | | | | cDNA, 5′ end |
| 1388 | GACGATGTATA | 3.12 | 0.96 | 3.25 | 1837 | Homo sapiens POU domain, |
| | | | | | | class 3, transcription factor 1 |
| | | | | | | (POU3F1), mRNA |
| 1389 | CAAGCATTCCC | 3.12 | 0.96 | 3.25 | 131620 | ov74g10.x1 Homo sapiens |
| | | | | | | cDNA, 3′ end |
| 1390 | ATGAAGAAGGA | 3.12 | 0.96 | 3.25 | 2780 | Homo sapiens jun D proto |
| | | | | | | oncogene (JUND), mRNA |
| 1391 | AGTAGCCGTGA | 3.12 | 0.96 | 3.25 | 286360 | 601655325R1 Homo sapiens |
| | | | | | | cDNA, 3′ end |
| 1392 | ACTTGATAAAT | 3.12 | 0.96 | 3.25 | 344484 | tj56h03.x1 Homo sapiens |
| | | | | | | cDNA, 3′ end |
| 1393 | ACCCGCGAGGA | 3.12 | 0.96 | 3.25 | 170294 | Homo sapiens, clone |
| | | | | | | IMAGE:3637796, mRNA, |
| | | | | | | partial cds |
| 1394 | AACCAGGGAGG | 3.12 | 0.96 | 3.25 | 265317 | Homo sapiens hypothetical |
| | | | | | | protein MGC2562 (MGC2562), |
| | | | | | | mRNA |
| 1395 | TAACCAAATCA | 3.12 | 0 | 3.12 | 145421 | qf27h11.x5 Homo sapiens |
| | | | | | | cDNA, 3′ end |
| | | | | | | /clone=IMAGE:1751301 |
| 1396 | GTGGTGGTGCC | 3.12 | 0 | 3.12 | 13197 | 603045818F1 Homo sapiens |
| | | | | | | cDNA, 5′ end |
| 1397 | GTGAAACTCAG | 3.12 | 0 | 3.12 | 178485 | Homo sapiens cDNA FLJ13919 |
| | | | | | | fis, clone Y79AA1000410 |
| 1398 | GGGCTCGGGGA | 3.12 | 0 | 3.12 | 105927 | “Homo sapiens stem cell |
| | | | | | | growth factor; lymphocyte |
| | | | | | | secreted C-type lectin (SCGF), |
| | | | | | | mRNA” |
| 1399 | GCGGCGGGTGC | 3.12 | 0 | 3.12 | 257719 | he36a05.x1 Homo sapiens |
| | | | | | | cDNA, 3+ end |
| 1400 | GCAAAATTCTG | 3.12 | 0 | 3.12 | 336728 | nk76b08.s1 Homo sapiens |
| | | | | | | cDNA, 3′ end |
| 1401 | GAAGGAGGCAT | 3.12 | 0 | 3.12 | 184771 | Homo sapiens nuclear factor |
| | | | | | | I/C (CCAAT-binding |
| | | | | | | transcription factor) (NFIC), |
| | | | | | | mRNA |
| 1402 | CTTTTAAGAAA | 3.12 | 0 | 3.12 | 86043 | Homo sapiens cDNA FLJ13558 |
| | | | | | | fis, clone PLACE1007743 |
| 1403 | CTCTACAGTGC | 3.12 | 0 | 3.12 | 24322 | Homo sapiens ATPase, H+ |
| | | | | | | transporting, lysosomal |
| | | | | | | (vacuolar proton pump) 9kD |
| | | | | | | (ATP6H), mRNA |
| 1404 | CGTTTTCTGAT | 3.12 | 0 | 3.12 | 82911 | Homo sapiens BM-008 mRNA, |
| | | | | | | complete cds |
| 1405 | CCCCGGGCCTC | 3.12 | 0 | 3.12 | 89901 | Homo sapiens |
| | | | | | | phosphodiesterase 4A, cAMP- |
| | | | | | | specific (dunce (Drosophila)- |
| | | | | | | homolog phosphodiesterase |
| | | | | | | E2) (PDE4A), mRNA |
| 1406 | CCCCCACCCGG | 3.12 | 0 | 3.12 | 78482 | Homo sapiens paralemmin |
| | | | | | | (PALM), mRNA |
| 1407 | CCATCTTGAGG | 3.12 | 0 | 3.12 | 110707 | Homo sapiens H326 (H326), |
| | | | | | | mRNA |
| 1408 | CAGACCGGTGC | 3.12 | 0 | 3.12 | 118397 | Homo sapiens AE-binding |
| | | | | | | protein 1 (AEBP1), mRNA |
| 1409 | AGTAACAAGAT | 3.12 | 0 | 3.12 | 118174 | Homo sapiens tetratricopeptide |
| | | | | | | repeat domain 3 (TTC3), |
| | | | | | | mRNA |
| 1410 | AGCCTAGGAGT | 3.12 | 0 | 3.12 | 37308 | yq9Be10.s1 Homo sapiens |
| | | | | | | cDNA, 3′ end |
| 1411 | AATTAACTCCG | 3.12 | 0 | 3.12 | 169517 | Homo sapiens, Similar to |
| | | | | | | aldehyde dehydrogenase 5, |
| | | | | | | clone MGC:2230 |
| 1412 | GCTTTATTTGT | 28.11 | 9.62 | 2.92 | 288061 | Homo sapiens actin, beta |
| | | | | | | (ACTB), mRNA |
| 1413 | TTTATCTTTTA | 0 | 2.89 | 2.89 | 303125 | Homo sapiens p53-induced |
| | | | | | | protein PIGPC1 (PIGPC1), |
| | | | | | | mRNA |
| 1414 | TGTTCCTGGAT | 0 | 2.89 | 2.89 | 287423 | Homo sapiens cDNA FLJ11556 |
| | | | | | | fis, clone HEMBA1003079 |
| 1415 | TGCGCTGGCCC | 0 | 2.89 | 2.89 | 289019 | Homo sapiens latent |
| | | | | | | transforming growth factor beta |
| | | | | | | binding protein 3 (LTBP3), |
| | | | | | | mRNA |
| 1416 | TCGGAGCTGCT | 0 | 2.89 | 2.89 | 1030 | Homo sapiens ras inhibitor |
| | | | | | | (RIN1), mRNA |
| 1417 | GTTTCCAATGC | 0 | 2.89 | 2.89 | 57672 | Homo sapiens leucine rich |
| | | | | | | repeat (in FLII) interacting |
| | | | | | | protein 2 (LRRFIP2), mRNA |
| 1418 | GTTCAGCTGTC | 0 | 2.89 | 2.89 | 78902 | Homo sapiens voltage- |
| | | | | | | dependent anion channel 2 |
| | | | | | | (VDAC2), mRNA |
| 1419 | GTGGTGCAAGC | 0 | 2.89 | 2.89 | 306411 | Homo sapiens cDNA FLJ20846 |
| | | | | | | fis, clone ADKA01802 |
| 1420 | GTGGTGAGTAC | 0 | 2.89 | 2.89 | 182999 | yx41g11.s1 Homo sapiens |
| | | | | | | cDNA, 3′ end |
| 1421 | GTGAGCCCATT | 0 | 2.89 | 2.89 | 74335 | Homo sapiens heat shock |
| | | | | | | 90kD protein 1, beta (HSPCB), |
| | | | | | | mRNA |
| 1422 | GGCTATGCCAA | 0 | 2.89 | 2.89 | 3688 | Homo sapiens mRNA for |
| | | | | | | cisplatin resistance-associated |
| | | | | | | overexpressed protein, |
| | | | | | | complete cds |
| 1423 | GGCAGCTGGCA | 0 | 2.89 | 2.89 | 3487 | Homo sapiens hypothetical |
| | | | | | | protein FLJ10439 (FLJ10439), |
| | | | | | | mRNA |
| 1424 | GGCAGACAATC | 0 | 2.89 | 2.89 | 335880 | 601277672F1 Homo sapiens |
| | | | | | | cDNA, 5′ end |
| 1425 | GCTAAAAACAA | 0 | 2.89 | 2.89 | 134590 | oy67c11.x1 Homo sapiens |
| | | | | | | cDNA, 3′ end |
| 1426 | GCAGCTACGGC | 0 | 2.89 | 2.89 | 80828 | Homo sapiens keratin 1 |
| | | | | | | (epidermolytic hyperkeratosis) |
| | | | | | | (KRT1), mRNA |
| 1427 | GATCTTCTCGG | 0 | 2.89 | 2.89 | 73995 | Human profilaggrin mRNA, 3′ |
| | | | | | | end |
| 1428 | GAGGAGTCCAT | 0 | 2.89 | 2.89 | 1432 | Homo sapiens protein kinase C |
| | | | | | | substrate 80K-H (PRKCSH), |
| | | | | | | mRNA |
| 1429 | GACCACACACC | 0 | 2.89 | 2.89 | 86950 | qf57c11.x1 Homo sapiens |
| | | | | | | cDNA, 3′ end |
| 1430 | CGGTCATTCTC | 0 | 2.89 | 2.89 | 154396 | Homo sapiens cDNA: |
| | | | | | | FLJ22282 fis, clone HRC03861 |
| 1431 | CCTATGGTCCC | 0 | 2.89 | 2.89 | 344536 | cong1.P10.E3 Homo sapiens |
| | | | | | | cDNA, 3′ end |
| 1432 | CCTATAGTCTC | 0 | 2.89 | 2.89 | 328052 | ty52c06.x1 Homo sapiens |
| | | | | | | cDNA, 3′ end |
| 1433 | CCCTGTTGATA | 0 | 2.89 | 2.89 | 151254 | Homo sapiens kallikrein 7 |
| | | | | | | (chymotryptic, stratum |
| | | | | | | corneum) (KLK7), mRNA |
| 1434 | CCAGTGAATAG | 0 | 2.89 | 2.89 | 146825 | qb68e04.x1 Homo sapiens |
| | | | | | | cDNA, 3′ end |
| 1435 | CCACTGCACCA | 0 | 2.89 | 2.89 | 257594 | Homo sapiens mRNA for |
| | | | | | | KIAA1364 protein, partial cds |

TABLE 6

| | Unigene | | | |
|---|---|---|---|---|
| | accession | SWISSPROT | | |
| No. | No(s). | or TREMBL | EMBL/GENBANK | Description |
| 1 | Hs.279604 | | | DESMIN: DESMIN |
| 2 | Hs.119571 | | | COLLAGEN ALPHA1(III) |
| 3 | Hs.74649 | | | COX6C: CYTOCHROME C OXIDASE |
| | | | | POLYPEPTIDE VIC [PRECURSOR] EC |
| | | | | 1.9.3.1 |
| 4 | Hs.102948 | | | ENIGMA |
| 5 | Hs.76941 | | | NA K ATPASE B3: NA K ATPASE BETA3 |
| 6 | Hs.183760 | | | C3: (C3) COMPLEMENT C3 PRECURSOR |
| 7 | Hs.75748 | | | PSMB1: (PSMB1 OR PSC5) PROTEASOME |
| | | | | COMPONENT C5 |
| 8 | Hs.198427 | | | HK2: HEXOKINASE, TYPE II EC 2.7.1.1 HK |
| | | | | II |
| 9 | Hs.170171 | | | GLNA GLUTAMINE SYNTHETASE (EC |
| | | | | 6.3.1.2) (GLUTAMATE AMMONIA LIGASE) |
| 10 | Hs.81328 | | | IKBA: (NFKBIA OR NFKBI OR MAD3) I |
| | | | | KAPPA B ALPHA, MAJOR |
| | | | | HISTOCOMPATIBILITY COMPLEX |
| | | | | ENHANCER BINDING |
| 11 | Hs.197345 | | | G22P1: (G22P1) ATP DEPENDENT DNA |
| | | | | HELICASE II, 70 KDA SUBUNIT (LUPUS KU |
| | | | | AUTOANTIGEN PROTEIN P70) (KU70) (70 KDA |
| | | | | SUBUNIT OF KU ANTIGEN) |
| | | | | (THYROID LUPUS AUTOANTIGEN) (TLAA) |
| | | | | (CTC BOX BINDING FACTOR 75 KDA |
| | | | | SUBUNIT) (CTCBF) (CTC75). |
| 12 | Hs.155560 | | | CANX: (CANX) CALNEXIN PRECURSOR |
| | | | | (MAJOR HISTOCOMPATIBILITY COMPLEX |
| | | | | CLASS I ANTIGEN BINDING PROTEIN P88) |
| | | | | (P90) (IP90). |
| 13 | Hs.119222 | | | HIP: (HIP OR ST13 OR P48) HSC70 |
| | | | | INTERACTING PROTEIN |
| | | | | (PROGESTERONE RECEPTOR |
| | | | | ASSOCIATED P48 PROTEIN) (PUTATIVE |
| | | | | TUMOR SUPPRESSOR ST13). |
| 14 | Hs.4055 | | | ZF9: ZINC FINGER PROTEIN 9 |
| 15 | Hs.179573 | | | COLLAGEN ALPHA2 (I) |
| 16 | Hs.74077 | | | PROTEASOME IOTA CHAIN (EC 3.4.99.46) |
| | | | | (MACROPAIN IOTA CHAIN) |
| | | | | (MULTICATALYTIC ENDOPEPTIDASE |
| | | | | COMPLEX IOTA CHAIN) (27 KDA |
| | | | | PROSOMAL PROTEIN) (PROS 27) (P27K). |
| | | | | {GENE: PSMA6 OR PROS27} |
| 17 | Hs.83077 | | | INTERLEUKIN 18 |
| 18 | Hs.79387 | | | PSMC5: (PSMC5 OR S8) 26S |
| | | | | PROTEASOME REGULATORY ATPASE |
| | | | | SUBUNIT 8 (P45) (TRIP1). |
| 19 | Hs.750 | | | FBN1, FIBRILLIN 1 PRECURSOR |
| 20 | Hs.173125 | | | CYP3: PEPTIDYL PROLYL CIS TRANS |
| | | | | ISOMERASE, MITOCHONDRIAL |
| | | | | [PRECURSOR] EC |
| 21 | Hs.131255 | | | UQCRB: UBIQUINOL CYTOCHROME C |
| | | | | REDUCTASE COMPLEX 14 KDA PROTEIN |
| | | | | EC 1.10.2.2 |
| 22 | Hs.75511 | | | CTGF CONNECTIVE TISSUE GROWTH |
| | | | | FACTOR PRECURSOR |
| 23 | Hs.75410 | | | BIP: (HSPA5 OR GRP78) 78 KD GLUCOSE |
| | | | | REGULATED PROTEIN PRECURSOR |
| 24 | Hs.75360 | | | CBPH: CARBOXYPEPTIDASE H |
| | | | | PRECURSOR (EC 3.4.17.10) (CPH) |
| | | | | (CARBOXYPEPTIDASE E)(CPE) |
| | | | | (ENKEPHALIN CONVERTASE) |
| | | | | (PROHORMONE |
| | | | | PROCESSINGCARBOXYPEPTIDASE) |
| 25 | Hs.6101 | | | BMP6 BONE MORPHOGENETIC PROTEIN |
| | | | | 6 PRECURSOR (BMP 6) |
| 26 | Hs.154103 | | | LIM |
| 27 | Hs.110802 | | | VWF VON WILLEBRAND FACTOR |
| | | | | PRECURSOR |
| 28 | Hs.111301 | | | MMP2, 72 KD TYPE IV COLLAGENASE |
| | | | | PRECURSOR (EC 3.4.24.24) (72 KD |
| | | | | GELATINASE) |
| 29 | Hs.172928 | | | COLLAGEN ALPHA1(I) |
| 30 | Hs.78225 | | | ANX1: ANNEXIN I (LIPOCORTIN I) |
| | | | | (CALPACTIN II) (CHROMOBINDIN 9) |
| | | | | (P35)(PHOSPHOLIPASE A2 INHIBITORY |
| | | | | PROTEIN) |
| 31 | Hs.252189 | | | RYODOCAN, RYUDOCAN CORE PROTEIN |
| 32 | Hs.241257 | | | LTBP1, HSTGFB1B ORF FROM |
| | | | | TGFB_HUMAN |
| 33 | Hs.227751 | | | GALECTIN 1: (L14, LGALS1 OR GBP) |
| | | | | GALECTIN 1 |
| 34 | Hs.111779 | | | SPARC, SPARC PRECURSOR (SECRETED |
| | | | | PROTEIN ACIDIC AND RICH IN CYSTEINE) |
| 35 | Hs.79368 | | | EMP1: (EMP1 OR TMP OR B4B) |
| | | | | EPITHELIAL MEMBRANE PROTEIN 1 (EMP |
| | | | | 1) (TUMOR ASSOCIATED MEMBRANE |
| | | | | PROTEIN) (CL 20) (B4B PROTEIN). |
| 36 | Hs.77508 | | | GLUDP1: GLUTAMATE DEHYDROGENASE |
| | | | | 1/2 [PRECURSOR] EC 1.4.1.3 GDH |
| 37 | Hs.74471 | | | CXB1 GAP JUNCTION BETA 1 PROTEIN |
| | | | | (CONNEXIN 32) (CX32) (GAP JUNCTION |
| | | | | 28 KD LIVER PROTEIN) |
| 38 | Hs.23598 | | | CREBBP: (CREBBP OR CBP) CREB |
| | | | | BINDING PROTEIN. |
| 39 | Hs.184601 | | | MPE16 INTEGRAL MEMBRANE PROTEIN |
| | | | | E16 |
| 40 | Hs.180532 | | | HSP86 = HSP90 ALPHA HSPCA |
| 41 | Hs.149846 | | | INTEGRIN BETA5 |
| 42 | Hs.79732 | | | FIBULIN1, HS2444 ORF FROM |
| | | | | FBLD_HUMAN |
| 43 | Hs.79070 | | | CMYC: (MYC) MYC PROTO ONCOGENE |
| | | | | PROTEIN (C MYC) |
| 44 | Hs.75847 | | | ESTB2.2: |
| 45 | Hs.89761 | | | ATP5D: ATP SYNTHASE DELTA CHAIN, |
| | | | | MITOCHONDRIAL [PRECURSOR] |
| 46 | Hs.85289 | | | CD34 |
| 47 | Hs.83004 | | | INTERLEUKIN 14 |
| 48 | Hs.80475 | | | POLR2J: (POLR2J) DNA DIRECTED RNA |
| | | | | POLYMERASE II 13.3 KDA POLYPEPTIDE |
| | | | | (EC 2.7.7.6) (RPB11). |
| 49 | Hs.73965 | | | MRF1: SFRS10, SRFA, MYELIN |
| | | | | REGULATORY FACTOR 1 (PROBABLY |
| | | | | IDENTICAL TO SFRS2/SC 35) |
| 50 | Hs.199160 | | | MLL: (MLL OR HRX OR ALL1 OR TRX1 OR |
| | | | | HTRX) ZINC FINGER PROTEIN HRX (ALL |
| | | | | 1) (TRITHORAX LIKE PROTEIN). |
| 51 | Hs.180714 | | | COX6A1: CYTOCHROME C OXIDASE |
| | | | | POLYPEPTIDE VIA LIVER [PRECURSOR] |
| | | | | EC 1.9.3.1 |
| 52 | Hs.155433 | | | ATP5C1: (ATP5C1 OR ATP5C) ATP |
| | | | | SYNTHASE GAMMA CHAIN, |
| 53 | Hs.75445 | | | HEVIN (= SC1, ECM, HEVIN LIKE PROTEIN |
| | | | | !?) |
| 54 | Hs.180532 | | | HSP86 = HSP90 ALPHA HSPCA |
| 55 | Hs.83190 | | | FAS: FATTY ACID SYNTHASE (EC |
| | | | | 2.3.1.85) [INCLUDES: EC 2.3.1.38; EC |
| | | | | 2.3.1.39; EC 2.3.1.41; EC 1.1.1.100; EC |
| | | | | 4.2.1.61; EC 1.3.1.10; EC 3.1.2.14]. |
| 56 | Hs.9589 | | | PLIC 1: (PLIC 1 OR UBIQUILIN) UBIQUILIN |
| 57 | Hs.80986 | | | ATP5G1: ATP SYNTHASE LIPID BINDING |
| | | | | PROTEIN P1 PRECURSOR (EC 3.6.1.34) |
| | | | | (ATPASE |
| 58 | Hs.78781 | | | VEGB VASCULAR ENDOTHELIAL |
| | | | | GROWTH FACTOR B PRECURSOR (VEGF |
| | | | | B) (VEGF RELATED FACTOR) |
| 59 | Hs.78409 | | | COLLAGEN ALPHA1(XVIII) |
| 60 | Hs.76753 | | | CD105/ENDOGLIN |
| 61 | Hs.75516 | | | TYK2: (TYK2) NON RECEPTOR TYROSINE |
| | | | | PROTEIN KINASE TYK2 (EC 2.7.1.112). |
| 62 | Hs.268571 | | | APC1: APOLIPOPROTEIN C I |
| | | | | PRECURSOR (APO C1) |
| 63 | Hs.23960 | | | CYCLIN B1 G2/MITOTIC SPECIFIC CYCLIN |
| | | | | B1 (CCNB1 OR CCNB) |
| 64 | Hs.211579 | | | CD146/CELL SURFACE GLYCOPROTEIN |
| | | | | MUC18 |
| 65 | Hs.197114 | | | KIAA0324 |
| 66 | Hs.181028 | | | COX5A: CYTOCHROME C OXIDASE |
| | | | | POLYPEPTIDE VA, MITOCHONDRIAL |
| | | | | PRECURSOR (EC |
| 67 | Hs.148495 | | | PSMD4: (PSMD4 OR MCB1) 26S |
| | | | | PROTEASOME REGULATORY SUBUNIT |
| | | | | S5A (AF) (ASF). |
| 68 | Hs.125359 | | | CD90/THY 1 |
| 69 | Hs.1139 | | | DBPA: DBPA_HUMAN (CSDA OR DBPA) |
| | | | | DNA BINDING PROTEIN A (COLD SHOCK |
| | | | | DOMAIN |
| 70 | Hs.111076 | | | MDH2: MALATE DEHYDROGENASE, |
| | | | | MITOCHONDRIAL [PRECURSOR] EC |
| | | | | 1.1.1.37 |
| 71 | Hs.106673 | | | EIF3S6: (EIF3S6 OR INT6) EUKARYOTIC |
| | | | | TRANSLATION INITIATION FACTOR 3 |
| | | | | SUBUNIT 6 (EIF 3 P48) (MAMMARY |
| | | | | TUMOR ASSOCIATED PROTEIN INT 6) |
| | | | | (VIRAL INTEGMOUSEION SITE PROTEIN |
| | | | | INT 6). |
| 72 | Hs.89649 | | | EPOHY MI EPOXIDE HYDROXYLASE, |
| | | | | MICROSOMAL (EPHX1) |
| 73 | Hs.88474 | | | PGH1: PROSTAGLANDIN G/H SYNTHASE |
| | | | | 1 PRECURSOR (EC 1.14.99.1) |
| | | | | (CYCLOOXYGENASE 1) (COX 1) |
| | | | | (PROSTAGLANDIN ENDOPEROXIDE |
| | | | | SYNTHASE 1) (PROSTAGLANDIN |
| | | | | H2SYNTHASE 1) (PGH SYNTHASE 1) |
| | | | | (PGHS 1) (PHS 1)(PTGS1) |
| 74 | Hs.79172 | | | ANT2: ADP, ATP CARRIER PROTEIN, |
| | | | | FIBROBLAST ISOFORM ADP/ATP |
| | | | | TRANSLOCASE 2 |
| 75 | Hs.429 | | | ATP5G3: ATP SYNTHASE LIPID BINDING |
| | | | | PROTEIN P3 [PRECURSOR] EC 3.6.1.34 |
| | | | | ATPASE |
| 76 | Hs.8867 | | | CYR6 CYR61 PROTEIN PRECURSOR |
| | | | | (GIG1 PROTEIN) (INSULIN LIKE GROWTH |
| | | | | FACTOR BINDING PROTEIN 10) |
| 77 | Hs.861 | | | MITOGEN ACTIVATED PROTEIN KINASE 3 |
| | | | | (EC 2.7.1.) (EXTRACELLULAR SIGNAL |
| | | | | REGULATED KINASE 1) (ERK1) (INSULIN |
| | | | | STIMULATED MAP2 KINASE) (MAP |
| | | | | KINASE 1) (MAPK 1) (P44 ERK1) (ERT2) |
| | | | | (P44 MAPK) (MICROTUBULE ASSOCIATED |
| | | | | PROTEIN 2 KINASE) (MAPK3 OR PRKM3 |
| | | | | OR ERK1) |
| 78 | Hs.82112 | | | CD121A/INTERLEUKIN 1 RECEPTOR, |
| | | | | TYPE 1 |
| 79 | Hs.80645 | | | IRF1: INTERFERON REGULATORY |
| | | | | FACTOR |
| 80 | Hs.75617 | | | COLLAGEN ALPHA2(IV) |
| 81 | Hs.75608 | | | X104 |
| 82 | Hs.75428 | | | SOD 1 CU ZN SUPEROXIDASE |
| | | | | DISMUTASE (SOD 1) GENE |
| 83 | Hs.75334 | | | EXT2: (EXT2) EXOSTOSIN 2 (PUTATIVE |
| | | | | TUMOR SUPPRESSOR PROTEIN EXT2) |
| | | | | (MULTIPLE EXOSTOSES PROTEIN 2). |
| 84 | Hs.46468 | | | CKR6 C C CHEMOKINE RECEPTOR TYPE |
| | | | | 6 (C C CKR 6) (CC CKR 6) (CCR 6) (LARC |
| | | | | RECEPTOR) (GPR CY4) (GPRCY4) |
| | | | | (CHEMOKINE RECEPTOR LIKE 3) (CKR |
| | | | | L3) (DRY6) |
| 85 | Hs.245188 | | | TIMP3, METALLOPROTEINASE INHIBITOR |
| | | | | 3 PRECURSOR (TIMP 3) (TISSUE |
| | | | | INHIBITOR OF METALLOPROTEINASES 3) |
| 86 | Hs.237356 | | | CXCL12: SDF1_HUMAN (SDF1) STROMAL |
| | | | | CELL DERIVED FACTOR 1 PRECURSOR |
| | | | | (SDF 1) (PRE B CELL GROWTH |
| | | | | STIMULATING FACTOR) (PBSF) |
| 87 | Hs.183 | | | DUFF DUFFY ANTIGEN (FY |
| | | | | GLYCOPROTEIN) (GLYCOPROTEIN D) |
| | | | | (GPFY) |
| 88 | Hs.172180 | | | KIAA0440 |
| 89 | Hs.171825 | | | DEC1: DEC1 (STRA13) (STRA14) |
| 90 | Hs.146428 | | | COLLAGEN ALPHA1(V) |
| 91 | Hs.105700 | | | FRPHE FRPHE |
| 92 | Hs.75736 | | | APD: APOLIPOPROTEIN D PRECURSOR |
| | | | | APOD |
| 93 | Hs.83551 | | | MAGP1, HUMAN MICROFIBRIL |
| | | | | ASSOCIATED GLYCOPROTEIN (MFAP2) |
| 94 | Hs.75431 | | | FGG: (FGG) FIBRINOGEN GAMMA CHAIN |
| | | | | PRECURSOR. |
| 95 | Hs.75356 | | | TCF4: (TCF4 OR ITF2 OR |
| | | | | SEF2)(IMMUNOGLOBULIN |
| | | | | TRANSCRIPTION FACTOR 2) (ITF 2) |
| 96 | Hs.63236 | | | SNCG OR BCSG1 SYU3 (GAMMA |
| | | | | SYNUCLEIN) (PERSYN) (BREAST |
| | | | | CANCER SPECIFIC GENE 1 PROTEIN) |
| 97 | Hs.25313 | | | MSP58 NUCLEOLAR PROTEIN |
| 98 | Hs.167835 | | | CAOP: ACYL COENZYME A OXIDASE, |
| | | | | PEROXISOMAL (EC 1.3.3.6) (PALMITOYL |
| | | | | COAOXIDASE) (AOX) |
| 99 | Hs.119475 | | | CIRBP: (CIRBP OR CIRP OR A18HNRNP) |
| | | | | COLD INDUCIBLE RNA BINDING PROTEIN |
| | | | | (GLYCINE RICH RNA BINDING PROTEIN |
| | | | | CIRP) (A18 HNRNP). |
| 100 | Hs.116577 | | | MIC 1 HOMO SAPIENS MACROPHAGE |
| | | | | INHIBITORY CYTOKINE 1 (MIC 1) MRNA |
| 101 | Hs.105097 | | | TK1: (TK1) THYMIDINE KINASE, |
| | | | | CYTOSOLIC (EC 2.7.1.21). |
| 102 | Hs.81097 | | | COX8: CYTOCHROME C OXIDASE |
| | | | | POLYPEPTIDE VIII LIVER/HEART |
| | | | | [PRECURSOR] EC 1.9.3.1 |
| 103 | Hs.75106 | | | CLU: (CLU) CLUSTERIN PRECURSOR |
| | | | | (COMPLEMENT ASSOCIATED PROTEIN |
| | | | | SP 40, 40) (COMPLEMENT CYTOLYSIS |
| | | | | INHIBITOR) (CLI) (NA1 AND NA2) |
| | | | | (APOLIPOPROTEIN J) (APO J) (TRPM 2). |
| 104 | Hs.83623 | | | NR1I3: (NR1I3) ORPHAN NUCLEAR |
| | | | | RECEPTOR NR1I3 (CONSTITUTIVE |
| | | | | ANDROSTANE RECEPTOR) (CAR) |
| | | | | (ORPHAN NUCLEAR RECEPTOR MB67). |
| 105 | Hs.76722 | | | CEBPD: (NE IL6 BETA) CCAAT/ENHANCER |
| | | | | BINDING PROTEIN DELTA |
| 106 | Hs.76136 | | | TXN: (TXN OR TRDX OR TRX) |
| | | | | THIOREDOXIN (ATL DERIVED FACTOR) |
| | | | | (ADF) (SURFACE ASSOCIATED |
| | | | | SULPHYDRYL PROTEIN) (SASP). |
| 107 | Hs.278614 | | | LON: MITOCHONDRIAL LON PROTEASE |
| | | | | HOMOLOG PRECURSOR (EC 3.4.21.) |
| 108 | Hs.278242 | | | ALPHA TUBULIN |
| 109 | Hs.277401 | | | BAZ2B2: (BAZ2B) BROMODOMAIN |
| | | | | ADJACENT TO ZINC FINGER DOMAIN 2B |
| | | | | KIAA0314 |
| 110 | Hs.184161 | | | EXT1: (EXT1) EXOSTOSIN 1 (PUTATIVE |
| | | | | TUMOR SUPPRESSOR PROTEIN EXT1) |
| | | | | (MULTIPLE EXOSTOSES PROTEIN 1). |
| 111 | Hs.182429 | | | CABP1: (CABP1 OR ERP5) PROBABLE |
| | | | | PROTEIN DISULFIDE ISOMERASE P5 |
| | | | | PRECURSOR (EC 5.3.4.1). |
| 112 | Hs.173664 | | | ERBB2: RECEPTOR PROTEIN TYROSINE |
| | | | | KINASE ERBB 2 |
| 113 | Hs.1244 | | | CD9 |
| 114 | Hs.79516 | | | BASP1 MYRISTOYLATED PROTEINS |
| | | | | ABUNDANT IN AXONAL TERMINI |
| 115 | Hs.169476 | | | (GAPDH) GLYCERALDEHYDE 3 |
| | | | | PHOSPHATE DEHYDROGENASE, LIVER |
| | | | | (EC 1.2.1.12) (GAPD) |
| 116 | Hs.23582 | | | M1S1: (M1S1 OR GA733 1 OR TROP2) |
| | | | | PANCREATIC CARCINOMA MARKER |
| | | | | PROTEIN GA733 1 PRECURSOR (CELL |
| | | | | SURFACE GLYCOPROTEIN TROP 2). |
| 117 | Hs.195850 | | | KRT5: (KRT5) KERATIN, TYPE II |
| | | | | CYTOSKELETAL 5 (CYTOKERATIN 5) (K5) |
| | | | | (CK 5) (58 KDA CYTOKERATIN) |
| 118 | Hs.15977 | | | NDUFB9: NADH UBIQUINONE |
| | | | | OXIDOREDUCTASE B22 SUBUNIT EC |
| | | | | 1.6.5.3 EC 1.6.99.3 |
| 119 | Hs.150580 | | | SUI1KURZ: SUI1TRANSLATION |
| | | | | INITIATION FACTOR |
| 120 | Hs.76307 | | | DAN ZINC FINGER PROTEIN DAN (N03) |
| 121 | Hs.177486 | | | APP ALZHEIMER PRECURSOR PROTEIN |
| | | | | A4 |
| 122 | Hs.57929 | | | MEGF5 MRNA FOR MEGF5 SLIT 3 |
| 123 | Hs.155101 | | | ATPA ATP SYNTHASE ALPHA CHAIN, |
| | | | | MITOCHONDRIAL PRECURSOR |
| 124 | Hs.8762 | | | FKBP63: (FKBP63) FK506 BINDING |
| | | | | PROTEIN (FRAGMENT) FKBP9. |
| 125 | Hs.848 | | | FKBP4: (FKBP4) P59 PROTEIN (HSP |
| | | | | BINDING IMMUNOPHILIN) (HBI) |
| | | | | (POSSIBLE PEPTIDYL PROLYL CIS |
| | | | | TRANS ISOMERASE) (EC 5.2.1.8) (PPIASE) |
| | | | | (ROTAMASE) (FKBP52 PROTEIN) (52 KDA |
| | | | | FK506 BINDING PROTEIN) (P52) (FKBP59) |
| | | | | (HSP56). |
| 126 | Hs.54457 | | | CD81/AAPA1 |
| 127 | Hs.230 | | | FIBROMODULIN |
| 128 | Hs.173902 | | | PPP2R1A: (PPP2R1A) |
| | | | | SERINE/THREONINE PROTEIN |
| | | | | PHOSPHATASE 2A, 65 KDA REGULATORY |
| | | | | SUBUNIT A, ALPHA ISOFORM (PP2A, |
| | | | | SUBUNIT A, PR65 ALPHA ISOFORM) |
| | | | | (PP2A, SUBUNIT A, R1 ALPHA ISOFORM) |
| | | | | (MEDIUM TUMOR ANTIGEN ASSOCIATED |
| | | | | 61 KDA PROTEIN) |
| 129 | Hs.9194 | | | GBDR1: (GBDR1) PUTATIVE |
| | | | | GLIALBLASTOMA CELL |
| | | | | DIFFERENTIATION RELATED PROTEIN. |
| 130 | Hs.83469 | | | NRF1: (NFE2L1 OR NRF1 OR TCF11 OR |
| | | | | HBZ17) NFE2 RELATED FACTOR 1 |
| 131 | Hs.82120 | | | NR4A2: (NR4A2 OR NURR1 OR TINUR OR |
| | | | | NOT) ORPHAN NUCLEAR RECEPTOR |
| | | | | NURR1 |
| 132 | Hs.77171 | | | MCM5: (MCM5 OR CDC46) DNA |
| | | | | REPLICATION LICENSING FACTOR MCM5 |
| | | | | (CDC46 HOMOLOG) (P1 CDC46). |
| 133 | Hs.77054 | | | BTG1: (BTG1) BTG1 PROTEIN (B CELL |
| | | | | TRANSLOCATION GENE 1 PROTEIN). |
| 134 | Hs.76686 | | | GPX1 GLUTATHIONE PEROXIDASE 1 |
| 135 | Hs.69745 | | | FDXR: NADPH: ADRENODOXIN |
| | | | | OXIDOREDUCTASE [PRECURSOR] EC |
| | | | | 1.18.1.2 ADRENODOXIN |
| 136 | Hs.279554 | | | PSMD13: (PSMD13) 26S PROTEASOME |
| | | | | SUBUNIT S11 (P40.5) |
| 137 | Hs.239663 | | | AFX1: (AFX1 OR AFX OR MLLT7) |
| | | | | PUTATIVE FORK HEAD DOMAIN |
| | | | | TRANSCRIPTION FACTOR AFX1 |
| 138 | Hs.199067 | | | ERBB3: ERBB 3 RECEPTOR PROTEIN |
| | | | | TYROSINE KINASE PRECURSOR (EC |
| | | | | 2.7.1.112) (TYROSINE KINASE TYPE CELL |
| | | | | SURFACE RECEPTOR HER3) |
| 139 | Hs.182937 | | | CYCLOPHILIN 1 PEPTIDYL PROLYL CIS |
| | | | | TRANS ISOMERASE A (EC 5.2.1.8) |
| | | | | (PPIASE) (ROTAMASE) (CYCLOPHILIN A) |
| | | | | (CYCLOSPORIN A BINDING PROTEIN) |
| | | | | {PPIA OR CYPA} |
| 140 | Hs.178658 | | | RAD23B: (RAD23B) UV EXCISION REPAIR |
| | | | | PROTEIN PROTEIN RAD23 HOMOLOG B |
| | | | | (HHR23B) (XP C REPAIR |
| | | | | COMPLEMENTING COMPLEX 58 KDA |
| | | | | PROTEIN) (P58). |
| 141 | Hs.155543 | | | PSMD7: (PSMD7 OR MOV34L) 26S |
| | | | | PROTEASOME REGULATORY SUBUNIT |
| | | | | S12 (MOV34 PROTEIN). |
| 142 | Hs.15071 | | | CCT8: (CCT8 OR CCTQ) T COMPLEX |
| | | | | PROTEIN 1, THETA SUBUNIT (TCP 1 |
| | | | | THETA) (CCT THETA) (KIAA0002). |
| 143 | Hs.149923 | | | XBP1: (XBP1 OR XBP2 OR TREB5) X BOX |
| | | | | BINDING PROTEIN 1 |
| 144 | Hs.142258 | | | STAT3: (STAT3 OR APRF) SIGNAL |
| | | | | TRANSDUCER AND ACTIVATOR OF |
| | | | | TRANSCRIPTION 3 (ACUTE PHASE |
| | | | | RESPONSE FACTOR) |
| 145 | Hs.129943 | | | KIAA0545 |
| 146 | Hs.12068 | | | CACP: CARNITINE O |
| | | | | ACETYLTRANSFERASE (EC 2.3.1.7) |
| | | | | (CARNITINE ACETYLASE)(CAT) |
| | | | | (FRAGMENT) |
| 147 | Hs.118174 | | | TPRD: TETRATRICOPEPTIDREPEAT |
| | | | | PROTEIN |
| 148 | Hs.11669 | | | LAMININ ALPHA 5 |
| 149 | Hs.1119 | | | NR4A1: (NR4A1 OR HMR OR NAK1 OR |
| | | | | GFRP1) ORPHAN NUCLEAR RECEPTOR |
| | | | | HMR (EARLY RESPONSE PROTEIN NAK1) |
| | | | | (TR3 ORPHAN RECEPTOR) |
| 150 | Hs.108809 | | | CCT7: (CCT7 OR CCTH OR NIP7 1) T |
| | | | | COMPLEX PROTEIN 1, ETA SUBUNIT |
| | | | | (TCP 1 ETA) (CCT ETA) (HIV 1 NEF |
| | | | | INTERACTING PROTEIN). |
| 151 | Hs.106070 | | | CDKN1C: (CDKN1C OR KIP2) CYCLIN |
| | | | | DEPENDENT KINASE INHIBITOR 1C |
| | | | | (CYCLIN DEPENDENT KINASE INHIBITOR |
| | | | | P57) (P57KIP2). |
| 152 | Hs.738 | | | EGR1: (EGR1 OR ZNF225) EARLY |
| | | | | GROWTH RESPONSE PROTEIN 1 (EGR 1) |
| | | | | (KROX24) (ZIF268) (TRANSCRIPTION |
| | | | | FACTOR ETR103) (ZINC FINGER PROTEIN |
| | | | | 225) (AT225). |
| 153 | Hs.250723 | | | FRAP: (FRAP) FKBP RAPAMYCIN |
| | | | | ASSOCIATED PROTEIN (FRAP) |
| | | | | (RAPAMYCIN TARGET |
| 154 | Hs.226795 | | | GSTP1 GLUTATHIONE S TRANSFERASE, |
| | | | | PI FORM |
| 155 | Hs.82646 | | | HSPF1: (HSPF1 OR DNAJ1 OR HDJ1) |
| | | | | HEAT SHOCK 40 KDA PROTEIN 1 (HEAT |
| | | | | SHOCK PROTEIN 40) (HSP40) (DNAJ |
| | | | | PROTEIN HOMOLOG 1) (HDJ 1). |
| 156 | Hs.153179 | | | FABE: FATTY ACID BINDING PROTEIN, |
| | | | | EPIDERMAL (E FABP) (PSORIASIS |
| | | | | ASSOCIATED FATTY ACID BINDING |
| | | | | PROTEIN HOMOLOG) (PA FABP)(FABP5) |
| 157 | Hs.99936 | | | KRT10: (KRT10) KERATIN, TYPE I |
| | | | | CYTOSKELETAL 10 (CYTOKERATIN 10) |
| | | | | (K10) |
| 158 | Hs.149609 | | | CD49E/INTEGRIN ALPHA 5 |
| 159 | Hs.9736 | | | PSMD3: (PSMD3) 26S PROTEASOME |
| | | | | REGULATORY SUBUNIT S3 |
| | | | | (PROTEASOME SUBUNIT P58). |
| 160 | Hs.97203 | | | CCL22: SY22_HUMAN (SCYA22 OR MDC |
| | | | | OR A 152E5.1) SMALL INDUCIBLE |
| | | | | CYTOKINE A22 PRECURSOR |
| | | | | (MACROPHAGE DERIVED CHEMOKINE) |
| | | | | (STIMULATED T CELL CHEMOTACTIC |
| | | | | PROTEIN 1) (CC CHEMOKINE STCP 1) |
| 161 | Hs.89466 | | | D3D2: 3,2 TRANS ENOYL COA |
| | | | | ISOMERASE, MITOCHONDRIAL |
| | | | | PRECURSOR (EC 5.3.3.8)(DODECENOYL |
| | | | | COA DELTA ISOMERASE) |
| 162 | Hs.87539 | | | ALDH8: (ALDH8) ALDEHYDE |
| | | | | DEHYDROGENASE 8 (EC 1.2.1.5). |
| 163 | Hs.75424 | | | ID1: (ID1 OR ID) DNA BINDING PROTEIN |
| | | | | INHIBITOR ID 1 (ID) |
| 164 | Hs.75379 | | | EAT1 (SLC1A3 OR EAAT1) EXCITATORY |
| | | | | AMINO ACID TRANSPORTER 1 (SODIUM |
| | | | | DEPENDENT GLUTAMATE/ASPARTATE |
| | | | | TRANSPORTER 1) (GLIAL GLUTAMATE |
| | | | | TRANSPORTER) (GLAST1) |
| 165 | Hs.75212 | | | ODC1: (ODC1) ORNITHINE |
| | | | | DECARBOXYLASE (EC 4.1.1.17) (ODC). |
| 166 | Hs.74631 | | | CD147/BASIGIN |
| 167 | Hs.74619 | | | PSMD2: (PSMD2 OR TRAP2) 26S |
| | | | | PROTEASOME REGULATORY SUBUNIT |
| | | | | S2 (P97) (TUMOR NECROSIS FACTOR |
| | | | | TYPE 1 RECEPTOR ASSOCIATED |
| | | | | PROTEIN 2). |
| 168 | Hs.724 | | | NR1D1: (NR1D1 OR THRAL OR EAR1 OR |
| | | | | HREV) ORPHAN NUCLEAR RECEPTOR |
| | | | | NR1D1 (V ERBA RELATED PROTEIN EAR |
| | | | | 1) (REV ERBA ALPHA). |
| 169 | Hs.63290 | | | 2HPCL: 2 HYDROXYPHYTANOYL COA |
| | | | | LYASE |
| 170 | Hs.6216 | | | TID1: (TID1 OR TID 1) TUMOROUS |
| | | | | IMAGINAL DISCS HOMOLOG |
| | | | | PRECURSOR (HTID 1). |
| 171 | Hs.56874 | | | CVHSP: (CVHSP) CARDIOVASCULAR |
| | | | | HEAT SHOCK PROTEIN. |
| 172 | Hs.5566 | | | CXB2 GAP JUNCTION BETA 2 PROTEIN |
| | | | | (CONNEXIN 26) (CX26) |
| 173 | Hs.52081 | | | KIAA0867: (KIAA0867) KIAA0867 PROTEIN |
| 174 | Hs.206097 | | | RRAS2 |
| 175 | Hs.19545 | | | FZD4 FRIZZLED HOMOLOG 4 |
| | | | | (TRANSMEMBRANE RECEPTOR) |
| 176 | Hs.182423 | | | KNP I: ES1 PROTEIN HOMOLOG, |
| | | | | MITOCHONDRIAL [PRECURSOR] |
| | | | | PROTEIN KNP I GT335 HES1 |
| 177 | Hs.181373 | | | CDM: CDM PROTEIN (6C6 AG TUMOR |
| | | | | ASSOCIATED ANTIGEN) (DXS1357E) |
| | | | | BAP31 |
| 178 | Hs.172674 | | | NFATX4: (A 67A1.1) TRANSCRIPTION |
| | | | | FACTOR NFATX4. |
| 179 | Hs.1708 | | | CCT3: (CCT3 OR CCTG OR TRIC5) T |
| | | | | COMPLEX PROTEIN 1, GAMMA SUBUNIT |
| | | | | (TCP 1 GAMMA) (CCT GAMMA). |
| 180 | Hs.169610 | | | CD44 |
| 181 | Hs.156007 | | | ZAKI4 THYROID HORMONE RESPONSIVE |
| | | | | GENE ZAKI 4 |
| 182 | Hs.155402 | | | DBP: D BINDING PROTEIN (DBP) |
| 183 | Hs.13456 | | | OASIS: (OASIS) OASIS PROTEIN, BZIP |
| | | | | TRANSCRIPTION FACTOR |
| 184 | Hs.119663 | | | CD59 |
| 185 | Hs.111039 | | | NMYRIST N MYRISTOYLTRANSFERASE 1 |
| | | | | MRNA |
| 186 | Hs.78575 | | | SGP1: SULFATED GLYCOPROTEIN 1, |
| | | | | PROSAPOSIN |
| 187 | Hs.79432 | | | FBN2, FIBRILLIN 2 PRECURSOR |
| 188 | Hs.118162 | | | FN, FIBRONECTIN PRECURSOR (FN) |
| | | | | (FRAGMENTS). |
| 189 | Hs.80828 | | | Keratin 1 |
| 190 | | | XM_008578 | Keratin 14 |
| 191 | | X58531 | | LAMININ ALPHA 1 |
| 192 | Hs.75279 | | | LAMININ ALPHA 2 (MEROSIN) |
| 193 | Hs.83450 | | | LAMININ ALPHA 3 (EPILIGRIN 170 KD |
| | | | | SUBUNIT) |
| 194 | Hs.78672 | | | LAMININ ALPHA 4 |
| 195 | Hs.82124 | | | LAMININ BETA 1 |
| 196 | Hs.90291 | | | LAMININ BETA 2 |
| 197 | Hs.75517 | | | LAMININ BETA 3 |
| 198 | Hs.214982 | | | LAMININ GAMMA 1 |
| 199 | Hs.69954 | | | LAMININ GAMMA 3 LAMC3 |
| 200 | Hs.83169 | | | MMP1, INTERSTITIAL COLLAGENASE |
| 201 | Hs.1695 | | | MMP12, MACROPHAGE |
| | | | | METALLOELASTASE PRECURSOR |
| | | | | (MATRIX METALLOPROTEINASE 12) |
| 202 | Hs.99863 | | | NELASTASE: NEUTROPHIL ELASTASE |
| 203 | Hs.62041 | | | NIDOGEN, NIDOGEN PRECURSOR |
| | | | | (ENTACTIN) |
| 204 | Hs.76152 | | | PGS2, BONE PROTEOGLYCAN II |
| | | | | PRECURSOR (PG S2) (DECORIN) (PG40) |
| 205 | Hs.177781 | | | SOD2: SUPEROXIDE DISMUTASE [MN], |
| | | | | MITOCHONDRIAL PRECURSOR (EC |
| | | | | 1.15.1.1) |
| 206 | Hs.5831 | | | TIMP1, METALLOPROTEINASE INHIBITOR |
| | | | | 1 PRECURSOR (TIMP 1) |
| 207 | Hs.6441 | | | TIMP2, METALLOPROTEINASE INHIBITOR |
| | | | | 2 PRECURSOR (TIMP 2) (TISSUE |
| | | | | INHIBITOR OF METALLOPROTEINASES 2) |
| 208 | Hs.190787 | | | TIMP4 |
| | | P12718 | | (ACTG2 OR ACTA3 OR ACTSG) ACTIN, |
| | | | | GAMMA-ENTERIC SMOOTH MUSCLE |
| | | | | (ALPHA-ACTIN 3). |
| 209 | | P20290 | | (BTF3) TRANSCRIPTION FACTOR BTF3 |
| | | | | (RNA POLYMERASE B TRANSCRIPTION |
| | | | | FACTOR 3). |
| 210 | | P40121 | | (CAPG OR MCP) MACROPHAGE CAPPING |
| | | | | PROTEIN (ACTIN-REGULATORY PROTEIN |
| | | | | CAP-G). |
| 211 | | Q9UMW5 | | (CCAM) CEREBRAL CELL ADHESION |
| | | | | MOLECULE (RELATED TO LYSINE |
| | | | | OXIDASES). |
| 212 | | P12277 | | (CKB OR CKBB) CREATINE KINASE, B |
| | | | | CHAIN (EC 2.7.3.2) (B-CK). |
| 213 | | P51911 | | (CNN1) CALPONIN H1, SMOOTH MUSCLE |
| | | | | (BASIC CALPONIN) (CALPONIN 1). |
| 214 | | Q99439 | | (CNN2) CALPONIN H2, SMOOTH MUSCLE |
| | | | | (NEUTRAL CALPONIN). |
| 215 | | P21291 | | (CSRP1 OR CSRP OR CYRP) CYSTEINE- |
| | | | | RICH PROTEIN 1 (CRP1) (CRP). |
| 216 | | O00571 | | (DDX3 OR DBX) DEAD-BOX PROTEIN 3 |
| | | | | (HELICASE-LIKE PROTEIN 2) (HLP2) |
| | | | | (DEAD-BOX, X ISOFORM). |
| 217 | | | AL137555 | unknown cDNA DKFZp434H0820 (from |
| | | | | clone DKFZp434H0820). |
| 218 | | Q08554 | | (DSC1) DESMOCOLLIN 1A/1B |
| | | | | PRECURSOR (DESMOSOMAL |
| | | | | GLYCOPROTEIN 2/3) (DG2/DG3). |
| 219 | | Q15056 | | (EIF4H OR WBSCR1 OR WSCR1) |
| | | | | EUKARYOTIC TRANSLATION INITIATION |
| | | | | FACTOR 4H (EIF-4H) (KIAA0038). |
| 220 | | O15197 | | (EPHB6) EPHRIN TYPE-B RECEPTOR 6 |
| | | | | PRECURSOR (TYROSINE-PROTEIN |
| | | | | KINASE-DEFECTIVE RECEPTOR EPH-6) |
| | | | | (HEP). |
| 221 | | P22607 | | (FGFR3 OR JTK4) FIBROBLAST GROWTH |
| | | | | FACTOR RECEPTOR 3 PRECURSOR |
| | | | | (FGFR-3) (EC 2.7.1.112). |
| 222 | | P21333 | | (FLN1 OR FLN) ENDOTHELIAL ACTIN- |
| | | | | BINDING PROTEIN (ABP-280) |
| | | | | (NONMUSCLE FILAMIN) (FILAMIN 1). |
| 223 | | P01100 | | (FOS) P55-C-FOS PROTO-ONCOGENE |
| | | | | PROTEIN (CELLULAR ONCOGENE C-FOS) |
| | | | | (G0S7 PROTEIN). |
| 224 | | Q16186 | | (GP110) 110 KDA CELL MEMBRANE |
| | | | | GLYCOPROTEIN. |
| 225 | | P22352 | | (GPX3 OR GPXP) PLASMA GLUTATHIONE |
| | | | | PEROXIDASE PRECURSOR (EC 1.11.1.9) |
| | | | | (GSHPX-P). |
| 226 | | O95819 | | (HGK) HPK/GCK-LIKE KINASE HGK. |
| 227 | | O75166 | | (KIAA0679) KIAA0679 PROTEIN |
| | | | | (FRAGMENT). |
| 228 | | O94979 | | (KIAA0905) KIAA0905 PROTEIN (SEC31 |
| | | | | PROTEIN). |
| 229 | | Q9Y2J6 | | (KIAA0992) KIAA0992 PROTEIN |
| | | | | (FRAGMENT). |
| 230 | | P33176 | | (KIF5B OR KNS1 OR KNS) KINESIN HEAVY |
| | | | | CHAIN (UBIQUITOUS KINESIN HEAVY |
| | | | | CHAIN) (UKHC). |
| 231 | | O00301 | | (KSRP) KSRP. |
| 232 | | Q15012 | | (MTRP) GOLGI 4-TRANSMEMBRANE |
| | | | | SPANNING TRANSPORTER MTP |
| | | | | (KIAA0108). |
| 233 | | P35749 | | (MYH11) MYOSIN HEAVY CHAIN, |
| | | | | SMOOTH MUSCLE ISOFORM (SMMHC) |
| | | | | (FRAGMENT). |
| 234 | | Q15746 | | (MYLK OR MLCK) MYOSIN LIGHT CHAIN |
| | | | | KINASE, SMOOTH MUSCLE AND NON- |
| | | | | MUSCLE ISOZYMES (EC 2.7.1.117) (MLCK) |
| 235 | | Q9UGL9 | | (NICE-1) NICE-1 PROTEIN. |
| 236 | | P30086 | | (PEBP OR PBP) |
| | | | | PHOSPHATIDYLETHANOLAMINE-BINDING |
| | | | | PROTEIN (PEBP) (NEUROPOLYPEPTIDE |
| | | | | H3). |
| 237 | | P36955 | | (PEDF) PIGMENT EPITHELIUM-DERIVED |
| | | | | FACTOR PRECURSOR (PEDF) (EPC-1). |
| 238 | | P14786 | | (PKM2 OR PKM) PYRUVATE KINASE, M2 |
| | | | | ISOZYME (EC 2.7.1.40). |
| 239 | | P41222 | | (PTGDS OR PDS) PROSTAGLANDIN-H2 D- |
| | | | | ISOMERASE PRECURSOR (EC 5.3.99.2) |
| | | | | (PROSTAGLANDIN-D SYNTHASE) |
| | | | | (GLUTATHIONE-INDEPENDENT PGD |
| | | | | SYNTHETASE) (PROSTAGLANDIN D2 |
| | | | | SYNTHASE) (PGD2 SYNTHASE) (PGDS2) |
| | | | | (PGDS) (BETA-TRACE PROTEIN). |
| 240 | | | AL365373 | hypothetical protein R33729_1 |
| 241 | | P39030 | | (RPL15) 60S RIBOSOMAL PROTEIN L15. |
| 242 | | P31151 | | (S100A7 OR PSOR1) S100 CALCIUM- |
| | | | | BINDING PROTEIN A7 (PSORIASIN). |
| 243 | | O14778 | | (SARP1) SECRETED APOPTOSIS |
| | | | | RELATED PROTEIN 1 (FRAGMENT). |
| 244 | | P31947 | | (SFN OR HME1) 14-3-3 PROTEIN SIGMA |
| | | | | (STRATIFIN) (EPITHELIAL CELL MARKER |
| | | | | PROTEIN 1). |
| 245 | | O75368 | | (SH3BGRL) SH3 DOMAIN BINDING |
| | | | | GLUTAMIC ACID-RICH-LIKE PROTEIN. |
| 246 | | P11166 | | (SLC2A1 OR GLUT1) GLUCOSE |
| | | | | TRANSPORTER TYPE 1, |
| | | | | ERYTHROCYTE/BRAIN. |
| 247 | | P37108 | | (SRP14) SIGNAL RECOGNITION |
| | | | | PARTICLE 14 KDA PROTEIN (SRP14) (18 KDA |
| | | | | ALU RNA BINDING PROTEIN). |
| 248 | | Q01995 | | (TAGLN OR SM22 OR WS3-10) |
| | | | | TRANSGELIN (SMOOTH MUSCLE |
| | | | | PROTEIN 22-ALPHA) (SM22-ALPHA) (WS3- |
| | | | | 10) (22 KDA ACTIN-BINDING PROTEIN). |
| 249 | | P11387 | | (TOP1) DNA TOPOISOMERASE I (EC |
| | | | | 5.99.1.2). |
| 250 | | P07951 | | (TPM2 OR TMSB) TROPOMYOSIN BETA |
| | | | | CHAIN, SKELETAL MUSCLE. |
| 251 | | P07919 | | (UQCRH) UBIQUINOL-CYTOCHROME C |
| | | | | REDUCTASE COMPLEX 11 KDA PROTEIN |
| | | | | PRECURSOR (EC 1.10.2.2) |
| | | | | (MITOCHONDRIAL HINGE PROTEIN) |
| | | | | (CYTOCHROME C1, NONHEME 11 KDA |
| | | | | PROTEIN) (COMPLEX III SUBUNIT VIII). |
| 252 | | P15311 | | (VIL2) EZRIN (P81) (CYTOVILLIN) (VILLIN- |
| | | | | 2). |
| 253 | | P31946 | | (YWHAB) 14-3-3 PROTEIN BETA/ALPHA |
| | | | | (PROTEIN KINASE C INHIBITOR PROTEIN- |
| | | | | 1) (KCIP-1) (PROTEIN 1054). |

TABLE 8

| | UniGene | | |
|---|---|---|---|
| | accession | SWISSPROT | |
| No.: | No. | or TREMBL | Name of gene/Description |
| 1 | | | - ESTB2.2: PTD014 (C15ORF3). |
| 2 | Hs.63290 | Q9UJ83 | 2HPCL: 2-HYDROXYPHYTANOYL-COA LYASE. |
| 3 | Hs.195851 | P03996 | ACTA2: (ACTA2 OR ACTSA OR ACTVS) AORTIC |
| | | | SMOOTH MUSCLE (ALPHA-ACTIN 2). |
| 4 | Hs.239663 | Q13720 | AFX1: (AFX1 OR AFX OR MLLT7) PUTATIVE FORK HEAD |
| | | | DOMAIN TRANSCRIPTION FACTOR AFX1. |
| 5 | Hs.87539 | P48448 | ALDH8: (ALDH8) ALDEHYDE DEHYDROGENASE 8 (EC |
| | | | 1.2.1.5). |
| 6 | Hs.79172 | P05141 | ANT2: (SLC25A5 OR ANT2) ADP, ATP CARRIER |
| | | | PROTEIN, FIBROBLAST ISOFORM (ADP/ATP |
| | | | TRANSLOCASE 2) (ADENINE NUCLEOTIDE |
| | | | TRANSLOCATOR 2) (ANT 2). |
| 7 | Hs.78225 | P04083 | ANX1: (ANXA1 OR ANX1 OR LPC1) ANNEXIN I |
| | | | (LIPOCORTIN I) (CALPACTIN II) (CHROMOBINDIN 9) |
| | | | (P35)(PHOSPHOLIPASE A2 INHIBITORY PROTEIN). |
| 8 | Hs.182778 | P02654 | APC1: (APOC1) APOLIPOPROTEIN C-I PRECURSOR |
| | | | (APO-C1). |
| 9 | Hs.75736 | P05090 | APD: (APOD) APOLIPOPROTEIN D PRECURSOR. |
| 10 | Hs.177486 | P05067 | APP: (APP OR A4 OR CVAP OR AD1) ALZHEIMER'S |
| | | | DISEASE AMYLOID A4 PROTEIN PRECURSOR |
| | | | (PROTEASE NEXIN-II) (PN-II) (APPI) [CONTAINS: BETA- |
| | | | AMYLOID PROTEIN (BETA-APP) (A-BETA)]. |
| 11 | Hs.155101 | P25705 | ATP5A1: (ATP5A1) ATP SYNTHASE ALPHA CHAIN, |
| | | | MITOCHONDRIAL PRECURSOR (EC 3.6.1.34). |
| 12 | Hs.155433 | P36542 | ATP5C1: (ATP5C1 OR ATP5C) ATP SYNTHASE GAMMA |
| | | | CHAIN, MITOCHONDRIAL PRECURSOR (EC 3.6.1.34). |
| 13 | Hs.80986 | P05496 | ATP5G1: (ATP5G1) ATP SYNTHASE LIPID-BINDING |
| | | | PROTEIN P1 PRECURSOR (EC 3.6.1.34) (ATPASE |
| | | | PROTEIN 9) (SUBUNIT C). |
| 14 | Hs.429 | P48201 | ATP5G3: (ATP5G3) ATP SYNTHASE LIPID-BINDING |
| | | | PROTEIN P3 PRECURSOR (EC 3.6.1.34) (ATPASE |
| | | | PROTEIN 9) (SUBUNIT C). |
| 15 | Hs.79516 | P80723 | BASP1: (BASP1 OR NAP22) BRAIN ACID SOLUBLE |
| | | | PROTEIN 1 (BASP1 PROTEIN) (NEURONAL AXONAL |
| | | | MEMBRANE PROTEIN NAP-22) (NEURONAL TISSUE- |
| | | | ENRICHED ACIDIC PROTEIN). |
| 16 | Hs.229405 | Q9UIF9 | BAZ2B2: (BAZ2B) BROMODOMAIN ADJACENT TO ZINC |
| | | | FINGER DOMAIN 2B KIAA0314 |
| 17 | Hs.75410 | P11021 | BIP: (HSPA5 OR GRP78) 78 KDA GLUCOSE-REGULATED |
| | | | PROTEIN PRECURSOR (GRP 78) (IMMUNOGLOBULIN |
| | | | HEAVY CHAIN BINDING PROTEIN) (BIP). |
| 18 | Hs.6101 | P22004 | BMP6: (BMP6 OR BMP-6 OR VGR1) BONE |
| | | | MORPHOGENETIC PROTEIN 6 PRECURSOR (BMP 6). |
| 19 | Hs.74631 | P35613 | BSG: (BSG) BASIGIN PRECURSOR (LEUKOCYTE |
| | | | ACTIVATION ANTIGEN M6) (COLLAGENASE |
| | | | STIMULATORY FACTOR) (EXTRACELLULAR MATRIX |
| | | | METALLOPROTEINASE INDUCER) (EMMPRIN) (5F7) |
| | | | (CD147 ANTIGEN). |
| 20 | Hs.77054 | P31607 | BTG1: (BTG1) BTG1 PROTEIN (B-CELL |
| | | | TRANSLOCATION GENE 1 PROTEIN). |
| 21 | | P01024 | C3: (C3) COMPLEMENT C3 PRECURSOR |
| 22 | Hs.12068 | P43155 | CACP: (CRAT OR CAT1) CARNITINE O- |
| | | | ACETYLTRANSFERASE (EC 2.3.1.7) (CARNITINE |
| | | | ACETYLASE) (CAT) (FRAGMENT). |
| 23 | Hs.155560 | P27824 | CANX: (CANX) CALNEXIN PRECURSOR (MAJOR |
| | | | HISTOCOMPATIBILITY COMPLEX CLASS I ANTIGEN- |
| | | | BINDING PROTEIN P88) (P90) (IP90). |
| 24 | Hs.167835 | Q15067 | CAOP: (ACOX1 OR ACOX) ACYL-COENZYME A |
| | | | OXIDASE 1, PEROXISOMAL (EC 1.3.3.6) (PALMITOYL- |
| | | | COAOXIDASE) (AOX) MUSPAOX. |
| 25 | Hs.75360 | P16870 | CBPH: (CPE) CARBOXYPEPTIDASE H PRECURSOR (EC |
| | | | 3.4.17.10) (CPH) (CARBOXYPEPTIDASE E) (CPE) |
| | | | (ENKEPHALIN CONVERTASE) (PROHORMONE |
| | | | PROCESSING CARBOXYPEPTIDASE). |
| 26 | Hs.340 | P13500 | CCL2_HUMAN: (SCYA2 OR MCP1) SMALL INDUCIBLE |
| | | | CYTOKINE A2 PRECURSOR (MONOCYTE |
| | | | CHEMOTACTIC PROTEIN 1) (MCP-1) (MONOCYTE |
| | | | CHEMOATTRACTANT PROTEIN-1) (MONOCYTE |
| | | | CHEMOTACTIC AND ACTIVATING FACTOR) (MCAF) |
| | | | (MONOCYTE SECRETORY PROTEIN JE) (HC11). |
| 27 | Hs.97203 | O00626 | CCL22: (SCYA22 OR MDC OR A-152E5.1) SMALL |
| | | | INDUCIBLE CYTOKINE A22 PRECURSOR |
| | | | (MACROPHAGE-DERIVED CHEMOKINE) (STIMULATED |
| | | | T CELL CHEMOTACTIC PROTEIN 1) (CC CHEMOKINE |
| | | | STCP-1). |
| 28 | Hs.46468 | P51684 | CCR6: (CCR6 OR CMKBR6 OR STRL22 OR GPR29 OR |
| | | | CKRL3) C-C CHEMOKINE RECEPTOR TYPE 6 (C-C CKR- |
| | | | 6) (CC-CKR-6) (CCR-6) (LARC RECEPTOR) (GPR-CY4) |
| | | | (GPRCY4) (CHEMOKINE RECEPTOR-LIKE 3) (CKR-L3) |
| | | | (DRY6). |
| 29 | Hs.1708 | P49368 | CCT3: (CCT3 OR CCTG OR TRIC5) T-COMPLEX |
| | | | PROTEIN 1, GAMMA SUBUNIT (TCP-1-GAMMA) (CCT- |
| | | | GAMMA). |
| 30 | Hs.108809 | Q99832 | CCT7: (CCT7 OR CCTH OR NIP7-1) T-COMPLEX |
| | | | PROTEIN 1, ETA SUBUNIT (TCP-1-ETA) (CCT-ETA) (HIV-1 |
| | | | NEF INTERACTING PROTEIN). |
| 31 | Hs.15071 | P50990 | CCT8: (CCT8 OR CCTQ) T-COMPLEX PROTEIN 1, |
| | | | THETA SUBUNIT (TCP-1-THETA) (CCT-THETA) |
| | | | (KIAA0002). |
| 32 | Hs.85289 | P28906 | CD34: (CD34) HEMATOPOIETIC PROGENITOR CELL |
| | | | ANTIGEN CD34 PRECURSOR. |
| 33 | Hs.169610 | P16070 | CD44_EX10-12: (CD44 OR LHR) CD44 ANTIGEN |
| | | | PRECURSOR (PHAGOCYTIC GLYCOPROTEIN I)(PGP- |
| | | | 1)(HUTCH-I)(EXTRACELLULAR MATRIX RECEPTOR- |
| | | | III)(GP90 LYMPHOCYTE HOMING/ADHESION |
| | | | RECEPTOR)(HERMES ANTIGEN)(HYALURONATE |
| | | | RECEPTOR)(HEPARAN SULFATE |
| | | | PROTEOGLYCAN)(EPICAN). |
| 34 | Hs.170121 | P08575 | CD45_EX29-31: (PTPRC OR CD45) LEUKOCYTE |
| | | | COMMON ANTIGEN PRECURSOR (EC 3.1.3.48) (L-CA) |
| | | | (CD45 ANTIGEN) (T200). |
| 35 | Hs.119663 | P13987 | CD59: (CD59) CD59 GLYCOPROTEIN PRECURSOR |
| | | | (MEMBRANE ATTACK COMPLEX INHIBITION FACTOR) |
| | | | (MACIF) (MAC-INHIBITORY PROTEIN) (MAC-IP) (MEM43 |
| | | | ANTIGEN) (PROTECTIN) (MEMBRANE INHIBITOR OF |
| | | | REACTIVE LYSIS) (MIRL) (HRF-20) (1F5 ANTIGEN). |
| 36 | Hs.54457 | P18582 | CD81: (CD81 OR TAPA1) CD81 ANTIGEN (26 KDA CELL |
| | | | SURFACE PROTEIN TAPA-1). |
| 37 | Hs.1244 | P21926 | CD9: (CD9 OR MIC3) CD9 ANTIGEN (P24) (LEUKOCYTE |
| | | | ANTIGEN MIC3) (MOTILITY-RELATED PROTEIN) (MRP- |
| | | | 1). |
| 38 | Hs.106070 | P49918 | CDKN1C: (CDKN1C OR KIP2) CYCLIN-DEPENDENT |
| | | | KINASE INHIBITOR 1C (CYCLIN-DEPENDENT KINASE |
| | | | INHIBITOR P57) (P57KIP2). |
| 39 | Hs.181373 | P51572 | CDM: (BCAP31 OR BAP31) B-CELL RECEPTOR- |
| | | | ASSOCIATED PROTEIN 31 (CDM PROTEIN) (6C6-AG |
| | | | TUMOR-ASSOCIATED ANTIGEN) (DXS1357E). |
| 40 | Hs.119475 | Q14011 | CIRBP: (CIRBP OR CIRP OR A18HNRNP) COLD- |
| | | | INDUCIBLE RNA-BINDING PROTEIN (GLYCINE-RICH |
| | | | RNA-BINDING PROTEIN CIRP) (A18 HNRNP). |
| 41 | Hs.79070 | P01107 | CMYC: (MYC) MYC PROTO-ONCOGENE PROTEIN (C- |
| | | | MYC). |
| 42 | Hs.78409 | P39060 | COL18A1_2: (COL18A1) COLLAGEN ALPHA 1(XVIII) |
| | | | CHAIN [CONTAINS: ENDOSTATIN]. |
| 43 | Hs.172928 | P02452 | COL1A1: (COL1A1) COLLAGEN ALPHA 1(I) CHAIN |
| | | | PRECURSOR. |
| 44 | Hs.179573 | P08123 | COL1A2: (COL1A2) COLLAGEN ALPHA 2(I) CHAIN |
| | | | PRECURSOR. |
| 45 | Hs.119571 | P02461 | COL3A1: (COL3A1) COLLAGEN ALPHA 1(III) CHAIN |
| | | | PRECURSOR. |
| 46 | Hs.75617 | P08572 | COL4A2: (COL4A2) COLLAGEN ALPHA 2(IV) CHAIN |
| | | | PRECURSOR. |
| 47 | Hs.146428 | P20908 | COL5A1: (COL5A1) PRO-ALPHA-1 TYPE V COLLAGEN. |
| 48 | Hs.181028 | P20674 | COX5A: (COX5A) CYTOCHROME C OXIDASE |
| | | | POLYPEPTIDE VA, MITOCHONDRIAL PRECURSOR (EC |
| | | | 1.9.3.1). |
| 49 | Hs.180714 | P12074 | COX6A1: (COX6A1 OR COX6AL) CYTOCHROME C |
| | | | OXIDASE POLYPEPTIDE VIA-LIVER PRECURSOR (EC |
| | | | 1.9.3.1). |
| 50 | Hs.74649 | P09669 | COX6C: (COX6C) CYTOCHROME C OXIDASE |
| | | | POLYPEPTIDE VIC PRECURSOR (EC 1.9.3.1). |
| 51 | Hs.23598 | Q92793 | CREBBP: (CREBBP OR CBP) CREB-BINDING PROTEIN. |
| 52 | Hs.75511 | P29279 | CTGF: (CTGF) CONNECTIVE TISSUE GROWTH FACTOR |
| | | | PRECURSOR. |
| 53 | Hs.56874 | Q9UBY9 | CVHSP: (CVHSP) CARDIOVASCULAR HEAT SHOCK |
| | | | PROTEIN. |
| 54 | Hs.237356 | P48061 | CXCL12: (SDF1) STROMAL CELL-DERIVED FACTOR 1 |
| | | | PRECURSOR (SDF-1) (PRE-B CELL GROWTH |
| | | | STIMULATING FACTOR) (PBSF) |
| 55 | Hs.182937 | P05092 | CYPA: (PPIA OR CYPA) CYCLOPHILIN 1 PEPTIDYL- |
| | | | PROLYL CIS-TRANS ISOMERASE A (EC 5.2.1.8) |
| | | | (PPIASE) (ROTAMASE) (CYCLOPHILIN A) |
| | | | (CYCLOSPORIN A-BINDING PROTEIN). |
| 56 | Hs.8867 | O00622 | CYR61: (CYR61 OR IGFBP10 OR GIG1) CYR61 PROTEIN |
| | | | PRECURSOR (GIG1 PROTEIN) (INSULIN-LIKE GROWTH |
| | | | FACTOR-BINDING PROTEIN 10). |
| 57 | Hs.89466 | P42126 | D3D2: (DCI) 3,2-TRANS-ENOYL-COA ISOMERASE, |
| | | | MITOCHONDRIAL PRECURSOR (EC 5.3.3.8) |
| | | | (DODECENOYL-COA DELTA-ISOMERASE). |
| 58 | Hs.155402 | Q10586 | DBP: (DBP) D-SITE-BINDING PROTEIN (ALBUMIN D |
| | | | BOX-BINDING PROTEIN) (TAXREB302). |
| 59 | Hs.1139 | P16989 | DBPA: (CSDA OR DBPA) DNA-BINDING PROTEIN A |
| | | | (COLD SHOCK DOMAIN |
| 60 | Hs.171825 | O14503 | DEC1: (BHLHB2 OR SHARP-2 OR STRA14) STIMULATED |
| | | | BY RETINOIC ACID 14 (BASIC-HELIX-LOOP-HELIX |
| | | | PROTEIN) (FRAGMENT) STRA13 (SHARP-2) |
| | | | ENHANCER-OF-SPLIT AND HAIRY-RELATED PROTEIN |
| | | | 2. |
| 61 | | P43003 | EAAT1: (SLC1A3 OR EAAT1) EXCITATORY AMINO ACID |
| | | | TRANSPORTER 1 (SODIUM-DEPENDENT |
| | | | GLUTAMATE/ASPARTATE TRANSPORTER 1) (GLIAL |
| | | | GLUTAMATE TRANSPORTER) (GLAST1) |
| 62 | Hs.738 | P18146 | EGR1: (EGR1 OR ZNF225) EARLY GROWTH RESPONSE |
| | | | PROTEIN 1 (EGR-1) (KROX-24 PROTEIN) (ZIF268) |
| | | | (NERVE GROWTH FACTOR-INDUCED PROTEIN A) |
| | | | (NGFI-A) (TRANSCRIPTION FACTOR ETR103) (ZINC |
| | | | FINGER PROTEIN 225) (AT225). |
| 63 | Hs.106673 | Q64252 | EIF3S6: (EIF3S6 OR INT6) EUKARYOTIC TRANSLATION |
| | | | INITIATION FACTOR 3 SUBUNIT 6 (EIF-3 P48) |
| | | | (MAMMARY TUMOR-ASSOCIATED PROTEIN INT-6) |
| | | | (VIRAL INTEGMOUSEION SITE PROTEIN INT-6). |
| 64 | | P54849 | EMP1: (EMP1 OR TMP OR B4B) EPITHELIAL MEMBRANE |
| | | | PROTEIN-1 (EMP-1) (TUMOR-ASSOCIATED MEMBRANE |
| | | | PROTEIN) (CL-20) (B4B PROTEIN). |
| 65 | Hs.76753 | Q14248 | ENG: (ENG OR END) ENDOGLIN PRECURSOR (CD105 |
| | | | ANTIGEN). |
| 66 | Hs.102948 | Q14250 | ENIGMA: ENIGMA PROTEIN (LIM-DOMAIN PROTEIN |
| | | | LMP-1). |
| 67 | Hs.89649 | P07099 | EPHX1: (EPHX1 OR EPHX OR EPOX) EPOXIDE |
| | | | HYDROLASE (EC 3.3.2.3) (MICROSOMAL EPOXIDE |
| | | | HYDROLASE) (EPOXIDE HYDRATASE). |
| 68 | Hs.173664 | P04626 | ERBB2: (ERBB2 OR HER2 OR NGL OR NEU) RECEPTOR |
| | | | PROTEIN-TYROSINE KINASE ERBB-2 PRECURSOR (EC |
| | | | 2.7.1.112) (P185ERBB2) (NEU PROTO-ONCOGENE) (C- |
| | | | ERBB-2) (TYROSINE KINASE-TYPE CELL SURFACE |
| | | | RECEPTOR HER2) (MLN 19). |
| 69 | Hs.199067 | P21860 | ERBB3: (ERBB3 OR HER3) ERBB-3 RECEPTOR |
| | | | PROTEIN-TYROSINE KINASE PRECURSOR (EC |
| | | | 2.7.1.112) (TYROSINE KINASE-TYPE CELL SURFACE |
| | | | RECEPTOR HER3). |
| 70 | Hs.182429 | Q15084 | ERP5: (CABP1 OR ERP5) PROBABLE PROTEIN |
| | | | DISULFIDE ISOMERASE P5 PRECURSOR (EC 5.3.4.1). |
| 71 | Hs.75334 | Q93063 | EXT2: (EXT2) EXOSTOSIN-2 (PUTATIVE TUMOR |
| | | | SUPPRESSOR PROTEIN EXT2) (MULTIPLE EXOSTOSES |
| | | | PROTEIN 2). |
| 72 | Hs.153179 | Q01469 | FABE: (FABP5) FATTY ACID-BINDING PROTEIN, |
| | | | EPIDERMAL (E-FABP) (PSORIASIS-ASSOCIATED FATTY |
| | | | ACID-BINDING PROTEIN HOMOLOG) (PA-FABP). |
| 73 | Hs.83190 | P49327 | “FAS: (FASN OR FAS) FATTY ACID SYNTHASE (EC |
| | | | 2.3.1.85) [INCLUDES: EC 2.3.1.38; EC 2.3.1.39; EC |
| | | | 2.3.1.41; EC 1.1.1.100; EC 4.2.1.61; EC 1.3.1.10; EC |
| | | | 3.1.2.14].” |
| 74 | Hs.69745 | P22570 | FDXR: (FDXR OR ADXR) NADPH: ADRENODOXIN |
| | | | OXIDOREDUCTASE PRECURSOR (EC 1.18.1.2) |
| | | | (ADRENODOXIN REDUCTASE) (FERREDOXIN-NADP(+) |
| | | | REDUCTASE). |
| 75 | Hs.75431 | P04469 | FGG: (FGG) FIBRINOGEN GAMMA CHAIN PRECURSOR. |
| 76 | Hs.750 | P35555 | FIBRILLIN1: (FBN1 OR FBN) FIBRILLIN 1 PRECURSOR. |
| 77 | Hs.79432 | P35556 | FIBRILLIN2: (FBN2) FIBRILLIN 2 PRECURSOR. |
| 78 | Hs.230 | Q06828 | FIBROMODULIN: (FMOD OR FM) FIBROMODULIN |
| | | | PRECURSOR (FM) (COLLAGEN-BINDING 59 KDA |
| | | | PROTEIN). |
| 79 | Hs.118162 | P02751 | FIBRONECTIN: (FN1 OR FN) FIBRONECTIN |
| | | | PRECURSOR (FN). |
| 80 | Hs.79732 | P37888 | FIBULIN1: (FBLN1) FIBULIN-1, ISOFORM D |
| | | | PRECURSOR. |
| 81 | Hs.848 | Q02790 | FKBP4: (FKBP4) P59 PROTEIN (HSP BINDING |
| | | | IMMUNOPHILIN) (HBI) (POSSIBLE PEPTIDYL-PROLYL |
| | | | CIS-TRANS ISOMERASE) (EC 5.2.1.8) (PPIASE) |
| | | | (ROTAMASE) (FKBP52 PROTEIN) (52 KDA FK506 |
| | | | BINDING PROTEIN) (P52) (FKBP59) (HSP56). |
| 82 | Hs.8762 | O95302 | FKBP63: (FKBP9 OR FKBP63) FK506-BINDING PROTEIN |
| | | | (FRAGMENT) FKBP9. |
| 83 | Hs.155952 | P42345 | FRAP: (FRAP) FKBP-RAPAMYCIN ASSOCIATED |
| | | | PROTEIN (FRAP) (RAPAMYCIN TARGET PROTEIN). |
| 84 | Hs.105700 | O14877 | FRPHE_1: (SFRP4) SECRETED FRIZZLED-RELATED |
| | | | SEQUENCE PROTEIN 4 FRPHE FRPAP. |
| 85 | Hs.183 | Q16570 | FY: (FY OR GPD OR DARC) DUFFY ANTIGEN (FY |
| | | | GLYCOPROTEIN) (GLYCOPROTEIN D) (GPFY) |
| 86 | Hs.19545 | Q9ULV1 | FZD4: (FZD4) WNT RECEPTOR FRIZZLED-4. |
| 87 | Hs.197345 | P12956 | G22P1: (G22P1) ATP-DEPENDENT DNA HELICASE II, 70 |
| | | | KDA SUBUNIT (LUPUS KU AUTOANTIGEN PROTEIN |
| | | | P70) (KU70) (70 KDA SUBUNIT OF KU ANTIGEN) |
| | | | (THYROID-LUPUS AUTOANTIGEN) (TLAA) (CTC BOX |
| | | | BINDING FACTOR 75 KDA SUBUNIT) (CTCBF) (CTC75). |
| 88 | Hs.227751 | P09382 | GALECTIN-1: (LGALS1) GALECTIN-1 (BETA- |
| | | | GALACTOSIDE-BINDING LECTIN L-14-I) (LACTOSE- |
| | | | BINDING LECTIN 1) (S-LAC LECTIN 1) (GALAPTIN) (14 KDA |
| | | | LECTIN) (HPL) (HBL). |
| 89 | Hs.169476 | P04406 | GAPD: (GAPD) (GAPDH) GLYCERALDEHYDE 3- |
| | | | PHOSPHATE DEHYDROGENASE, LIVER (EC 1.2.1.12). |
| 90 | Hs.9194 | Q9UMW7 | GBDR1: (GBDR1) PUTATIVE GLIALBLASTOMA CELL |
| | | | DIFFERENTIATION-RELATED PROTEIN. |
| 91 | Hs.74471 | P17302 | GJA1_2: (GJA1) GAP JUNCTION ALPHA-1 PROTEIN |
| | | | (CONNEXIN 43) (CX43) (GAP JUNCTION 43 KDA HEART |
| | | | PROTEIN). |
| 92 | | P29033 | GJB2: (GJB2) GAP JUNCTION BETA-2 PROTEIN |
| | | | (CONNEXIN 26) (CX26) |
| 93 | Hs.77508 | P49448 | GLUDP1: (GLUD2 OR GLUDP1) GLUTAMATE |
| | | | DEHYDROGENASE 2 PRECURSOR (EC 1.4.1.3) (GDH). |
| 94 | Hs.170171 | P15104 | GLUL: (GLUL OR GLNS) GLUTAMINE SYNTHETASE (EC |
| | | | 6.3.1.2) (GLUTAMATE —AMMONIA LIGASE). |
| 95 | Hs.76686 | P07203 | GPX1: (GPX1) GLUTATHIONE PEROXIDASE (EC |
| | | | 1.11.1.9) (GSHPX-1) (CELLULAR GLUTATHIONE |
| | | | PEROXIDASE). |
| 96 | Hs.226795 | P09211 | GSTP1: (GSTP1 OR GST3) GLUTATHIONE S- |
| | | | TRANSFERASE P (EC 2.5.1.18) (GST CLASS-PI) (GSTP1- |
| | | | 1). |
| 97 | Hs.75445 | Q14515 | HEVIN: (HEVIN) HIGH ENDOTHELIAL VENULE |
| | | | PRECURSOR. (MAST 9) HEVIN-LIKE PROTEIN. |
| 98 | Hs.119222 | P50502 | HIP: (HIP OR ST13 OR P48) HSC70-INTERACTING |
| | | | PROTEIN (PROGESTERONE RECEPTOR-ASSOCIATED |
| | | | P48 PROTEIN) (PUTATIVE TUMOR SUPPRESSOR ST13). |
| 99 | Hs.198427 | P52789 | HK2: (HK2) HEXOKINASE, TYPE II (EC 2.7.1.1) (HK II). |
| 100 | Hs.82314 | P00492 | HPRT: (HPRT1 OR HPRT) HYPOXANTHINE-GUANINE |
| | | | PHOSPHORIBOSYLTRANSFERASE (EC 2.4.2.8) |
| | | | (HGPRT) (HGPRTASE). |
| 101 | Hs.180414 | P11142 | HSC73: (HSPA8 OR HSPA10 OR HSC70 OR HSP73) |
| | | | HEAT SHOCK COGNATE 71 KD PROTEIN. |
| 102 | Hs.180532 | P07900 | HSPCA: (HSPCA OR HSPC1 OR HSP90A) HEAT SHOCK |
| | | | PROTEIN HSP 90-ALPHA (HSP 86). |
| 103 | Hs.82646 | P25685 | HSPF1: (HSPF1 OR DNAJ1 OR HDJ1) HEAT SHOCK 40 KDA |
| | | | PROTEIN 1 (HEAT SHOCK PROTEIN 40) (HSP40) |
| | | | DNAJ PROTEIN HOMOLOG 1) (HDJ-1). |
| 104 | Hs.81328 | P25963 | IKBA: (NFKBIA OR NFKBI OR MAD3) MAJOR |
| | | | HISTOCOMPATIBILITY COMPLEX ENHANCER-BINDING |
| | | | PROTEIN MAD3 (NUCLEAR FACTOR KAPPA-B |
| | | | INHIBITOR) (I-KAPPA-B-ALPHA) (IKBA). |
| 105 | Hs.83004 | P40222 | IL14: (IL14) INTERLEUKIN-14 PRECURSOR (IL-14) (HIGH |
| | | | MOLECULAR WEIGHT B-CELL GROWTH FACTOR) |
| | | | (HMW-BCGF). |
| 106 | Hs.83077 | Q14116 | IL18: (IL18 OR IGIF) INTERLEUKIN-18 PRECURSOR (IL- |
| | | | 18) (INTERFERON-GAMMA INDUCING FACTOR) (IFN- |
| | | | GAMMA-INDUCING FACTOR) INTERLEUKIN-1 GAMMA) |
| | | | (IL-1 GAMMA). |
| 107 | Hs.1722 | P01583 | IL1A: (IL1A) INTERLEUKIN-1 ALPHA PRECURSOR (IL-1 |
| | | | ALPHA) (HEMATOPOIETIN-1). |
| 108 | Hs.126256 | P01584 | IL1B: (IL1B) INTERLEUKIN-1 BETA PRECURSOR (IL-1 |
| | | | BETA) (CATABOLIN). |
| 109 | Hs.82112 | P14778 | IL1R1: (IL1R1 OR IL1RA OR IL1R) INTERLEUKIN-1 |
| | | | RECEPTOR, TYPE I PRECURSOR (IL-1R-1) (IL-1R- |
| | | | ALPHA) (P80) (ANTIGEN CD121A). |
| 110 | Hs.93913 | P05231 | IL6: (1L6 OR IFNB2) INTERLEUKIN-6 PRECURSOR (IL-6) |
| | | | (B-CELL STIMULATORY FACTOR 2) (BSF-2) |
| | | | (INTERFERON BETA-2) (HYBRIDOMA GROWTH |
| | | | FACTOR). |
| 111 | Hs.624 | P10145 | IL8: (IL8) INTERLEUKIN-8 PRECURSOR (IL-8) |
| | | | (MONOCYTE-DERIVED NEUTROPHIL CHEMOTACTIC |
| | | | FACTOR) (MDNCF) (T-CELL CHEMOTACTIC FACTOR) |
| | | | (NEUTROPHIL-ACTIVATING PROTEIN 1) (NAP-1) |
| | | | (LYMPHOCYTE-DERIVED NEUTROPHIL-ACTIVATING |
| | | | FACTOR) (LYNAP) (PROTEIN 3-10C). |
| 112 | Hs.149846 | P18084 | INTEGRINB5: (ITGB5) INTEGRIN BETA-5 PRECURSOR. |
| 113 | Hs.149609 | P08648 | ITGA5: (ITGA5 OR FNRA) INTEGRIN ALPHA-5 |
| | | | PRECURSOR (FIBRONECTIN RECEPTOR ALPHA |
| | | | SUBUNIT) (INTEGRIN ALPHA-F) (VLA-5) (CD49E). |
| 114 | Hs.172180 | O43166 | KIAA0440: (KIAA0440) KIAA0440 (FRAGMENT). SPA-1 |
| | | | LIKE PROTEIN P1294. PUTATIVE GAP PROTEIN ALPHA. |
| | | | HIGH-RISK HUMAN PAPILLOMA VIRUSES E6 |
| | | | ONCOPROTEINS TARGETED PROTEIN E6TP1 BETA. |
| 115 | Hs.129943 | O60292 | KIAA0545: (KIAA0545) KIAA0545 PROTEIN (FRAGMENT). |
| 116 | Hs.52081 | O94945 | KIAA0867: (KIAA0867) KIAA0867 PROTEIN |
| 117 | Hs.182423 | P30042 | KNP-I: (C21ORF33 OR HES1 OR KNPI) ES1 PROTEIN |
| | | | HOMOLOG, MITOCHONDRIAL PRECURSOR (PROTEIN |
| | | | KNP-I) (GT335 PROTEIN). |
| 118 | Hs.80828 | P04264 | KRT1: (KRT1 OR KRTA) KERATIN, TYPE II |
| | | | CYTOSKELETAL 1 (CYTOKERATIN 1) (K1) (CK 1) (67 KDA |
| | | | CYTOKERATIN) (HAIR ALPHA PROTEIN). |
| 119 | Hs.99936 | P13645 | KRT10: (KRT10) KERATIN, TYPE I CYTOSKELETAL 10 |
| | | | (CYTOKERATIN 10) (K10) (CK 10). |
| 120 | Hs.117729 | P02533 | KRT14: (KRT14) KERATIN, TYPE I CYTOSKELETAL 14 |
| | | | (CYTOKERATIN 14) (K14) (CK 14). |
| 121 | Hs.195850 | P13647 | KRT5: (KRT5) KERATIN, TYPE II CYTOSKELETAL 5 |
| | | | (CYTOKERATIN 5) (K5) (CK 5) (58 KDA CYTOKERATIN). |
| 122 | | P25391 | LAMA1: (LAMA1 OR LAMA) LAMININ ALPHA-1 CHAIN |
| | | | PRECURSOR (LAMININ A CHAIN). |
| 123 | Hs.75279 | Q14736 | LAMA2: (LAMA2 OR LAMM) LAMININ ALPHA-2 CHAIN |
| | | | PRECURSOR (LAMININ M CHAIN) (MEROSIN HEAVY |
| | | | CHAIN). |
| 124 | Hs.83450 | Q16787 | LAMA3: (LAMA3) LAMININ ALPHA-3 CHAIN PRECURSOR |
| | | | (EPILIGRIN 170 KDA SUBUNIT) (E170). |
| 125 | Hs.78672 | Q16363 | LAMA4: (LAMA4) LAMININ ALPHA-4 CHAIN |
| | | | PRECURSOR. |
| 126 | Hs.11669 | O15230 | LAMA5: (KIAA0533 OR LAMA5) KIAA0533 PROTEIN |
| | | | (LAMININ ALPHA 5 CHAIN) (FRAGMENT). |
| 127 | Hs.82124 | P07942 | LAMB1: (LAMB1) LAMININ BETA-1 CHAIN PRECURSOR |
| | | | (LAMININ B1 CHAIN). |
| 128 | Hs.75517 | Q13751 | LAMB3: (LAMB3) LAMININ BETA-3 CHAIN PRECURSOR |
| | | | (LAMININ B1K CHAIN) (KALININ B1 CHAIN). |
| 129 | Hs.214982 | P11047 | LAMG1: (LAMC1 OR LAMB2) LAMININ GAMMA-1 CHAIN |
| | | | PRECURSOR (LAMININ B2 CHAIN). |
| 130 | Hs.69954 | Q9Y6N6 | LAMG3: (LAMC3) LAMININ GAMMA 3 CHAIN |
| | | | PRECURSOR. |
| 131 | Hs.223014 | P36777 | LON: (PRSS15) MITOCHONDRIAL LON PROTEASE |
| | | | HOMOLOG PRECURSOR (EC 3.4.21.—). |
| 132 | Hs.241257 | P22064 | LTBP1: (LTBP1) LATENT TRANSFORMING GROWTH |
| | | | FACTOR BETA BINDING PROTEIN 1 PRECURSOR |
| | | | (TRANSFORMING GROWTH FACTOR BETA-1 BINDING |
| | | | PROTEIN 1) (TGF-BETA1-BP-1). |
| 133 | Hs.23582 | P09758 | M1S1: (M1S1 OR GA733-1 OR TROP2) PANCREATIC |
| | | | CARCINOMA MARKER PROTEIN GA733-1 PRECURSOR |
| | | | (CELL SURFACE GLYCOPROTEIN TROP-2). |
| 134 | Hs.83551 | P55001 | MAGP1: (MFAP2 OR MAGP1) MICROFIBRIL- |
| | | | ASSOCIATED GLYCOPROTEIN PRECURSOR (MAGP) |
| | | | (MAGP-1). |
| 135 | Hs.861 | P27361 | MAPK3: (MAPK3 OR PRKM3 OR ERK1) MITOGEN- |
| | | | ACTIVATED PROTEIN KINASE 3 (EC 2.7.1.—) |
| | | | (EXTRACELLULAR SIGNAL-REGULATED KINASE 1) |
| | | | (ERK-1) (INSULIN-STIMULATED MAP2 KINASE) (MAP |
| | | | KINASE 1) (MAPK 1) (P44-ERK1) (ERT2)(P44- |
| | | | MAPK)(MICROTUBULE-ASSOCIATED PROTEIN-2 |
| | | | KINASE) |
| 136 | Hs.211579 | P43121 | MCAM: (MCAM OR MUC18) CELL SURFACE |
| | | | GLYCOPROTEIN MUC18 PRECURSOR (MELANOMA- |
| | | | ASSOCIATED ANTIGEN MUC18) (MELANOMA- |
| | | | ASSOCIATED ANTIGEN A32) (S-ENDO 1 ENDOTHELIAL- |
| | | | ASSOCIATED ANTIGEN) (CD146 ANTIGEN) (MELANOMA |
| | | | ADHESION MOLECULE). |
| 137 | Hs.77171 | P33992 | MCM5: (MCM5 OR CDC46) DNA REPLICATION |
| | | | LICENSING FACTOR MCM5 (CDC46 HOMOLOG) (P1- |
| | | | CDC46). |
| 138 | Hs.111076 | P40926 | MDH2: (MDH2) MALATE DEHYDROGENASE, |
| | | | MITOCHONDRIAL PRECURSOR (EC 1.1.1.37). |
| 139 | Hs.199160 | Q03164 | MLL: (MLL OR HRX OR ALL1 OR TRX1 OR HTRX) ZINC |
| | | | FINGER PROTEIN HRX (ALL-1) (TRITHORAX-LIKE |
| | | | PROTEIN). |
| 140 | Hs.83169 | P03956 | MMP1: (MMP1 OR CLG) INTERSTITIAL COLLAGENASE |
| | | | PRECURSOR (EC 3.4.24.7) (MATRIX |
| | | | METALLOPROTEINASE-1) (MMP-1) (FIBROBLAST |
| | | | COLLAGENASE). |
| 141 | Hs.1695 | P39900 | MMP12: (MMP12 OR HME) MACROPHAGE |
| | | | METALLOELASTASE PRECURSOR (EC 3.4.24.65) (HME) |
| | | | (MATRIX METALLOPROTEINASE-12) (MMP-12). |
| 142 | Hs.111301 | P08253 | MMP2: (MMP2 OR CLG4A) 72 KDA TYPE IV |
| | | | COLLAGENASE PRECURSOR (EC 3.4.24.24) (72 KDA |
| | | | GELATINASE) (MATRIX METALLOPROTEINASE-2) |
| | | | (MMP-2) (GELATINASE A) (TBE-1). |
| 143 | Hs.184601 | Q01650 | MPE16: (MPE16) INTEGRAL MEMBRANE PROTEIN E16. |
| | | | (HLAT1 OR CD98LC) L-TYPE AMINO ACID |
| | | | TRANSPORTER 1. (4F2 LC) 4F2 LIGHT CHAIN. (SLC7A5). |
| 144 | Hs.73965 | Q01130 | MRF1: (SFRS2) SPLICING FACTOR, ARGININE/SERINE- |
| | | | RICH 2 (SPLICING FACTOR SC35) (SC-35) (SPLICING |
| | | | COMPONENT, 35 KDA) (PR264 PROTEIN). |
| 145 | Hs.25313 | O14742 | MSP58: (MSP58) NUCLEOLAR PROTEIN CELL CYCLE- |
| | | | REGULATED FACTOR P78. |
| 146 | Hs.76941 | P54709 | NA-K-ATPASE-B3: (ATP1B3) SODIUM/POTASSIUM- |
| | | | TRANSPORTING ATPASE BETA-3 CHAIN |
| | | | (SODIUM/POTASSIUM-DEPENDENT ATPASE BETA-3 |
| | | | SUBUNIT) (ATPB-3). |
| 147 | Hs.15977 | Q9Y6M9 | NDUFB9: (NDUFB9 OR UQOR22) NADH-UBIQUINONE |
| | | | OXIDOREDUCTASE B22 SUBUNIT (EC 1.6.5.3) (EC |
| | | | 1.6.99.3) (COMPLEX I-B22) (CI-B22). |
| 148 | Hs.172674 | Q99842 | NFATX4: (NFATC3 OR NFAT4) NUCLEAR FACTOR OF |
| | | | ACTIVATED T-CELLS, CYTOPLASMIC 3 (T CELL |
| | | | TRANSCRIPTION FACTOR NFAT4) (NF-ATC3) (NF-AT4) |
| | | | (NFATX). |
| 149 | Hs.62041 | P14543 | NIDOGEN: (NID) NIDOGEN PRECURSOR (ENTACTIN). |
| 150 | Hs.111039 | P30419 | NMT1: (NMT1 OR NMT) GLYCYLPEPTIDE N- |
| | | | TETRADECANOYLTRANSFERASE 1 (EC 2.3.1.97) |
| | | | (PEPTIDE N-MYRISTOYLTRANSFERASE 1) |
| | | | (MYRISTOYL-COA: PROTEIN N- |
| | | | MYRISTOYLTRANSFERASE 1) (NMT 1). |
| 151 | Hs.724 | P20393 | NR1D1: (NR1D1 OR THRAL OR EAR1 OR HREV) |
| | | | ORPHAN NUCLEAR RECEPTOR NR1D1 (V-ERBA |
| | | | RELATED PROTEIN EAR-1) (REV-ERBA-ALPHA). |
| 152 | Hs.1119 | P22736 | NR4A1: (NR4A1 OR HMR OR NAK1 OR GFRP1) ORPHAN |
| | | | NUCLEAR RECEPTOR HMR (EARLY RESPONSE |
| | | | PROTEIN NAK1) (TR3 ORPHAN RECEPTOR) |
| 153 | Hs.82120 | P43354 | NR4A2: (NR4A2 OR NURR1 OR TINUR OR NOT) |
| | | | ORPHAN NUCLEAR RECEPTOR NURR1 |
| 154 | Hs.83469 | Q14494 | NRF1: (NFE2L1 OR NRF1 OR TCF11 OR HBZ17) |
| | | | NUCLEAR FACTOR ERYTHROID 2 RELATED FACTOR 1 |
| | | | (NF-E2 RELATED FACTOR 1) (NFE2-RELATED FACTOR |
| | | | 1) (NUCLEAR FACTOR, ERYTHROID DERIVED 2, LIKE 1) |
| | | | (TRANSCRIPTION FACTOR 11) (TRANSCRIPTION |
| | | | FACTOR HBZ17) |
| 155 | Hs.75212 | P11926 | ODC1: (ODC1) ORNITHINE DECARBOXYLASE (EC |
| | | | 4.1.1.17) (ODC). |
| 156 | Hs.88474 | P23219 | PGH1: (PTGS1 OR COX1) PROSTAGLANDIN G/H |
| | | | SYNTHASE 1 PRECURSOR (EC 1.14.99.1) |
| | | | (CYCLOOXYGENASE-1) (COX-1) (PROSTAGLANDIN- |
| | | | ENDOPEROXIDE SYNTHASE 1) (PROSTAGLANDIN |
| | | | H2SYNTHASE 1) (PGH SYNTHASE 1) (PGHS-1) (PHS 1). |
| 157 | Hs.76152 | P07585 | PGS2: (DCN) BONE PROTEOGLYCAN II PRECURSOR |
| | | | (PG-S2) (DECORIN) (PG40). |
| 158 | Hs.9589 | Q9UMX0 | PLIC-1: (UBQLN1) PLIC-1 UBIQUILIN. (DA41) |
| 159 | Hs.173902 | P30153 | PPP2R1A: (PPP2R1A) SERINE/THREONINE PROTEIN |
| | | | PHOSPHATASE 2A, 65 KDA REGULATORY SUBUNIT A, |
| | | | ALPHA ISOFORM (PP2A, SUBUNIT A, PR65-ALPHA |
| | | | ISOFORM) (PP2A, SUBUNIT A, R1-ALPHA ISOFORM) |
| | | | (MEDIUM TUMOR ANTIGEN-ASSOCIATED 61 KDA |
| | | | PROTEIN) |
| 160 | | P34062 | PSMA6: (PSMA6 OR PROS27) PROTEASOME IOTA |
| | | | CHAIN (EC 3.4.99.46) (MACROPAIN IOTA CHAIN) |
| | | | (MULTICATALYTIC ENDOPEPTIDASE COMPLEX IOTA |
| | | | CHAIN) (27 KDA PROSOMAL PROTEIN) (PROS-27) |
| | | | (P27K). |
| 161 | Hs.75748 | P20618 | PSMB1: (PSMB1 OR PSC5) PROTEASOME COMPONENT |
| | | | C5 |
| 162 | Hs.79387 | P47210 | PSMC5: (PSMC5 OR S8) 26S PROTEASOME |
| | | | REGULATORY ATPASE SUBUNIT 8 (P45) (TRIP1). |
| 163 | Hs.18700 | O75831 | PSMD13: (PSMD13) 26S PROTEASOME SUBUNIT S11 |
| | | | (P40.5) |
| 164 | Hs.74619 | Q13200 | PSMD2: (PSMD2 OR TRAP2) 26S PROTEASOME |
| | | | REGULATORY SUBUNIT S2 (P97) (TUMOR NECROSIS |
| | | | FACTOR TYPE 1 RECEPTOR ASSOCIATED PROTEIN 2). |
| 165 | Hs.9736 | O43242 | PSMD3: (PSMD3) 26S PROTEASOME REGULATORY |
| | | | SUBUNIT S3 (PROTEASOME SUBUNIT P58). |
| 166 | Hs.148495 | P55036 | PSMD4: (PSMD4 OR MCB1) 26S PROTEASOME |
| | | | REGULATORY SUBUNIT S5A (AF) (ASF). |
| 167 | Hs.155543 | P51665 | PSMD7: (PSMD7 OR MOV34L) 26S PROTEASOME |
| | | | REGULATORY SUBUNIT S12 (MOV34 PROTEIN). |
| 168 | Hs.178658 | P54727 | RAD23B: (RAD23B) UV EXCISION REPAIR PROTEIN |
| | | | PROTEIN RAD23 HOMOLOG B (HHR23B) (XP-C REPAIR |
| | | | COMPLEMENTING COMPLEX 58 KDA PROTEIN) (P58). |
| 169 | Hs.206097 | P17082 | RRAS2: (RRAS2) RAS-RELATED PROTEIN R-RAS2 |
| | | | (RAS-LIKE PROTEIN TC21) (TERATOCARCINOMA |
| | | | ONCOGENE). |
| 170 | Hs.252189 | P31431 | RYODOCAN: (SDC4) SYNDECAN-4 PRECURSOR |
| | | | (AMPHIGLYCAN) (SYND4) (RYUDOCAN CORE |
| | | | PROTEIN). |
| 171 | Hs.78575 | P07602 | “SGP1: (PSAP) PROACTIVATOR POLYPEPTIDE |
| | | | PRECURSOR [CONTAINS: SAPOSIN A (PROTEIN A); |
| | | | SAPOSIN B (SPHINGOLIPID ACTIVATOR PROTEIN 1) |
| | | | (SAP-1) (DISPERSIN) (SULFATIDE/GM1 ACTIVATOR); |
| | | | SAPOSIN C (CO-BETA-GLUCOSIDASE) (A1 ACTIVATOR) |
| | | | (GLUCOSYLCERAMIDASE ACTIVATOR)...” |
| 172 | Hs.63236 | O76070 | SNCG: (SNCG OR BCSG1) GAMMA-SYNUCLEIN |
| | | | (PERSYN) (BREAST CANCER-SPECIFIC GENE 1 |
| | | | PROTEIN). |
| 173 | Hs.75428 | P00441 | SOD1: (SOD1) SUPEROXIDE DISMUTASE [CU-ZN] (EC |
| | | | 1.15.1.1). |
| 174 | Hs.177781 | P04179 | SOD2: (SOD2 OR SOD-2) SUPEROXIDE DISMUTASE |
| | | | [MN], MITOCHONDRIAL PRECURSOR (EC 1.15.1.1). |
| 175 | Hs.111779 | P09486 | SPARC: (SPARC OR ON) SPARC PRECURSOR |
| | | | (SECRETED PROTEIN ACIDIC AND RICH IN CYSTEINE) |
| | | | (OSTEONECTIN) (ON) (BASEMENT MEMBRANE |
| | | | PROTEIN BM-40). |
| 176 | Hs.142258 | P40763 | STAT3: (STAT3 OR APRF) SIGNAL TRANSDUCER AND |
| | | | ACTIVATOR OF TRANSCRIPTION 3 (ACUTE-PHASE |
| | | | RESPONSE FACTOR). |
| 177 | Hs.150580 | P41567 | SUI1: (SUI1) PROTEIN TRANSLATION FACTOR SUI1 |
| | | | HOMOLOG (SUI1ISO1). |
| 178 | Hs.75356 | P15884 | TCF4: (TCF4 OR ITF2 OR SEF2) TRANSCRIPTION |
| | | | FACTOR 4 (IMMUNOGLOBULIN TRANSCRIPTION |
| | | | FACTOR 2) (ITF-2) (SL3-3 ENHANCER FACTOR 2) (SEF- |
| | | | 2). |
| 179 | Hs.125359 | P04216 | THY1: (THY1) THY-1 MEMBRANE GLYCOPROTEIN |
| | | | PRECURSOR (THY-1 ANTIGEN) (CDW90) (CD90 |
| | | | ANTIGEN). |
| 180 | Hs.6216 | O75472 | TID1: (TID1 OR TID-1) TUMOROUS IMAGINAL DISCS |
| | | | HOMOLOG PRECURSOR (HTID-1). |
| 181 | Hs.5831 | P01033 | TIMP1: (TIMP1 OR TIMP OR CLGI) |
| | | | METALLOPROTEINASE INHIBITOR 1 PRECURSOR |
| | | | (TIMP-1) (ERYTHROID POTENTIATING ACTIVITY) (EPA) |
| | | | (TISSUE INHIBITOR OF METALLOPROTEINASES) |
| | | | (FIBROBLAST COLLAGENASE INHIBITOR) |
| | | | (COLLAGENASE INHIBITOR). |
| 182 | Hs.6441 | P16035 | TIMP2: (TIMP2) METALLOPROTEINASE INHIBITOR 2 |
| | | | PRECURSOR (TIMP-2) (TISSUE INHIBITOR OF |
| | | | METALLOPROTEINASES-2) (CSC-21 K). |
| 183 | Hs.245188 | P35625 | TIMP3: (TIMP3) METALLOPROTEINASE INHIBITOR 3 |
| | | | PRECURSOR (TIMP-3) (TISSUE INHIBITOR OF |
| | | | METALLOPROTEINASES-3) (MIG-5 PROTEIN). |
| 184 | Hs.190787 | Q99727 | TIMP4: (TIMP4) METALLOPROTEINASE INHIBITOR 4 |
| | | | PRECURSOR (TIMP-4) (TISSUE INHIBITOR OF |
| | | | METALLOPROTEINASES-4). |
| 185 | Hs.105097 | P04183 | TK1: (TK1) THYMIDINE KINASE, CYTOSOLIC (EC |
| | | | 2.7.1.21). |
| 186 | Hs.118174 | P53804 | TPRD: (TTC3 OR TPRD) TETRATRICOPEPTIDE REPEAT |
| | | | PROTEIN 3 (TPR REPEAT PROTEIN D) MTPRD. |
| 187 | Hs.278242 | P04687 | TUBA: (TUBA1) TUBULIN ALPHA-1 CHAIN. |
| 188 | Hs.179661 | P07437 | TUBB: (TUBB1) TUBULIN BETA-1 CHAIN. |
| 189 | Hs.76136 | P10599 | TXN: (TXN OR TRDX OR TRX) THIOREDOXIN (ATL- |
| | | | DERIVED FACTOR) (ADF) (SURFACE ASSOCIATED |
| | | | SULPHYDRYL PROTEIN (SASP). |
| 190 | Hs.110802 | P04275 | VWF: (F8VWF OR VWF) VON WILLEBRAND FACTOR |
| | | | PRECURSOR. |
| 191 | Hs.75608 | Q15883 | X104: (X104 OR ZO-2) X104 (TIGHT JUNCTION PROTEIN |
| | | | ZO-2 ISOFORM A) (TIGHT JUNCTION PROTEIN ZO-2 |
| | | | ISOFORM C) (TIGHT JUNCTION PROTEIN ZO-2) |
| 192 | Hs.149923 | P17861 | XBP1: (XBP1 OR XBP2 OR TREB5) X BOX BINDING |
| | | | PROTEIN-1 (XBP-1) (TREB5 PROTEIN). (HTF) |
| | | | HEPATOCARCINOGENESIS-RELATED TRANSCRIPTION |
| | | | FACTOR (HTF). |
| 193 | Hs.4055 | Q99612 | ZF9: (COPEB OR BCD1 OR CPBP) CORE PROMOTER |
| | | | ELEMENT-BINDING PROTEIN (B-CELL DERIVED |
| | | | PROTEIN 1) (PROTO-ONCOGENE BCD1) (KRUEPPEL- |
| | | | LIKE FACTOR ZF9) (TRANSCRIPTION FACTOR ZF9) |
| | | | (GC-RICH SITES BINDING FACTOR GBF). |

TABLE 9

| | UniGene | | |
|---|---|---|---|
| | Accession | SWISSPROT | |
| No.: | No. | or TREMBL | Name of gene/Description |
| 1 | | | ACTA1_HUMAN: (ACTA1 OR ACTA) ACTIN, ALPHA |
| | | | SKELETAL MUSCLE (ALPHA-ACTIN 1). |
| 2 | | | ACTA1_MOUSE: (ACTA1 OR ACTA) ACTIN, ALPHA |
| | | | SKELETAL MUSCLE (ALPHA-ACTIN 1). |
| 3 | | | ADD3: (ADD3 OR ADDL) GAMMA ADDUCIN (ADDUCIN- |
| | | | LIKE PROTEIN 70). |
| 4 | | | AHCYL1: (AHCYL1 OR XPVKONA) PUTATIVE |
| | | | ADENOSYLHOMOCYSTEINASE (EC 3.3.1.1) (S- |
| | | | ADENOSYL-L-HOMOCYSTEINE HYDROLASE) |
| | | | (ADOHCYASE). |
| 5 | | | AK025194: AK025194 |
| 6 | | | APM2: (APM2) ADIPOSE MOST ABUNDANT GENE |
| | | | TRANSCRIPT 2. |
| 7 | | | ARHGAP1: (ARHGAP1 OR RHOGAP1 OR CDC42GAP) |
| | | | RHO-GTPASE-ACTIVATING PROTEIN 1 (GTPASE- |
| | | | ACTIVATING PROTEIN RHOOGAP) (RHO-RELATED |
| | | | SMALL GTPASE PROTEIN ACTIVATOR) (CDC42 |
| | | | GTPASE-ACTIVATING PROTEIN) (P50-RHOGAP). |
| 8 | | | ARPC4: (ARPC4 OR ARC20) ARP2/3 COMPLEX 20 KDA |
| | | | SUBUNIT (P20-ARC) (ACTIN-RELATED PROTEIN 2/3 |
| | | | COMPLEX SUBUNIT 4). |
| 9 | | | ATP1A1: (ATP1A1) SODIUM/POTASSIUM- |
| | | | TRANSPORTING ATPASE ALPHA-1 CHAIN PRECURSOR |
| | | | (EC 3.6.3.9) (SODIUM PUMP) (NA+/K+ ATPASE). |
| 10 | | | ATP6S14: (ATP6S14 OR VATF) VACUOLAR ATP |
| | | | SYNTHASE SUBUNIT F (EC 3.6.1.34) (V-ATPASE F |
| | | | SUBUNIT) (VACUOLAR PROTON PUMP F SUBUNIT) (V- |
| | | | ATPASE 14 KDA SUBUNIT). |
| 11 | | | B4-2: B4-2 PROTEIN. |
| 12 | | | BA217H1.1: (BA217H1.1) BA217H1.1 (SIMILAR TO N33 |
| | | | PROTEIN) (FRAGMENT). (DKFZP564K142). (IAG2) |
| | | | IMPLANTATION-ASSOCIATED PROTEIN. |
| 13 | | | BHMT2: (BHMT2) BETAINE-HOMOCYSTEINE |
| | | | METHYLTRANSFERASE 2. |
| 14 | | | BLP: (BLP OR KM23) BITHORAXOID-LIKE PROTEIN |
| | | | (HSPC162) (DYNEIN-ASSOCIATED PROTEIN HKM23) |
| | | | (HSPC162 PROTEIN). |
| 15 | | | BM-002: BM-002 (HYPOTHETICAL 9.1 KDA PROTEIN). |
| 16 | | | BM045: UNCHARACTERIZED BONE MARROW PROTEIN |
| | | | BM045. |
| 17 | | | C11ORF24: (C11ORF24) DM4E3. |
| 18 | | | C1QA: (C1QA) COMPLEMENT C1Q SUBCOMPONENT, A |
| | | | CHAIN PRECURSOR. |
| 19 | | | CG8989: (CG8989)) ((H3F3A OR HIS3.3A OR CG5825) |
| | | | AND (H3F3B OR HISH3-3Q OR HIS3) HISTONE H3.3 |
| | | | (H3.A) (H3.B) (H3.3Q). |
| 20 | | | CGBP: (CGBP) CPG BINDING PROTEIN. |
| 21 | | | CGI-149: CGI-149 PROTEIN. |
| 22 | | | CTSH: (CTSH) CATHEPSIN H PRECURSOR (EC |
| | | | 3.4.22.16). |
| 23 | | | CYBA_HUMAN: (CYBA) CYTOCHROME B-245 LIGHT |
| | | | CHAIN (P22 PHAGOCYTE B-CYTOCHROME) |
| | | | (NEUTROPHIL CYTOCHROME B, 22 KDA POLYPEPTIDE) |
| | | | (P22-PHOX) (CYTOCHROME B(558) ALPHA CHAIN) |
| | | | (SUPEROXIDE-GENERATING NADPH OXIDASE LIGHT |
| | | | CHAIN SUBUNIT). |
| 24 | | | CYBA_MOUSE: (CYBA) CYTOCHROME B-245 LIGHT |
| | | | CHAIN (P22 PHAGOCYTE B-CYTOCHROME) |
| | | | (NEUTROPHIL CYTOCHROME B, 22 KDA POLYPEPTIDE) |
| | | | (P22-PHOX) (CYTOCHROME B(558) ALPHA CHAIN) |
| | | | (SUPEROXIDE-GENERATING NADPH OXIDASE LIGHT |
| | | | CHAIN SUBUNIT). |
| 25 | | | DAXX: FAS-BINDING PROTEIN DAXX. |
| 26 | | | DJ1009E24.7: (DJ1009E24.7) DJ1009E24.7. |
| 27 | | | DJ159A19.3: (DJ159A19.3) DJ159A19.3 (NOVEL PROTEIN) |
| | | | (HYPOTHETICAL 26.4 KDA PROTEIN). |
| 28 | | | DKFZP434B044: (DKFZP434B044) HYPOTHETICAL 55.9 KDA |
| | | | PROTEIN. |
| 29 | | | DKFZP434M242: DKFZP434M242 |
| 30 | | | DKFZP547A023: (DKFZP547A023) HYPOTHETICAL 19.5 KDA |
| | | | PROTEIN (FRAGMENT). |
| 31 | | | DKFZP566B193: DKFZP566B193 |
| 32 | | | DKFZP761D0211: (DKFZP761D0211) HYPOTHETICAL 59.7 KDA |
| | | | PROTEIN. |
| 33 | | | DNCI2: (DNCI2 OR DNCIC2) DYNEIN INTERMEDIATE |
| | | | CHAIN 2, CYTOSOLIC (DH IC-2) (CYTOPLASMIC DYNEIN |
| | | | INTERMEDIATE CHAIN 2) (FRAGMENT). |
| 34 | | | DPM2: (DPM2) DOLICHOL PHOSPHATE-MANNOSE |
| | | | BIOSYNTHESIS REGULATORY PROTEIN. |
| 35 | | | DQ2A: HLA CLASS II HISTOCOMPATIBILITY ANTIGEN, |
| | | | DQ(2) ALPHA CHAIN PRECURSOR. H-2 CLASS II |
| | | | HISTOCOMPATIBILITY ANTIGEN, ALPHA CHAIN. |
| 36 | | | DSP: (DSP) DESMOPLAKIN (DP) (250/210 KDA |
| | | | PARANEOPLASTIC PEMPHIGUS ANTIGEN). |
| 37 | | | ECGF1: (ECGF1) THYMIDINE PHOSPHORYLASE |
| | | | PRECURSOR (EC 2.4.2.4) (TDRPASE) (TP) (PLATELET- |
| | | | DERIVED ENDOTHELIAL CELL GROWTH FACTOR) (PD- |
| | | | ECGF) (GLIOSTATIN). |
| 38 | | | EDAG: (EDAG) EDAG-1 -LIKE PROTEIN (HEMOGEN-1). |
| 39 | | | EFEMP2: (EFEMP2 OR FBLN4) EGF-CONTAINING |
| | | | FIBULIN-LIKE EXTRACELLULAR MATRIX PROTEIN 2 |
| | | | PRECURSOR (FIBULIN-4) (FIBL-4) (UPH1 PROTEIN). |
| 40 | | | EIF4G1: (EIF4G1 OR EIF4G) EUKARYOTIC TRANSLATION |
| | | | INITIATION FACTOR 4 GAMMA (EIF-4-GAMMA) (EIF-4G) |
| | | | (EIF4G) (P220). |
| 41 | | | EMI: (EMI) EMILIN PRECURSOR. |
| 42 | | | FB19: (FB19) FB19 PROTEIN (PNUTS). |
| 43 | | | FLJ11346: FLJ11346 |
| 44 | | | GTAR: (GTAR) GENE TRAP ANKYRIN REPEAT |
| | | | CONTAINING PROTEIN. |
| 45 | | | HSD17B7: (HSD17B7) ESTRADIOL 17 BETA- |
| | | | DEHYDROGENASE 7 (EC 1.1.1.62) (17-BETA-HSD 7) (17- |
| | | | BETA-HYDROXYSTEROID DEHYDROGENASE 7). |
| 46 | | | ICAT: (ICAT) BETA-CATENIN-INTERACTING PROTEIN |
| | | | ICAT. |
| 47 | | | KLF5: (KLF5 OR IKLF OR CKLF OR BTEB2) KRUEPPEL- |
| | | | LIKE FACTOR 5 (INTESTINAL-ENRICHED KRUEPPEL- |
| | | | LIKE FACTOR) (COLON KRUEPPEL-LIKE FACTOR) |
| | | | (TRANSCRIPTION FACTOR BTEB2) (BASIC |
| | | | TRANSCRIPTION ELEMENT BINDING PROTEIN 2) (GC |
| | | | BOX BINDING PROTEIN 2). |
| 48 | | | LAP: (LAP) CYTOSOL AMINOPEPTIDASE (EC 3.4.11.1) |
| | | | (LEUCINE AMINOPEPTIDASE) (LAP) (LEUCYL |
| | | | AMINOPEPTIDASE) (PROLINE AMINOPEPTIDASE) (EC |
| | | | 3.4.11.5) (PROLYL AMINOPEPTIDASE). |
| 49 | | | LMOD1: (LMOD1) LEIOMODIN 1 (LEIOMODIN, MUSCLE |
| | | | FORM) (64 KDA AUTOANTIGEN D1) (64 KDA |
| | | | AUTOANTIGEN 1D) (64 KDA AUTOANTIGEN 1D3) |
| | | | (THYROID-ASSOCIATED OPHTHALMOPATHY |
| | | | AUTOANTIGEN) (SMOOTH MUSCLE LEIOMODIN) (SM- |
| | | | LMOD). |
| 50 | | | LNV: (LNV) LNV. |
| 51 | | | MAP17: (MAP17) 17 KDA MEMBRANE ASSOCIATED |
| | | | PROTEIN (DD96 PROTEIN). |
| 52 | | | MLN51: (MLN51) MLN 51 PROTEIN. |
| 53 | | | NAF1: (NAF1 BETA) NAF1 BETA PROTEIN (HUMAN |
| | | | FETAL CRANIOFACIAL MRNA, PARTIAL CDS). |
| 54 | | | NCBP1: (NCBP1 OR NCBP OR CBP80) 80 KDA NUCLEAR |
| | | | CAP BINDING PROTEIN (NCBP 80 KDA SUBUNIT) |
| | | | (CBP80). |
| 55 | | | NUCKS: (NUCKS) NUCLEAR UBIQUITOUS CASEIN AND |
| | | | CYCLIN-DEPENDENT KINASES SUBSTRATE. |
| 56 | | | OS9: (OS9) PROTEIN OS-9 PRECURSOR. |
| 57 | | | Q16465: HYPOTHETICAL PROTEIN (FRAGMENT). |
| | | | (RPL41) HOMOLOGUE TO YEAST RIBOSOMAL PROTEIN |
| | | | L41. |
| 58 | | | Q9BSM6: SIMILAR TO RIKEN CDNA 2310040G17 GENE |
| | | | (FRAGMENT). |
| 59 | | | R32184_3: R32184_3. |
| 60 | | | RAN: (RAN) GTP-BINDING NUCLEAR PROTEIN RAN |
| | | | (TC4). |
| 61 | | | RPL13: (RPL13 OR BBC1) 60S RIBOSOMAL PROTEIN L13 |
| | | | (BREAST BASIC CONSERVED PROTEIN 1). |
| 62 | | | RPLP2: (RPLP2) 60S ACIDIC RIBOSOMAL PROTEIN P2. |
| 63 | | | RPS16: (RPS16) 40S RIBOSOMAL PROTEIN S16. |
| 64 | | | RPS19: (RPS19) 40S RIBOSOMAL PROTEIN S19. |
| 65 | | | RTN-X: (KIAA0886 OR RTN-X) KIAA0886 PROTEIN (RTN- |
| | | | XL) (RETICULON 4A). TESTIS SPECIFIC RETICULON 5 |
| | | | PROTEIN. BRAIN MY043 PROTEIN. (NOGO OR RTN-X) |
| | | | FOOCEN-M (NOGO-B PROTEIN) (RTN-XS) (RETICULON |
| | | | 4B). (ASY) ASY PROTEIN. (NOGO) FOOCEN-S (NOGO-C |
| | | | PROTEIN) (HYPOTHET |
| 66 | | | S100A10: (S100A10 OR CAL1L OR ANX2LG OR CLP11) |
| | | | CALPACTIN I LIGHT CHAIN (P10 PROTEIN) (P11) |
| | | | (CELLULAR LIGAND OF ANNEXIN II). |
| 67 | | | S100A2: (S100A2 OR S100L) S100 CALCIUM-BINDING |
| | | | PROTEIN A2 (S-100L PROTEIN) (CAN19). |
| 68 | | | SEMA3C: (SEMA3C OR SEMAE) SEMAPHORIN 3C |
| | | | PRECURSOR (SEMAPHORIN E) (SEMA E). |
| 69 | | | SEPT2: (SEPT2 OR NEDD5 OR DIFF6 OR KIAA0158) |
| | | | SEPTIN 2 (NEDD5 PROTEIN HOMOLOG). |
| 70 | | | SET: (SET) SET PROTEIN (HLA-DR ASSOCIATED |
| | | | PROTEIN II) (PHAPII) (PHOSPHATASE 2A INHIBITOR |
| | | | I2PP2A). |
| 71 | | | SFRS11: (SFRS11) SPLICING FACTOR |
| | | | ARGININE/SERINE-RICH 11 (ARGININE-RICH 54 KDA |
| | | | NUCLEAR PROTEIN) (P54). |
| 72 | | | SIR2L: (SIR2L OR SIRT2 OR SIR2L2) SILENCING |
| | | | INFORMATION REGULATOR 2-LIKE PROTEIN (SIR2 |
| | | | (SILENT MATING TYPE INFORMATION REGULATION 2, |
| | | | S. CEREVISIAE, HOMOLOG)-LIKE). |
| 73 | | | SLC9A1: (SLC9A1 OR NHE1 OR APNH1) |
| | | | SODIUM/HYDROGEN EXCHANGER 1 (NA(+)/H(+) |
| | | | EXCHANGER 1) (NHE-1) (NA+/H+ ANTIPORTER, |
| | | | AMILORIDE-SENSITIVE) (APNH). |
| 74 | | | SLU7: STEP II SPLICING FACTOR SLU7. |
| 75 | | | SMT3H2: (SMT3H2 OR SMT3B) UBIQUITIN-LIKE PROTEIN |
| | | | SMT3B (SENTRIN 2). |
| 76 | | | SORD: (SORD OR SDH1) SORBITOL DEHYDROGENASE |
| | | | (EC 1.1.1.14) (L-IDITOL 2-DEHYDROGENASE). |
| 77 | | | SOX20: (SOX20 OR SOX15 OR SOX-15) SOX-20 |
| | | | PROTEIN. |
| 78 | | | SRP9: (SRP9) SIGNAL RECOGNITION PARTICLE 9 KDA |
| | | | PROTEIN (SRP9). |
| 79 | | | SSRP1: (SSRP1 OR CIIDBP) STRUCTURE-SPECIFIC |
| | | | RECOGNITION PROTEIN 1 (SSRP1) (RECOMBINATION |
| | | | SIGNAL SEQUENCE RECOGNITION PROTEIN) (T160) |
| | | | (CHROMATIN-SPECIFIC TRANSCRIPTION ELONGATION |
| | | | FACTOR 80 KDA SUBUNIT) (FACT 80 KDA SUBUNIT). |
| 80 | | | STAB1: (STAB1) STABILIN-1. |
| 81 | | | SUV3: (SUV3) PUTATIVE ATP-DEPENDENT |
| | | | MITOCHONDRIAL RNA HELICASE. |
| 82 | | | TE2: (TE2 OR ARD1) N-TERMINAL |
| | | | ACETYLTRANSFERASE COMPLEX ARD1 SUBUNIT |
| | | | HOMOLOG. |
| 83 | | | TGFBI: (TGFBI OR BIGH3) TRANSFORMING GROWTH |
| | | | FACTOR-BETA INDUCED PROTEIN IG-H3 PRECURSOR |
| | | | (BETA IG-H3) (KERATO-EPITHELIN) (RGD-CONTAINING |
| | | | COLLAGEN ASSOCIATED PROTEIN) (RGD-CAP). |
| 84 | | | TPMT: (TPMT) THIOPURINE S-METHYLTRANSFERASE |
| | | | (EC 2.1.1.67) (THIOPURINE METHYL TRANSFERASE). |
| 85 | | | TRIM29A: ATAXIA-TELANGIECTASIA GROUP D- |
| | | | ASSOCIATED PROTEIN (TRIPARTITE MOTIF PROTEIN |
| | | | TRIM29 ALPHA). |
| 86 | | | TSTA3: (TSTA3 OR TSTAP35B OR P35B) GDP-FUCOSE |
| | | | SYNTHETASE (FX PROTEIN) (RED CELL NADP(H)- |
| | | | BINDING PROTEIN. |
| 87 | | | TTF-I-IP12: (FKSG13 OR PTRF) LEUCINE-ZIPPER |
| | | | PROTEIN FKSG13 (TTF-I INTERACTING PEPTIDE 12) |
| | | | (POLYMERASE I-TRANSCRIPT RELEASE FACTOR). |
| 88 | | | TUBA4: (TUBA4) TUBULIN ALPHA-4 CHAIN. |
| 89 | | | TUFT1: (TUFT1 OR DKFZP586G2219) TUFTELIN 1 |
| | | | (HYPOTHETICAL 44.3 KDA PROTEIN). |
| 90 | | | CHST5_MOUSE: (CHST5 OR I-GLCNAC-6-ST) N- |
| | | | ACETYLGLUCOSAMINE 6-O-SULFOTRANSFERASE. |
| 91 | | | EEF1A1: (EEF1A1 OR EEF1A OR EF1A) ELONGATION |
| | | | FACTOR 1-ALPHA 1 (EF-1-ALPHA-1) (ELONGATION |
| | | | FACTOR 1 A-1) (EEF1A-1) (ELONGATION FACTOR TU) |
| | | | (EF-TU). |
| 92 | | | EMBL_AL133429: EMBL_AL133429 |
| 93 | | | EMBL_BC009757: EMBL_BC009757 |
| 94 | | | EST00098: (EST00098) EST00098 PROTEIN |
| | | | (FRAGMENT). |
| 95 | | | EST_AA548686: EST_AA548686 |
| 96 | | | EST_AA552025: EST_AA552025 |
| 97 | | | EST_AA584843: EST_AA584843 |
| 98 | | | EST_AA640108: EST_AA640108 |
| 99 | | | EST_AA649141: EST_AA649141 |
| 100 | | | EST_AA725246: EST_AA725246 |
| 101 | | | EST_AA804235: EST_AA804235 |
| 102 | | | EST_AA913191: EST_AA913191 |
| 103 | | | EST_AI075228: EST_AI075228 |
| 104 | | | EST_AI089822: EST_AI089822 |
| 105 | | | EST_AI242082: EST_AI242082 |
| 106 | | | EST_AI354540: EST_AI354540 |
| 107 | | | EST_AI418576: EST_AI418576 |
| 108 | | | EST_AI732274: EST_AI732274 |
| 109 | | | EST_AL045661: EST_AL045661 |
| 110 | | | EST_AV735432: EST_AV735432 |
| 111 | | | EST_AW016700: EST_AW016700 |
| 112 | | | EST_AW137203: EST_AW137203 |
| 113 | | | EST_AW205184: EST_AW205184 |
| 114 | | | EST_AW296183: EST_AW296183 |
| 115 | | | EST_AW970604: EST_AW970604 |
| 116 | | | FER1L3: (FER1L3) FER-1 LIKE PROTEIN 3. |
| 117 | | | FLJ00075: (FLJ00075) FLJ00075 PROTEIN (FRAGMENT). |
| 118 | | | FLJ12408: CDNA FLJ12408 FIS, CLONE MAMMA1002869, |
| | | | HIGHLY SIMILAR TO PINCH PROTEIN. |
| 119 | | | FLJ12671: CDNA FLJ12671 FIS, CLONE NT2RM4002323, |
| | | | WEAKLY SIMILAR TO ANTIGEN GOR (SIMILAR TO |
| | | | HYPOTHETICAL PROTEIN FLJ12484). |
| 120 | | | FLJ12750: CDNA FLJ12750 FIS, CLONE NT2RP2001168, |
| | | | WEAKLY SIMILAR TO VERPROLIN (HYPOTHETICAL 31.3 KDA |
| | | | PROTEIN). |
| 121 | | | FLJ12875: CDNA FLJ12875 FIS, CLONE NT2RP2003777. |
| 122 | | | FLJ13110: CDNA FLJ13110 FIS, CLONE NT2RP3002549, |
| | | | MODERATELY SIMILAR TO HYPOTHETICAL 26.6 KD |
| | | | PROTEIN T19C3.4 IN CHROMOSOME III. |
| 123 | | | FLJ13388: FLJ13388 |
| 124 | | | “FLJ13631: CDNA FLJ13631 FIS, CLONE PLACE1011090, |
| | | | HIGHLY SIMILAR TO HOMO SAPIENS MRNA; CDNA |
| | | | DKFZP586A0522 (FROM CLONE DKFZP586A0522) |
| | | | (UNKNOWN) (PROTEIN FOR MGC: 11081).” |
| 125 | | | FLJ13855: CDNA FLJ13855 FIS, CLONE THYRO1000983, |
| | | | WEAKLY SIMILAR TO UBIQUITIN-CONJUGATING |
| | | | ENZYME E2-17 KD 9 (EC 6.3.2.19), FLJ 13968, CLONE |
| | | | Y9AA1001493. |
| 126 | | | FLJ14318: FLJ14318 |
| 127 | | | FLJ20037: CDNA FLJ20037 FIS, CLONE COL00314. |
| 128 | | | FLJ20288: CDNA FLJ20288 FIS, CLONE HEP04414 |
| | | | (FRAGMENT). |
| 129 | | | FLJ20297: CDNA FLJ20297 FIS, CLONE HEP05942. |
| 130 | | | FLJ20321: CDNA FLJ20321 FIS, CLONE HEP09380. |
| 131 | | | FLJ20396: CDNA FLJ20396 FIS, CLONE KAT00561 |
| | | | (HYPOTHETICAL 20.4 KDA PROTEIN). |
| 132 | | | FLJ20895: FLJ20895 |
| 133 | | | FLJ21120: CDNA: FLJ21120 FIS, CLONE CAS05691. |
| 134 | | | FLJ21289: FLJ21289 |
| 135 | | | FLJ21296: FLJ21296 |
| 136 | | | FLJ21839: CDNA: FLJ21839 FIS, CLONE HEP01794. |
| 137 | | | FLJ22428: (FBXW5) CDNA: FLJ22428 FIS, CLONE |
| | | | HRC09055 (WD REPEAT-CONTAINING F-BOX PROTEIN |
| | | | FBW5) (F-BOX AND WD-40 DOMAINPROTEIN 5). |
| 138 | | | FLJ22955: CDNA: FLJ22955 FIS, CLONE KAT09907. |
| 139 | | | FLJ23558: CDNA: FLJ23558 FIS, CLONE LNG09703. |
| 140 | | | GJB3_HUMAN: (GJB3 OR CX31) GAP JUNCTION BETA-3 |
| | | | PROTEIN (CONNEXIN 31) (CX31). |
| 141 | | | GLIPR: (GLIPR OR RTVP1) GLIOMA PATHOGENESIS- |
| | | | RELATED PROTEIN (RTVP-1 PROTEIN). |
| 142 | | | GNAS1: (GNAS1 OR GNAS OR GSP) GUANINE |
| | | | NUCLEOTIDE-BINDING PROTEIN G(S), ALPHA SUBUNIT |
| | | | (ADENYLATE CYCLASE-STIMULATING G ALPHA |
| | | | PROTEIN). (XLAS) G-PROTEIN XLAS. |
| 143 | | | GNB1: (GNB1) GUANINE NUCLEOTIDE-BINDING |
| | | | PROTEIN G(I)/G(S)/G(T) BETA SUBUNIT 1 (TRANSDUCIN |
| | | | BETA CHAIN 1). |
| 144 | | | GST4BETA_HUMAN: (GST4BETA OR CHST6) N- |
| | | | ACETYLGLUCOSAMINE 6-O-SULFOTRANSFERASE GST- |
| | | | 4BETA (CORNEAL N-ACETYLGLUCOSAMINE-6-O- |
| | | | SULFOTRANSFERASE). |
| 145 | | | GSTM2: (GSTM2 OR GST4) GLUTATHIONE S- |
| | | | TRANSFERASE MU 2 (EC 2.5.1.18) (GSTM2-2) (GST |
| | | | CLASS-MU). |
| 146 | | | GTF2F1: (GTF2F1 OR RAP74) TRANSCRIPTION |
| | | | INITIATION FACTOR IIF, ALPHA SUBUNIT (TFIIF-ALPHA) |
| | | | (TRANSCRIPTION INITIATION FACTOR RAP74). |
| 147 | | | H-SP1: (H-SP1) PANTOPHYSIN. |
| 148 | | | HASPP28: (HASPP28) 28 KDA HEAT-AND ACID-STABLE |
| | | | PHOSPHOPROTEIN (PDGF-ASSOCIATED PROTEIN). |
| 149 | | | HNRPL: (HNRPL) HETEROGENEOUS NUCLEAR |
| | | | RIBONUCLEOPROTEIN L (HNRNP L). |
| 150 | | | HSPC170: ADRENAL GLAND PROTEIN AD-001 (HSPC170 |
| | | | PROTEIN) (HSPC152). |
| 151 | | | HSPC195: HSPC195. |
| 152 | | | HSPC254: HSPC254 (FRAGMENT). |
| 153 | | | HSPC300: HSPC300 (FRAGMENT). |
| 154 | | | HSPC330: HSPC330 (FRAGMENT). |
| 155 | | | IGFBP4: (IGFBP4 OR IBP4) INSULIN-LIKE GROWTH |
| | | | FACTOR BINDING PROTEIN 4 PRECURSOR (IGFBP-4) |
| | | | (IBP-4) (IGF-BINDING PROTEIN 4). |
| 156 | | | IGHA1: (IGHA1) IG ALPHA-1 CHAIN C REGION. |
| 157 | | | IL22R: (IL22R) IL-22 RECEPTOR. |
| 158 | | | ITM2B: (ITM2B OR BRI) INTEGRAL MEMBRANE PROTEIN |
| | | | 2B (TRANSMEMBRANE PROTEIN BRI). |
| 159 | | | ITPKB: (ITPKB) 1D-MYO-INOSITOL-TRISPHOSPHATE 3- |
| | | | KINASE B (EC 2.7.1.127) (INOSITOL 1,4,5- |
| | | | TRISPHOSPHATE 3-KINASE) (IP3K) (IP3 3-KINASE) |
| | | | (FRAGMENT). |
| 160 | | | JANUS-A: SEX-REGULATED PROTEIN JANUS-A (CGI- |
| | | | 202). |
| 161 | | | KCNK6: (KCNK6 OR TWIK2 OR TOSS) POTASSIUM |
| | | | CHANNEL SUBFAMILY K MEMBER 6 (INWARD |
| | | | RECTIFYING POTASSIUM CHANNEL PROTEIN TWIK-2) |
| | | | (TWIK-ORIGINATED SIMILARITY SEQUENCE). |
| 162 | | | KIAA0127: (KIAA0127) HYPOTHETICAL PROTEIN |
| | | | KIAA0127. |
| 163 | | | KIAA0252: (KIAA0252) MYELOBLAST KIAA0252 |
| | | | (FRAGMENT). |
| 164 | | | KIAA0302: (KIAA0302 OR SPTBN2) BETA-SPECTRIN III |
| | | | (FNTA III SPECTRIN). |
| 165 | | | KIAA0346: (KIAA0346) KIAA0346 PROTEIN (FRAGMENT). |
| 166 | | | KIAA0661: (KIAA0661) KIAA0661 PROTEIN (95 KDA |
| | | | RETINOBLASTOMA PROTEIN BINDING |
| | | | PROTEIN, KIAA0661 GENE PRODUCT). |
| 167 | | | KIAA0720: (KIAA0720) KIAA0720 PROTEIN (FRAGMENT). |
| 168 | | | KIAA0731: (KIAA0731) KIAA0731 PROTEIN (FRAGMENT). |
| 169 | | | KIAA0876: (KIAA0876) KIAA0876 PROTEIN (FRAGMENT). |
| 170 | | | KIAA0911: (KIAA0911) KIAA0911 PROTEIN.(CSTN1 OR |
| | | | CALSYNTENIN-1) CALSYNTENIN-1 |
| 171 | | | KIAA1063: (KIAA1063) KIAA1063 PROTEIN (FRAGMENT). |
| 172 | | | KIAA1096: (KIAA1096) KIAA1096 PROTEIN (FRAGMENT). |
| 173 | | | KIAA1175: (KIAA1175) KIAA1175 PROTEIN (FRAGMENT). |
| 174 | | | KIAA1440: (KIAA1440) KIAA1440 PROTEIN (FRAGMENT). |
| 175 | | | KIAA1564: (KIAA1564) KIAA1564 PROTEIN (FRAGMENT). |
| 176 | | | KIAA1753: (KIAA1753) KIAA1753 PROTEIN (FRAGMENT). |
| 177 | | | KIAA1841: KIAA1841 PROTEIN. |
| 178 | | | LDHA: (LDHA) L-LACTATE DEHYDROGENASE M CHAIN |
| | | | (EC 1.1.1.27) (LDH-A). |
| 179 | | | LIPHB: (LIPHB) LIPOPHILIN B PRECURSOR. |
| 180 | | | MAZ: (MAZ) MYC-ASSOCIATED ZINC FINGER PROTEIN |
| | | | (MAZI) (PURINE-BINDING TRANSCRIPTION FACTOR) |
| | | | (PUR-1) (ZF87) (ZIF87). |
| 181 | | | MDH1: (MDH1 OR MDHA) MALATE DEHYDROGENASE, |
| | | | CYTOPLASMIC (EC 1.1.1.37). |
| 182 | | | MEN1: MEN1 |
| 183 | | | MGC2532: UNKNOWN (PROTEIN FOR MGC: 5178) |
| | | | (PROTEIN FOR MGC:2532). |
| 184 | | | MGC2749: HYPOTHETICAL 19.6 KDA PROTEIN |
| | | | (UNKNOWN) (PROTEIN FOR MGC:2749). |
| 185 | | | MIG-2: MIG-2 PROTEIN (FRAGMENT). |
| 186 | | | MT2A_HUMAN: (MT2A OR MT2) METALLOTHIONEIN-II |
| | | | (MT-II). |
| 187 | | | NAPA: (NAPA) ALPHA-SOLUBLE NSF ATTACHMENT |
| | | | PROTEIN (SNAP-ALPHA). |
| 188 | | | NCL: (NCL) NUCLEOLIN (PROTEIN C23). |
| 189 | | | NDUFA3: (NDUFA3) NADH-UBIQUINONE |
| | | | OXIDOREDUCTASE B9 SUBUNIT (EC 1.6.5.3) (EC |
| | | | 1.6.99.3) (COMPLEX I-B9) (CI-B9). |
| 190 | | | O75394: RIBOSOMAL PROTEIN L33-LIKE PROTEIN. |
| 191 | | | PABP2: (PABP2) POLY(A) BINDING PROTEIN II. |
| 192 | | | PALLID: PALLID (PALLID (MOUSE) HOMOLOG, |
| | | | PALLIDIN). |
| 193 | | | PARPL: (PARPL) PUTATIVE POLY(ADP-RIBOSYL) |
| | | | TRANSFERASE PRECURSOR. VAULT PROTEIN. |
| | | | (ADPRTL1) BA169O17.3 (ADP-RIBOSYLTRANSFERASE |
| | | | (NAD+, POLY (ADP-RIBOSE) POLYMERASE)-LIKE 1). |
| | | | (KIAA0177) KIAA0177 PROTEIN (FRAGMENT). |
| 194 | | | PCOLCE: (PCOLCE) PROCOLLAGEN C-PROTEINASE |
| | | | ENHANCER PROTEIN PRECURSOR (PCPE) (TYPE I |
| | | | PROCOLLAGEN COOH-TERMINAL PROTEINASE |
| | | | ENHANCER) (TYPE 1 PROCOLLAGEN C-PROTEINASE |
| | | | ENHANCER PROTEIN). |
| 195 | | | PFKL: (PFKL) 6-PHOSPHOFRUCTOKINASE, LIVER TYPE |
| | | | (EC 2.7.1.11) (PHOSPHOFRUCTOKINASE 1) |
| | | | (PHOSPHOHEXOKINASE) (PHOSPHOFRUCTO-1-KINASE |
| | | | ISOZYME B) (PFK-B). |
| 196 | | | PI4KB: (PI4KB) PHOSPHATIDYLINOSITOL 4-KINASE. |
| 197 | | | PIR: (PIR) PIRIN. |
| 198 | | | PLASMOLIPIN: PLASMOLIPIN. |
| 199 | | | PNAS-110: PNAS-110. |
| 200 | | | PPL: (PPL OR KIAA0568) PERIPLAKIN (195 KDA |
| | | | CORNIFIED ENVELOPE PRECURSOR) (190 KDA |
| | | | PARANEOPLASTIC PEMPHIGUS ANTIGEN). |
| 201 | | | PQBP-1: (PQBP-1 OR JM26 OR NPW38) |
| | | | POLYGLUTAMINE BINDING PROTEIN 1 (JM26 PROTEIN) |
| | | | (PQBP-1). |
| 202 | | | PSMF1: (PSMF1) DJ545L17.3 (PROTEASOME |
| | | | (PROSOME, MACROPAIN) INHIBITOR SUBUNIT 1 (PI31)). |
| 203 | | | Q04323: HYPOTHETICAL 33.4 KDA PROTEIN. |
| 204 | | | Q9BRK3: SIMILAR TO RIKEN CDNA 1200013A08 GENE. |
| 205 | | | Q9BRX8: SIMILAR TO RIKEN CDNA 5730469M10 GENE. |
| 206 | | | Q9BV68: HYPOTHETICAL 35.6 KDA PROTEIN. |
| 207 | | | Q9BWN5: SIMILAR TO ILVB (BACTERIAL |
| | | | ACETOLACTATE SYNTHASE)-LIKE. |
| 208 | | | Q9Y475: INOSITOL 1,4,5-TRISPHOSPHATE 3-KINASE |
| | | | ISOENZYME (EC 2.7.1.127) (FRAGMENT). |
| 209 | | | RAB11A: (RAB11A OR RAB11) RAS-RELATED PROTEIN |
| | | | RAB-11A (RAB-11) (24KG) (YL8). |
| 210 | | | RAB2: (RAB2) RAS-RELATED PROTEIN RAB-2. |
| 211 | | | RALGDS: (RALGDS OR RGF) RAL GUANINE |
| | | | NUCLEOTIDE DISSOCIATION STIMULATOR (RALGEF) |
| | | | (RALGDS). |
| 212 | | | RBM6: (RBM6 OR DEF3) RNA-BINDING PROTEIN 6 (RNA |
| | | | BINDING MOTIF PROTEIN 6) (RNA-BINDING PROTEIN |
| | | | DEF-3) (LUNG CANCER ANTIGEN NY-LU-12) (PROTEIN |
| | | | G16). |
| 213 | | | RGS10: (RGS10) REGULATOR OF G-PROTEIN |
| | | | SIGNALING 10 (RGS10). |
| 214 | | | RHOIP3: (RHOIP3) RHO-INTERACTING PROTEIN 3 |
| | | | (P116RIP) (RIP3). (KIAA0864) KIAA0864 PROTEIN |
| | | | (FRAGMENT). |
| 215 | | | RIS: (RIS) RIS. |
| 216 | | | RLIP76: (RIP1) RLIP76 PROTEIN, RAL-INTERACTING |
| | | | PROTEIN 1 (RIP1 PROTEIN) RALBP1. |
| 217 | | | RNPS1: (RNPS1) SR PROTEIN (RIBONUCLEIC ACID |
| | | | BINDING PROTEIN S1 (RNA/DNA-BINDING PROTEIN)). |
| 218 | | | RPL14: (RPL14) 60S RIBOSOMAL PROTEIN L14 (CAG-ISL7). |
| 219 | | | SB135: (MYADM OR MUG) MYELOID-ASSOCIATED |
| | | | DIFFERENTIATION MARKER (MYELOID UPREGULATED |
| | | | PROTEIN) (SB135). |
| 220 | | | SEPP1: (SEPP1 OR SELP) SELENOPROTEIN P |
| | | | PRECURSOR (SEP). |
| 221 | | | SF3B2: (SF3B2 OR SAP145) SPLICING FACTOR 3B |
| | | | PROTEIN, SUBUNIT 2 (SF3B150) (SPLICEOSOME |
| | | | ASSOCIATED PROTEIN 145) (SAP 145). |
| 222 | | | SOD3: (SOD3) EXTRACELLULAR SUPEROXIDE |
| | | | DISMUTASE PRECURSOR (EC 1.15.1.1) (EC-SOD). |
| 223 | | | SQSTM1_HUMAN: (SQSTM1 OR OSI) OXIDATIVE |
| | | | STRESS INDUCED PHOSPHOTYROSINE INDEPENDENT |
| | | | LIGAND FOR THE LCK SH2 DOMAIN P62 |
| | | | (SEQUESTOSOME 1). EBI3-ASSOCIATED PROTEIN P60. |
| | | | PKC-ZETA-INTERACTING PROTEIN (ZIP). |
| 224 | | | SQSTM1_MOUSE: (SQSTM1 OR OSI) OXIDATIVE |
| | | | STRESS INDUCED PHOSPHOTYROSINE INDEPENDENT |
| | | | LIGAND FOR THE LCK SH2 DOMAIN P62 |
| | | | (SEQUESTOSOME 1). EBI3-ASSOCIATED PROTEIN P60. |
| | | | PKC-ZETA-INTERACTING PROTEIN (ZIP). |
| 225 | | | SUN2: (SUN2) SAD1 UNC-84 DOMAIN PROTEIN 2 |
| | | | (FRAGMENT). (KIAA0668 OR DJ508115.4) KIAA0668 |
| | | | PROTEIN (FRAGMENT). |
| 226 | | | TM4SF2: (TM4SF2 OR MXS1 OR A15) TRANSMEMBRANE |
| | | | 4 SUPERFAMILY, MEMBER 2 (CELL SURFACE |
| | | | GLYCOPROTEIN A15) (T-CELL ACUTE LYMPHOBLASTIC |
| | | | LEUKEMIA ASSOCIATED ANTIGEN 1) (TALLA-1) |
| | | | (MEMBRANE COMPONENT, X CHROMOSOME, |
| | | | SURFACE MARKER 1). |
| 227 | | | UGP2: (UGP2) UTP —GLUCOSE-1-PHOSPHATE |
| | | | URIDYLYLTRANSFERASE 2 (EC 2.7.7.9) (UDP-GLUCOSE |
| | | | PYROPHOSPHORYLASE 2) (UDPGP 2) (UGPASE 2). |
| 228 | | | VAMP5: (VAMP5) VESICULE-ASSOCIATED MEMBRANE |
| | | | PROTEIN 5 (VAMP-5) (MYOBREVIN) (HSPC191). |
| 229 | | | WDR1: (WDR1) WD-REPEAT PROTEIN 1 (ACTIN |
| | | | INTERACTING PROTEIN 1) (NORI-1). |
| 230 | | | XAB2: (XAB2) XAB2. |
| 231 | | | ZB42D04: ZB42D04 |
| 232 | | | ZNF220: (ZNF220 OR MOZ) MONOCYTIC LEUKEMIA ZINC |
| | | | FINGER PROTEIN (ZINC FINGER PROTEIN 220). |
| 233 | | | ZNF6: (ZNF6) ZINC FINGER TRANSCRIPTION FACTOR. |
| 234 | | | ZNFN2A1: (ZNFN2A1) DOUBLE FYVE-CONTAINING |
| | | | PROTEIN 1. |

1. A process for the in vitro identification of the genes relevant to ageing of the skin and/or to skin stress in human beings or animals, characterized in that
a) a first mixture of genetically coded factors expressed, i.e. transcribed and optionally translated, in human or animal skin, i.e. a mixture of proteins, mRNA molecules or fragments of proteins or mRNA molecules, is isolated from young human or animal skin,
b) a second mixture of genetically coded factors expressed, i.e. transcribed and optionally translated, in human or animal skin, i.e. a mixture of proteins, mRNA molecules or fragments of proteins or mRNA molecules, is isolated from old human or animal skin,
c) the mixtures isolated in a) and b) are subjected to a serial analysis of gene expression (SAGE) so that the genes expressed to different extents (differentially) in old and young skin are identified.

2. A process for the in vitro determination of skin stress and/or ageing of the skin in human beings or animals, characterized in that
a) a mixture of proteins, mRNA molecules or fragments of proteins or mRNA molecules is isolated from human or animal skin,
b) the mixture isolated is tested for the presence and optionally the quantity of at least one of the proteins, mRNA molecules or fragments of proteins or mRNA molecules identified as expressed differentially in old and young skin by serial analysis of gene expression (SAGE),
c) the test results from b) are compared with the expression patterns identified by serial analysis of gene expression (SAGE) and
d) the mixture tested in b) is assigned to old or stressed skin if it predominantly contains proteins, mRNA molecules or fragments of proteins or mRNA molecules which are expressed more strongly in old or stressed skin than in young or unstressed skin or the mixture tested in b) is assigned to young or unstressed skin if it predominantly contains proteins, mRNA molecules or fragments of proteins or mRNA molecules which are expressed more strongly in young or unstressed skin than in old or stressed skin.

3. A process as claimed in claim 2, characterized in that, in step b), the mixture isolated is tested for the presence and optionally the quantity of at least one of the proteins, mRNA molecules or fragments of proteins or mRNA molecules defined by their UniGene Accession Number in Tables 1 to 4, column 7; in step c), the test results from b) are compared with the relative expression frequencies shown in Tables 1 to 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least twice as strongly in old or stressed skin as in young or unstressed skin or the mixture tested in b) is assigned to young or unstressed skin if it predominantly contains proteins, mRNA molecules or fragments of proteins or mRNA molecules which are expressed at least twice as strongly in young or unstressed skin as in old or stressed skin.

4. A process as claimed in claim 2 or 3, characterized in that, in step b), the mixture isolated is tested for the presence and optionally the quantity of at least one of the proteins, mRNA molecules or fragments of proteins or mRNA molecules defined by their UniGene Accession Number in Tables 2 to 4, column 7; in step c), the test results from b) are compared with the relative expression frequencies shown in Tables 2 to 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least five times as strongly in old or stressed skin as in young or unstressed skin or the mixture tested in b) is assigned to young or unstressed skin if it predominantly contains proteins, mRNA molecules or fragments of proteins or mRNA molecules which are expressed at least five times as strongly in young or unstressed skin as in old or stressed skin.

5. A process as claimed in any of claims 2 to 4, characterized in that, in step b), the mixture isolated is tested for the presence and optionally the quantity of at least one of the proteins, mRNA molecules or fragments of proteins or mRNA molecules defined by their UniGene Accession Number in Tables 3 and 4, column 7; in step c), the test results from b) are compared with the relative expression frequencies shown in Tables 3 and 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least seven times as strongly in old or stressed skin as in young or unstressed skin or the mixture tested in b) is assigned to young or unstressed skin if it predominantly contains proteins, mRNA molecules or fragments of proteins or mRNA molecules which are expressed at least seven times as strongly in young or unstressed skin as in old or stressed skin.

6. A process as claimed in any of claims 2 to 5, characterized in that, in step b), the mixture isolated is tested for the presence and optionally the quantity of at least one of the proteins, mRNA molecules or fragments of proteins or mRNA molecules defined by their UniGene Accession Number in Table 4, column 7; in step c), the test results from b) are compared with the relative expression frequencies shown in Table 4, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least ten times as strongly in old or stressed skin as in young or unstressed skin or the mixture tested in b) is assigned to young or unstressed skin if it predominantly contains proteins, mRNA molecules or fragments of proteins or mRNA molecules which are expressed at least ten times as strongly in young or unstressed skin as in old or stressed skin.

7. A process as claimed in claim 2, characterized in that, in step b), the mixture isolated is tested for the presence and optionally the quantity of at least one of the proteins, mRNA molecules or fragments of proteins or mRNA molecules defined by their 11-base tag sequence in Table 5 or in Table 7, column 2; in step c), the test results from b) are compared with the relative expression frequencies shown in Table 5 or in Table 7, columns 3 and 4, and the expression quotients indicated in column 5; and in step d), the mixture tested in b) is assigned to old or stressed skin if it predominantly contains proteins, mRNA molecules or fragments of proteins or mRNA molecules which are expressed at least twice, more particularly five times, preferably seven times and more preferably ten times as strongly in old or stressed skin as in young or unstressed skin or the mixture tested in b) is assigned to young or unstressed skin if it predominantly contains proteins, mRNA molecules or fragments of proteins or mRNA molecules which are expressed at least twice, more particularly five times, preferably seven times and more preferably ten times as strongly in young or unstressed skin as in old or stressed skin.

8. A process for the in vitro determination of skin stress and/or ageing of the skin in human beings or animals, characterized in that
a) a mixture of proteins, mRNA molecules or fragments of proteins or mRNA molecules is isolated from human or animal skin,
b) in the mixture isolated, at least two of the proteins, mRNA molecules or fragments of proteins or mRNA molecules identified as important to skin ageing and/or skin stress by the process claimed in claim 1 are quantified,
c) the expression ratios of the at least two proteins, mRNA molecules or fragments of proteins or mRNA molecules to one another are determined,
d) the expression ratios from c) are compared with the expression ratios typically present in young skin or in old skin for the molecules quantified in b), more particularly with the expression ratios shown in Tables 1 to 5 and 7, columns 3 and 4 and
e) the mixture isolated in a) is assigned to old or stressed skin if the expression ratios of the skin under analysis correspond to the expression ratios in old skin or the mixture isolated in a) is assigned to young or unstressed skin if the expression ratios of the skin under analysis correspond to from the expression ratios in young skin.

9. A process as claimed in any of claims 1 to 8, characterized in that, in step a), the mixture is isolated from a skin sample, more particularly from a whole skin sample or from an epidermis sample.

10. A process as claimed in any of claims 2 to 8, characterized in that, in step a), the mixture is obtained by microdialysis.

11. A process as claimed in any of claims 2 to 7, 9 and 10, characterized in that, in step b), testing for the presence and optionally the quantity of at least one of the proteins or protein fragments is carried out by a method selected from
i. one- or two-dimensional gel electrophoresis
ii. affinity chromatography
iii. protein/protein complexing in solution
iv. mass spectrometry, more particularly Matrix Assisted Laser Desorption Ionization (MALDI) and, more particularly,
v. the use of protein chips,
or suitable combinations of these methods.

12. A process as claimed in any of claims 8 to 10, characterized in that, in step b), the quantification of at least two proteins or protein fragments is carried out by a method selected from
i. one- or two-dimensional gel electrophoresis
ii. affinity chromatography
iii. protein/protein complexing in solution
iv. mass spectrometry, more particularly Matrix Assisted Laser Desorption Ionization (MALDI) and, more particularly,
v. the use of protein chips,
or suitable combinations of these methods.

13. A process as claimed in any of claims 2 to 7, 9 and 10, characterized in that, in step b), testing for the presence and optionally the quantity of at least one of the mRNA molecules or mRNA molecule fragments is carried out by a method selected from
i. northern blots,
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA)
xi. serial analysis of gene expression (SAGE) and, more particularly,
xii. the use of nucleic acid chips
or suitable combinations of these methods.

14. A process as claimed in any of claims 8 to 10, characterized in that in step b), the quantification of at least two mRNA molecules or mRNA molecule fragments is carried out by a method selected from
i. northern blots,
ii. reverse transcriptase polymerase chain reaction (RT-PCR),
iii. RNase protection experiments,
iv. dot blots,
v. cDNA sequencing,
vi. clone hybridization,
vii. differential display,
viii. subtractive hybridization,
ix. cDNA fragment fingerprinting,
x. total gene expression analysis (TOGA)
xi. serial analysis of gene expression (SAGE) and, more particularly,
xii. the use of nucleic acid chips
or suitable combinations of these methods.

15. A process as claimed in any of claims 2 to 7, 9, 10, 11 and 13, characterized in that step b) comprises testing for the presence and optionally the quantity of 1 to about 5,000, preferably 1 to about 1,000, more preferably about 10 to about 500, most preferably about 10 to about 250, more particularly about 10 to about 100 and most particularly about 10 to about 50 of the proteins, mRNA molecules or fragments of proteins or mRNA molecules which are defined
i. by their Unigene Accession Number in Tables 1 to 4, column 7, or
ii. by their UniGene Accession Number in Table 6 or 8, column 2, or by their Swissprot or TREMBL number in column 3 or by their EMBL/Genbank number in column 4 or by the name of the gene in Table 9 or
iii. by their 11-base tag sequence in Table 5 or in Table 7, column 2.

16. A process as claimed in any of claims 8 to 10, 12 and 14, characterized in that 1 to about 5,000, preferably 1 to about 1,000, more preferably about 10 to about 500, most preferably about 10 to about 250, more particularly about 10 to about 100 and most particularly about 10 to about 50 of the proteins, mRNA molecules or fragments of proteins or mRNA molecules which are defined
i. by their Unigene Accession Number in Tables 1 to 4, column 7, or
ii. by their UniGene Accession Number in Table 6 or 8, column 2, or by their Swissprot or TREMBL number in column 3 or by their EMBL/Genbank number in column 4 or by the name of the gene in Table 9 or
iii. by their 11-base tag sequence in Table 5 or in Table 7, column 2
are quantified in step b).

17. A test kit for the in vitro determination of skin stress and/or ageing of the skin in human beings or animals comprising means for carrying out the process claimed in any of claims 2 to 16.

18. A biochip for the in vitro determination of skin stress and/or ageing of the skin in human beings or animals comprising
i. a support and,
ii. immobilized thereon, probes which are capable of binding specifically to at least one of the proteins, mRNA molecules or fragments of proteins or mRNA molecules which are defined by their UniGene Accession Number in Tables 1 to 4, column 7, or by their 11-base tag sequence in Table 5 or in Table 7, column 2.

19. A biochip as claimed in claim 18 comprising 1 to about 5,000, preferably 1 to about 1,000, more preferably about 10 to about 500, most preferably about 10 to about 250, more particularly about 10 to about 100 and most particularly about 10 to about 50 different probes.

20. A biochip as claimed in claim 18 or 19 comprising nucleic acid probes, more particularly RNA or PNA probes and most particularly DNA probes.

21. A biochip as claimed in claim 20 comprising probes with a length of about 10 to about 1,000, more preferably with a length of about 10 to about 800, most preferably with a length of about 100 to about 600 and, in one most particularly preferred embodiment, with a length of about 200 to about 400 nucleotides.

22. A biochip as claimed in claim 18 or 19 comprising peptide or protein probes, more particularly antibodies.

23. A biochip as claimed in any of claims 18 to 22 comprising probes which are capable of binding specifically to at least one of the proteins, mRNA molecules or fragments of proteins or mRNA molecules which are identified by their UniGene Accession Number in Table 6 or 8, column 2, or by their Swissprot or TREMBL number in column 3 or by their EMBL/Genbank number in column 4 or by the name of the gene in Table 9.

24. The use of the proteins, mRNA molecules or fragments of proteins or mRNA molecules which are defined
i. by their Unigene Accession Number in Tables 1 to 4, column 7, or
ii. by their UniGene Accession Number in Table 6 or 8, column 2, or by their Swissprot or TREMBL number in column 3 or by their EMBL/Genbank number in column 4 or by the name of the gene in Table 9 or
iii. by their 11-base tag sequence in Table 5 or in Table 7, column 2,
as skin stress and/or skin ageing markers in human beings or animals.

25. A test for demonstrating the effectiveness of cosmetic or pharmaceutical active substances against skin stress and/or ageing of the skin in vitro, characterized in that
a) the status of the skin is determined by the process claimed in any of claims 2 to 16 or with the aid of a test kit claimed in claim 17 or with the aid of the biochip claimed in any of claims 18 to 23,
b) an active substance against skin stress and/or ageing of the skin is applied one or more times to the skin,
c) the status of the skin is re-determined by the process claimed in any of claims 2 to 16 or with the aid of a test kit claimed in claim 17 or with the aid of the biochip claimed in any of claims 18 to 23 and
d) the effectiveness of the active substance is determined by comparing the results from a) and c).

26. A test kit for demonstrating the effectiveness of cosmetic or pharmaceutical active substances against skin stress and/or ageing of the skin in vitro comprising means for carrying out the test claimed in claim 25.

27. The use of the proteins, mRNA molecules or fragments of proteins or mRNA molecules which are defined
i. by their Unigene Accession Number in Tables 1 to 4, column 7, or
ii. by their UniGene Accession Number in Table 6 or 8, column 2, or by their Swissprot or TREMBL number in column 3 or by their EMBL/Genbank number in column 4 or by the name of the gene in Table 9 or
iii. by their 11-base tag sequence in Table 5 or in Table 7, column 2,
for demonstrating the effectiveness of cosmetic or pharmaceutical active substances against skin stress and/or ageing of the skin.

28. A screening process for identifying cosmetic or pharmaceutical active substances against skin stress and/or ageing of the skin in vitro, characterized in that
a) the status of the skin is determined by the process claimed in any of claims 2 to 16 or with the aid of a test kit claimed in claim 17 or with the aid of the biochip claimed in any of claims 18 to 23,
b) a potential active substance against skin stress and/or ageing of the skin is applied one or more times to the skin,
c) the status of the skin is re-determined by the process claimed in any of claims 2 to 16 or with the aid of a test kit claimed in claim 17 or with the aid of the biochip claimed in any of claims 18 to 23 and
d) effective active substances are determined by comparing the results from a) and c).

29. The use of the proteins, mRNA molecules or fragments of proteins or mRNA molecules which are defined
i. by their Unigene Accession Number in Tables 1 to 4, column 7, or
ii. by their UniGene Accession Number in Table 6 or 8, column 2, or by their Swissprot or TREMBL number in column 3 or by their EMBL/Genbank number in column 4 or by the name of the gene in Table 9 or
iii. by their 11-base tag sequence in Table 5 or in Table 7, column 2,
for identifying cosmetic or pharmaceutical active substances against skin stress and/or ageing of the skin.

30. A process for the production of a cosmetic or pharmaceutical preparation against skin stress and/or ageing of the skin, characterized in that
a) active substances are determined by the process claimed in claim 28 or by the use claimed in claim 29 and
b) active substances found to be effective are mixed with cosmetically and pharmacologically suitable and compatible carriers.

31. A cosmetic or pharmaceutical preparation against skin stress and/or ageing of the skin containing at least one nucleic acid construct which is capable of suppressing or reducing the activity of at least one of the proteins that are expressed more strongly in old or stressed skin than in young or unstressed skin or of inducing or strengthening the activity of at least one of the proteins that are expressed more strongly in young or unstressed skin than in old or stressed skin.

32. A preparation as claimed in claim 31, characterized in that the proteins(s) is/are preferably selected from those which are defined
a) by their Unigene Accession Number in Tables 1 to 4, column 7, or
b) by their UniGene Accession Number in Table 6 or 8, column 2, or by their Swissprot or TREMBL number in column 3 or by their EMBL/Genbank number in column 4 or by the name of the gene in Table 9 or
c) by their 11-base tag sequence in Table 5 or in Table 7, column 2.

33. A preparation as claimed in claim 31 or 32, characterized in that the nucleic acid construct is selected from DNA, RNA or PNA.

34. A preparation as claimed in any of claims 31 to 33, characterized in that it contains about 1,000, more particularly about 10 to about 500, preferably about 10 to about 250, more preferably about 10 to about 100 and most preferably about 10 to about 50 different nucleic acid constructs.

35. A preparation as claimed in any of claims 31 to 34, characterized in that the nucleic acid construct is selected from protein-coding sequences, ribozymes, antisense nucleic acids, triple helix formers and rRNA.

36. A preparation as claimed in any of claims 31 to 35, characterized in that the nucleic acid construct is encapsulated in lipid vesicles, for example in liposomes, niosomes or transfersomes, preferably in liposomes.
